(19) United States
(12) Patent Application Publication
(10) Pub. No.: US 2026/0258086 A1
(43) Pub. Date: Sep. 3, 2026

(54) MODIFIED AMATOXINS AND USES THEREOF

(71) Applicant: THE UNIVERSITY OF BRITISH COLUMBIA, Vancouver (CA)

(72) Inventors: David Perrin, Vancouver (CA); Mihajlo Todorovic, Cambridge, MA (US); Kayla C. Newell, Vancouver (CA)

(21) Appl. No.: 18/715,290

(22) PCT Filed: Dec. 2, 2022
(86) PCT No.: PCT/CA2022/051775

§ 371 Date: May 31, 2024

Related U.S. Application Data
(60) Provisional application No. 63/285,414, filed on Dec. 2, 2021.

(51) Int. Cl. C07K 7/64; A01N 37/46; A01P 15/00; A61K 38/00; A61K 47/68

(52) U.S. Cl. CPC C07K 7/64; A01N 37/46; A01P 15/00; A61K 47/6831; A61K 47/6889; A61K 38/00

(57) ABSTRACT

The present disclosure relates to amatoxin analogs comprising one or more modifications of eastern ring residues, constructs comprising such amatoxin analogs coupled to a linker and conjugates comprising such amatoxin analogs or compound-linker constructs. The present disclosure also relates to uses of such amatoxin analogs, for example, in treatment of cancer. For example, the amatoxin analog can be a compound of Formula (I).

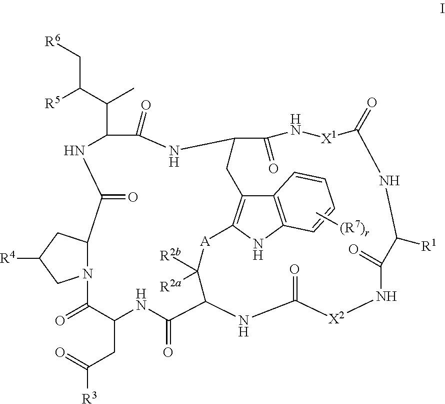

### CROSS-REFERENCE TO RELATED APPLICATIONS

The present application claims the benefit of priority from co-pending U.S. provisional application No. 63/285,414 filed on Dec. 2, 2021, the contents of which are incorporated herein by reference in their entirety.

### FIELD

The present disclosure relates to amatoxin analogs, conjugates comprising such amatoxin analogs and their use, for example, in the treatment of cancer.

### BACKGROUND

Alpha-amanitin, produced by A. phalloides, the leading cause of fatal mushroom poisonings, is a natural product with a rich history and pharmacological utility (Wieland et al., 1991; Vetter, 1998). A bicyclic octapeptide with a unique hydroxytryptathionine-(R)-sulfoxide staple (Scheme 1), it shows oral bioavailability, target specificity, and extraordinary toxicity (0.05-0.1 mg/kg).

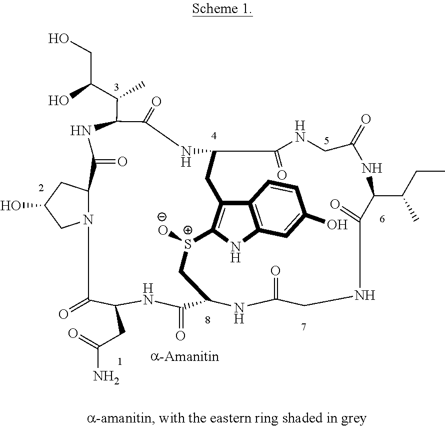

Within the amatoxins group, nine related structures have been identified. All of these peptides represent the same amino acid sequence with the only difference being degrees of oxidation of the side chain. Each mushroom in the Amanita family produces these peptides in varying amounts, which leads to differing physiological effects of the toxin for each of the different mushrooms (Table 1). The isolation and characterization of α-amanitin as an allosteric inhibitor of RNA Polymerase II (Pol II) in the early 1970s represented a key milestone in chemical biology and molecular pharmacology (Cochetme & Chambon, 1974; Kedinger et al., 1970); by 1980, α-amanitin had become the primary reagent required to implicate Pol II in the biosynthetic origin of specific mRNA sequences. Intense research culminated in x-ray (Bushnell et al., 2002; Wang et al., 2009) and cryo-EM structures (Brueckner & Cramer, 2008; Cheung & Cramer, 2012; Liu et al., 2018) of α-amanitin-bound Pol II that elucidated an extensive array of non-covalent interactions (e.g. H-bonding, hydrophobic, pi-stacking, pi-cation) by which α-amanitin contacts Pol II.

Amatoxins have, for example, been identified as attractive candidates for use as chemotherapeutics in cancer treatment, given that they are selectively delivered to cancer cells. Since transcription is required for cellular homeostasis and growth, α-amanitin is unique in its ability to kill both dividing and quiescent cells (Andera & Wasylyk, 1997; Arima et al., 2005; Casse et al., 1999; Lee et al., 2002). α-Amanitin may be useful, for example, as a payload in antibody drug conjugates (Davis & Preston, 1981; Moldenhauer et al., 2012; Figueroa-Vazquez et al., 2021; Pahl et al., 2018). Examples include an anti-EpCAM antibody against pancreatic carcinoma (Moldenhauer et al., 2012), and an anti-B-cell maturation antigen antibody against lymphoma (Figueroa-Vazquez et al., 2021) that is advancing in clinic. Since fermentation yields are low (Luo et al., 2015) and synthetic derivatives of high cytotoxicity have been elusive, most α-amanitin bioconjugates have relied on the naturally sourced toxin since (Bodero et al., 2018; Park et al, 2019a; Park et al., 2019b).

For over 70 years, α-amanitin eluded total synthesis until 2018 (Matinkhoo et al., 2018). Two other reports followed in 2020 (Lutz et al., 2020; Siegert et al., 2020). A fourth disclosed improved stereoselective sulfoxidation (Pryyma et al., 2020). Key challenges included i) synthesis of (2S,3R,4R)-dihydroxyisoleucine, a deceptively simple amino acid with high functional density and challenging stereochemical configuration (Hambira et al., 2021) and ii) an oxidatively delicate 6′-hydroxytrypathionine-(R)-sulfoxide staple that rigidifies the highly compact bicycle.

Given the significance of α-amanitin to fundamental and applied science, new analogs would be desirable. Yet given the notable array of noncovalent interactions between α-amanitin and Pol II, designing amatoxins with increased toxicity has neither been trivial nor obvious. To wit, essentially all known reports have been limited to amatoxins constructed from natural amino acids or obtained by modifying the natural product. A recent structure-activity relationship (SAR) study of hydroxyproline showed significant intolerance to OH replacement (Matinkhoo et al., 2021). An array of 25 dideoxy α-amanitin analogs (lacking both the dispensable 6-indole hydroxyl and sulfoxide) has also been disclosed (Yao et al., 2021). None was more than 10% as cytotoxic as α-amanitin or its dideoxy analog (Yao et al., 2021). With the exception of 7′-iodo-α-amanitin, no amatoxin to date has superseded the cytotoxicity of α-amanitin.

### SUMMARY

The synthesis, modelling and biochemical evaluation of amatoxin analogs comprising modifications of eastern ring residues is disclosed herein. Several of the amatoxin analogs were as toxic as α-amanitin and one example was 3-fold more toxic than α-amanitin. Such modified residues may be used alone, or in combination, for example, to tune activity, bioavailability and/or cell uptake of amatoxin analogs and conjugates thereof.

Accordingly, the present disclosure includes an amatoxin analog comprising at least one of the following eastern ring residues:
(a) a Gly⁵ residue of the formula:

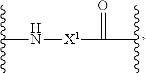

wherein X¹ is (CH₂)ₘ, CHCH₃, CHCH₂CH₃, C(CH₃)₂ or NR′;
(b) an Ile⁶ residue of the formula:

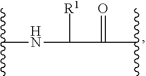

wherein R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S;
each Z is independently (CH₂)ₙO(CH₂)ₚ, (CH₂)ₙS(CH₂)ₚ, (CH₂)ₙSe(CH₂)ₚ or (CH₂)_{q};
each n is independently an integer from 0 to 4;
each p is independently an integer from 0 to 4;
n+p in each Z, is independently≤4;
each q is independently an integer from 0 to 4; and
wherein one or more single bonds between two available carbon atoms in R¹ is/are optionally replaced by a double bond;
(c) a Gly⁷ residue of the formula:

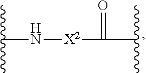

wherein X² is (CH₂)ₘ, CHCH₃, CHCH₂CH₃, C(CH₃)₂ or NR′; and
(d) a Cys⁸ residue of the formula:

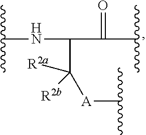

wherein
A is S, S(O), SO₂, Se, Se(O) or SeO₂; and
R^{2a} and R^{2b} are each independently H or C₁₋₁₀alkyl; or R^{2a} and R^{2b} together with the carbon atom to which they are attached, together form C₃₋₁₀cycloalkyl;
each m is independently an integer from 1 to 5; and
each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
or a pharmaceutically acceptable salt thereof,
provided that the amatoxin analog or the pharmaceutically acceptable salt thereof has one or more of the following:
   (i) R¹ other than

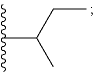 [SMILES: *C(C)CC]

(ii) X¹ other than CH₂;
(iii) X² other than CH₂;
(iv) R^{2a} other than H; and
(v) R^{2b} other than H, and
wherein in the amatoxin analog or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In an embodiment, the amatoxin analog is a compound of Formula I.

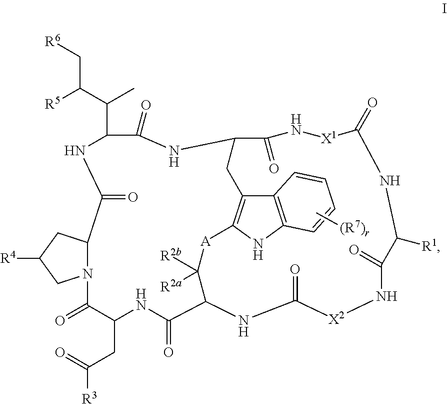

wherein
   A is S, S(O), SO₂, Se, Se(O) or SeO₂;
   X¹ and X² are each independently (CH₂)ₘ, CHCH₃, CHCH₂CH₃, C(CH₃)₂ or NR′;
   R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S; and wherein one or more single bonds between two available carbon atoms in R¹ is/are optionally replaced by a double bond;
   R^{2a} and R^{2b} are each independently H or C₁₋₁₀alkyl; or R^{2a} and R^{2b} together with the carbon atom to which they are attached, together form C₃₋₁₀cycloalkyl;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₘS(CH₂)ₚ, (CH₂)ₙSe(CH₂)ₚ or (CH₂)_{q};
   each m is independently an integer from 1 to 5;
   each n is independently an integer from 0 to 4;
   each p is independently an integer from 0 to 4;
   n+p in each Z, is independently ≤4;
   each q is independently an integer from 0 to 4;
   r is an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
or a pharmaceutically acceptable salt thereof,
provided that the compound of Formula I or the pharmaceutically acceptable salt thereof has one or more of the following:

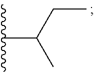 [SMILES: *C(C)CC]

(i) R¹ other than;
(ii) X¹ other than CH₂;
(iii) X² other than CH₂;
(iv) R^{2a} other than H; and
(v) R^{2b} other than H, and
wherein in the compound of Formula I or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In another embodiment, the amatoxin analog has the following stereochemistry:

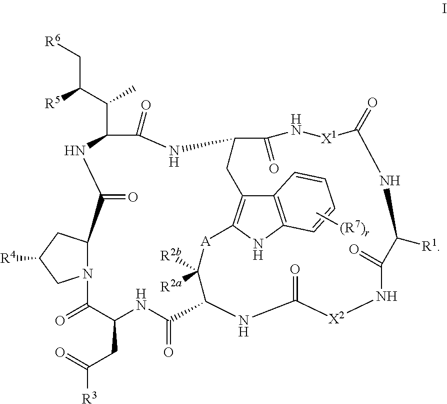

In an embodiment, R³ is NH₂.

In an embodiment, R⁴, R⁵ and R⁶ are each OH.

In an embodiment, r is 0. In another embodiment, r is 1.

In an embodiment, R⁷ is 4-OH, 5-OH, 6-OH or 7-OH. In another embodiment, R⁷ is 5-OH.

In an embodiment, R¹ is:
(a) CH(Z—CH₃)₂ other than

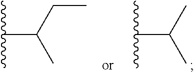

or
(b) unsubstituted C₃₋₁₀cycloalkyl.

In another embodiment, R¹ is

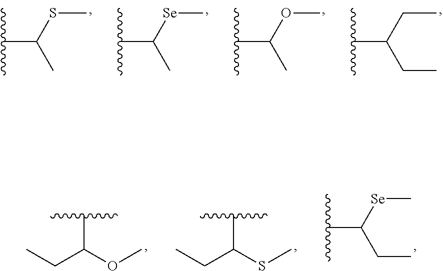

cyclopentyl,

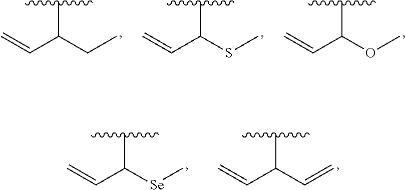

tetrahydrofuranyl, or tetrahydrothiophenyl. In a further embodiment, R¹ is

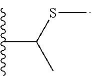 [SMILES: *C(C)SC]

In an embodiment R¹ is

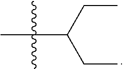 [SMILES: *C(CC)CC]

In an embodiment, X¹ is NH.

In an embodiment, X² is NH.

In an embodiment, X² is N—CH(CH₃)₂.

In an embodiment, at least one of R^{2a} and R^{2b} is C₁₋₁₀alkyl. In another embodiment, R^{2a} is CH₃ and R^{2b} is CH₃. In a further embodiment, R^{2a} is H and R^{2b} is CH₃.

In an embodiment, A is S or (R)—S(O). In another embodiment, A is S. In a further embodiment, A is (R)—S(O).

In an embodiment, the amatoxin analog is:

[chemical structure]

In an embodiment, the amatoxin analog is:

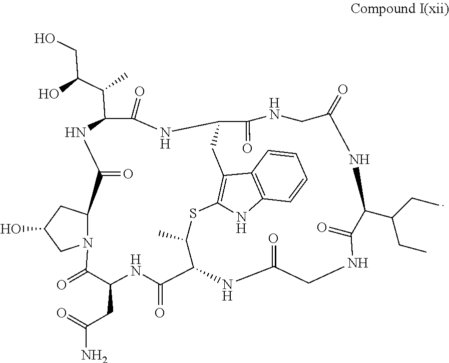 [SMILES: CCC(CC)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

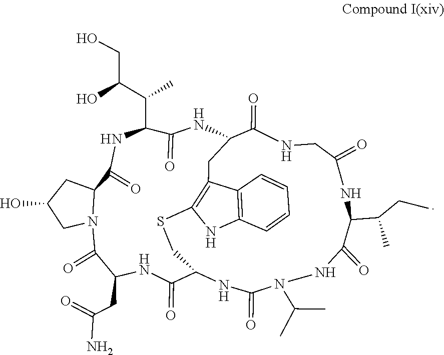 [SMILES: CC[C@H](C)[C@@H]1NC(=O)CNC(=O)[C@@H]2Cc3c([nH]c4ccccc34)SC[C@H](NC(=O)N(C(C)C)NC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is a compound of Formula II:

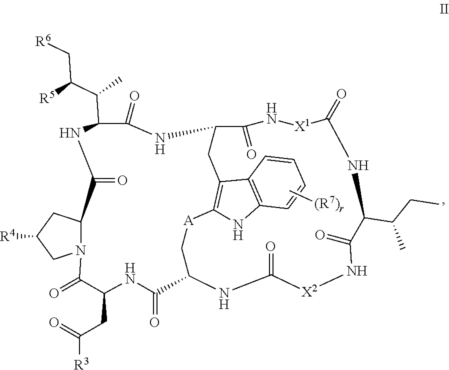

wherein
   X¹ and X² are each independently (CH₂)ₘ, CHCH₃, CHCH₂CH₃, C(CH₃)₂ or NR′, wherein m is an integer from 1 to 5 and provided that at least one of X¹ and X² is NR′;
   A is S, S(O), SO₂, Se, Se(O) or SeO₂;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   r is an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
   or a pharmaceutically acceptable salt thereof,
   wherein in the compound of Formula II or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In an embodiment, X¹ is NH.

In an embodiment, X² is NH.

In an embodiment, X² is N—CH(CH₃)₂.

In an embodiment, the amatoxin analog is a compound of Formula III:

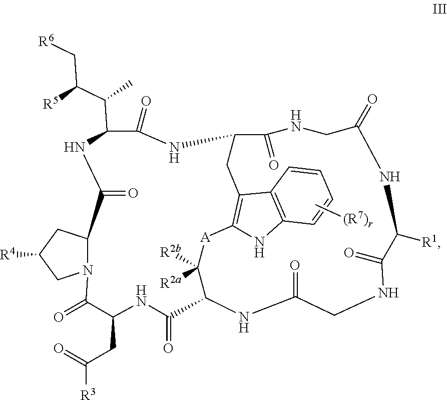

wherein
   A is S, S(O), SO₂, Se, Se(O) or SeO₂;
   R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S; and wherein one or more single bonds between two available carbon atoms in R¹ is/are optionally replaced by a double bond;
   R^{2a} and R^{2b} are each independently H or C₁₋₁₀alkyl, provided that at least one of R^{2a} and R^{2b} is C₁₋₁₀alkyl; or R^{2a} and R^{2b} together with the carbon atom to which they are attached, together form C₃₋₁₀cycloalkyl;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₙS(CH₂)ₚ, (CH₂)ₙSe(CH₂)ₚ or (CH₂)_{q};
   each n is independently an integer from 0 to 4;
   each p is independently an integer from 0 to 4;
   n+p in each Z, is independently ≤4;
   each q is independently an integer from 0 to 4;
   r is an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
   or a pharmaceutically acceptable salt thereof,
   wherein in the compound of Formula III or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In an embodiment, R¹ is

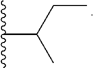 [SMILES: *C(C)CC]

In another embodiment, R¹ is

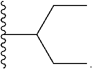 [SMILES: *C(CC)CC]

In an embodiment, at least one of R² and R^{2b} is C₁₋₁₀alkyl. In another embodiment, R^{2a} is CH₃ and R^{2b} is CH₃. In a further embodiment, R^{2a} is H and R^{2b} is CH₃.

In an embodiment, the amatoxin analog is a compound of Formula IV:

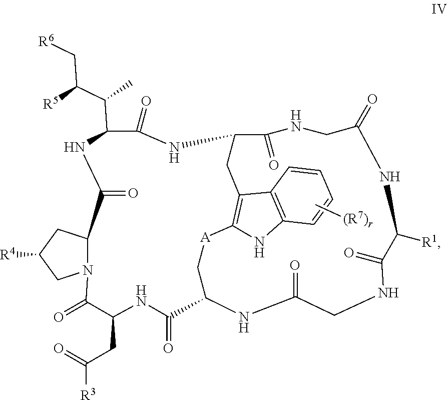

wherein
   A is S, S(O), SO₂, Se, Se(O) or SeO₂;
   R¹ is CH(Z—CH₃)₂, wherein one or more available hydrogens is/are optionally substituted with SH; or R¹ is C₃₋₁₀cycloalkyl, wherein one or more available hydrogens on the cycloalkyl are optionally substituted with C₁₋₁₀alkyl and/or one or more available carbons in the cycloalkyl is optionally replaced by O or S; and wherein one or more single bonds between two available carbon atoms in R¹ is/are optionally replaced by a double bond;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₙS(CH₂)ₚ, (CH₂)ₙSe(CH₂)ₚ or (CH₂)_{q};
   each n is independently an integer from 0 to 4;
   each p is independently an integer from 0 to 4;
   n+p in each Z, is independently ≤4;
   each q is independently an integer from 0 to 4,
   r is independently an integer from 0 to 4;
   provided that R¹ is other than

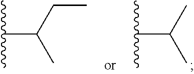

each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl;
or a pharmaceutically acceptable salt thereof,
wherein in the compound of Formula IV or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

The present disclosure also includes a compound-linker construct comprising an amatoxin analog as described herein coupled to a linker, wherein the linker comprises a reactive group R⁸ for conjugating the compound-linker construct to a target-binding moiety.

In an embodiment, the linker is coupled to the amatoxin analog or the salt thereof at a site indicated by R³, R⁴, R⁵, R⁶ and/or R⁷ in an amatoxin analog as described herein.

In an embodiment, the linker is coupled to the amatoxin analog or the salt thereof through a moiety obtained from the reaction of R³, R⁴, R⁵, R⁶ and/or R⁷ as described herein with a complementary functional group on the linker.

In an embodiment, at a site indicated by R³, R⁴, R⁵, R⁶ and/or R⁷ in the amatoxin analog as described herein, the compound-linker construct comprises a group of the formula —O-L, wherein L represents the linker; at a site indicated by R³, R⁴, R⁵, R⁶ and/or R⁷ in the amatoxin analog as described herein, the compound-linker construct comprises a group of the formula —O—C(O)-L, wherein L represents the linker; or at a site indicated by R³, R⁵, R⁶ and/or R⁷ in the amatoxin analog as described herein, the compound-linker construct comprises a group of the formula —NR′-L, wherein R′ is as described herein and L represents the linker.

In an embodiment, the linker is a stable linker.

In an embodiment, the linker is a cleavable linker.

In an embodiment, the linker further comprises a self-immolating moiety.

In an embodiment, the linker is cleavable by at least one agent selected from the group consisting of cysteine protease, metalloproteinase, serine protease, threonine protease, aspartic protease, glycosidase, phosphodiesterase, and reductase.

In an embodiment, the linker comprises a motif selected from the group consisting of: Val-Ala, Val-Cit, Val-Lys, Val-Arg, Phe-Lys-Gly-Pro-Leu-Gly, Ala-Ala-Pro-Val, β-glucuronide and β-galactoside.

In an embodiment:
(a) at a site indicated by R³, R⁴, R⁵, R⁶ or R⁷ in an amatoxin analog as described herein, the compound-linker construct comprises a group of the formula:

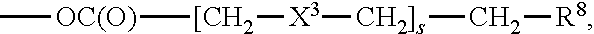

wherein X³ is O, CH₂ or S, s is an integer from 1 to 8 and R⁸ is the reactive group for conjugating the compound-linker construct to a target-binding moiety;
(b) at a site indicated by R³, R⁵, R⁶ or R⁷ in an amatoxin analog as described herein, the compound-linker construct comprises a group of the formula:

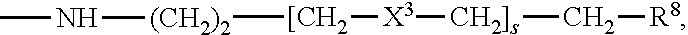

wherein X³ is O, CH₂ or S, s is an integer from 1 to 8 and R⁸ is the reactive group for conjugating the compound-linker construct to a target-binding moiety;
(c) at a site indicated by R³, R⁵, R⁶ or R⁷ in an amatoxin analog as described herein, the compound-linker construct comprises a group of the formula:

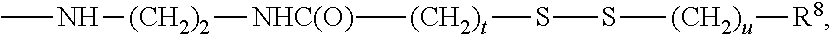

wherein t and u are independently an integer from 1 to 6 and R⁸ is the reactive group for conjugating the compound-linker construct to a target-binding moiety;
(d) at a site indicated by R³, R⁵, R⁶ or R⁷ in an amatoxin analog as described herein, the compound-linker construct comprises a group of the formula:

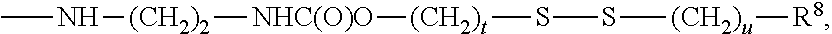

wherein t and u are independently an integer from 1 to 6 and R⁸ is the reactive group for conjugating the compound-linker construct to a target-binding moiety;
(e) at a site indicated by R³, R⁵, R⁶ or R⁷ in an amatoxin analog as described herein, the compound-linker construct comprises a group of the formula:

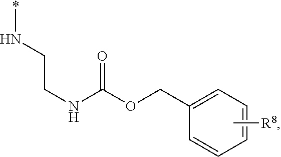

wherein * represents the site of attachment to the remainder of the compound-linker construct and R⁸ is the reactive group for conjugating the compound-linker construct to a target-binding moiety;
(f) at a site indicated by R³, R⁵, R⁶ or R⁷ in an amatoxin analog as described herein, the compound-linker construct comprises a group of the formula:

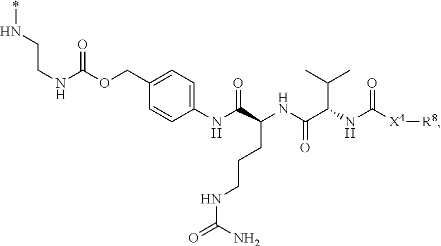

wherein * represents the site of attachment to the remainder of the compound-linker construct, X⁴ is C₁₋₁₀alkylene and R⁸ is the reactive group for conjugating the compound-linker construct to a target-binding moiety; or
(g) at a site indicated by R³, R⁴, R⁵, R⁶ or R⁷ in an amatoxin analog as described herein, the compound-linker construct comprises a group of the formula:

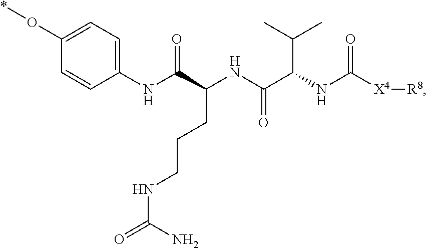

wherein * represents the site of attachment to the remainder of the compound-linker construct, X⁴ is C₁₋₁₀alkylene and R⁸ is the reactive group for conjugating the compound-linker construct to a target-binding moiety.

In an embodiment, at a site indicated by R³, R⁴, R⁵, R⁶ or R⁷ in the amatoxin analog as described herein, the compound-linker construct comprises a group of the formula:

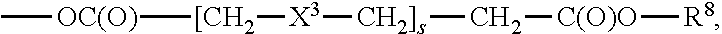

wherein X³ is a bond, O, CH₂ or S, s is an integer from 1 to 8 and R⁸ is the reactive group for conjugating the compound-linker construct to a target-binding moiety.

In an embodiment, R⁸ is selected from:

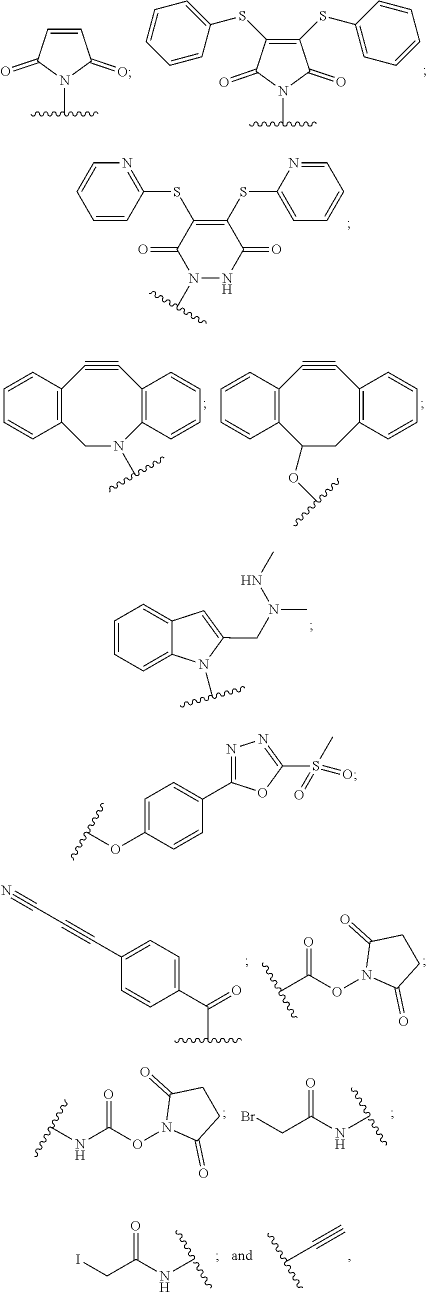

wherein [symbol] represents the site of attachment of R⁸ to the remainder of the linker; or R⁸ comprises an azide, thiol, tetrazine, trans-cyclooctene or acrylamide functional group.

In another embodiment, R⁸ is

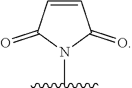 [SMILES: *N1C(=O)C=CC1=O]

In an embodiment, at a site indicated by R⁶ in the amatoxin analog as described herein, the compound-linker construct comprises a group of the formula:

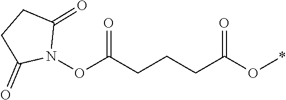 [SMILES: *OC(=O)CCCC(=O)ON1C(=O)CCC1=O]

wherein * represents the site of attachment to the remainder of the compound-linker construct.

The present disclosure also includes a conjugate comprising a target-binding moiety conjugated to an amatoxin analog or a compound-linker construct as described herein.

In an embodiment, the conjugate comprises the compound-linker construct, the target-binding moiety comprises an engineered acceptor residue, R⁸ is a reactive group to a moiety comprised in the engineered acceptor residue, and the compound-linker construct is conjugated to the target-binding moiety via a moiety resulting from the reaction of the moiety comprised in the engineered acceptor residue with R⁸. In another embodiment, the engineered acceptor residue is an engineered cystine residue and R⁸ is a thiol-reactive group. In a further embodiment, the engineered cysteine residue is selected from the group consisting of heavy chain 118Cys, heavy chain 239Cys, and heavy chain 265Cys. In another embodiment, the engineered cysteine residue is heavy chain 265Cys.

In an embodiment, the target-binding moiety comprises a biorthogonal functional group, R⁸ is a complementary biorthogonal functional group, and the compound-linker construct is conjugated to the target-binding moiety via a moiety resulting from the reaction of the biorthogonal functional group on the target-binding moiety with R⁸.

In an embodiment, the target-binding moiety comprises a primary amino group, R⁸ is a reactive group to the primary amino group, and the compound-linker construct is conjugated to the target-binding moiety via a moiety resulting from the reaction of the primary amino group with R⁸. In another embodiment, the primary amino group is a lysine side chain.

In an embodiment, the target-binding moiety is a peptide, an oligonucleotide, a lipid, a lipid carrier, a nanoparticle or combinations thereof. In another embodiment, the target-binding moiety is an antibody, an antigen-binding fragment thereof, an antibody-like protein, an aptamer, a vitamin, an oligonucleotide, an affibody, a minibody, a lipid, a lipid carrier, a nanoparticle or combinations thereof. In a further embodiment, the target-binding moiety is an antibody.

The present disclosure also includes a pharmaceutical composition comprising an amatoxin analog or conjugate of the present disclosure and a pharmaceutically acceptable carrier.

The present disclosure also includes a use of an effective amount of an amatoxin analog or a conjugate of the present disclosure for treatment of a disease associated with cells presenting a target in a subject in need thereof, wherein the target-binding moiety is specific for the target. The present disclosure also includes a use of an effective amount of an amatoxin analog or a conjugate of the present disclosure for preparation of a medicament for treatment of a disease associated with cells presenting a target in a subject in need thereof, wherein the target-binding moiety is specific for the target. In an embodiment, the disease associated with cells presenting a target is cancer, an autoimmune disease or a viral infection.

The present disclosure also includes a use of an effective amount of an amatoxin analog or a conjugate of the present disclosure for treatment of cancer in a subject in need thereof. The present disclosure also includes a use of an effective amount of an amatoxin analog or a conjugate of the present disclosure for preparation of a medicament for treatment of cancer in a subject in need thereof. In an embodiment, the cancer is breast cancer.

The present disclosure also includes an amatoxin analog or a conjugate of the present disclosure for use to treat a disease associated with cells presenting a target in a subject, wherein the target-binding moiety is specific for the target. In an embodiment, the disease associated with cells presenting a target is cancer, an autoimmune disease or a viral infection.

The present disclosure also includes an amatoxin analog or a conjugate of the present disclosure for use to treat cancer in a subject. In an embodiment, the cancer is breast cancer.

The present disclosure also includes a method of treating a disease associated with cells presenting a target in a subject in need thereof, the method comprising administering an effective amount of an amatoxin analog or a conjugate of the present disclosure to the subject, wherein the target-binding moiety is specific for the target. In an embodiment, the disease associated with cells presenting a target is cancer, an autoimmune disease or a viral infection.

The present disclosure also includes a method of treating cancer in a subject in need thereof, the method comprising administering an effective amount of an amatoxin analog or a conjugate of the present disclosure to the subject.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows circular dichroism (CD) spectra of erythro-β-methyl Cys⁸ containing analogues I(i) and I(xii), threo-β-methyl Cys⁸ analogue, I(ii), and Pen⁸ analogue, I(iii).

FIG. 2 shows CD spectra of Ile⁶ amanitin analogues I(v), I(vi), I(vii) and I(viii).

FIG. 3 shows CD spectra of compounds I(x), I(xi) and I(xiii) with 5′ OH Trp⁴ staples.

FIG. 4 shows CD spectra of aza-Gly amanitins I(iv) and I(ix).

FIG. 5 shows overlays of energy minimized models of eastern ring analogs of α-amanitin (turquoise in colour images) and S-deoxyamaninamide (green in color images); structures labeled a-j are: a) Compound I(i) (erythro-β-methylcysteine⁸); b) Compound I(ii) (threo-β-methylcysteine⁸); c) Compound I(iii) (penicillamine⁸); d) Compound I(xii) (erythro-β-methylcysteine⁸ and diethylalanine⁶); e) Compound I(v) (thia-isoleucine⁶); f) Compound I(vi) (oxo-isoleucine⁶); g) Compound I(viii) (cyclopentylglycine⁶); h) Compound I(vii) (diethylalanine⁶); i) Compound I(ix) (aza-gly⁵); and j) Compound I(iv) (aza-gly⁷).

FIG. 6 shows [³²P]-autoradiograms of transcription runoff assay resolved by 8% denaturing PAGE, from top to bottom: α-amanitin, Compound I(i) and Compounds I(iii-ix).

FIG. 7 shows [³²P]-autoradiograms of transcription runoff assay resolved by 8% denaturing PAGE, from top to bottom: α-amanitin, Compounds I(x-xiii) and Compound I(ii).

FIG. 8 shows Kᵢ curves of, from top to bottom: α-amanitin (top) and its analogs, Compound I(i) and Compounds I(iii-ix).

FIG. 9 shows Kᵢ curves of, from top to bottom: α-amanitin (top) and its analogs, Compounds I(x-xiii).

FIG. 10 shows cell viability on HEK293 cells as a plot of % of alive cells as a function of log[concentration, pM] for Compound I(xiv) (AzaVal7, IC₅₀=0.31±μM) according to an example of the present disclosure in comparison to α-amanitin (IC₅₀=0.55±0. μM) according to a comparative example of the present disclosure.

FIG. 11 shows cell viability on SK-BR-3 cells as a plot of % of alive cells as a function of log[concentration, fM] for α-amanitin (IC₅₀=0.35±0.08 μM), α-amanitin conjugated to trastuzumab (Amanitin-ADC; drug-to-antibody ratio, DAR=2, IC₅₀=9.78±3.77 pM) and Herceptin™ (IC₅₀>1 μM) according to comparative examples of the present disclosure.

FIG. 12 shows cell viability on SK-BR-3 cells as a plot of % of alive cells as a function of log[concentration, fM] for Compound I(v) conjugated to trastuzumab (ThiaIle6 ADC; DAR=4.9, IC₅₀=12.64±7.28 pM), Compound I(xii) conjugated to trastuzumab (DiethAla6, Eβ-MeCys8 ADC; DAR=0.9, IC₅₀=8.32±3.39 pM) and Compound I(iv) conjugated to trastuzumab (AzaGly7 ADC; DAR=1.4, IC₅₀=2.16±1.23 pM) according to examples of the present disclosure.

FIG. 13 shows cell viability on SK-BR-3 cells as a plot of % of alive cells as a function of log[concentration, fM] for Compound I(v) conjugated to trastuzumab (ThiaIle6 ADC; DAR=4.9, IC₅₀=12.64±7.28 pM), Compound I(xii) conjugated to trastuzumab (DiethAla6, Eβ-MeCys8 ADC; DAR=0.9, IC₅₀=8.32±3.39 pM) and Compound I(iv) conjugated to trastuzumab (AzaGly7 ADC; DAR=1.4, IC₅₀=2.16±1.23 pM) according to examples of the present disclosure in comparison to α-amanitin (IC₅₀=0.25±0.05 μM) and Herceptin™ (IC₅₀>1 μM) according to comparative examples of the present disclosure.

FIG. 14 shows cell viability on SK-BR-3 cells as a plot of % of alive cells as a function of log[concentration, fM] for Compound I(xii) conjugated to trastuzumab with two values of DAR (DiethAla6, Eβ-MeCys8 ADC; DAR=2.1, IC₅₀=0.41±0.18 pM) and (DiethAla6, Eβ-MeCys8 ADC; DAR=2.7, IC₅₀=0.39±0.22 nM) according to examples of the present disclosure.

FIG. 15 shows cell viability on SK-BR-3 cells as a plot of % of alive cells as a function of log[concentration, fM] for Compound I(xii) conjugated to trastuzumab with two values of DAR (DiethAla6, Eβ-MeCys8 ADC; DAR=2.1, IC₅₀=0.41±0.18 pM) and (DiethAla6, Eβ-MeCys8 ADC; DAR=2.7, IC₅₀=0.39±0.22 nM) according to examples of the present disclosure in comparison to α-amanitin (IC₅₀=0.30±0.07 μM) and Herceptin™ (IC₅₀>1 μM) according to comparative examples of the present disclosure.

FIG. 16 shows cell viability on SK-BR-3 cells as a plot of % of alive cells as a function of log[concentration, fM] for Compound I(xii) conjugated to trastuzumab with two values of DAR (DiethAla6, Eβ-MeCys8 ADC; DAR=3.5, IC₅₀=37.91±16.15 fM) and (DiethAla6, Eβ-MeCys8 ADC; DAR=5.8, IC₅₀=7.09±5.55 fM) according to examples of the present disclosure.

FIG. 17 shows cell viability on SK-BR-3 cells as a plot of % of alive cells as a function of log[concentration, fM] for Compound I(xii) conjugated to trastuzumab with two values of DAR (DiethAla6, Eβ-MeCys8 ADC; DAR=3.5, IC₅₀=37.91±16.15 fM) and (DiethAla6, Eβ-MeCys8 ADC; DAR=5.8, IC₅₀=7.09±5.55 fM) according to examples of the present disclosure in comparison to α-amanitin (IC₅₀=0.19±0.06 μM) and Herceptin™ (IC₅₀>1 μM) according to comparative examples of the present disclosure.

FIG. 18 shows cell viability on SK-BR-3 cells as a plot of % of alive cells as a function of log[concentration, fM] for Compound I(xii) conjugated to trastuzumab with three values of DAR (DiethAla6, Eβ-MeCys8 ADC; DAR=3.5, IC₅₀=87.31±33.77 fM), (DiethAla6, Eβ-MeCys8 ADC; DAR=5.8, IC₅₀=19.32±5.18 fM) and (DiethAla6, Eβ-MeCys8 ADC; DAR=6.25, IC₅₀=6.08±1.41 fM) according to examples of the present disclosure.

FIG. 19 shows cell viability on SK-BR-3 cells as a plot of % of alive cells as a function of log[concentration, fM] for Compound I(xii) conjugated to trastuzumab with three values of DAR (DiethAla6, Eβ-MeCys8 ADC; DAR=3.5, IC₅₀=87.31±33.77 fM), (DiethAla6, Eβ-MeCys8 ADC; DAR=5.8, IC₅₀=19.32±5.18 fM) and (DiethAla6, Eβ-MeCys8 ADC; DAR=6.25, IC₅₀=6.08±1.41 fM) according to examples of the present disclosure in comparison to α-amanitin (IC₅₀=0.47±0.11 μM) and Herceptin (IC₅₀>1 μM) according to comparative examples of the present disclosure.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least +5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₆alkyl means an alkyl group having 1, 2, 3, 4, 5, 6, 7, 8, 9 or 10 carbon atoms.

The term “alkylene” as used herein, means a straight or branched chain, bivalent form of an alkane. The number of carbon atoms that are possible in the referenced alkylene group are indicated by the numerical prefix “Cₙ₁₋ₙ₂”. For example, the term C₁₋₁₀alkylene means an alkylene group having 1, 2, 3, 4, 5, 6, 7, 8, 9 or 10 carbon atoms.

The term “alkenyl” as used herein, whether it is used alone or as part of another group, means straight or branched chain, unsaturated alkenyl groups. The number of carbon atoms that are possible in the referenced alkenyl group are indicated by the numerical prefix “Cₙ₁₋ₙ₂”. For example, the term C₂₋₂₀alkenyl means an alkenyl group having 2, 3, 4, 5, 6, 7, 8, 9, 10, 11, 12, 13, 14, 15, 16, 17, 18, 19 or 20 carbon atoms and at least one double bond, for example 1-3, 1-2 or 1 double bond.

The term “alkynyl” as used herein, whether it is used alone or as part of another group, means straight or branched chain, unsaturated alkynyl groups. The number of carbon atoms that are possible in the referenced alkynyl group are indicated by the numerical prefix “Cₙ₁₋ₙ₂”. For example, the term C₂₋₂₀alkynyl means an alkenyl group having 2, 3, 4, 5, 6, 7, 8, 9, 10, 11, 12, 13, 14, 15, 16, 17, 18, 19 or 20 carbon atoms and at least one triple bond, for example 1-3, 1-2 or 1 triple bond.

The term “cycloalkyl” as used herein, whether it is used alone or as part of another group, means a mono- or bicyclic, saturated cycloalkyl group. The number of carbon atoms that are possible in the referenced cycloalkyl group are indicated by the numerical prefix “Cₙ₁₋ₙ₂”. For example, the term C₃₋₁₀-cycloalkyl means a cycloalkyl group having 3, 4, 5, 6, 7, 8, 9 or 10 carbon atoms. When a cycloalkyl group contains more than one cyclic structure or rings, the cyclic structures may be fused, bridged, spiro connected or linked by a single bond. The term “fused” as used herein in reference to a first cyclic structure being “fused” with a second cyclic structure means the first cyclic structure and the second cyclic structure share at least two adjacent atoms therebetween. The term “bridged” as used herein in reference to a first cyclic structure being “bridged” with a second cyclic structure means the first cyclic structure and the second cyclic structure share at least two non-adjacent atoms therebetween. The term “spiro-connected” in reference to a first cyclic structure being “spiro connected” with a second cyclic structure means the first cyclic structure and the second cyclic structure share one atom therebetween.

The term “aryl” as used herein, whether it is used alone or as part of another group, refers to cyclic groups that contain at least one aromatic ring. The number of carbon atoms that are possible in the referenced aryl group are optionally indicated by the numerical prefix “Cₙ₁₋ₙ₂”. For example, the term C₆₋₃₀aryl means an aryl group having 6, 7, 8, 9, 10, 11, 12, 13, 14, 15, 16, 17, 18, 19, 20, 21, 22, 23, 24, 25, 26, 27, 28, 29 or 30 carbon atoms. In an embodiment, the aryl group contains from 6, 9, 10 or 14 carbon atoms, such as phenyl, naphthyl, indanyl or anthracenyl.

The term “heterocycloalkyl” as used herein, whether it is used alone or as part of another group, refers to a non-aromatic, ring-containing group having one or more multivalent heteroatoms, for example, independently selected from O, S and Se, as a part of the ring structure and including at least 3 and up to 20 atoms in the ring(s). Heterocycloalkyl groups are either saturated or unsaturated (i.e. contain one or more double bonds) and may contain more than one ring.

The term “heteroaryl” as used herein, whether it is used alone or as part of another group, refers to an aromatic, ring-containing group having one or more multivalent heteroatoms, for example, independently selected from O, S and Se, as apart of the ring structure and including at least 5 and up to 20 atoms in the ring(s). Heteroaryl groups may contain more than one ring.

The term “halo” as used herein refers to a halogen atom and includes F, C₁ and Br.

The term “available”, as used herein in reference to “available hydrogens”, “available carbon atoms”, “available atoms” and the like refers to atoms that would be known to a person skilled in the art to be capable of modification and/or replacement by another atom or substituent.

The term “replaced with its radioactive isotope” as used herein in reference to one or more available atoms in the amatoxin analogs of the present disclosure means that a non-radioactive isotope of the atom is replaced with the corresponding radioactive isotope. For example, in an embodiment, a non-radioactive isotope of hydrogen is replaced with ³H (tritium). In another embodiment, a non-radioactive isotope of carbon is replaced with ¹¹C or ¹⁴C. In a further embodiment, a non-radioactive isotope of iodine is replaced with ¹²³I, ¹²⁵I or ¹³¹I. In another embodiment, a non-radioactive isotope of sulfur is replaced with ³⁵S. It will be appreciated by a person skilled in the art that depending, for example, on the radioactive isotope, such radiolabelled compounds may, for example, be useful as radiopharmaceuticals for treatment and/or diagnostic applications. The selection of a suitable radioactive isotope and methods for the preparation of radiolabelled compounds can be made by a person skilled in the art.

The term “subject” as used herein includes all members of the animal kingdom including mammals. In an embodiment, the subject is a human.

The term “pharmaceutically acceptable” as used herein means compatible with the treatment of subjects, for example, mammals such as humans.

The term “pharmaceutically acceptable salt” as used herein means either an acid addition salt or a base addition salt that is compatible with the treatment of subjects.

An “acid addition salt that is compatible with the treatment of subjects” is any suitable non-toxic organic or inorganic salt of any basic compound. Basic compounds that form an acid addition salt include, for example, compounds comprising an amine group susceptible to protonation. Illustrative inorganic acids which form suitable salts include but are not limited to hydrochloric, hydrobromic, sulfuric and phosphoric acids, as well as metal salts such as but not limited to sodium monohydrogen orthophosphate and potassium hydrogen sulfate. Illustrative organic acids that may form suitable salts include but are not limited to mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as but not limited to p-toluene sulfonic and methanesulfonic acids. The selection of a suitable salt can be made by a person skilled in the art. The formation of a desired acid addition salt is, for example, achieved using standard techniques. For example, in an embodiment, the neutral compound is treated with the desired acid in a suitable solvent and the salt which is thereby formed then isolated by filtration, extraction and/or any other suitable method.

A “base addition salt that is compatible with the treatment of subjects” is any suitable nontoxic inorganic or organic salt of any acidic compound. Acidic compounds that form a base addition salt include, for example, compounds comprising a carboxylic, phosphonic or sulfonic acid group. Inorganic bases that may form suitable salts include, without limitation, lithium, sodium, potassium, calcium, magnesium or barium hydroxide. Organic bases that may form suitable salts include, without limitation, aliphatic, alicyclic or aromatic organic amines such as methylamine, trimethylamine and picoline or ammonia. The selection of a suitable salt can be made by a person skilled in the art. The formation of a desired base addition salt is, for example, achieved using standard techniques. For example, in an embodiment, the neutral compound is treated with the desired base in a suitable solvent and the salt which is thereby formed then isolated by filtration, extraction and/or any other suitable method.

The terms “to treat”, “treating” and “treatment” as used herein and as is well understood in the art, mean an approach for obtaining beneficial or desired results, including clinical results. Beneficial or desired clinical results include but are not limited to alleviation or amelioration of one or more symptoms of a disease (e.g. cancer), diminishment of the extent of the disease, stabilization (i.e. not worsening) of the disease, delay or slowing of the progression of the disease, amelioration or palliation of the state of the disease, diminishment of the recurrence of the disease and/or remission (whether partial or total) of the disease, whether detectable or undetectable. To “treat”, “treating” and “treatment” as used herein also include prophylactic treatment of the disease. For example, in an embodiment, a subject with early-stage disease is treated to prevent progression or alternatively a subject in remission is treated to prevent recurrence.

As used herein, the term “effective amount” means an amount effective, at dosages and for periods of time necessary to achieve a desired result. For example, in the context of treating cancer, an effective amount of an amatoxin analog or conjugate of the present disclosure is an amount that, for example, reduces the cancer compared to the cancer without administration or use of the amatoxin analog or conjugate, respectively. Effective amounts may vary according to factors such as the disease state, age, sex and/or weight of the subject. The amount of a given amatoxin analogue or conjugate, as the case may be, that will correspond to such an amount will vary depending upon various factors, such as the given amatoxin analog or conjugate, the pharmaceutical formulation, the type of disease being treated, the identity of the subject being treated, and the like, but can nevertheless be routinely determined by one skilled in the art.

### II. Amatoxin Analogs, Compound-Linker Constructs and Conjugates

The synthesis, modelling and biochemical evaluation of amatoxin analogs comprising modifications of eastern ring residues is disclosed herein. Several of the amatoxin analogs were as toxic as α-amanitin and one example was 3-fold more toxic than α-amanitin. Such modified residues may be used alone, or in combination, for example, to tune activity, bioavailability and/or cell uptake of amatoxin analogs and conjugates thereof.

Accordingly, the present disclosure includes an amatoxin analog comprising at least one of the following eastern ring residues:
(a) a Gly⁵ residue of the formula:

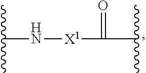

wherein X¹ is (CH₂)ₘ, CHCH₃, CHCH₂CH₃, C(CH₃)₂ or NR′;
(b) an Ile⁶ residue of the formula:

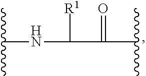

wherein R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S;
each Z is independently (CH₂)ₙO(CH₂)ₚ, (CH₂)ₙS(CH₂)ₚ, (CH₂)ₙSe(CH₂)ₚ or (CH₂)_{q};
each n is independently an integer from 0 to 4;
each p is independently an integer from 0 to 4;
n+p in each Z, is independently ≤4;
each q is independently an integer from 0 to 4; and
wherein one or more single bonds between two available carbon atoms in R¹ is/are optionally replaced by a double bond;
(c) a Gly⁷ residue of the formula:

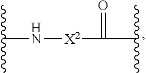

wherein X² is (CH₂)ₘ, CHCH₃, CHCH₂CH₃, C(CH₃)₂ or NR′; and
(d) a Cys⁸ residue of the formula:

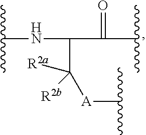

wherein
A is S, S(O), SO₂, Se, Se(O) or SeO₂; and
R^{2a} and R^{2b} are each independently H or C₁₋₁₀alkyl; or R^{2a} and R^{2b} together with the carbon atom to which they are attached, together form C₃₋₁₀cycloalkyl;
each m is independently an integer from 1 to 5; and
each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁-20alkyl, C₂₋₂₀alkenyl, C₂₋₂₀alkynyl, C₃₋₂₀cycloalkyl, C₆₋₃₀aryl, heterocycloalkyl or heteroaryl, or a pharmaceutically acceptable salt thereof,
provided that the amatoxin analog or the pharmaceutically acceptable salt thereof has one or more of the following:

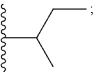 [SMILES: *C(C)CC]

(i) R¹ other than;
(ii) X¹ other than CH₂;
(iii) X² other than CH₂;
(iv) R^{2a} other than H; and
(v) R^{2b} other than H, and
wherein in the amatoxin analog or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

The amatoxin can be any suitable amatoxin. The term “amatoxin” as used herein includes all cyclic peptides composed of 8 amino acids as isolated from the genus Amanita and described, for example, by Wieland, T. and Faulstich, H. in CRC Critical Reviews in Biochemistry 1978, 5 (3), 185-260 as well as all suitable chemical derivatives, semisynthetic analogs and synthetic analogs thereof built from building blocks according to the master structure of the natural compounds (cyclic, 8 amino acids). The term “amatoxin” as used herein also includes all suitable synthetic or semisynthetic analogs of amatoxins containing non-hydroxylated amino acids instead of the hydroxylated amino acids and/or amino acids comprising beta-alkyl groups such as beta-methyl groups and/or in which the sulfoxide moiety is replaced by a sulfone, or thioether, or by atoms different from sulfur, e.g., a carbon atom as in a carbanalog of amanitin. In an embodiment, the amatoxin has a functional group (e.g. a carboxylic group, an amino group, a hydroxy group, a thiol and/or a thiol-capturing group) that can be reacted with a linker and/or a target-binding moiety as defined herein. In an embodiment of the present disclosure, the amatoxin is an α-amanitin, β-amanitin, γ-amanitin, ε-amanitin, amanullin, amanullinic acid, amanin, amaninamide, γ-amanin or γ-amaninamide or a derivative thereof.

In an embodiment, the amatoxin analog is a compound of Formula I.

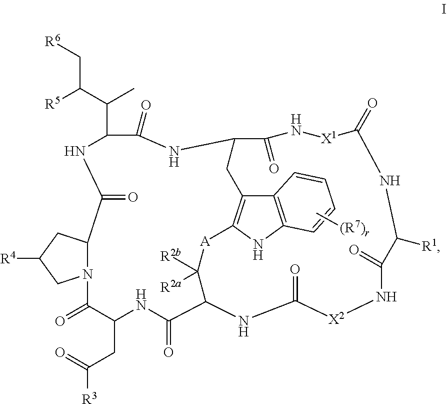

wherein
   A is S, S(O), SO₂, Se, Se(O) or SeO₂;
   X¹ and X² are each independently (CH₂)ₘ, CHCH₃, CHCH₂CH₃, C(CH₃)₂ or NR′;
   R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S; and wherein one or more single bonds between two available carbon atoms in R¹ is/are optionally replaced by a double bond;
   R^{2a} and R^{2b} are each independently H or C₁₋₁₀alkyl; or R^{2a} and R^{2b} together with the carbon atom to which they are attached, together form C₃₋₁₀cycloalkyl;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₙS(CH₂)ₚ, (CH₂)ₙSe(CH₂)ₚ or (CH₂)_{q};
   each m is independently an integer from 1 to 5;
   each n is independently an integer from 0 to 4;
   each p is independently an integer from 0 to 4;
   n+p in each Z, is independently ≤4;
   each q is independently an integer from 0 to 4;
   r is independently an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
or a pharmaceutically acceptable salt thereof,
provided that the compound of Formula I or the pharmaceutically acceptable salt thereof has one or more of the following:
   (i) R¹ other than

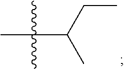 [SMILES: *C(C)CC]

(ii) X¹ other than CH₂;
(iii) X² other than CH₂;
(iv) R^{2a} other than H; and
(v) R^{2b} other than H, and
wherein in the compound of Formula I or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In an embodiment, the amatoxin analog of Formula I has the following stereochemistry:

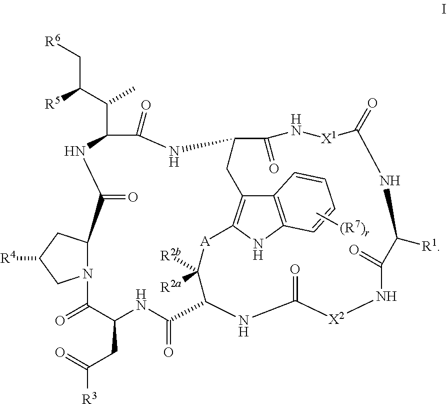

In an embodiment, R¹ is:
(a) CH(Z—CH₃)₂ other than

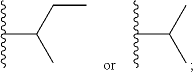

or
(b) unsubstituted C₃₋₁₀cycloalkyl.

In some embodiments, R¹ is saturated; i.e. no single bonds between carbon atoms have been replaced by a double bond. In other embodiments, one or more single bonds between two available carbon atoms in R¹ is/are replaced by a double bond.

In an embodiment, R¹ is CH(Z—CH₃)₂, wherein one or more available hydrogens is/are optionally substituted with SH. In some embodiments, R¹ is devoid of SH groups; i.e. no hydrogens are substituted with SH. In some embodiments, R¹ comprises one or more SH groups. In an embodiment, each Z is independently (CH₂)ₙO(CH₂)ₚ, (CH₂)ₙS(CH₂) or (CH₂)_{q}. In another embodiment, each Z is independently (CH₂)_{q}. In a further embodiment, one Z is (CH₂)ₙO(CH₂)ₚ and the other Z is (CH₂)_{q}. In another embodiment, one Z is (CH₂)ₙS(CH₂)ₚ and the other Z is (CH₂)_{q}. In an embodiment, each n is independently an integer from 0 to 3. In another embodiment, each n is independently an integer from 0 to 2. In another embodiment, n is 0. In an embodiment, each p is independently an integer from 0 to 3. In another embodiment, each p is independently an integer from 0 to 2. In another embodiment, p is 0. In an embodiment, n+p in each Z, is independently ≤3. In another embodiment, n+p in each Z, is independently; 2. In a further embodiment, n+p=0. In an embodiment, each q is independently an integer from 0 to 3. In another embodiment, each q is independently an integer from 0 to 1. In another embodiment, q is 1.

In an embodiment, R¹ is C₃₋₁₀cycloalkyl. In another embodiment, R¹ is C₃₋₆cycloalkyl. In a further embodiment, R¹ is cyclopentyl. In some embodiments, R¹ is carbocyclic; i.e. no carbon atoms in the cycloalkyl (e.g. the C₃₋₁₀cycloalkyl such as cyclopentyl) have been replaced by O or S. In other embodiments, one or more available carbon atoms in the cycloalkyl (e.g. the C₃₋₁₀cycloalkyl such as cyclopentyl) is/are replaced by S or O. In an embodiment, R¹ is a heterocycloalkyl comprising S and/or O atoms such as tetrahydrofuranyl or tetrahydrothiophenyl. In some embodiments, R¹ is unsubstituted. In some embodiments, one or more available hydrogens on the C₃₋₁₀cycloalkyl is/are optionally substituted with C₁₋₁₀alkyl.

In an embodiment, R¹ is

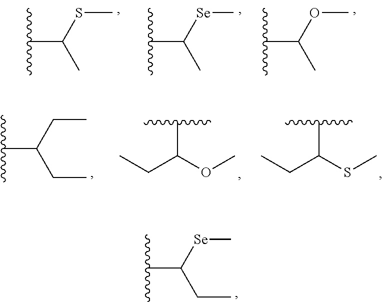

cyclopentyl,

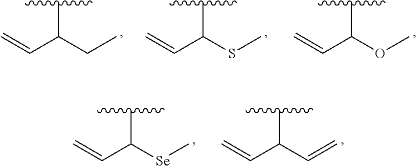

tetrahydrofuranyl, or tetrahydrothiophenyl. In another embodiment, R¹ is

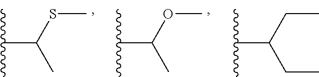

or cyclopentyl. In another embodiment, R¹ is

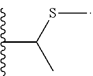 [SMILES: *C(C)SC]

In a further embodiment, R¹ is

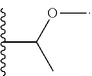 [SMILES: *C(C)OC]

In another embodiment, R¹ is

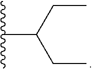 [SMILES: *C(CC)CC]

In an embodiment, R¹ is

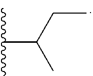 [SMILES: *C(C)CC]

In another embodiment, R¹ is other than

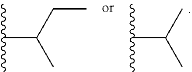

In an embodiment, R¹ is cyclopentyl.

In an embodiment, R^{2a} and R^{2b} are each independently H or C₁₋₁₀alkyl. In another embodiment, at least one of R^{2a} and R^{2b} is C₁₋₁₀alkyl. In a further embodiment, at least one of R² and R^{2b} is C₁₋₆alkyl. In another embodiment, at least one of R^{2a} and R^{2b} is C₁₋₃alkyl. In another embodiment, at least one of R^{2a} and R^{2b} is methyl. In an embodiment, R^{2a} is CH₃ and R^{2b} is CH₃. In another embodiment, R^{2a} is H and R^{2b} is CH₃. In a further embodiment, R^{2a} is CH₃ and R^{2b} is H.

In an embodiment, R^{2a} and R^{2b} together with the carbon atom to which they are attached, together form C₃₋₁₀cycloalkyl. In another embodiment, R^{2a} and R^{2b} together with the carbon atom to which they are attached, together form C₃₋₆cycloalkyl. In a further embodiment, R^{2a} and R^{2b} together with the carbon atom to which they are attached, together form cyclopropyl.

In an embodiment, R³ is OR′ or N(R′)₂. In another embodiment, R³ is OR′ or N(R′)₂, wherein R′ is H. In a further embodiment, R³ is NH₂.

In an embodiment, R⁴ is H. In another embodiment, R⁴ is OH.

In an embodiment, R⁵ and R⁶ are each independently H or OR′. In another embodiment, R⁵ and R⁶ are each independently H or OH. In an embodiment, R⁵ is H and R⁶ is OH. In another embodiment, R⁵ is OH and R⁶ is H. In an embodiment, R⁵ and R⁶ are each H. In another embodiment, R⁵ and R⁶ are each OH. In an embodiment, R⁵ is H. In another embodiment of the present disclosure, R⁵ is OR′. In a further embodiment, R⁵ is OH. In an embodiment, R⁶ is H. In another embodiment, R⁶ is OR′. In a further embodiment, R⁶ is OH.

In an embodiment, r is an integer from 0 to 3. In another embodiment, r is 0, 1 or 2. In another embodiment, r is 0 or 1. In an embodiment, r is 0. In another embodiment, r is 1.

In an embodiment, R⁴, R⁵ and R⁶ are each OH.

In an embodiment, each R⁷ is independently OR′. In another embodiment, R⁷ is OH. In a further embodiment, R⁷ is 4-OH, 5-OH, 6-OH or 7-OH. In an embodiment, R⁷ is 5-OH.

In an embodiment, X¹ is (CH₂)ₘ or NR′. In another embodiment, X¹ is (CH₂)ₘ. In an embodiment, X¹ is CH₂. In a further embodiment, X¹ is NR′. In an embodiment, X¹ is NH or NC₁₋₂₀alkyl. In another embodiment, X¹ is NH or NC₁₋₆alkyl. In a further embodiment, X¹ is NH or N—CH(CH₃)₂. In another embodiment, X¹ is N—CH(CH₃)₂. In an embodiment, X¹ is NH.

In an embodiment, X² is (CH₂)ₘ or NR′. In another embodiment, X² is (CH₂)ₘ. In an embodiment, X² is CH₂. In a further embodiment, X² is NR′. In an embodiment, X² is NH or NC₁₋₂₀alkyl. In another embodiment, X² is NH or NC₁₋₆alkyl. In a further embodiment, X² is NH or N—CH(CH₃)₂. In another embodiment, X² is N—CH(CH₃)₂. In an embodiment, X² is NH.

In an embodiment, X¹ is CH₂ and X² is NR′. In an embodiment, X¹ is CH₂ and X² is NH or NC₁₋₂₀alkyl. In another embodiment, X¹ is CH₂ and X² is NH or NC₁₋₆alkyl. In a further embodiment, X¹ is CH₂ and X² is NH or N—CH(CH₃)₂. In another embodiment, X¹ is CH₂ and X² is N—CH(CH₃)₂. In another embodiment, X¹ is CH₂ and X₂ is NH. In a further embodiment, X¹ is NR′ and X² is CH₂. In an embodiment, X¹ is NH or NC₁₋₂₀alkyl and X² is CH₂. In another embodiment, X¹ is NH or NC₁₋₆alkyl and X² is CH₂. In a further embodiment, X¹ is NH or N—CH(CH₃)₂ and X² is CH₂. In another embodiment, X¹ is N—CH(CH₃)₂ and X² is CH₂. In another embodiment, X¹ is NH and X² is CH₂. In a further embodiment, X¹ and X² are CH₂.

In an embodiment, each m is independently an integer from 1 to 4. In another embodiment, each m is independently an integer from 1 to 3. In a further embodiment, each m is independently 1 or 2. In another embodiment, m is 1.

In an embodiment, A is S, (R)—S(O), Se or (R)—Se(O). In an embodiment, A is S or (R)—S(O). In another embodiment, A is S. In a further embodiment, A is (R)—S(O).

In an embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is unsubstituted. In another embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl. In a further embodiment, each R′ is independently H or C₁₋₂₀alkyl. In a further embodiment, each R′ is independently H or C₁₋₆alkyl. In some embodiments, R′ is H. In some embodiments, R′ is C₁₋₆alkyl.

In an embodiment, the amatoxin analog is:

[chemical structure]

In an embodiment, the amatoxin analog is:

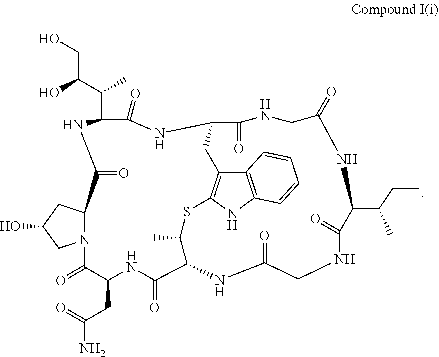 [SMILES: CC[C@H](C)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

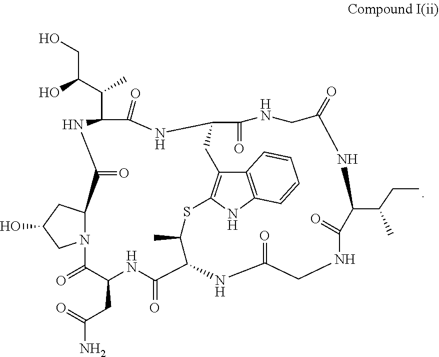 [SMILES: CC[C@H](C)[C@@H]1NC(=O)CNC(=O)[C@H]2Cc3c([nH]c4ccccc34)S[C@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

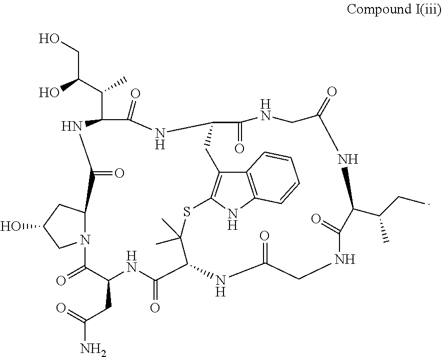 [SMILES: CC[C@H](C)[C@@H]1NC(=O)CNC(=O)[C@H]2Cc3c([nH]c4ccccc34)SC(C)(C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

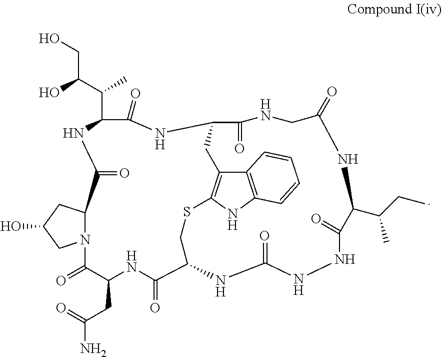 [SMILES: CC[C@H](C)[C@@H]1NC(=O)CNC(=O)[C@H]2Cc3c([nH]c4ccccc34)SC[C@H](NC(=O)NNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

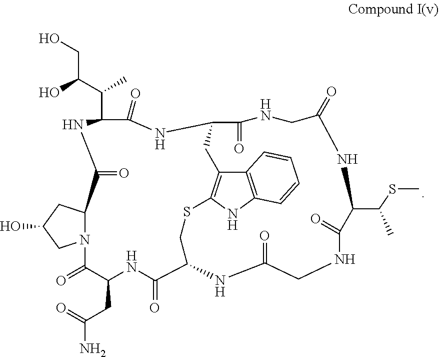 [SMILES: CS[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

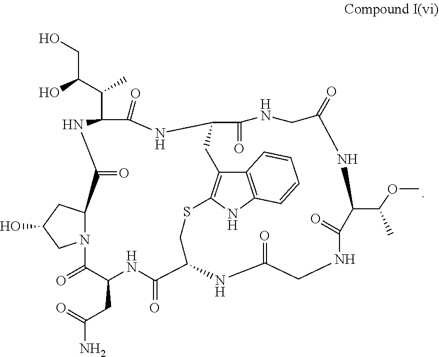 [SMILES: CO[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

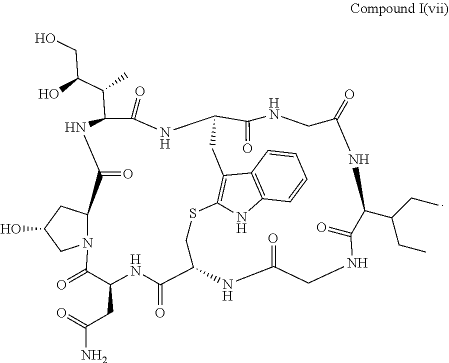 [SMILES: CCC(CC)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

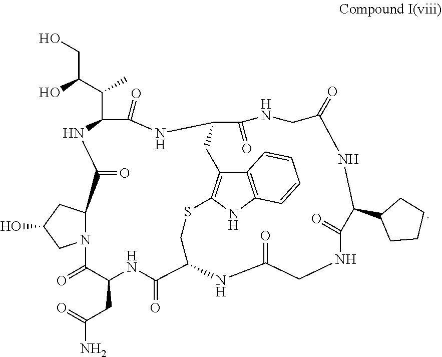 [SMILES: C[C@H]([C@@H]1NC(=O)[C@@H]2C[C@@H](O)CN2C(=O)[C@H](CC(N)=O)NC(=O)[C@@H]2CSc3[nH]c4ccccc4c3C[C@@H](NC1=O)C(=O)NCC(=O)N[C@@H](C1CCCC1)C(=O)NCC(=O)N2)[C@@H](O)CO]

In an embodiment, the amatoxin analog is:

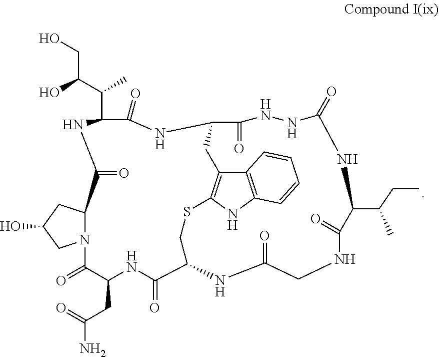 [SMILES: CC[C@H](C)[C@@H]1NC(=O)NNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

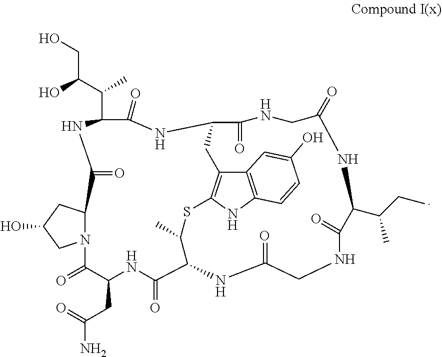 [SMILES: CC[C@H](C)[C@@H]1NC(=O)CNC(=O)[C@H]2Cc3c([nH]c4ccc(O)cc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

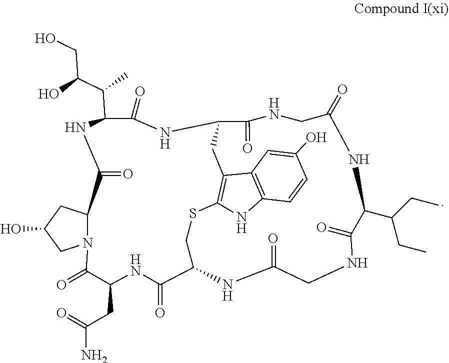 [SMILES: CCC(CC)[C@@H]1NC(=O)CNC(=O)[C@H]2Cc3c([nH]c4ccc(O)cc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

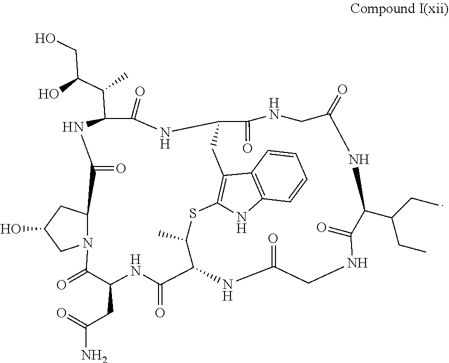 [SMILES: CCC(CC)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

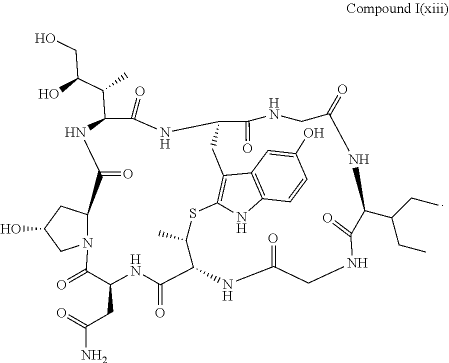 [SMILES: CCC(CC)[C@@H]1NC(=O)CNC(=O)[C@H]2Cc3c([nH]c4ccc(O)cc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is:

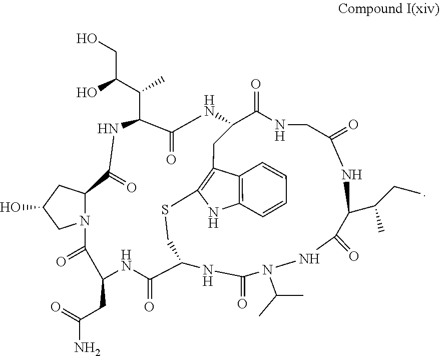 [SMILES: CC[C@H](C)[C@@H]1NC(=O)CNC(=O)[C@@H]2Cc3c([nH]c4ccccc34)SC[C@H](NC(=O)N(C(C)C)NC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the amatoxin analog is Compound I(i), Compound I(ii), Compound I(iii), Compound I(iv), Compound I(v), Compound I(vi), Compound I(vii), Compound I(viii), Compound I(ix), Compound I(x), Compound I(xi), Compound I(xii), Compound I(xiii) or Compound I(xiv).

In some embodiments, the amatoxin analog comprises:
(a) a Gly⁵ residue of the formula:

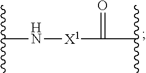

and
(b) a Gly⁷ residue of the formula:

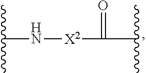

wherein
   X¹ and X² are each independently (CH₂)ₘ, CHCH₃, CHCH₂CH₃, C(CH₃)₂ or NR′, wherein m is an integer from 1 to 5 and provided that at least one of X¹ and X² is NR′; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
   or a pharmaceutically acceptable salt thereof,
   wherein in the amatoxin analog or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

Accordingly, the present disclosure also includes an amatoxin analogue that is a compound of Formula II:

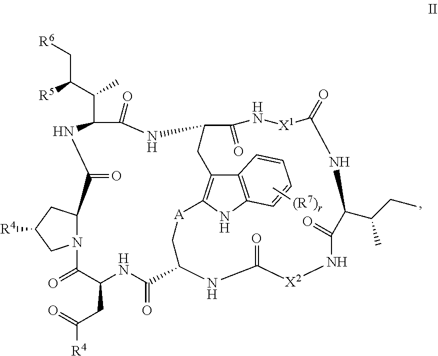

wherein
   X¹ and X² are each independently (CH₂)ₘ, CHCH₃, CHCH₂CH₃, C(CH₃)₂ or NR′, wherein m is an integer from 1 to 5 and provided that at least one of X¹ and X² is NR′;
   A is S, S(O), SO₂, Se, Se(O) or SeO₂;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   r is an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
   or a pharmaceutically acceptable salt thereof;
   wherein in the compound of Formula II or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In an embodiment, R³ is OR′ or N(R′)₂. In another embodiment, R³ is OR′ or N(R′)₂, wherein R′ is H. In a further embodiment, R³ is NH₂.

In an embodiment, R⁴ is H. In another embodiment, R⁴ is OH.

In an embodiment, R⁵ and R⁶ are each independently H or OR′. In another embodiment, R⁵ and R⁶ are each independently H or OH. In an embodiment, R⁵ is H and R⁶ is OH. In another embodiment, R⁵ is OH and R⁶ is H. In an embodiment, R⁵ and R⁶ are each H. In another embodiment, R⁵ and R⁶ are each OH. In an embodiment, R⁵ is H. In another embodiment of the present disclosure, R⁵ is OR′. In a further embodiment, R⁵ is OH. In an embodiment, R⁶ is H. In another embodiment, R⁶ is OR′. In a further embodiment, R⁶ is OH.

In an embodiment, r is an integer from 0 to 3. In another embodiment, r is 0, 1 or 2. In another embodiment, r is 0 or 1. In an embodiment, r is 0. In another embodiment, r is 1.

In an embodiment, R⁴, R⁵ and R⁶ are each OH.

In an embodiment, each R⁷ is independently OR′. In another embodiment, R⁷ is OH. In a further embodiment, R⁷ is 4-OH, 5-OH, 6-OH or 7-OH. In an embodiment, R⁷ is 5-OH.

In an embodiment, X¹ and X² are each independently (CH₂)ₘ or NR′ provided that at least one of X¹ and X² is NR′. In a further embodiment, X¹ is NR′. In an embodiment, X¹ is NH or NC₁₋₂₀alkyl. In another embodiment, X¹ is NH or NC₁₋₆alkyl. In a further embodiment, X¹ is NH or N—CH(CH₃)₂. In an embodiment, X¹ is NH. In another embodiment, X¹ is N—CH(CH₃)₂. In a further embodiment, X² is NR′. In an embodiment, X² is NH or NC₁₋₂₀alkyl. In another embodiment, X² is NH or NC₁₋₆alkyl. In a further embodiment, X² is NH or N—CH(CH₃)₂. In another embodiment, X² is N—CH(CH₃)₂. In another embodiment, X² is NH. In an embodiment, X¹ is CH₂ and X² is NR′. In an embodiment, X¹ is CH₂ and X² is NH or NC₁₋₂₀alkyl. In another embodiment, X¹ is CH₂ and X² is NH or NC₁₋₆alkyl. In a further embodiment, X¹ is CH₂ and X² is NH or N—CH(CH₃)₂. In another embodiment, X¹ is CH₂ and X² is N—CH(CH₃)₂. In another embodiment, X¹ is CH₂ and X₂ is NH. In a further embodiment, X¹ is NR′ and X² is CH₂. In an embodiment, X¹ is NH or NC₁₋₂₀alkyl and X² is CH₂. In another embodiment, X¹ is NH or NC₁₋₆alkyl and X² is CH₂. In a further embodiment, X¹ is NH or N—CH(CH₃)₂ and X² is CH₂. In another embodiment, X¹ is N—CH(CH₃)₂ and X² is CH₂. In another embodiment, X¹ is NH and X² is CH₂.

In an embodiment, each m is independently an integer from 1 to 4. In another embodiment, each m is independently an integer from 1 to 3. In a further embodiment, each m is independently 1 or 2. In another embodiment, m is 1.

In an embodiment, A is S, (R)—S(O), Se or (R)—Se(O). In an embodiment, A is S or (R)—S(O). In another embodiment, A is S. In a further embodiment, A is (R)—S(O).

In an embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is unsubstituted. In another embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl. In a further embodiment, each R′ is independently H or C₁₋₂₀alkyl. In a further embodiment, each R′ is independently H or C₁₋₆alkyl. In some embodiments, R′ is H. In some embodiments, R′ is C₁₋₆alkyl.

In some embodiments, the amatoxin analogue comprises:
(a) an Ile⁶ residue of the formula:

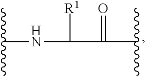

wherein R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S;
each Z is independently (CH₂)ₙO(CH₂)ₚ, (CH₂)ₙS(CH₂)ₚ, (CH₂)ₙSe(CH₂)ₚ or (CH₂)_{q};
each n is independently an integer from 0 to 4;
each p is independently an integer from 0 to 4;
n+p in each Z, is independently ≤4;
each q is independently an integer from 0 to 4; and
wherein one or more single bonds between two available carbon atoms in R¹ is/are optionally replaced by a double bond; and
(b) a Cys⁸ residue of the formula:

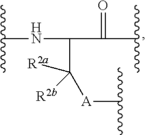

wherein
A is S, S(O), SO₂, Se, Se(O) or SeO₂; and
R^{2a} and R^{2b} are each independently H or C₁₋₁₀alkyl, provided that at least one of R^{2a} and R^{2b} is C₁₋₁₀alkyl; or R^{2a} and R^{2b} together with the carbon atom to which they are attached, together form C₃₋₁₀cycloalkyl;
each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
or a pharmaceutically acceptable salt thereof,
wherein in the amatoxin analog or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

Accordingly, the present disclosure also includes an amatoxin analogue that is a compound of Formula III:

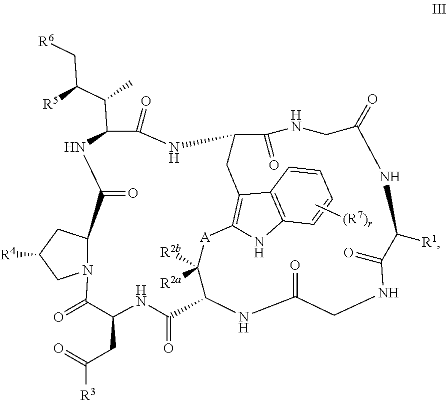

wherein
   A is S, S(O), SO₂, Se, Se(O) or SeO₂;
   R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S; and wherein one or more single bonds between two available carbon atoms in R¹ is/are optionally replaced by a double bond;
   R^{2a} and R^{2b} are each independently H or C₁₋₁₀alkyl, provided that at least one of R^{2a} and R^{2b} is C₁₋₁₀alkyl; or R^{2a} and R^{2b} together with the carbon atom to which they are attached, together form C₃₋₁₀cycloalkyl;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₙS(CH₂)ₚ, (CH₂)ₙSe(CH₂)ₚ or (CH₂)_{q};
   each n is independently an integer from 0 to 4;
   each p is independently an integer from 0 to 4;
   n+p in each Z, is independently ≤4;
   each q is independently an integer from 0 to 4;
   r is an integer from 0 to 4; and
   each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
   or a pharmaceutically acceptable salt thereof,
   wherein in the compound of Formula III or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In some embodiments, R¹ is saturated; i.e. no single bonds between carbon atoms have been replaced by a double bond. In other embodiments, one or more single bonds between two available carbon atoms in R¹ is/are replaced by a double bond.

In an embodiment, R¹ is CH(Z—CH₃)₂, wherein one or more available hydrogens is/are optionally substituted with SH. In some embodiments, R¹ is devoid of SH groups; i.e. no hydrogens are substituted with SH. In some embodiments, R¹ comprises one or more SH groups. In an embodiment, each Z is independently (CH₂)ₙO(CH₂)ₚ, (CH₂)ₙS(CH₂) or (CH₂)_{q}. In another embodiment, each Z is independently (CH₂)_{q}. In a further embodiment, one Z is (CH₂)ₙO(CH₂)ₚ and the other Z is (CH₂)_{q}. In another embodiment, one Z is (CH₂)ₙS(CH₂)ₚ and the other Z is (CH₂)_{q}. In an embodiment, each n is independently an integer from 0 to 3. In another embodiment, each n is independently an integer from 0 to 2. In another embodiment, n is 0. In an embodiment, each p is independently an integer from 0 to 3. In another embodiment, each p is independently an integer from 0 to 2. In another embodiment, p is 0. In an embodiment, n+p in each Z, is independently 3. In another embodiment, n+p in each Z, is independently 2. In a further embodiment, n+p=0. In an embodiment, each q is independently an integer from 0 to 3. In another embodiment, each q is independently an integer from 0 to 1. In another embodiment, q is 1.

In an embodiment, R¹ is C₃₋₁₀cycloalkyl. In another embodiment, R¹ is C₃₋₆cycloalkyl. In a further embodiment, R¹ is cyclopentyl. In some embodiments, R¹ is carbocyclic; i.e. no carbon atoms in the cycloalkyl (e.g. the C₃₋₁₀cycloalkyl such as cyclopentyl) have been replaced by O or S. In other embodiments, one or more available carbon atoms in the cycloalkyl (e.g. the C₃₋₁₀cycloalkyl such as cyclopentyl) is/are replaced by S or O. In an embodiment, R¹ is a heterocycloalkyl comprising S and/or O atoms such as tetrahydrofuranyl or tetrahydrothiophenyl. In some embodiments, R¹ is unsubstituted. In some embodiments, one or more available hydrogens on the C₃₋₁₀cycloalkyl is/are optionally substituted with C₁₋₁₀alkyl.

In an embodiment, R¹ is

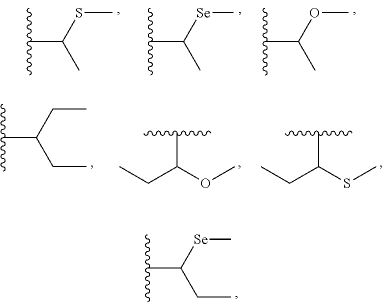

cyclopentyl,

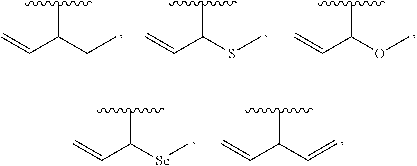

tetrahydrofuranyl, or tetrahydrothiophenyl. In another embodiment, R¹ is

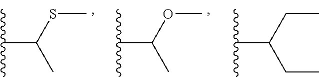

or cyclopentyl. In another embodiment, R¹ is

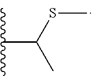 [SMILES: *C(C)SC]

In a further embodiment, R¹ is

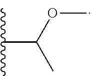 [SMILES: *C(C)OC]

In an embodiment, R¹ is

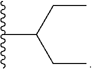 [SMILES: *C(CC)CC]

In another embodiment, R¹ is

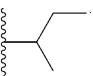 [SMILES: *C(C)CC]

In another embodiment, R¹ is other than

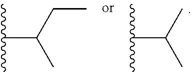

In an embodiment, R¹ is cyclopentyl.

In an embodiment, R^{2a} and R^{2b} are each independently H or C₁₋₁₀alkyl, provided that at least one of R^{2a} and R^{2b} is C₁₋₁₀alkyl. In a further embodiment, at least one of R^{2a} and R^{2b} is C₁₋₆alkyl. In another embodiment, at least one of R^{2a} and R is C₁₋₃alkyl. In another embodiment, at least one of R^{2a} and R^{2b} is methyl. In an embodiment, R^{2a} is CH₃ and R² is CH₃. In another embodiment, R^{2a} is H and R is CH₃. In a further embodiment, R^{2a} is CH₃ and R is H.

In an embodiment, R^{2a} and R^{2b} together with the carbon atom to which they are attached, together form C₃₋₁₀cycloalkyl. In another embodiment, R^{2a} and R^{2b} together with the carbon atom to which they are attached, together form C₃₋₆cycloalkyl. In a further embodiment, R^{2a} and R^{2b} together with the carbon atom to which they are attached, together form cyclopropyl.

In an embodiment, R³ is OR′ or N(R′)₂. In another embodiment, R³ is OR′ or N(R′)₂, wherein R′ is H. In a further embodiment, R³ is NH₂.

In an embodiment, R⁴ is H. In another embodiment, R⁴ is OH.

In an embodiment, R⁵ and R⁶ are each independently H or OR′. In another embodiment, R⁵ and R⁶ are each independently H or OH. In an embodiment, R⁵ is H and R⁶ is OH. In another embodiment, R⁵ is OH and R⁶ is H. In an embodiment, R⁵ and R⁶ are each H. In another embodiment, R⁵ and R⁶ are each OH. In an embodiment, R⁵ is H. In another embodiment of the present disclosure, R⁵ is OR′. In a further embodiment, R⁵ is OH. In an embodiment, R⁶ is H. In another embodiment, R⁶ is OR′. In a further embodiment, R⁶ is OH.

In an embodiment, r is an integer from 0 to 3. In another embodiment, r is 0, 1 or 2. In another embodiment, r is 0 or 1. In an embodiment, r is 0. In another embodiment, r is 1.

In an embodiment, R⁴, R⁵ and R⁶ are each OH.

In an embodiment, each R⁷ is independently OR′. In another embodiment, R⁷ is OH. In a further embodiment, R⁷ is 4-OH, 5-OH, 6-OH or 7-OH. In an embodiment, R⁷ is 5-OH.

In an embodiment, A is S, (R)—S(O), Se or (R)—Se(O). In an embodiment, A is S or (R)—S(O). In another embodiment, A is S. In a further embodiment, A is (R)—S(O).

In an embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is unsubstituted. In another embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl. In a further embodiment, each R′ is independently H or C₁₋₂₀alkyl. In a further embodiment, each R′ is independently H or C₁₋₆alkyl. In some embodiments, R′ is H. In some embodiments, R′ is C₁₋₆alkyl.

The present disclosure also includes an amatoxin analog that is a compound of Formula IV:

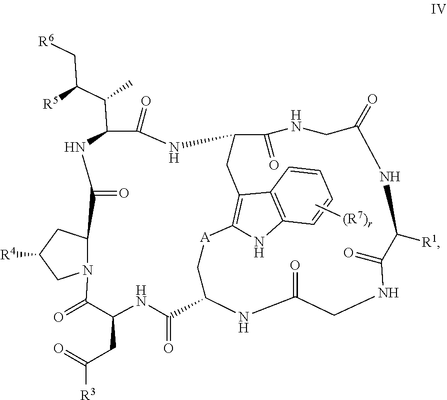

wherein
   A is S, S(O), SO₂, Se, Se(O) or SeO₂;
   R¹ is CH(Z—CH₃)₂, wherein one or more available hydrogens is/are optionally substituted with SH; or R¹ is C₃₋₁₀cycloalkyl, wherein one or more available hydrogens on the cycloalkyl are optionally substituted with C₁₋₁₀alkyl and/or one or more available carbons in the cycloalkyl is optionally replaced by O or S; and wherein one or more single bonds between two available carbon atoms in R¹ is/are optionally replaced by a double bond;
   R³ is OR′, NH(OR′), NH—NHR′ or N(R′)₂;
   R⁴ is H or OH;
   R⁵ and R⁶ are each independently H, OR′, NHR′, SR′ or halo;
   each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
   each Z is independently (CH₂)ₙO(CH₂)ₚ, (CH₂)ₙS(CH₂)ₚ, (CH₂)ₙSe(CH₂)ₚ or (CH₂)_{q};
   each n is independently an integer from 0 to 4;
   each p is independently an integer from 0 to 4;
   n+p in each Z, is independently ≤4;
   each q is independently an integer from 0 to 4,
   r is independently an integer from 0 to 4;
   provided that R¹ is other than

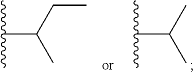

each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl;
or a pharmaceutically acceptable salt thereof,
wherein in the compound of Formula IV or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

In some embodiments, R¹ is saturated; i.e. no single bonds between carbon atoms have been replaced by a double bond. In other embodiments, one or more single bonds between two available carbon atoms in R¹ is/are replaced by a double bond.

In an embodiment, R¹ is CH(Z—CH₃)₂, wherein one or more available hydrogens is/are optionally substituted with SH provided that R¹ is other than

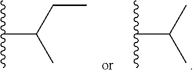

In some embodiments, R¹ is devoid of SH groups; i.e. no hydrogens are substituted with SH. In some embodiments, R¹ comprises one or more SH groups. In an embodiment, each Z is independently (CH₂)ₙO(CH₂)ₚ, (CH₂)ₙS(CH₂) or (CH₂)_{q} provided that R¹ is other than

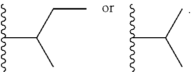

In another embodiment, each Z is independently (CH₂)_{q} provided that R¹ is other than

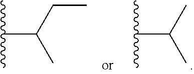

In a further embodiment, one Z is (CH₂)ₙO(CH₂)ₚ and the other Z is (CH₂)_{q}. In another embodiment, one Z is (CH₂)S(CH₂)ₚ and the other Z is (CH₂)_{q}. In an embodiment, each n is independently an integer from 0 to 3. In another embodiment, each n is independently an integer from 0 to 2. In another embodiment, n is 0. In an embodiment, each p is independently an integer from 0 to 3. In another embodiment, each p is independently an integer from 0 to 2. In another embodiment, p is 0. In an embodiment, n+p in each Z, is independently ≤3. In another embodiment, n+p in each Z, is independently ≤2. In a further embodiment, n+p=0. In an embodiment, each q is independently an integer from 1 to 3. In another embodiment, each q is 1.

In an embodiment, R¹ is C₃₋₁₀cycloalkyl. In another embodiment, R¹ is C₃₋₆cycloalkyl. In a further embodiment, R¹ is cyclopentyl. In some embodiments, R¹ is carbocyclic; i.e. no carbon atoms in the cycloalkyl (e.g. the C₃₋₁₀cycloalkyl such as cyclopentyl) have been replaced by O or S. In other embodiments, one or more available carbon atoms in the cycloalkyl (e.g. the C₃₋₁₀cycloalkyl such as cyclopentyl) is/are replaced by S or O. In an embodiment, R¹ is a heterocycloalkyl comprising S and/or O atoms such as tetrahydrofuranyl or tetrahydrothiophenyl. In some embodiments, R¹ is unsubstituted. In some embodiments, one or more available hydrogens on the C₃₋₁₀cycloalkyl is/are optionally substituted with C₁₋₁₀alkyl.

In an embodiment, R¹ is

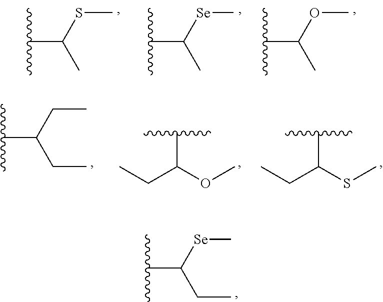

cyclopentyl,

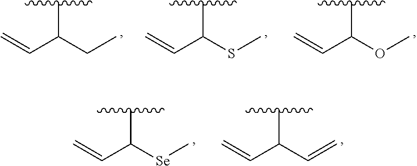

tetrahydrofuranyl, or tetrahydrothiophenyl. In another embodiment, R¹ is

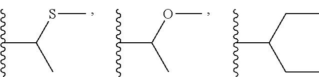

or cyclopentyl. In another embodiment, R¹ is

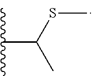 [SMILES: *C(C)SC]

In a further embodiment, R¹ is

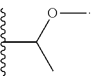 [SMILES: *C(C)OC]

In another embodiment, R¹ is

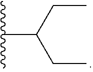 [SMILES: *C(CC)CC]

In an embodiment, R¹ is cyclopentyl.

In an embodiment, R³ is OR′ or N(R′)₂. In another embodiment, R³ is OR′ or N(R′)₂, wherein R′ is H. In a further embodiment, R³ is NH₂.

In an embodiment, R⁵ and R⁶ are each independently H or OR′. In another embodiment, R⁵ and R⁶ are each independently H or OH. In an embodiment, R⁵ is H and R⁶ is OH. In another embodiment, R⁵ is OH and R⁶ is H. In an embodiment, R⁵ and R⁶ are each H. In another embodiment, R⁵ and R⁶ are each OH. In an embodiment, R⁵ is H. In another embodiment of the present disclosure, R⁵ is OR′. In a further embodiment, R⁵ is OH. In an embodiment, R⁶ is H. In another embodiment, R⁶ is OR′. In a further embodiment, R⁶ is OH.

In an embodiment, r is an integer from 0 to 3. In another embodiment, r is 0, 1 or 2. In another embodiment, r is 0 or 1. In an embodiment, r is 0. In another embodiment, r is 1.

In an embodiment, R⁴, R⁵ and R⁶ are each OH.

In an embodiment, each R⁷ is independently OR′. In another embodiment, R⁷ is OH. In a further embodiment, R⁷ is 4-OH, 5-OH, 6-OH or 7-OH. In an embodiment, R⁷ is 5-OH.

In an embodiment, A is S, (R)—S(O), Se or (R)—Se(O). In an embodiment, A is S or (R)—S(O). In another embodiment, A is S. In a further embodiment, A is (R)—S(O).

In an embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is unsubstituted. In another embodiment, each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl. In a further embodiment, each R′ is independently H or C₁₋₂₀alkyl. In a further embodiment, each R′ is independently H or C₁₋₆alkyl. In some embodiments, R′ is H. In some embodiments, R′ is C₁₋₆alkyl.

The amatoxin analogs of the present disclosure such as the compounds of Formulae I, II, III and IV, and salts thereof, as described herein may, for example, be coupled to a linker to provide a compound-linker construct that may, for example, be used in the manufacture of conjugates which may, for example, be useful in the treatment of diseases such as cancer.

Accordingly, the present disclosure also includes a compound-linker construct comprising an amatoxin analog as described herein coupled to a linker, wherein the linker comprises a reactive group R⁸ for conjugating the compound-linker construct to a target-binding moiety.

The amatoxin analogs of the present disclosure are coupled to the linker in any suitable configuration, the selection of which can be made by a person skilled in the art. For example, the location of the coupling may depend, for example, on the identity of the amatoxin analog and/or the linker. In an embodiment, the linker is coupled to the amatoxin analog or the salt thereof at a site indicated by R³, R⁴, R⁵, R⁶ and/or R⁷ in an amatoxin analog as described herein. In another embodiment, the linker is coupled to the amatoxin analog or the salt thereof through a moiety obtained from the reaction of R³, R⁴, R⁵, R⁶ and/or R⁷ in an amatoxin analog as described herein with a complementary functional group on the linker.

In an embodiment, at a site indicated by R³, R⁴, R⁵, R⁶ and/or R⁷ in an amatoxin analog as described herein, the compound-linker construct comprises a group of the formula —O-L, wherein L represents the linker. In another embodiment, at a site indicated by R³, R⁴, R⁵, R⁶ and/or R⁷ in an amatoxin analog described herein, the compound-linker construct comprises a group of the formula —O—C(O)-L, wherein L represents the linker. In a further embodiment, at a site indicated by R³, R⁵, R⁶ and/or R⁷ in an amatoxin analog as described herein, the compound-linker construct comprises a group of the formula —NR′-L, wherein R′ is H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂-20alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl; and L represents the linker.

In an embodiment, R′ is H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is unsubstituted. In another embodiment, R′ is H, C₁₋₂₀alkyl, C₂₋₂₀alkenyl, C₂-20alkynyl, C₃₋₂₀cycloalkyl, C₆₋₃₀aryl, heterocycloalkyl or heteroaryl. In a further embodiment, each R′ is independently H or C₁₋₂₀alkyl. In an embodiment, R′ is H.

The linker can be any suitable linker. For example, it will be appreciated by a person skilled in the art that amatoxins are relatively non-toxic when coupled to a biomolecule carrier such as a target-binding moiety, and advantageously only exert their cytotoxic activity after internalization in the target cells. Accordingly, a conjugate comprising the target-binding moiety conjugated to the compound coupled to the linker is advantageously substantially stable in the plasma after administration and a suitable linker may desirably allow, for example, for substantially releasing the compound subsequent to internalization in the target cells.

In an embodiment, the linker is stable linker. The term “stable linker” as used herein refers to a linker that typically releases the compound after the target-binding moiety to which it is conjugated is degraded intracellularly, for example, in the lysosomes. In other words, the linker is substantially stable in an intracellular reducing environment and in the presence of enzymes such as lysosomal peptidases (e.g. Cathepsin B). In an embodiment, the stable linker is devoid of an enzyme-cleavable structure (e.g. a dipeptide sequence cleavable by Cathepsin B) and/or a disulfide group. In another embodiment, the stable linker comprises an —[CH₂—X³—CH₂]ₛ—CH₂— moiety, wherein X³ is O, CH₂ or S, and s is an integer from 1 to 8.

In another embodiment of the present disclosure, the linker is a cleavable linker. The term “cleavable linker” as used herein refers to a linker that is cleavable by an enzyme and/or in a reducing environment. In an embodiment of the present disclosure, the cleavable linker is cleavable by an intracellular protease. In another embodiment of the present disclosure, the linker is cleavable by at least one agent selected from the group consisting of cysteine protease, metalloproteinase, serine protease, threonine protease, aspartic protease, glycosidase, phosphodiesterase, and reductase. Such linkers may, for example, comprise a peptide motif cleavable by such an enzyme. In another embodiment of the present disclosure, the linker comprises a motif selected from the group consisting of: Val-Ala, Val-Cit, Val-Lys, Val-Arg, Phe-Lys-Gly-Pro-Leu-Gly, Ala-Ala-Pro-Val, β-glucuronide and β-galactoside.

In an embodiment, the linker further comprises a self-immolating moiety. The term “self-immolating moiety” as used herein, refers to a moiety, which, after enzymatic cleavage of the linker, spontaneously cleaves from the remainder of the compound-linker construct, thereby releasing the compound. In an embodiment, the self-immolating moiety is a p-aminobenzyl alcohol (PAB) moiety. In another embodiment, the PAB is conjugated to a peptide (e.g. a dipeptide) portion of the linker via the aromatic amine group of the PAB. In another embodiment, the PAB is conjugated to the compound via a carbamate group coupled to a primary or secondary amine or in an alternative embodiment, the PAB is coupled directly to the amatoxin analogue.

In an embodiment:
(a) at a site indicated by R³, R⁴, R⁵, R⁶ or R⁷ in an amatoxin analog as described herein, the compound-linker construct comprises a group of the formula:

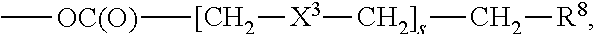

wherein X³ is O, CH₂ or S, s is an integer from 1 to 8 and R⁸ is the reactive group for conjugating the compound-linker construct to a target-binding moiety;
(b) at a site indicated by R³, R⁵, R⁶ or R⁷ in an amatoxin analog as described herein, the compound-linker construct comprises a group of the formula:

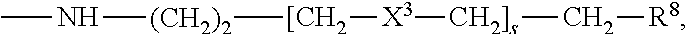

wherein X³ is O, CH₂ or S, s is an integer from 1 to 8 and R⁸ is the reactive group for conjugating the compound-linker construct to a target-binding moiety;
(c) at a site indicated by R³, R⁵, R⁶ or R⁷ in an amatoxin analog as described herein, the compound-linker construct comprises a group of the formula:

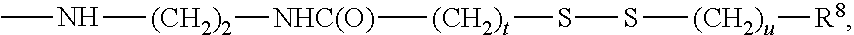

wherein t and u are independently an integer from 1 to 6 and R⁸ is the reactive group for conjugating the compound-linker construct to a target-binding moiety;
(d) at a site indicated by R³, R⁵, R⁶ or R⁷ in an amatoxin analog as described herein, the compound-linker construct comprises a group of the formula:

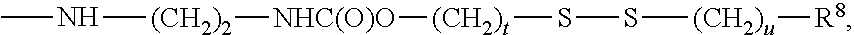

wherein t and u are independently an integer from 1 to 6 and R⁸ is the reactive group for conjugating the compound-linker construct to a target-binding moiety;
(e) at a site indicated by R³, R⁵, R⁶ or R⁷ in an amatoxin analog as described herein, the compound-linker construct comprises a group of the formula:

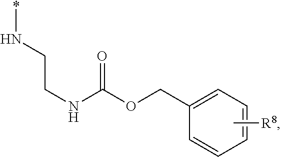

wherein * represents the site of attachment to the remainder of the compound-linker construct and R⁸ is the reactive group for conjugating the compound-linker construct to a target-binding moiety;
(f) at a site indicated by R³, R⁵, R⁶ or R⁷ in an amatoxin analog as described herein, the compound-linker construct comprises a group of the formula:

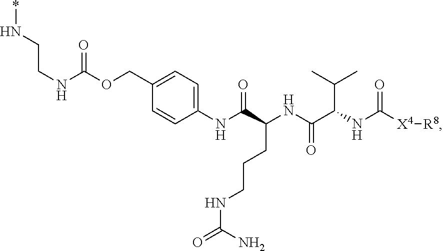

wherein * represents the site of attachment to the remainder of the compound-linker construct, X⁴ is C₁₋₁₀alkylene and R⁸ is the reactive group for conjugating the compound-linker construct to a target-binding moiety; or
(g) at a site indicated by R³, R⁴, R⁵, R⁶ or R⁷ in an amatoxin analog as described herein, the compound-linker construct comprises a group of the formula:

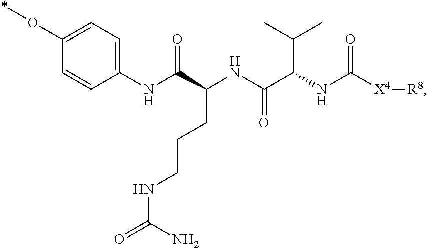

wherein * represents the site of attachment to the remainder of the compound-linker construct, X⁴ is C₁₋₁₀alkylene and R⁸ is the reactive group for conjugating the compound-linker construct to a target-binding moiety.

In an embodiment, at a site indicated by R³, R⁴, R⁵, R⁶ or R⁷ in an amatoxin analog as described herein, the compound-linker construct comprises a group of the formula:

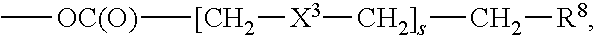

wherein X³ is O, CH₂ or S, s is an integer from 1 to 8 and R⁸ is the reactive group for conjugating the compound-linker construct to a target-binding moiety. In an embodiment, X³ is O. In another embodiment, X³ is S. In a further embodiment, X³ is CH₂. In an embodiment, the site is indicated by R³ in the amatoxin analog as described herein. In another embodiment, the site is indicated by R⁴ in the amatoxin analog as described herein. In another embodiment, the site is indicated by Rin the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁵, R⁶ or R⁷ in an amatoxin analog as described herein, the compound-linker construct comprises a group of the formula:

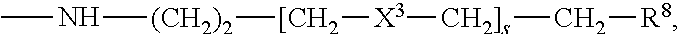

wherein X³ is O, CH₂ or S, s is an integer from 1 to 8 and R⁸ is the reactive group for conjugating the compound-linker construct to a target-binding moiety. In an embodiment, X³ is O. In another embodiment, X³ is S. In a further embodiment, X³ is CH₂. In an embodiment, the site is indicated by R³ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁵, R⁶ or R⁷ in an amatoxin analog as described herein, the compound-linker construct comprises a group of the formula:

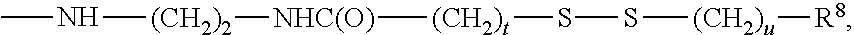

wherein t and u are independently an integer from 1 to 6 and R is the reactive group for conjugating the compound-linker construct to a target-binding moiety. In an embodiment, t and u are independently an integer from 1 to 4. In another embodiment, t is 3. In a further embodiment, u is 3. In an embodiment, the site is indicated by R³ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁵, R⁶ or R⁷ in an amatoxin analog as described herein, the compound-linker construct comprises a group of the formula:

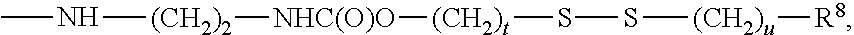

wherein t and u are independently an integer from 1 to 6 and R⁸ is the reactive group for conjugating the compound-linker construct to a target-binding moiety. In an embodiment, t and u are independently an integer from 1 to 4. In another embodiment, t is 2. In a further embodiment, u is 3. In an embodiment, the site is indicated by R³ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁵, R⁶ or R⁷ in an amatoxin analog as described herein, the compound-linker construct comprises a group of the formula:

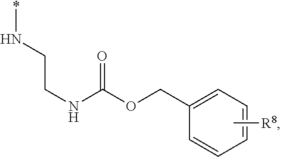

wherein * represents the site of attachment to the remainder of the compound-linker construct and R⁸ is the reactive group for conjugating the compound-linker construct to a target-binding moiety. In an embodiment, the compound-linker construct comprises a group of the formula:

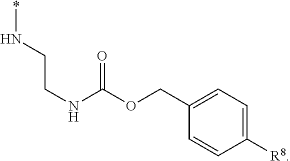

In an embodiment, the site is indicated by R³ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁵, R⁶ or R⁷ in an amatoxin analog as described herein, the compound-linker construct comprises a group of the formula:

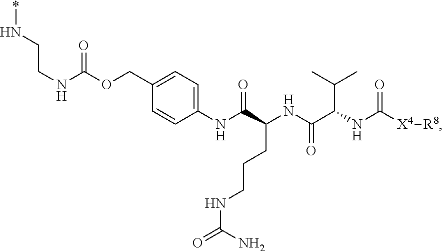

wherein * represents the site of attachment to the remainder of the compound-linker construct, X⁴ is C₁₋₁₀alkylene and R⁸ is the reactive group for conjugating the compound-linker construct to a target-binding moiety. In an embodiment, X⁴ is —CH₂CH₂—. In an embodiment, the site is indicated by R³ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In another embodiment, at a site indicated by R³, R⁴, R⁵, R⁶ or R⁷ in an amatoxin analog as described herein, the compound-linker construct comprises a group of the formula:

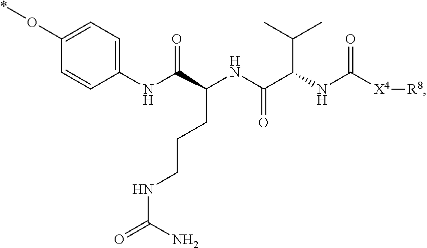

wherein * represents the site of attachment to the remainder of the compound-linker construct, X⁴ is C₁₋₁₀alkylene and R⁸ is the reactive group for conjugating the compound-linker construct to a target-binding moiety. In an embodiment, X⁴ is —CH₂CH₂—. In an embodiment, the site is indicated by R³ in the amatoxin analog as described herein. In another embodiment, the site is indicated by R⁴ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

In an embodiment, at a site indicated by R³, R⁴, R⁵, R⁶ or R⁷ in the amatoxin analog as described herein, the compound-linker construct comprises a group of the formula:

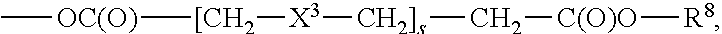

wherein X³ is a bond, O, CH₂ or S, s is an integer from 1 to 8 and R⁸ is the reactive group for conjugating the compound-linker construct to a target-binding moiety. In an embodiment, X³ is CH₂ or a bond. In another embodiment, X₃ is a bond. In a further embodiment, s is 1. In an embodiment, the site is indicated by R³ in the amatoxin analog as described herein. In another embodiment, the site is indicated by R⁴ in the amatoxin analog as described herein. In another embodiment, the site is indicated by R⁵ in the amatoxin analog as described herein. In another embodiment, the site is indicated by R⁶ in the amatoxin analog as described herein. In another embodiment, the site is indicated by R⁷ in the amatoxin analog as described herein.

The reactive group R⁸ is any reactive group suitable for conjugating the compound-linker construct to a target-binding moiety and may depend, for example, on the identity of the target-binding moiety and/or a complimentary reactive group thereon.

In an embodiment of the present disclosure, R⁸ is selected from:

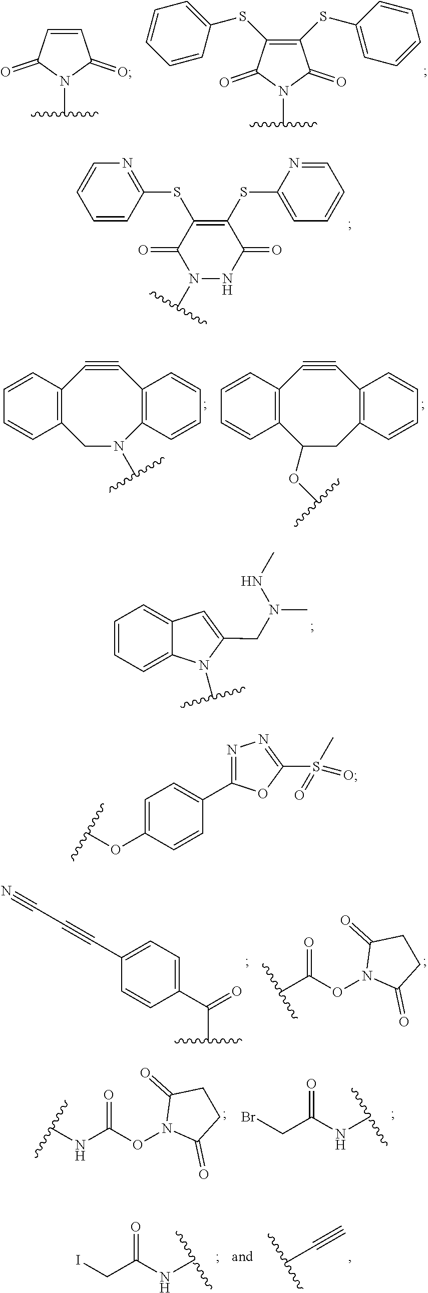

wherein [symbol] represents the site of attachment of R⁸ to the remainder of the linker; or R⁸ comprises an azide, thiol, tetrazine, trans-cyclooctene or acrylamide functional group.

In another embodiment, R⁸ is

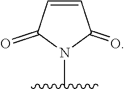 [SMILES: *N1C(=O)C=CC1=O]

In an embodiment, at a site indicated by R⁶ in the amatoxin analog as described herein, the compound-linker construct comprises a group of the formula:

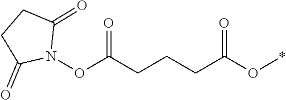 [SMILES: *OC(=O)CCCC(=O)ON1C(=O)CCC1=O]

wherein * represents the site of attachment to the remainder of the compound-linker construct.

The present disclosure also includes a conjugate comprising a target-binding moiety conjugated to an amatoxin analog or a compound-linker construct as described herein.

In an embodiment, the conjugate comprises the target-binding moiety conjugated to the amatoxin analog. In another embodiment, the conjugate comprises the target-binding moiety conjugated to the compound-linker construct.

The term “target-binding moiety” as used herein refers to any suitable moiety that can specifically bind to a target molecule (e.g. protein) or epitope. In an embodiment, the target-binding moiety is a peptide, an oligonucleotide, a lipid, a lipid carrier, a nanoparticle or combinations thereof. In another embodiment, the target-binding moiety is an antibody, an antigen-binding fragment thereof, an antibody-like protein, an aptamer, a vitamin, an oligonucleotide, an affibody, a minibody, a lipid, a lipid carrier, a nanoparticle or combinations thereof. In another embodiment, the target-binding moiety is an antibody, an antigen-binding fragment thereof, an antibody-like protein, an aptamer, a vitamin, an oligonucleotide, an affibody, a minibody, a lipid, a lipid carrier or a nanoparticle. In a further embodiment, the target-binding moiety is an antibody, antigen-binding fragment thereof or an antibody-like protein. In a further embodiment, the target-binding moiety is an antibody. In an embodiment, the conjugate has a drug-to-target-binding moiety ratio e.g., a drug-to-antibody ratio (DAR) of 0.9 to 6.25. In another embodiment, the conjugate has a drug-to-target-binding moiety ratio e.g., a drug-to-antibody ratio (DAR) of 0.9, 1.4, 2.1, 2.7, 3.5, 4.9, 5.8 or 6.25. In an embodiment, the conjugate has a drug-to-target-binding moiety ratio e.g., a drug-to-antibody ratio (DAR) of 0.9. In another embodiment, the conjugate has a drug-to-target-binding moiety ratio e.g., a drug-to-antibody ratio (DAR) of 1.4. In another embodiment, the conjugate has a drug-to-target-binding moiety ratio e.g., a drug-to-antibody ratio (DAR) of 2.1. In another embodiment, the conjugate has a drug-to-target-binding moiety ratio e.g., a drug-to-antibody ratio (DAR) of 2.7. In another embodiment, the conjugate has a drug-to-target-binding moiety ratio e.g., a drug-to-antibody ratio (DAR) of 3.5. In another embodiment, the conjugate has a drug-to-target-binding moiety ratio e.g., a drug-to-antibody ratio (DAR) of 4.9. In another embodiment, the conjugate has a drug-to-target-binding moiety ratio e.g., a drug-to-antibody ratio (DAR) of 5.8. In another embodiment, the conjugate has a drug-to-target-binding moiety ratio e.g., a drug-to-antibody ratio (DAR) of 6.25. The term “antigen-binding fragment thereof” as used herein means that the fragment of the antibody comprises at least a functional antigen-binding domain. The term “antibody-like protein” as used herein refers to a protein that is not strictly an antibody but has the capability of binding to a target molecule (e.g. protein) or epitope. The “antibody-like protein” is a protein that has been engineered (e.g. by mutagenesis of Ig loops) to specifically bind to the target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. In an embodiment, the scaffold protein is a small globular protein. The scaffold of antibody-like proteins can be based on, for example, without limitation, affilin proteins, affibodies, anti-calins, lipocalins, ubiquitin, leucine-rich repeat proteins, and designed ankyrin repeat proteins (see, for example: Binz et al., “Engineering novel binding proteins from nonimmunoglobulin domains” Nat Biotechnol. 2005, 23:10, 1257-68). The antibody or antigen-binding fragment thereof can be from any suitable immunoglobulin type (e.g. IgG, IgE, IgM, IgD, IgA and/or IgY), class (e.g., IgG1, IgG2, IgG3, IgG4, IgAQ1 and/or IgA2) or subclass. Suitable antibodies and/or antigen-binding fragments thereof may include but are not limited to polyclonal, monoclonal, monovalent, bispecific, heteroconjugate, multispecific, human, humanized (e.g. CDR-grafted), deimmunized, and/or chimeric antibodies, single chain antibodies (e.g. scFv), Fab fragments, F(ab′)₂ fragments, fragments produced by a Fab expression library, diabodies or tetrabodies, nanobodies, anti-idiotypic (anti-Id) antibodies (including, but not limited to anti-Id antibodies to antibodies of the present disclosure), and epitope-binding fragments of any of the above.

In an embodiment, the antigen-binding fragments are human antigen-binding antibody fragments and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. Also included are antigen-binding fragments also comprising any combination of variable domain(s) with a hinge region, CL, CH1, CH2, and CH3 domains. The antibody, antigen-binding fragment thereof or antibody-like protein may be from any animal origin including birds and mammals. For example, in an embodiment, the antibody, antigen-binding fragment thereof or antibody-like protein is from human, rodent (e.g. mouse, rat, guinea pig, or rabbit), chicken, pig, sheep, goat, camel, cow, horse, donkey, cat, or dog origin. In another embodiment, the antibody, antigen-binding fragment thereof or antibody-like protein is of human or murine origin. The term “human” as used herein in reference to antibodies includes antibodies having the amino acid sequence of a human immunoglobulin and also includes antibodies isolated from human immunoglobulin libraries or from animals transgenic for one or more human immunoglobulin and that do not express endogenous immunoglobulins, as described for example in U.S. Pat. No. 5,939,598.

The term “specifically bind” as used herein in reference to a target-binding moiety specifically binding to a target molecule or epitope means, for example, that it has a dissociation constant K_{D} to the target molecule or epitope of at most about 100 μM. In an embodiment, K_{D} is about 100 PM or lower, about 50 μM or lower, about 30 μM or lower, about 20 μM or lower, about 10 μM or lower, about 5 μM or lower, about 1 μM or lower, about 900 nM or lower, about 800 nM or lower, about 700 nM or lower, about 600 nM or lower, about 500 nM or lower, about 400 nM or lower, about 300 nM or lower, about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower or about 10 nM or lower, about 1 nM or lower, about 900 pM or lower, about 800 pM or lower, about 700 pM or lower, about 600 pM or lower, about 500 pM or lower, about 400 pM or lower, about 300 pM or lower, about 200 pM or lower, about 100 pM or lower, about 90 pM or lower, about 80 pM or lower, about 70 pM or lower, about 60 pM or lower, about 50 pM or lower, about 40 pM or lower, about 30 pM or lower, about 20 pM or lower or about 10 pM or lower.

The terms “target molecule” and “target epitope” as used herein may refer to an antigen and an epitope of an antigen, respectively, that is specifically bound by a target-binding moiety such as an antibody, antigen-binding fragment thereof or antibody-like protein. The term “epitope” as used herein, also sometimes referred to in the art as an “antigenic determinant”, refers to a molecular structure to which, in the context of an adaptive immune response, antibodies or T cell receptors are directed and/or generated, and/or which can elicit a specific immune response. In the context of antibodies or antigen-binding fragments thereof, the term “epitope” as used herein relates to the specific molecular structure on the antigen to which the antigen-recognition site or paratop of the antibody or antigen-binding fragment thereof, binds. In an embodiment, the target molecule or target epitope is associated with cancer, non-cancerous neoplasms, an autoimmune disease, an inflammatory disease or a viral infection. In an embodiment, the target molecule or target epitope is associated with cancer, an autoimmune disease, or a viral infection. In an embodiment, the target molecule or epitope is associated with cancer. In another embodiment, the target molecule or epitope associated with cancer or non-cancerous neoplasms is present on the surface of one or more tumor cell types or tumor-associated cells in an increased concentration and/or in a different steric configuration as compared to the surface of non-tumor cells. In an embodiment, the target molecule or epitope associated with cancer is an epitope of human epidermal growth receptor 2 (HER2), prostate-specific membrane antigen (PSMA), CD20, CD269, sialyl Lewis^{X}, HER-2/neu or epithelial cell adhesion molecule (EpCAM). In a further embodiment, the target molecule or epitope associated with cancer is an epitope of human epidermal growth receptor 2 (HER2). In another embodiment, the target-binding moiety is an anti-HER2 antibody. In another embodiment, the anti-HER2 antibody is trastuzumab. In an embodiment, the target molecule or target epitope is associated with autoimmune disease. In another embodiment, the target molecule or epitope associated with autoimmune disease is preferentially expressed on cells involved in an autoimmune disease. In an embodiment, the target molecule or target epitope is associated with a viral infection. In another embodiment, the target molecule or epitope associated with a viral infection is preferentially expressed on cells involved in a viral infection.

In an embodiment, the conjugate comprises the compound-linker construct, the target-binding moiety comprises an engineered acceptor residue, R⁸ is a reactive group to a moiety comprised in the engineered acceptor residue, and the compound-linker construct is conjugated to the target-binding moiety via a moiety resulting from the reaction of the moiety comprised in the engineered acceptor residue with R⁸. The term “engineered acceptor residue” as used herein refers to a residue that is introduced into a peptide sequence of a suitable target-binding moiety that is generally not present in the native peptide sequence of the target-binding moiety. Such residues are available for reaction with R⁸ but desirably do not substantially affect, for example, immunoglobulin folding, antibody assembly, antigen binding and/or Fc domain effector functions. In an embodiment, the engineered acceptor residue is an engineered cystine residue and R⁸ is a thiol-reactive group. The term “engineered cysteine residue” as used herein refers to a cysteine residue that is introduced into the peptide sequence of a suitable target-binding moiety that is generally not present in the native peptide sequence of the target-binding moiety. Such cysteine residues are available for conjugation but desirably do not substantially affect, for example, immunoglobulin folding, antibody assembly, antigen binding and/or Fc domain effector functions. For example, the engineered cysteine residue can take the place of the amino acid that naturally occurs at a given position in the peptide sequence; i.e. in an embodiment of the present disclosure, the engineered cysteine residue is a cysteine substitution. Engineered cysteine residues can be introduced into the peptide sequence of the target-binding moiety through suitable techniques such as site-directed mutagenesis, the selection of which can be made by a person skilled in the art. In an embodiment, the target-binding moiety comprises one engineered cysteine residue. In another embodiment, the target-binding moiety comprises greater than one engineered cysteine residue, for example, two engineered cysteine residues. For example, antibodies wherein the amino acid aspartic acid at position 265 has been exchanged to cysteine (D265) contain two introduced cysteines at each chain of the Fc region, which can serve as conjugation sites for the compound-linker construct to produce a conjugate with DAR=2.

In an embodiment, the engineered cysteine residue is selected from the group consisting of heavy chain 118Cys, heavy chain 239Cys, and heavy chain 265Cys. In another embodiment, the engineered cysteine residue is heavy chain 265Cys.

In an embodiment, the target-binding moiety comprises a biorthogonal functional group, R⁸ is a complementary biorthogonal functional group, and the compound-linker construct is conjugated to the target-binding moiety via a moiety resulting from the reaction of the biorthogonal functional group on the target-binding moiety with R⁸. The term “biorthogonal” as used herein refers to a chemical reaction capable of occurring inside of a living system without interfering with native biochemical processes. Biorthogonal reactions are well known in the art and include but are not limited to reaction between an azide and a soft nucleophile such as a phosphine; reaction between an azide and a suitable acetylene, and 1,3-dipolar cycloaddition between an azide and a suitable cyclooctyne or azacyclooctyne (copper-free click chemistry).

In an embodiment, the target-binding moiety comprises a primary amino group, R⁸ is a reactive group to the primary amino group, and the compound-linker construct is conjugated to the target-binding moiety via a moiety resulting from the reaction of the primary amino group with R⁸. In another embodiment, the primary amino group is a lysine side chain.

### III. Uses

The present disclosure also includes a composition comprising an amatoxin analog or conjugate of the present disclosure and a carrier. In an embodiment, the composition comprises the amatoxin analog of the present disclosure and the carrier. In another embodiment, the composition comprises the conjugate of the present disclosure and the carrier. The amatoxin analogs and/or conjugates of the present disclosure are optionally formulated into pharmaceutical compositions for administration or use in a biologically compatible form, for example, a form suitable for administration to or for use in subjects in vivo. Accordingly, the present disclosure also includes a pharmaceutical composition comprising an amatoxin analog or conjugate of the present disclosure and a pharmaceutically acceptable carrier. In an embodiment, the pharmaceutical composition comprises the amatoxin analog of the present disclosure and the pharmaceutically acceptable carrier. In another embodiment, the pharmaceutical composition comprises the conjugate of the present disclosure and the pharmaceutically acceptable carrier.

The amatoxin analog or conjugate of the present disclosure can be administered to a subject or used in a variety of forms depending on the selected route of administration or use, as will be understood by a person skilled in the art. For example, the amatoxin analog or conjugate of the present disclosure is suitably administered to the subject or for use parenterally; i.e. taken into the body or administered or used in a manner other than through the gastrointestinal tract. In an embodiment, the amatoxin analog or conjugate of the present disclosure is administered or for use as an injectable or infusion. Injectables can be formulated in the form of ampules and/or as a ready-for-use injectable such as a ready-to-use syringe, a single-use syringe and/or in a puncturable flask for multiple withdrawal. In another embodiment, the injectable is administered or for use in the form of a subcutaneous (s.c.), intramuscular (i.m.), intravenous (i.v.) or intracutaneous (i.e.) injection. In an embodiment, the infusion is in the form of an isotonic solution, fatty emulsion, liposomal formulation or micro-emulsion. A person skilled in the art would know how to prepare suitable formulations. In some embodiments, the injectable or infusion formulation is in the form of a concentrate which can be dissolved or dispersed with aqueous isotonic diluents. Injectable formulations can also be administered or for use in the form of a permanent infusion e.g. via a mini-pump.

In some embodiments, the parenteral formulation further comprises albumin, plasma, expander, surface-active substances, organic diluents, pH-influencing substances, complexing substances, polymeric substances or combinations thereof, for example to influence the adsorption of the amatoxin analog or conjugate of the present disclosure to proteins or polymers and/or to reduce the adsorption of the amatoxin analog or conjugate of the present disclosure to materials like injection instruments or packaging-materials, for example, plastic or glass.

In some embodiments, adjuvants and carriers in the pharmaceutical compositions formulated as parenterals are one or more of aqua sterilisata (sterilized water), pH value influencing substances (for example, suitable organic or inorganic acids or bases and salts thereof or suitable combinations thereof), buffering substances for adjusting pH values, substances for isotonization (for example, sodium chloride, sodium hydrogen carbonate, glucose, fructose or combinations thereof), surfactants (for example, partial esters of fatty acids of polyoxyethylene sorbitans such as a Tween™ surfactant or fatty acid esters of polyoxyethylenes such as a Cremophor™ surfactant or combinations thereof), fatty oils (for example, soybean oil, castor oil or combinations thereof), synthetic esters of fatty acids (for example, ethyl oleate, isopropyl myristate or combinations thereof), polymeric adjuvants (for example, gelatine, dextran, polyvinylpyrrolidone or combinations thereof), additives which increase the solubility of organic solvents (for example, propylene glycol, ethanol, N,N-dimethylacetamide or combinations thereof), complex forming substances (for example, citrate, urea or combinations thereof), preservatives (for example, benzoic acid hydroxypropyl ester, benzoic acid methyl ester, benzyl alcohol or combinations thereof), antioxidants (for example, sodium sulfite) and stabilizers (for example, ethylenediaminetetraacetic acid, EDTA).

In another embodiment, the amatoxin analog or conjugate is orally administered or used, for example, with an inert diluent or with an assimilable edible carrier, or enclosed in hard- or soft-shell gelatin capsules, or compressed into tablets, or incorporated directly with the food of the diet. In an embodiment, for oral therapeutic administration or use, the amatoxin analog or conjugate is incorporated with excipient and administered or used in the form of ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as molecular sieving type particles, or, a fine hollow permeable fiber bundle, or chopped hollow permeable fibers, agglomerated or held in a fibrous packet. Timed-release compositions can be formulated, e.g. liposomes or those wherein the amatoxin analog or conjugate is protected with differentially degradable coatings, such as by microencapsulation, multiple coatings, etc.

The present disclosure also includes all uses for the amatoxin analogs, compound-linker constructs and conjugates of the present disclosure, including, for example, use in therapeutic methods, diagnostic assays and as research tools whether alone or in combination with another active pharmaceutical ingredient.

The synthesis, modelling and biochemical evaluation of amatoxin analogs comprising modifications of eastern ring residues is disclosed herein. Several of the amatoxin analogs were as toxic as α-amanitin and one example was 3-fold more toxic than α-amanitin. Therefore, the amatoxin analogs and conjugates of the present disclosure may be useful as medicaments. Accordingly, the present disclosure also includes an amatoxin analog or conjugate of the present disclosure for use as a medicament. In an embodiment, the amatoxin analog of the present disclosure is for use as a medicament. In another embodiment, the conjugate of the present disclosure is for use as a medicament.

The present disclosure also includes a method of treating a disease associated with cells presenting a target in a subject in need thereof, the method comprising administering an effective amount of an amatoxin analog or a conjugate of the present disclosure to the subject, wherein the target-binding moiety is specific for the target. The present disclosure also includes a method of treating a disease associated with cells presenting a target in a subject in need thereof, the method comprising administering an effective amount of an amatoxin analog of the present disclosure to the subject, wherein the target-binding moiety is specific for the target. The present disclosure also includes a method of treating a disease associated with cells presenting a target in a subject in need thereof, the method comprising administering an effective amount of a conjugate of the present disclosure to the subject, wherein the target-binding moiety is specific for the target.

The present disclosure also includes a use of an effective amount of an amatoxin analog or a conjugate of the present disclosure for treatment of a disease associated with cells presenting a target in a subject in need thereof, wherein the target-binding moiety is specific for the target. The present disclosure also includes a use of an effective amount of an amatoxin analog of the present disclosure for treatment of a disease associated with cells presenting a target in a subject in need thereof, wherein the target-binding moiety is specific for the target. The present disclosure also includes a use of an effective amount of a conjugate of the present disclosure for treatment of a disease associated with cells presenting a target in a subject in need thereof, wherein the target-binding moiety is specific for the target.

The present disclosure also includes a use of an effective amount of an amatoxin analog or a conjugate of the present disclosure for preparation of a medicament for treatment of a disease associated with cells presenting a target in a subject in need thereof, wherein the target-binding moiety is specific for the target. The present disclosure also includes a use of an effective amount of an amatoxin analog of the present disclosure for preparation of a medicament for treatment of a disease associated with cells presenting a target in a subject in need thereof, wherein the target-binding moiety is specific for the target. The present disclosure also includes a use of an effective amount of a conjugate of the present disclosure for preparation of a medicament for treatment of a disease associated with cells presenting a target in a subject in need thereof, wherein the target-binding moiety is specific for the target.

The present disclosure also includes an amatoxin analog or a conjugate of the present disclosure for use to treat a disease associated with cells presenting a target in a subject, wherein the target-binding moiety is specific for the target. The present disclosure also includes an amatoxin analog of the present disclosure for use to treat a disease associated with cells presenting a target in a subject, wherein the target-binding moiety is specific for the target. The present disclosure also includes a conjugate of the present disclosure for use to treat a disease associated with cells presenting a target in a subject, wherein the target-binding moiety is specific for the target.

The term “disease associated with cells presenting a target” as used herein refers, for example, to a pathological state characterized by a cellular marker such as but not limited to a receptor and/or enzyme which can be used in a targeted fashion. In an embodiment, the disease associated with cells presenting a target is cancer, a disease associated with non-cancerous neoplasms, an autoimmune disease, an inflammatory disease or a viral infection. In another embodiment, the disease associated with cells presenting a target is cancer, an autoimmune disease or a viral infection. The term “cancer” as used herein refers to diseases caused by uncontrolled cell division and/or the ability of cells to metastasize, or to establish new growth in additional sites. In an embodiment, the cancer is a skin cancer (e.g., melanoma), a connective tissue cancer (e.g., sarcomas), an adipose cancer, a breast cancer, a head and neck cancer, a lung cancer (e.g., mesothelioma), a stomach cancer, a pancreatic cancer, an ovarian cancer, a cervical cancer, a uterine cancer, an anogenital cancer (e.g., testicular cancer), a kidney cancer, a bladder cancer, a colon cancer, a prostate cancer, a central nervous system (CNS) cancer, retinal cancer, a blood cancer, a neuroblastoma, multiple myeloma, or a lymphoid cancer (e.g., Hodgkin's and non-Hodgkin's lymphomas). In an embodiment, the cancer is breast cancer. In a further embodiment, the cancer is HER2-positive breast cancer. The term “autoimmune disease” can be used interchangeably with the term “autoimmune disorder” and refers to a condition in a subject characterized by cellular, tissue and/or organ injury caused by an immunologic reaction of the subject to its own cells, tissues and/or organs. The term “inflammatory disease” can be used interchangeably with the term “inflammatory disorder” and refers to a condition in a subject characterized by inflammation, such as chronic inflammation. Autoimmune disorders may or may not be associated with inflammation. Moreover, inflammation may or may not be caused by an autoimmune disorder. Thus, the skilled person would appreciate that certain disorders may e.g. be characterized as both autoimmune and inflammatory disorders. In an embodiment, the inflammatory disease is systemic Lupus Erythematosus (SLE), Rheumatoid Arthritis (RA), Autoimmune Hemolytic Anaemia (AIHA), or Sjögren's syndrome. In an embodiment wherein the disease associated with cells presenting a target is a viral infection, the cells presenting a target are virally infected cells. In another embodiment, the viral infection is human immunodeficiency virus (HIV).

The present disclosure also includes a method of treating cancer in a subject in need thereof, the method comprising administering an effective amount of an amatoxin analog or a conjugate of the present disclosure to the subject. The present disclosure also includes a method of treating cancer in a subject in need thereof, the method comprising administering an effective amount of an amatoxin analog of the present disclosure to the subject. The present disclosure also includes a method of treating cancer in a subject in need thereof, the method comprising administering an effective amount of a conjugate of the present disclosure to the subject.

The present disclosure also includes a use of an effective amount of an amatoxin analog or a conjugate of the present disclosure for treatment of cancer in a subject in need thereof. The present disclosure also includes a use of an effective amount of an amatoxin analog of the present disclosure for treatment of cancer in a subject in need thereof. The present disclosure also includes a use of an effective amount of a conjugate of the present disclosure for treatment of cancer in a subject in need thereof. The present disclosure also includes a use of an effective amount of an amatoxin analog or a conjugate of the present disclosure for preparation of a medicament for treatment of cancer in a subject in need thereof. The present disclosure also includes a use of an effective amount of an amatoxin analog of the present disclosure for preparation of a medicament for treatment of cancer in a subject in need thereof. The present disclosure also includes a use of an effective amount of a conjugate of the present disclosure for preparation of a medicament for treatment of cancer in a subject in need thereof. The present disclosure also includes an amatoxin analog or a conjugate of the present disclosure for use to treat cancer in a subject. The present disclosure also includes an amatoxin analog of the present disclosure for use to treat cancer in a subject. The present disclosure also includes a conjugate of the present disclosure for use to treat cancer in a subject.

Treatment methods or uses comprise administering to a subject or use of an effective amount of an amatoxin analog or conjugate of the present disclosure, optionally consisting of a single administration or use, or alternatively comprising a series of administrations or uses. For example, the amatoxin analogs or conjugates of the present disclosure are administered or used at least once a week. However, in another embodiment, the amatoxin analog or conjugate is administered to the subject or for use from one time per three weeks, or one time per week to once daily for a given treatment or use. The length of the treatment period or use depends on a variety of factors, such as the severity of the disease, the age of the subject, the activity of the amatoxin analog or conjugate of the present disclosure and/or a combination thereof. It will also be appreciated by the person skilled in the art that the effective amount of an amatoxin analog or conjugate used for the treatment or use may increase or decrease over the course of a particular treatment regime or use. Changes in dosage may result and become apparent by standard diagnostic assays known in the art. In some instances, chronic administration or use is required. For example, the amatoxin analog or conjugate of the present disclosure is administered or for use in an amount and for a duration suitable to treat the subject.

The amatoxin analog or conjugate of the present disclosure may be administered or used alone or in combination with other therapeutic agents useful for treating a disease (e.g. cancer). When administered or for use in combination with other known therapeutic agents, it is an embodiment that the amatoxin analog or conjugate of the present disclosure is administered or for use contemporaneously with those therapeutic agents. As used herein, the term “contemporaneous” in reference to administration of two substances to a subject or use means providing each of the two substances so that they are both biologically active in the individual at the same time. The exact details of the administration or use will depend on the pharmacokinetics of the two substances in the presence of each other, and can include administration or use of the two substances within a few hours of each other, or even administration or use of one substance within 24 hours of administration or use of the other, if the pharmacokinetics are suitable. Design of suitable dosing regimens is routine for a person skilled in the art. In some embodiments of the present disclosure, two substances will be administered or for use substantially simultaneously, i.e. within minutes of each other, or in a single composition that includes both substances. It is a further embodiment of the present disclosure that a combination of the two substances is administered to a subject or for use in a non-contemporaneous fashion.

The dosage of the amatoxin analog or conjugate of the disclosure can vary depending on many factors such as the pharmacodynamic properties of the amatoxin analog or conjugate, the age, health and/or weight of the subject, the nature and/or extent of the symptoms of the disease, the frequency of the treatment or use and the type of concurrent treatment or use, if any, and the clearance rate of the amatoxin analog or conjugate in the subject. A person skilled in the art can determine the appropriate dosage based on the above factors. In an embodiment, the amatoxin analog or conjugate of the present disclosure is administered or for use initially in a suitable dosage that is optionally adjusted as required, depending on the clinical response.

The present disclosure also includes a method for controlling pests, the method comprising contacting a pest with an amatoxin analog or conjugate of the present disclosure. The present disclosure also includes a method for controlling pests, the method comprising contacting a pest with an amatoxin analog of the present disclosure. The present disclosure also includes a method for controlling pests, the method comprising contacting a pest with a conjugate of the present disclosure. The present disclosure also includes a use of an amatoxin analog or conjugate of the present disclosure for controlling pests. The present disclosure also includes a use of an amatoxin analog of the present disclosure for controlling pests. The present disclosure also includes a use of a conjugate of the present disclosure for controlling pests. The present disclosure also includes an amatoxin analog or conjugate of the present disclosure for use in controlling pests. The present disclosure also includes an amatoxin analog of the present disclosure for use in controlling pests. The present disclosure also includes a conjugate of the present disclosure for use in controlling pests. In an embodiment, the pest is a rodent e.g. a mouse or rat.

The present disclosure also includes a kit comprising an amatoxin analog as described herein, a linker as described herein and optionally instructions for preparing a compound-linker construct as described herein from the amatoxin analog and the linker. The present disclosure also includes a kit comprising an amatoxin analog as described herein, a linker as described herein, a target-binding moiety as described herein and optionally instructions for preparing a conjugate as described herein from the amatoxin analog, the linker and the target-binding moiety. The present disclosure also includes a kit comprising a compound-linker construct as described herein, a target-binding moiety and optionally instructions for preparing a conjugate as described herein from the compound-linker construct and the target-binding moiety.

The following non-limiting examples are illustrative of the present disclosure:

### EXAMPLES

### Example 1: Synthesis and Characterization of Amanitin Eastern Fragment Analogs

### I. Materials & Methods

General: All reactions were performed under argon atmosphere in flame-dried glassware if dry solvents were used, unless otherwise stated. Temperature controlled reactions were performed using a mineral oil bath on a stir plate (either an IKA Ceramag Midi or Fisher Scientific Isotemp® stir plate). Temperatures below room temperature were achieved in an ice/water bath (4° C.), or in a cold room (1° C.) if low temperature was required for overnight reaction. Solvents were removed under reduced pressure using a Heidolph Collediate rotary evaporator. Anhydrous solvents were dried over molecular sieves: 4 Å for methanol (MeOH), dichloromethane (DCM) and dimethylformamide (DMF), and 5 Å sieves were used for pyridine. Tosyl chloride was recrystallized from benzene/petroleum (pet.) ether and potassium thioacetate was freshly made before every reaction from thioacetic acid and potassium hydroxide (KOH). All reagents and solvents were purchased from Sigma-Aldrich, Alfa Aesar, Acros Organics, Matrix Scientifics, ArkPharm Chemicals, AK Scientific, EMD Millipore, Oakwood Chemicals, Ontario Chemicals, Gyros Protein Technologies or TCI America, unless otherwise stated. Authentic α-amanitin was purchased from Sigma-Aldrich. Thin-layer chromatography (TLC) was performed using silica gel 60 F254 precoated aluminum plates (EM Science). Detection of TLC spots was performed using an ultraviolet (UV) lamp at 254 nm, or by staining with potassium permanganate, ninhydrin or Hanessian's stain, prepared according to literature procedures. Flash column chromatography was performed using silica gel 60 (230-400 mesh, Silicycle, Quebec). High-Resolution mass spectra (HRMS) in electrospray ionization (ESI) mode were obtained from a Waters/Micromass LCT spectrometer. Proton (¹H) and carbon (¹³C) nuclear magnetic resonance (NMR) spectra were obtained using Bruker AV-300 (300 MHz), AV-400Dir (400 MHz) spectrometers.

High Performance Liquid Chromatography (HPLC) purification: All HPLC purifications were performed on an Agilent 1260 Infinity II (HPLC 1) Prep HPLC outfitted with the following modules: G7161A Prep Pump, G7157A Prep Autosampler, G7115A DAD, G1364E Fraction Collector. One of the following columns was used; Column 1: Agilent 5 Prep C18 50×21.2 mm (length, L×internal diameter, ID), Column 2: Agilent 5 Prep C18 50×10 mm (L×ID). Solvent A was 0.1% formic acid H₂O (filtered through a 0.2 μm Pall™ filter). Solvent B was 0.1% formic acid acetonitrile (MeCN), purchased at HPLC grade, formic acid then added. Absorbance was monitored simultaneously at 230 nm, 260 nm and 290 nm. HPLC reinjection: All analytical HPLC chromatographs were acquired on an Agilent 1100 Series (HPLC 2) outfitted with the following modules: G1379A Degasser, G1311A Quat Pump, G1313A Autosampler, G1316A COLCOM, G1315B DAD, G1364C Analytical Fraction Collector. Column 3, an Agilent Eclipse XDB-C18 9.4×250 mm, was used. Solvent A was 0.1% formic acid H₂O (filtered through a 0.2 μm PALL filter). Solvent B was 0.1% formic acid MeCN, purchased at HPLC grade, formic acid then added. Absorbance was monitored simultaneously at 230 nm, 260 nm and 290 nm. HPLC Method A: HPLC 2, 2 mL/min, Column 3. Solvent A: 0.1% formic acid H₂O, Solvent B: 0.1% formic acid MeCN. 0 min-32 min 95:5 A/B-65:35 A/B; 32 min-35 min 65:35 A/B-0:1 A/B; 35 min-40 min 0:1 A/B; 40 min-41 min 0:1 A/B-95:5 A/B; 41 min-49 min 95:5 A/B. HPLC Method B: 2 mL/min, Column 3. Solvent A: 0.1% formic acid H₂O, Solvent B: 0.1% formic acid MeCN. 0 min-32 min 90:10 A/B-50:50 A/B; 32 min-35 min 50:50 A/B-0:1 A/B; 35 min-40 min 0:1 A/B; 40 min-41 min 0:1 A/B-90:10 A/B; 41 min-49 min 90:10 A/B. HPLC Method C: 2 mL/min, Column 3. Solvent A: 0.1% formic acid H₂O, Solvent B: 0.1% formic acid MeCN. 0 min-24 min 90:10 A/B-60:40 A/B; 32 min-35 min 60:40 A/B-0:1 A/B; 35 min-40 min 0:1 A/B; 40 min-41 min 0:1 A/B-90:10 A/B; 41 min-49 min 90:10 A/B. HPLC Method D: HPLC 1, 15 mL/min, Column 1 or 2. Solvent A: 0.1% formic acid H₂O, Solvent B: 0.1% formic acid MeCN. 0 min-7 min 95:5 A/B-65:35 A/B; 7 min-8 min 65:35 A/B-0:1 A/B; 8 min-9.5 min 0:1 A/B. HPLC Method E: HPLC 1, 15 mL/min, Column 1 or 2. Solvent A: 0.1% formic acid H₂O, Solvent B: 0.1% formic acid MeCN. 0 min-7 min 90:10 A/B-75:25 A/B; 7 min-8 min 75:25 A/B-0:1 A/B; 8 min-9.5 min 0:1 A/B.

Peptide quantification: All peptides were quantified by UV spectrophotometry on a Cary 5000 UV-Vis-NIR Spectrophotometer. Lyophilized peptides were dissolved in a known volume of H₂O or 0.1% formic acid H₂O, transferred to a quartz cuvette and absorbance was measured at a λₘₐₓ of 290 nm for peptides with unsubstituted tryptathionine staples (Zanotti et al., 1978; Matinkhoo et al., 2018) or 288 nm for peptides with 5′ OH tryptathionine staples (Pryyma et al., 2020).

Circular dichroism (CD) spectral acquisition: All CD spectra were acquired on a JASCO J-815 CD Spectrometer. All peptides were dissolved in MeOH to a concentration of 50 mM and read at room temperature. Three scans were accumulated for each peptide and a smoothing algorithm was applied.

Sample preparation and cytotoxicity assays: Following quantification, peptides were re-lyophilized and re-suspended in H₂O to yield a solution of 1 mM, which was then used in cell toxicity assays. Cell culture: Cells were cultured in α-Minimum Essential Medium (MEM) or high-sucrose Dulbecco's Modified Eagle Medium (DMEM), purchased from Gibco. Fetal bovine serum (FBS), 0.25% trypsin (with 1.3 mM ethylenediaminetetraacetic acid, EDTA), 0.85% Trypan blue, and the antibiotic mixture Pen/Strep (10K U/mL penicillin, 10K mg/mL streptomycin) were also purchased from Gibco. All cell culture plastic ware was obtained from Corning or Falcon. Cells were cultured at 37° C. in a humidified chamber with 5% CO₂. When used in cell culture, dimethylsulfoxide (DMSO) was purified by filtration through a 0.2 mm filter. All experiments were carried out in a laminar flow culture cabinet, unless otherwise noted. Absorbance measurements of the 96-well plates were obtained using a Beckman-Coulter DTX 880 multimode detector, equipped with an excitation filter of 595 nm. S5 Immortalized CHO cells had been stored in liquid nitrogen. To revive cells, a 1 mL tube of the frozen cells in medium containing 10% DMSO was warmed in a 30° C. water bath, and diluted with 9 mL of fresh media. Media contained 10% FBS, 100 U/mL penicillin and 100 mg/mL streptomycin, unless otherwise indicated. The cells were incubated in a T-25 flask at 37° C. at 5% CO₂. After 24 hours, the medium was aspirated and replaced with fresh medium. When cells reached a level of 90-100% confluence, they were subcultured. The medium was removed, and the cells were treated with 0.25% trypsin containing 1.3 mM EDTA in the incubator. Once the cells were detached from the tissue culture flask, 3-5 mL media was added to quench the trypsin, and transferred to a 10 mL centrifuge tube. The mixture was centrifuged for 5 min at 8000 rpm, and the supernatant was discarded. The cells were suspended in fresh medium, diluted as required, and transferred to a new culture flask. To assay cell viability, a nearly confluent tissue culture flask was trypsinized, and the cells were counted following treatment with Trypan blue, using a hemacytometer. The cells were then diluted to the appropriate stock concentrations in fresh medium, and transferred in 100 μL to a 96-well plate using a multi-channel pipette. The number of cells plated varied from experiment-to-experiment. These were incubated a 37° C. and 5% CO₂ for a 24-hour period to allow for adherence. The medium was aspirated, and fresh medium was added, which contained the desired additives in DMSO. The cells were then re-incubated for 72 h. At the completion of the experiment, a 100 μL aliquot of 25 mg/mL 3-(4,5-dimethylthiazol-2-yl)-2,5-diphenyltetrazolium bromide (MTT) in phosphate-buffered saline (PBS) was added to each well. The plate was incubated three hours further, to allow for the formation of the formazan product in viable cells. The media was carefully aspirated, and the purple product was solubilized in DMSO. The absorbance of each well was recorded at 595 nm, and an image of the plate was generated using a scanner. Data was processed in Microsoft™ Excel and GraphPad Prism. Experiments were performed in triplicate, unless otherwise noted, and the error bars were calculated as the standard error of the mean. Trypsinized cells were diluted to a concentration of 3.3×10⁴ cells/mL for CHO cells. Each cell line was plated in a 96-well plate, with 100 μL of the stock solution per well (5000 cells per well), and incubated for 24 hours. Stocks of α-amanitin or analogs were prepared at various concentrations, and added to various wells, according to the desired final concentration: 20, 4, 0.8, 0.16, 0.032, 0.0064, 0.00128, and 0.000256 μM. The cells were incubated for 72 hours, at which point viability was assessed as described.

General methods for transcription runoff assays: The majority of this was adapted from previous protocols (Matinkhoo et al., 2021b; Pryyma et al., 2020; Matinkhoo et al., 2021). Chemicals and reagents: Primers were purchased from IDT via standard phosphoramidite chemistry. dNTP and rNTP were purchased from Thermofisher. GoTaq polymerase and HeLaScribe® Nuclear Extract in vitro Transcription System was purchased from Promega. Taq polymerase was purchased from NEB. [³²P]α-dGTP (3000Ci/mmol 10 mCi/ml EasyTide) and [³²P]α-GTP (3000Ci/mmol 10 mCi/ml EasyTide) were purchased from Perkin Elmer. QIAquick PCR purification kit was purchased from Qiagen. AcGFP1-N1 was from the Davidson lab (Addgene plasmid #54705; http://n2t.net/addgene:54705; RRID:Addgene_54705). DNA primer sequences were:

P1 AcGFP1.FOR. V4
CAGTCGACGGTACCGC
P2 AcGFP1.REV.V4
GCCCTCGAACTTCACCTC
P3 AcGFP1.FOR.V2
CGCGGGCCCGGGATCCAC
P4 AcGFP.REV.V2
ACCTCGGCGCGCGACTTGT

Synthesis of the DNA template for transcription runoff: The DNA template for transcription runoff was synthesized by PCR with template pAcGFP-N1, and primers P1 and P2. To a final volume of 20 μL, 1×GoTaq Buffer, 250 μM of each dNTP, 500 nM of each of primers P1 and P2, 10 μg/μL pAcGFP-N1 and 0.05 U/μL GoTaq were employed to thermocycle for 30 cycles (30 s at 95° C., 30 s at 50° C. and 60 s at 72° C., Bio-Rad). A 1 μL aliquot of the amplified solution was resolved with 6×DNA loading dye in a 1% agarose gel containing 1% ethidium bromide and then visualized using GelDoc XR imager (Bio-Rad). Purification was completed via QIAquick PCR purification kit (Qiagen). Synthesis of the radioactive chromatographic standard: The radioactive chromatographic standard was synthesized by PCR with template pAcGFP-N1, and primers P3 and P4. To a final volume of 50 μL, 1×Thermopol Buffer, 200 μM of each dNTP, 500 nM of each primers P3 and P4, 10 μg/μL pAcGFP-N1 and 0.02 U/μL Vent polymerase, 1 μL [³²P] α-dGTP were employed to thermocycle for 30 cycles (30 s at 95° C., 30 s at 62° C. and 30 s at 72° C., Bio-Rad). A 1 μL aliquot of the amplified solution was resolved with 6×DNA loading dye in 8% denaturing PAGE and then visualized by autoradiography via the Typhoon 9200 imager (Molecular Dynamics-Amersham-GE). Purification was completed via QIAquick PCR purification kit (Qiagen). Transcription runoffassay and statistical analysis: The transcription runoff assay was modified from HeLaScribe® Nuclear Extract in vitro Transcription System (Promega). A 1.35× master mixture was formulated with 1.35×HeLa Nuclear Extract Transcription Buffer, 4.05 mM MgCl₂, 540 μM rATP, 540 μM rCTP, 540 μM rUTP and 21.6 μM rGTP, 5.4 ng/μL DNA template and 3 μL [³²P]α-GTP. To a final volume of 8 μL, 1× master mixture, 0-3 μM aqueous solutions containing α-amanitin or amanitin analogs, 0.32 U/μL HeLaScribe® Nuclear Extract were combined to transcribe for 60 min. The reaction was quenched by 56 μL HeLa Extract Stop Solution, followed by phenol-chloroform extraction and EtOH precipitation. The pellet was resolved by 8% denaturing PAGE and then visualized by autoradiography via the Typhoon 9200 imager (Molecular Dynamics-Amersham-GE). Dosimetry was calculated by ImageJ (Schneider et al., 2012), and the 3 parameter logistic fit was completed by Origin 2019 (OriginLab). For inhibitor I that demonstrates transcription activity E with inhibitory constant Kᵢ,

$$E = \frac{{K}_{i}}{{K}_{i} + [ I ]} \qquad ( 1 )$$

This assumes symmetry around Kᵢ (asymmetry factor S=1) and no cooperativity (Hill's slope H=−1). All data were presented in mean±standard deviation (n=3). For amanitin analogs with Kᵢ≥1000 nM, upon rearrangement of equation 1, with transcription activity Ē,

$${K}_{i} = ( \frac{E}{1 - E} ) [ I ] \qquad ( 2 )$$

Using [I]=100 μM, the averaged transcription activities Ē was used to estimate high values of Kᵢ.

### II. Synthesis

### Synthesis of Fmoc-Thialle-OH

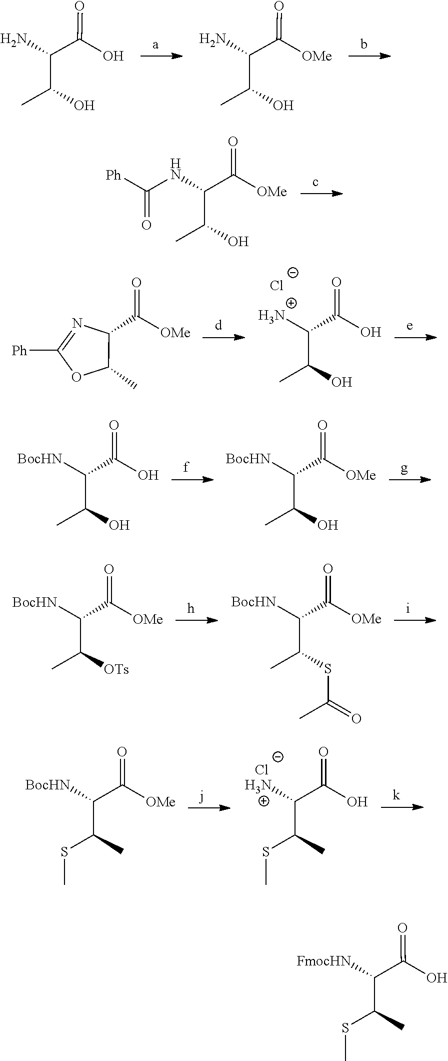

Reagents and conditions: (a) SOCl₂ (1.64 eq), MeOH, reflux, 2 h (b) benzoyl chloride (1 eq), triethylamine (TEA, 2 eq), dry DCM, 2 h, 4° C., 49% over two steps (c) SOCl₂, 4° C. to room temperature (rt), overnight (O.N.) 70% (d) 6M HCl_{(aq)}, reflux, 5 h (e) di-tert-butyl dicarbonate (Boc₂O, 1.2 eq), Na₂CO₃ (3.1 eq), H₂O/tetrahydrofuran (THF). 4° C. to rt, O.N. (f) iodomethane (Mel, 5.7 eq), K₂CO₃ (1.7 eq), DMF, rt 2 h, 73% over three steps (g) p-toluenesulfonyl chloride (TsCl, 1.3 eq), dry pyridine, 4° C., O.N. 88% (h) potassium thioacetate (KSAc, 1.55 eq), dry DMF, 4° C. to rt, O.N. 55% (i) 0.2M NaOH (1 eq), Mel (10 eq), H₂O/MeOH, rt, 3 h, 70% (j) 6M HCl_{(aq)}, reflux, 1 h (k) Fmoc-Oxyma (1 eq), NaHCO₃ (3.5 eq), H₂O/acetone, rt, O.N. 68% over two steps.

### Methyl L-threoninate

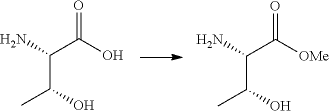

A stirring 100 mL MeOH solution was chilled in an ice bath and 10 mL (137.8 mmol) SOCl₂ was added slowly. The mixture was stirred, at 4° C., for 10 minutes. 10 g (83.95 mmol) of L-threonine was added to the stirring reaction mixture which was then allowed to warm up to room temperature. It was then refluxed for 2 hours and the solvent was subsequently removed in vacuo. The residual MeOH and SOCl₂ were removed by 2 rounds of co-evaporation with DCM to yield a clear syrup after overnight drying on vacuum pump. The product was then used in the next step without further purification.

Methyl benzoyl-L-threoninate

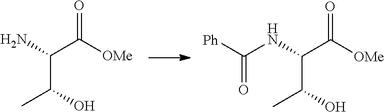

The crude product from the previous step was dissolved in 120 mL dry DCM. 23.4 mL Et₃N (167.8 mmol) was added to the reaction mixture and it was sonicated vigorously to dissolve the syrup. The resulting solution was a white suspension which was then cooled in an ice bath and stirred. 14.3 mL benzoyl chloride (84.0 mmol) was added dropwise over the course of 15 minutes and the reaction was then allowed to proceed at 4° C. for 2 hours. Upon completion, the reaction mixture was filtered through a Celite® sandwich and evaporated under reduced pressure to yield a white solid. The crude product was purified by flash chromatography on silica (2.75″ diameter, 6″ height silica) using isocratic elution with 1:1 (v/v) ethyl acetate (EtOAc)/Hexanes. The pure fractions were pooled and evaporated under reduced pressure to yield a white solid (9.78 g, 41.3 mmol, 49% yield over 2 steps). TLC (EtOAc/Hexanes 1:1 v/v): R_{f}=0.10; ¹H NMR (300 MHz, CD₂Cl₂) δ 7.88-7.76 (m, 2H), 7.52 (t, J=7.2 Hz, 1H), 7.43 (t, J=7.6 Hz, 2H), 7.15 (d, J=7.6 Hz, 1H), 4.74 (dd, J=8.8, 2.5 Hz, 1H), 4.41 (m, 1H), 3.74 (s, 3H), 3.25 (s, 1H), 1.24 (d, J=6.4 Hz, 3H); ¹³C NMR (75 MHz, CD₂Cl₂) 171.89 (s), 168.22 (s), 132.21 (s), 128.95 (s), 127.56 (s), 68.45 (s), 58.27 (s), 52.87 (s), 20.30 (s); HRMS-ESI (m/z): [M+Na]⁺ calcd. for C₁₂H₁₅NO₄Na, 260.0899; found 260.0891.

### Methyl (4S,5S)-5-methyl-2-phenyl-4,5-dihydrooxazole-4-carboxylate

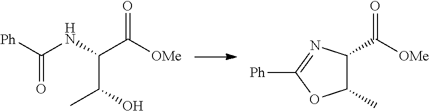 [SMILES: COC(=O)[C@@H](NC(=O)c1ccccc1)[C@@H](C)O ; COC(=O)[C@H]1N=C(c2ccccc2)O[C@H]1C]

Methyl benzoyl-L-threoninate (9.78 g, 41.3 mmol) was dissolved in 30 mL SOCl₂ (413.55 mmol) and stirred at 4° C. for 6 hours. The reaction mixture was then allowed to warm up to room temperature and stir overnight. The SOCl₂ was then removed under reduced pressure and the residual solvent was removed by two rounds of co-evaporation with DCM. The residue was then dissolved in 100 mL DCM and 200 mL saturated (sat.) NaHCO_{3(aq)} was slowly added to the flask while entire mixture was stirred. This solution was transferred to a separatory funnel and the layers were separated. The organic layer was then washed with 200 mL sat. NaHCO_{3(aq)} and then 100 mL H₂O. The organic layer was then dried over magnesium sulfate, filtered into a round bottom flask and the solvent was removed under reduced pressure to yield a yellow oil. (5.97 g, 27.2 mmol, 70% yield). TLC (EtOAc/Hexanes 1:1 v/v): R_{f}=0.38; ¹H NMR (300 MHz, CD₂Cl₂) δ 8.01-7.91 (m, 2H), 7.57-7.48 (m, 1H), 7.47-7.38 (m, 2H), 5.07 (dq, J=10.2, 6.3 Hz, 1H), 4.95 (d, J=10.2 Hz, 1H), 3.75 (s, 3H), 1.36 (d, J=6.3 Hz, 3H); ¹³C NMR (75 MHz, CD₂Cl₂) δ 170.69 (s), 166.20 (s), 132.19 (s), 128.78 (s), 128.75 (s), 78.25 (s), 72.01 (s), 52.28 (s), 16.41 (s). HRMS-ESI (m/z): [M+Na]⁺ calcd. for C₁₂H₁₄NO₃, 220.0974; found 220.0980.

### L-allothreonine

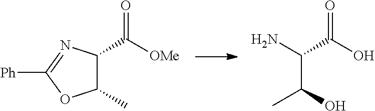

Methyl (4S,5S)-5-methyl-2-phenyl-4,5-dihydrooxazole-4-carboxylate (5.97 g, 27.2 mmol) was refluxed in 100 mL 6M HCl_{(aq)} at about 120° C. for 5 hours. Upon completion of the reaction, the mixture was allowed to cool to room temperature and was then transferred to a separatory funnel, where it was extracted twice with 100 mL 1:1 (v/v) EtOAc/petroleum ether (Pet. Et.) The aqueous layer was evaporated to dryness under reduced pressure to yield a white solid. (Quantitative Yield)¹H NMR (300 MHz, D₂O) δ 4.37 (qd, J=6.7, 3.6 Hz, 1H), 4.09 (d, J=3.6 Hz, 1H), 1.30 (d, J=6.7 Hz, 3H); ¹³C NMR (75 MHz, D₂O) δ 169.91 (s), 65.39 (s), 58.25 (s), 17.20 (s). HRMS-ESI (m/z): [M+H]⁺ calcd. for C₄H₁₀NO₃, 120.0661; found 120.0662.

### (tert-Butoxycarbonyl)-L-allothreonine

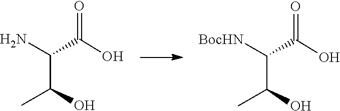

L-Allothreonine (27.2 mmol) was dissolved in 120 mL H₂O and stirred at 4° C. Na₂CO₃ (8.94 g, 84.35 mmol) was added to the stirring mixture. Boc₂O (7.13 g, 32.65 mmol) was dissolved in 150 mL THF and this solution was added to the aqueous mixture containing the amino acid, dropwise. The reaction mixture was then allowed to warm up to room temperature overnight. Upon completion of the reaction, the solution was acidified to a pH of about 2 with ortho-phosphoric acid. It was extracted with 150 mL EtOAc and then 2 further 75 mL portions of EtOAc. The organic layers were pooled and washed with 100 mL sat. KH₂PO_{4(aq)}. The organic layer was then dried over magnesium sulfate, filtered into a round bottom flask and the solvent was evaporated under reduced pressure to yield a dark yellow oil. The crude product was purified by flash chromatography on silica (2.5″ diameter, 5″ height silica). It was loaded with 0.1:0.5:9.4 (v/v/v) acetic acid (AcOH)/EtOH/DCM and eluted with 0.1:0.8:9.1 (v/v/v) AcOH/EtOH/DCM. The pure fractions were pooled and evaporated under reduced pressure to yield a clear oil (6.85 g, quantitative yield over 2 steps). TLC (AcOH/EtOH/DCM 0.1:0.5:9.4 v/v/v): R_{f}=0.13; ¹H NMR ¹H NMR (300 MHz, CD₂Cl₂) δ 5.55 (bs, 1H), 4.30 (m, 1H), 4.13 (m, 1H), 1.44 (s, 9H), 1.25 (m, 3H); ¹³C NMR (101 MHz, CD₂Cl₂) δ 173.37, 156.90, 81.10, 69.27, 59.42, 28.39, 19.26; HRMS-ESI (m/z): [M+Na]⁺ calcd. for C₉H₁₇NO₅Na, 242.1004; found 242.1003.

### Methyl (tert-butoxycarbonyl)-L-allothreoninate

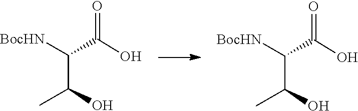

(tert-Butoxycarbonyl)-L-allothreonine (27.2 mmol) was dissolved in 75 mL DMF and K₂CO₃ (6.43 g, 46.5 mmol) was added to solution, which was stirred at 4° C. for about 5 minutes. Mel (9.65 mL, 154.96 mmol) was added to the reaction mixture, dropwise. The reaction mixture was then allowed to warm up to room temperature and it was stirred for 2 hours. The solution was then diluted with 500 mL EtOAc and washed with two 250 mL portions of H₂O and one 250 mL portion of brine. The organic layer was dried over magnesium sulfate, filtered into a round bottom flask and the solvent was evaporated under reduced pressure to yield a thick yellow oil. This material was found to be contaminated with significant amounts of DMF; hence the product was dissolved in 100 mL EtOAc and washed two 50 mL portions of H₂O and one 50 mL portion of brine. The organic layer was dried over magnesium sulfate, filtered into a round bottom flask and the solvent was evaporated under reduced pressure to yield a thick yellow oil. (5.235 g, 22.5 mmol, 73% yield). TLC (EtOAc/Hexanes 1:1 v/v): R_{f}=0.33; ¹H NMR (300 MHz, CD₂Cl₂) δ 5.45 (bs, 1H), 4.48-4.26 (m, 1H), 4.14-4.03 (m, 1H), 3.74 (s, 3H), 2.94 (bs, 1H), 1.43 (s, 9H), 1.16 (d, J=6.4 Hz, 3H). ¹³C NMR (75 MHz, CD₂Cl₂) δ 171.44 (s), 156.43 (s), 80.56 (s), 69.20 (s), 59.57 (s), 52.70 (s), 28.39 (s), 19.07 (s); HRMS-ESI (m/z): [M+Na]⁺ calcd. for C₁₀H₁₉NO₅Na, 256.1161; found 256.1158.

### Methyl N-(tert-butoxycarbonyl)-O-tosyl-L-allothreoninate

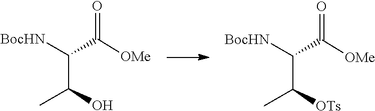 [SMILES: COC(=O)[C@@H](NC(=O)OC(C)(C)C)[C@H](C)O ; COC(=O)[C@@H](NC(=O)OC(C)(C)C)[C@H](C)OS(=O)(=O)c1ccc(C)cc1]

Methyl (tert-butoxycarbonyl)-L-allothreoninate (1.78 g, 7.63 mmol) was dissolved in 5 mL dry pyridine and transferred to a flame dried round bottom flask under argon. The solution was stirred and cooled to 1° C. in an ice bath. Recrystallized tosyl chloride (1.89 g, 9.92 mmol) was added to the reaction mixture, under argon, and the reaction was stirred overnight at 1° C. Upon completion of the reaction, the reaction mixture was diluted with 200 mL Et₂O and washed with one 200 mL portion of 0.01M HCl_{(aq)} and two 200 mL portions of H₂O. The organic layer then was dried over magnesium sulfate, filtered into a round bottom flask and the solvent was evaporated under reduced pressure to yield a clear gum speckled with a white solid. The crude product was purified by flash chromatography on silica (1.75″ diameter, 9″ height silica) using gradient elution from 1:9 (v/v) EtOAc/Hexanes to 1:2 (v/v) EtOAc/Hexanes. The pure fractions were pooled and evaporated under reduced pressure to yield a clear oil (2.61 g, 6.75 mmol, 88% yield). TLC (EtOAc/Hexanes 1:1 v/v): R_{f}=0.55; ¹H NMR (300 MHz, CD₂Cl₂) δ 7.77 (d, J=8.3 Hz, 2H), 7.38 (d, J=8.1 Hz, 2H), 5.20 (d, J=7.2 Hz, 1H), 4.88-4.73 (m, 1H), 4.38 (dd, J=8.5, 3.3 Hz, 1H), 3.73 (s, 3H), 2.45 (s, 3H), 1.40 (s, 9H), 1.32 (d, J=6.6 Hz, 3H); ¹³C NMR (75 MHz, CD₂Cl₂) δ 169.27 (s), 155.17 (s), 145.62 (s), 133.94 (s), 130.34 (s), 128.16 (s), 80.48 (s), 79.32 (s), 57.76 (s), 52.98 (s), 28.33 (s), 21.83 (s), 17.86 (s); HRMS-ESI (m/z): [M+Na]⁺ calcd. for C₁₇H₂₅NO₇Na, 410.1249; found 410.1245.

### Methyl (2R,3R)-3-(acetylthio)-2-((tert-butoxycarbonyl)amino)butanoate

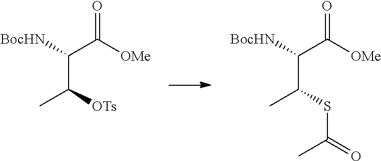 [SMILES: COC(=O)[C@@H](NC(=O)OC(C)(C)C)[C@H](C)OS(=O)(=O)c1ccc(C)cc1 ; COC(=O)[C@@H](NC(=O)OC(C)(C)C)[C@@H](C)SC(C)=O]

Methyl N-(tert-butoxycarbonyl)-O-tosyl-L-allothreoninate (2.59 g, 6.69 mmol) was dissolved in 8.5 mL DMF and transferred to a flame dried round bottom flask under argon. Freshly made potassium thioacetate (1.2 g, about 10.5 mmol) was added to the reaction vessel under argon and the solution was stirred overnight at room temperature. Upon completion of the reaction, the reaction mixture was diluted with 200 mL Et₂O and washed with one 100 mL portion of H₂O. The aqueous layer was then extracted with one 100 mL portion of Et₂O. The organic layers were pooled, dried over magnesium sulfate, filtered into a round bottom flask and the solvent was evaporated under reduced pressure to yield a yellow oil. The crude product was purified by two successive rounds of flash chromatography on silica (1.75″ diameter, 9″ height silica) using isocratic elution with 1:9 (v/v) EtOAc/Hexanes. The pure fractions were pooled and evaporated under reduced pressure to yield a clear oil (1.06 g, 3.65 mmol, 55% yield). TLC (EtOAc/Hexanes 1:1 v/v): R_{f}=0.56; ¹H NMR (300 MHz, CD₂Cl₂) δ 5.20 (d, J=7.6 Hz, 1H), 4.46 (dd, J=9.1, 3.5 Hz, 1H), 4.10-3.95 (m, 1H), 3.70 (s, 3H), 2.29 (s, 3H), 1.42 (s, 9H), 1.34 (d, J=7.2 Hz, 3H); ¹³C NMR (75 MHz, CD₂Cl₂) δ 194.54 (s), 171.30 (s), 155.78 (s), 58.03 (s), 52.78 (s), 42.25 (s), 30.81 (s), 28.36 (s), 19.05 (s); HRMS-ESI (m/z): [M+Na]⁺ calcd. for C₁₂H₂₁NO₅Na, 314.1038; found 314.1043.

### Methyl (2R,3R)-2-((tert-butoxycarbonyl)amino)-3(methylthio)butanoate

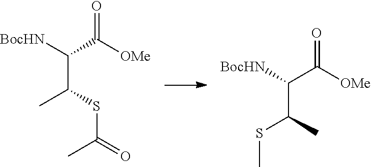 [SMILES: COC(=O)[C@@H](NC(=O)OC(C)(C)C)[C@@H](C)SC(C)=O ; COC(=O)[C@@H](NC(=O)OC(C)(C)C)[C@@H](C)SC]

Methyl (2R,3R)-3-(acetylthio)-2-((tert-butoxycarbonyl)amino)butanoate (0.37 g, 1.27 mmol) was dissolved in 6.4 mL MeOH and 2.6 mL 0.2M NaOH_{(aq)} was added to the stirring mixture at room temperature. This was immediately followed by the addition of Mel (0.79 mL, 12.7 mmol). The pH of the reaction was monitored and when it dropped below 9, additional 0.2M NaOH_{(aq)} was added to restore basicity. The reaction was allowed to proceed for a total of 3 hours at room temperature. The MeOH was then removed in vacuo and the remaining aqueous solution was diluted to about 20 mL with H₂O and extracted with three 20 mL portions of DCM. The organic layers were pooled, washed with one 20 mL portion of brine and then dried over magnesium sulfate, filtered into a round bottom flask and the solvent was evaporated under reduced pressure to yield a yellow oil. The crude product was purified by flash chromatography on silica (0.75″ diameter, 10.5″ height silica) using isocratic elution with 2:8 (v/v) Et₂O/Toluene. The pure fractions were pooled and evaporated under reduced pressure to yield a clear oil (0.23 g, 0.89 mmol, 70% yield). TLC (Et₂O/Toluene 2:8 v/v): R_{f}=0.45; ¹H NMR (300 MHz, CD₂Cl₂) δ 4.40 (dd, J=9.3, 3.6 Hz, 1H), 3.73 (s, 3H), 3.32-3.21 (m, H), 2.05 (s, 3H), 1.43 (s, 9H), 1.31 (d, J=7.1 Hz, 3H); ¹³C NMR (75 MHz, CD₂Cl₂) δ 172.01 (s), 155.99 (s), 80.12 (s), 57.97 (s), 52.57 (s), 44.76 (s), 28.39 (s), 19.11 (s), 14.54 (s); HRMS-ESI (m/z): [M+Na]⁺ calcd. for C₁₁H₂₁NO₄Na, 286.1089; found 286.1088.

### (2R,3R)-2-Amino-3-(methylthio)butanoic acid

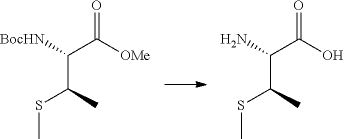

Methyl (2R,3R)-2-((tert-butoxycarbonyl)amino)-3-(methylthio)butanoate (0.23 g, 0.88 mmol) was refluxed in 10 mL 6M HCl_{(aq)} at about 120° C. for 1 hour. Upon completion of the reaction, the mixture was allowed to cool to room temperature, diluted to about 20 mL with H₂O, and then transferred to a separatory funnel, where it was extracted twice with 20 mL 1:1 (v/v) EtOAc/Pet. Et. and then once with a 20 mL portion of Et₂O. The aqueous layer was evaporated to dryness under reduced pressure to yield a white solid. (Quantitative Yield). ¹H NMR (300 MHz, D₂O) δ 3.94 (d, J=5.0 Hz, 1H), 3.40 (qd, J=7.3, 5.0 Hz, 1H), 2.13 (s, 3H), 1.41 (d, J=7.3 Hz, 3H). ¹³C NMR (75 MHz, D₂O) δ 171.10 (s), 57.35 (s), 41.34 (s), 17.88 (s), 13.06 (s). HRMS-ESI (m/z): [M+H]⁺ calcd. for C₅H₁₂NO₂S, 150.0589; found 150.0583.

### (2R,3R)-2-((((9H-fluoren-9-yl)methoxy)carbonyl)amino)-3-(methylthio)butanoic acid

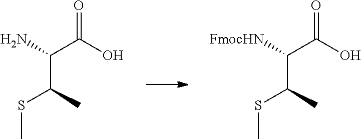

(2R,3R)-2-Amino-3-(methylthio)butanoic acid (0.88 mmol) was dissolved in 0.88 mL H₂O and 3.53 mL sat. NaHCO_{3(aq)} was added to the stirring mixture. The pH of the resultant solution was about 9. A suspension of Fmoc-Oxyma (0.32 g, 0.88 mmol) in 4.4 mL acetone was added dropwise to the stirring mixture and it was allowed to proceed at room temperature, overnight. The acetone was removed in vacuo and the remaining solution was diluted with 40 mL H₂O and acidified to a pH of about 1 with 1M HCl_{(aq)}. It was then extracted with five 50 mL portions of DCM and the organic layers were pooled and dried over magnesium sulfate, filtered into a round bottom flask and the solvent was evaporated under reduced pressure to yield a clear oil. The crude product was purified by flash chromatography on silica (1.25″ diameter, 12″ height silica) using gradient elution from 8:92 (v/v) MeOH/DCM to 12:88 (v/v) MeOH/DCM. The pure fractions were pooled and evaporated under reduced pressure to yield a white solid (0.227 g, 0.61 mmol, 69% yield). TLC (MeOH/DCM 1:9 v/v): R_{f}=0.14; ¹H NMR (300 MHz, CD₂Cl₂) δ 7.78 (d, J=7.5 Hz, 2H), 7.61 (d, J=6.8 Hz, 2H), 7.40 (t, J=7.4 Hz, 2H), 7.31 (t, J=7.4 Hz, 2H), 5.70 (d, J=7.5 Hz, 1H), 4.52-4.34 (m, 3H), 4.24 (t, J=6.7 Hz, 1H), 3.41-3.29 (m, 1H), 2.12 (s, 3H), 1.32 (d, J=6.7 Hz, 3H). ¹³C NMR (75 MHz, CD₂Cl₂) δ 174.02 (s), 156.77 (s), 144.18 (s), 141.69 (s), 128.11 (s), 127.47 (s), 125.48 (s), 120.35 (s), 67.56 (s), 58.30 (s), 47.59 (s), 43.92 (s), 18.72 (s), 14.57 (s); HRMS-ESI (m/z): [M+Na]⁺ calcd. for C₂₀H₂₁NO₄Na, 394.1089; found 394.1086.

### Synthesis of Fmoc-threo-β-MeCys-OH

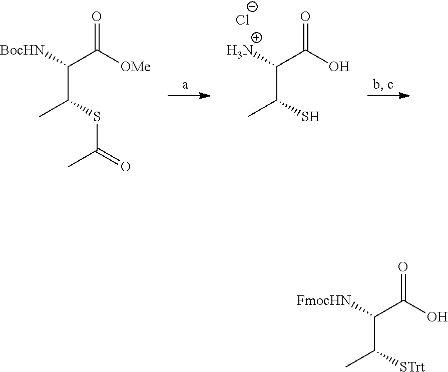

Reagents and conditions: (a) 6M HCl_{(aq)}, reflux, 1 h (b) triphenylmethanol (1 eq), TFA, rt, 15 min (c) Fmoc-Oxyma (1 eq), NaHCO₃ (3.5 eq), H₂O/acetone, rt, O.N. 52% over three steps.

### (2R,3R)-2-Amino-3-mercaptobutanoic Acid

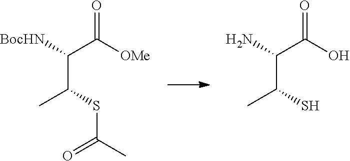

Methyl (2R,3R)-3-(acetylthio)-2-((tert-butoxycarbonyl)amino)butanoate (0.31 g, 1.08 mmol) was refluxed in 10 mL 6M HCl_{(aq)} at about 120° C. for 1 hour. Upon completion of the reaction, the mixture was allowed to cool to room temperature, diluted to about 20 mL with H₂O, and then transferred to a separatory funnel, where it was extracted twice with 20 mL 1:1 (v/v) EtOAc/Pet. Et. and then once with a 20 mL portion of Et₂O. The aqueous layer was evaporated to dryness under reduced pressure to yield a white solid (Quantitative Yield). ¹H NMR (300 MHz, D₂O) δ 4.17 (d, J=4.1 Hz, 1H), 3.71 (qd, J=7.3, 4.1 Hz, 1H), 1.47 (d, J=7.3 Hz, 3H). ¹³C NMR (75 MHz, D₂O) δ 170.78 (s), 59.56 (s), 34.48 (s), 20.90 (s); HRMS-ESI (m/z): [M+Na]⁺ calcd. for C₄H₁₀NO₂S, 136.0432; found 136.0432.

### (2R,3R)-2-((((9H-fluoren-9-yl)methoxy)carbonyl)amino)-3-(tritylthio)butanoic Acid

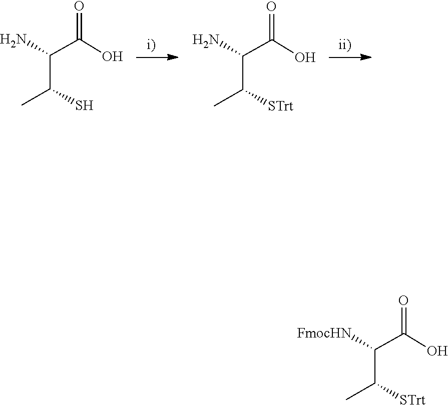

i) (2R,3R)-2-Amino-3-mercaptobutanoic acid (0.185 g, 1.08 mmol) and triphenylmethanol (0.281 g, 1.08 mmol) dissolved in 2 mL trifluoroacetic acid (TFA). The reaction mixture was stirred for 15 minutes at room temperature. Upon completion of the reaction, the solvent was removed under reduced pressure and the solid was left on a vacuum line overnight, until all traces of yellow disappeared from the white solid. ii) The crude product from the previous step was dissolved in 1.08 mL H₂O and 4.32 mL sat. NaHCO_{3(aq)} was added to the stirring mixture. The pH of the resultant solution was about 9. A suspension of Fmoc-Oxyma (0.394 g, 1.08 mmol) in 5.4 mL acetone was added dropwise to the stirring mixture and it was allowed to proceed at room temperature overnight. The acetone was removed in vacuo and the remaining solution was diluted with 40 mL H₂O and acidified to a pH of about 2 with ortho-phosphoric acid. It was then extracted with three 50 mL portions of DCM and the organic layers were pooled and washed with one 100 mL portion of sat. NaHCO_{3(aq)}. This mixture took 30 minutes to resolve. The organic layer was then dried over magnesium sulfate, filtered into a round bottom flask and the solvent was evaporated under reduced pressure to yield a white solid. The crude product was purified by two successive rounds of flash chromatography on silica (1.25″ diameter, 12″ height silica) using isocratic elution with 1:9 (v/v) MeOH/DCM and then 4:96 (v/v) MeOH/DCM. The pure fractions were pooled and evaporated under reduced pressure to yield a white solid (0.333 g, 0.56 mmol, 52% yield over three steps). TLC (MeOH/DCM 1:9 v/v): R_{f}=0.40; ¹H NMR (300 MHz, CD₂Cl₂) δ 7.78 (d, J=7.8 Hz, 2H), 7.62-7.58 (m, 2H), 7.53-7.47 (m, 6H), 7.44-7.36 (m, 2H), 7.34-7.13 (m, 11H), 4.46-4.34 (m, 2H), 4.28-4.16 (m, 2H), 2.84-2.72 (m, 1H), 0.93 (d, J=5.7 Hz, 3H). ¹³C NMR (75 MHz, CD₂Cl₂) δ 174.91 (s), 156.47 (s), 144.98 (s), 144.18 (s), 141.67 (s), 129.93 (s), 128.33 (s), 128.10 (s), 127.48 (s), 127.16 (s), 125.46 (s), 120.33 (s), 68.23 (s), 67.43 (s), 59.78 (s), 47.55 (s), 42.81 (s), 19.93 (s); HRMS-ESI (m/z): [M+Na]⁺ calcd. for C₃₈H₃₃NO₄SNa, 622.2028; found 622.2022.

### Synthesis of Fmoc-erythro-β-MeCys-OH

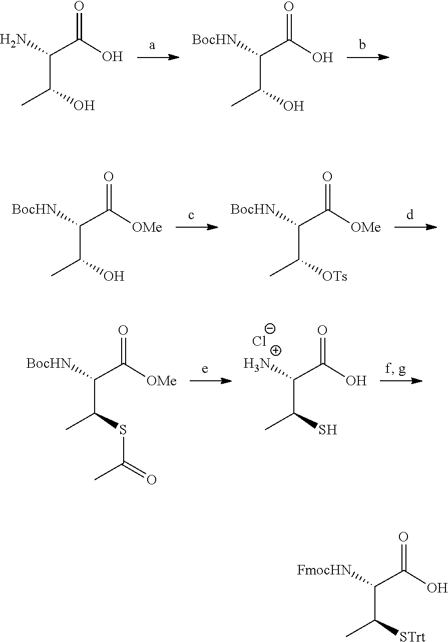

Reagents and conditions: (a) Boc₂O (1.2 eq), Na₂CO₃ (2.1 eq), H₂O/THF. 4° C. to rt, O.N. (b) Mel (5.7 eq), K₂CO₃ (1.7 eq), DMF, rt 2 h, 69% over two steps (c) TsCl (1.3 eq), dry pyridine, 4° C., O.N. 55% (d) KSAc (1.55 eq), dry DMF, 4° C. to rt, O.N. 46% (e) 6M HCl_{(aq)}, reflux, 1 h (f) triphenylmethanol (1 eq), TFA, rt, 15 min (g) Fmoc-Oxyma (1 eq), NaHCO₃ (3.5 eq), H₂O/acetone, rt, O.N. 64% over three steps.

### (tert-Butoxycarbonyl)-L-threonine

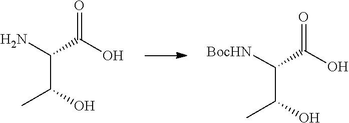

L-Threonine (5 g, 41.97 mmol) was dissolved in 175 mL H₂O and stirred at 4° C. Na₂CO₃ (9.34 g, 88.15 mmol) was added to the stirring mixture. Boc₂O (10.99 g, 50.37 mmol) was dissolved in 225 mL THF and this solution was added to the aqueous mixture containing the amino acid, dropwise. The reaction mixture was then allowed to warm up to room temperature overnight. Upon completion of the reaction, the solution was acidified to a pH of about 2 with ortho-phosphoric acid. It was extracted with 300 mL EtOAc and then 2 further 150 mL portions of EtOAc. The organic layers were pooled and washed with 200 mL sat. KH₂PO_{4(aq)}. The organic layer was then dried over magnesium sulfate, filtered into a round bottom flask and the solvent was evaporated under reduced pressure to yield a clear gum. The crude product was purified by flash chromatography on silica (2.5″ diameter, 7″ height silica). It was loaded with 0.1:0.5:9.4 (v/v/v) AcOH/EtOH/DCM and eluted with 0.1:0.8:9.1 (v/v/v) AcOH/EtOH/DCM. The pure fractions were pooled and evaporated under reduced pressure to yield a clear oil (Quantitative yield). TLC (AcOH/EtOH/DCM 0.1:0.5:9.4 v/v/v): R_{f}=0.13; ¹H NMR (300 MHz, CD₂Cl₂) δ 5.54 (d, J=7.9 Hz, 1H), 4.38 (broad singlet, 1H), 4.25 (d, J=7.5 Hz, 1H), 1.45 (s, 9H), 1.24 (d, J=6.1 Hz, 3H); ¹³C NMR (75 MHz, CD₂Cl₂) δ 174.83 (s), 157.00 (s), 80.78 (s), 68.21 (s), 59.03 (s), 28.41 (s), 19.53 (s). HRMS-ESI (m/z): [M+Na]⁺ calcd. for C₉H₁₇NO₅Na, 242.1004; found 242.1006.

### Methyl (tert-butoxycarbonyl)-L-threoninate

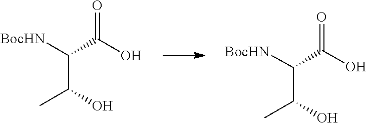

(tert-Butoxycarbonyl)-L-threonine (41.97 mmol) was dissolved in 100 mL DMF and K₂CO₃ (9.87 g, 71.46 mmol) was added to solution, which was stirred at 4° C. for about 5 minutes. Mel (14.83 mL, 238.21 mmol) was added to the reaction mixture, dropwise. The reaction mixture was then allowed to warm up to room temperature and it was stirred for 2 hours. The solution was then diluted with 500 mL EtOAc and washed with two 250 mL portions of H₂O and one 250 mL portion of brine. The organic layer was dried over magnesium sulfate, filtered into a round bottom flask and the solvent was evaporated under reduced pressure to yield a thick yellow oil. The crude product was purified by flash chromatography on silica (2.75″ diameter, 7″ height silica). using isocratic elution with 1:2 (v/v) EtOAc/Hexanes. The pure fractions were pooled and evaporated under reduced pressure to yield a clear oil (6.79 g. 29.14 mmol, 69% yield). TLC (EtOAc/Hexanes 1:1 v/v): R_{f}=0.32; ¹H NMR (300 MHz, CD₂Cl₂) δ 5.26 (s, 1H), 4.34-4.14 (m, 2H), 3.74 (s, 3H), 1.44 (s, 9H), 1.21 (d, J=6.3 Hz, 6H); ¹³C NMR (101 MHz, CD₂Cl₂) δ 172.29 (s), 156.38 (s), 80.17 (s), 68.43 (s), 59.17 (s), 52.70 (s), 28.40 (s), 20.12 (s); HRMS-ESI (m/z): [M+Na]⁺ calcd. for C₉H₁₇NO₅Na, 256.1161; found 256.1160.

### Methyl N-(tert-butoxycarbonyl)-O-tosyl-L-threoninate

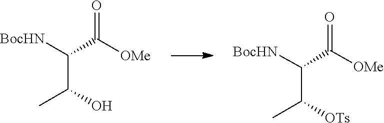 [SMILES: COC(=O)[C@@H](NC(=O)OC(C)(C)C)[C@@H](C)O ; COC(=O)[C@@H](NC(=O)OC(C)(C)C)[C@@H](C)OS(=O)(=O)c1ccc(C)cc1]

Methyl (tert-butoxycarbonyl)-L-threoninate (2.02 g, 8.65 mmol) was dissolved in 5.6 mL dry pyridine and transferred to a flame dried round bottom flask under argon. The solution was stirred and cooled to 1° C. in an ice bath. Recrystallized tosyl chloride (2.14 g, 11.24 mmol) was added to the reaction mixture, under argon, and the reaction was stirred overnight at 1° C. Upon completion of the reaction, the reaction mixture was diluted with 150 mL Et₂O and washed with one 150 mL portion of 0.01M HCl_{(aq)} and two 150 mL portions of H₂O. The organic layer then was dried over magnesium sulfate, filtered into a round bottom flask and the solvent was evaporated under reduced pressure to yield a clear gum speckled with a white solid. The crude product was purified by flash chromatography on silica (1.75″ diameter, 6.5″ height silica) using gradient elution from 1:9 (v/v) EtOAc/Hexanes to 1:2 (v/v) EtOAc/Hexanes. The pure fractions were pooled and evaporated under reduced pressure to yield a clear oil (1.834 g, 4.74 mmol, 55% yield). TLC (EtOAc/Hexanes 1:1 v/v): R_{f}=0.55; ¹H NMR (400 MHz, CD₂Cl₂) δ 7.73 (d, J=8.3 Hz, 2H), 7.37 (d, J=8.1 Hz, 2H), 5.23 (d, J=9.3 Hz, 1H), 5.07 (qd, J=6.3, 2.0 Hz, 1H), 4.38 (dd, J=9.7, 1.8 Hz, 1H), 3.54 (s, 3H), 2.45 (s, 3H), 1.42 (s, 9H), 1.31 (d, J=6.4 Hz, 3H). ¹³C NMR (101 MHz, CD₂Cl₂) δ 169.93 (s), 155.98 (s), 145.69 (s), 134.04 (s), 130.29 (s), 128.18 (s), 80.54 (s), 79.31 (s), 57.93 (s), 52.91 (s), 28.33 (s), 21.78 (s), 18.29 (s). HRMS-ESI (m/z): [M+Na]⁺ calcd. for C₁₇H₂₅NO₇SNa, 410.1249; found 410.1246.

### Methyl (2R,3S)-3-(acetylthio)-2-((tert-butoxycarbonyl)amino)butanoate

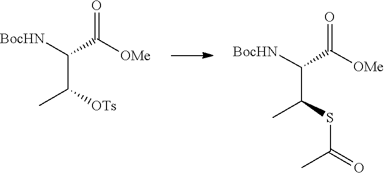 [SMILES: COC(=O)[C@@H](NC(=O)OC(C)(C)C)[C@@H](C)OS(=O)(=O)c1ccc(C)cc1 ; COC(=O)[C@@H](NC(=O)OC(C)(C)C)[C@H](C)SC(C)=O]

Methyl N-(tert-butoxycarbonyl)-O-tosyl-L-threoninate (1.834 g, 4.74 mmol) was dissolved in 6 mL DMF and transferred to a flame dried round bottom flask under argon. Freshly made potassium thioacetate (0.839 g, 7.34 mmol) was added to the reaction vessel under argon and the solution was stirred for three days at room temperature. Upon completion of the reaction, the reaction mixture was diluted with 200 mL Et₂O and washed with one 100 mL portion of H₂O. The aqueous layer was then extracted with one 100 mL portion of Et₂O. The organic layers were pooled, dried over magnesium sulfate, filtered into a round bottom flask and the solvent was evaporated under reduced pressure to yield a yellow oil. The crude product was purified by flash chromatography on silica (1.25″ diameter, 13″ height silica) using isocratic elution with 2:8 (v/v) Et₂O/Toluene. The pure fractions were pooled and evaporated under reduced pressure to yield a clear oil (0.632 g, 2.17 mmol, 46% yield). TLC (EtOAc/Hexanes 1:1 v/v): R_{f}=0.56; ¹H NMR (300 MHz, CD₂Cl₂) δ 5.20 (s, 1H), 4.56 (dd, J=8.9, 3.3 Hz, 1H), 3.99 (qd, J=7.3, 4.0 Hz, 1H), 2.31 (s, 3H), 1.43 (s, 9H), 1.25 (d, J=7.3 Hz, 3H); ¹³C NMR (75 MHz, CD₂Cl₂) δ 195.13 (s), 171.15 (s), 155.58 (s), 57.22 (s), 52.82 (s), 41.63 (s), 30.80 (s), 28.36 (s), 16.86 (s). HRMS-ESI (m/z): [M+Na]⁺ calcd. for C₁₂H₂₁NO₅SNa, 314.1038; found 314.1034.

### (2R,3S)-2-Amino-3-mercaptobutanoic acid

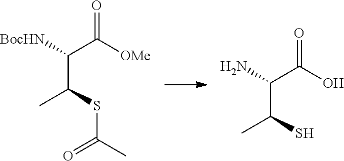

Methyl (2R,3S)-3-(acetylthio)-2-((tert-butoxycarbonyl)amino)butanoate (0.630 g, 2.16 mmol) was refluxed in 20 mL 6M HCl_{(aq)} at about 120° C. for 1 hour. Upon completion of the reaction, the mixture was allowed to cool to room temperature, diluted to about 40 mL with H₂O, and then transferred to a separatory funnel, where it was extracted twice with 40 mL 1:1 (v/v) EtOAc/Pet. Et. and then once with a 40 mL portion of Et₂O. The aqueous layer was evaporated to dryness under reduced pressure to yield a white solid. (0.36 g, 2.10 mmol, 97% Yield). ¹H NMR (300 MHz, D₂O) δ 4.08 (d, J=3.5 Hz, 1H), 3.62 (qd, J=7.2, 3.6 Hz, 1H), 1.42 (d, J=7.2 Hz, 3H); ¹³C NMR (75 MHz, D₂O) δ 170.13 (s), 59.50 (s), 34.36 (s), 19.58 (s); HRMS-ESI (m/z): [M+Na]⁺ calcd. for C₄H₁₀NO₂S, 136.0432; found 136.0437.

### (2R,3S)-2-((((9H-Fluoren-9-yl)methoxy)carbonyl)amino)-3-(tritylthio)butanoic acid

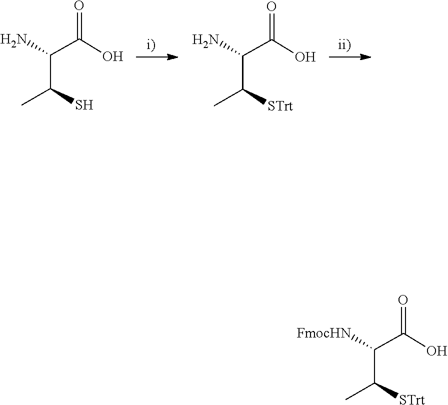

i) (2R,SR)-2-Amino-3-mercaptobutanoic acid (0.357 g, 2.08 mmol) and triphenylmethanol (0.542 g, 2.08 mmol) were dissolved in 4 mL TFA. The reaction mixture was stirred for 15 minutes at room temperature. Upon completion of the reaction, the solvent was removed under reduced pressure and the solid was left on a vacuum line overnight, until all traces of yellow disappeared from the white solid. ii) The crude product from the previous step was dissolved in 2.08 mL H₂O and 8.33 mL sat. NaHCO_{3(aq)} was added to the stirring mixture. The pH of the resultant solution was about 9. A suspension of Fmoc-Oxyma (0.758 g, 2.08 mmol) in 10.4 mL acetone was added dropwise to the stirring mixture and it was allowed to proceed at room temperature, overnight. The acetone was removed in vacuo and the remaining solution was diluted with 40 mL H₂O and acidified to a pH of about 2 with ortho-phosphoric acid. It was then extracted with three 50 mL portions of DCM and the organic layers were pooled and washed with one 100 mL portion of sat. NaHCO_{3(aq)}. This mixture took 30 minutes to resolve. The organic layer was then washed with 100 mL portion of sat. KH₂PO_{4(aq)} and dried over magnesium sulfate, filtered into a round bottom flask and the solvent was evaporated under reduced pressure to yield a white solid. The crude product was purified by two successive rounds of flash chromatography on silica (1.25″ diameter, 12″ height silica) using gradient elution from 4:96 (v/v) MeOH/DCM to 1:9 (v/v) MeOH/DCM. The pure fractions were pooled and evaporated under reduced pressure to yield a white solid (0.792 g, 1.32 mmol, 64% yield over three steps). TLC (MeOH/DCM 1:9 v/v): R_{f}=0.33; ¹H NMR (300 MHz, CD₂Cl₂) δ 7.78 (d, J=7.5 Hz, 2H), 7.61 (d, J=6.6 Hz, 2H), 7.54-7.47 (m, 6H), 7.39 (t, J=7.5 Hz, 2H), 7.34-7.13 (m, 11H), 5.22 (d, J=7.7 Hz, 1H), 4.44-4.31 (m, 2H), 4.27-4.19 (m, 1H), 3.90 (d, J=6.4 Hz, 1H), 2.91-2.80 (m, 1H), 1.07 (d, J=7.2 Hz, 6H). ¹³C NMR (75 MHz, CD₂Cl₂) δ 173.59 (s), 156.61 (s), 144.95 (s), 141.68 (s), 129.87 (s), 128.47 (s), 128.09 (s), 127.48 (s), 127.23 (s), 125.53 (s), 125.46 (s), 120.33 (s), 68.46 (s), 67.54 (s), 58.43 (s), 47.52 (s), 41.90 (s), 17.92 (s); HRMS-ESI (m/z): [M+Na]⁺ calcd. for C₃₈H₃₃NO₄Na, 622.2028.1004; found 622.2026.

### Synthesis of Fmoc-Oxo-Ile-OH

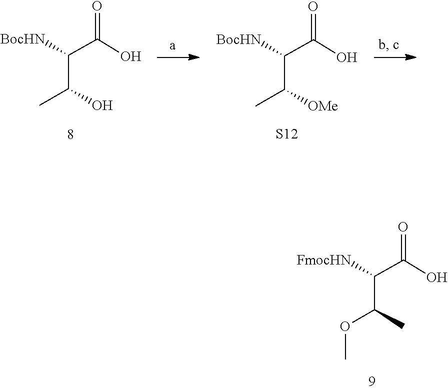

Reagents and conditions: (a) NaH (3 eq), Mel (1 eq), dry DMF, 4° C., 5 h 48% (b) 1:2 TFA/DCM, rt, 1 h (c) Fmoc-OSu (1.5 eq), DIEA (1 eq), MeCN, 4° C. to rt, O.N. 31% over two steps.

### N-(tert-butoxycarbonyl)-O-methyl-L-threonine

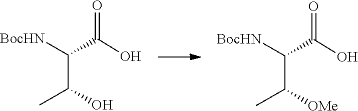 [SMILES: C[C@@H](O)[C@H](NC(=O)OC(C)(C)C)C(=O)O ; CO[C@H](C)[C@H](NC(=O)OC(C)(C)C)C(=O)O]

(tert-Butoxycarbonyl)-L-threonine (0.5 g, 2.283 mmol) was dissolved in 20 mL dry DMF and stirred at 4° C. under argon. Three portions of 60% NaH suspended in mineral oil (0.092 g, 2.3 mmol for each portion, 6.9 mmol total) were added to the stirring mixture and it was left to stir at 4° C. for 2.5 hours. Mel (0.142 mL, 2.283 mmol) was added to the stirring mixture, dropwise. After a further 3 hours of stirring at 4° C. the reaction mixture was acidified with ortho-phosphoric acid to a pH of 2 and diluted with 100 mL sat. KH₂PO_{4(aq)} and the solution was extracted with ten 20 mL portions of EtOAc. The organic layers were pooled and washed with one 50 mL portion of brine and dried over magnesium sulfate, filtered into around bottom flask and the solvent was evaporated under reduced pressure to yield a yellow oil. The crude product was purified by flash chromatography on silica (1.25″ diameter, 7″ height silica) using gradient elution from 0.1:0.2:9.7 (v/v/v) AcOH/EtOH/DCM to 0.1:0.5:9.4 (v/v/v) AcOH/EtOH/DCM. The pure fractions were pooled and evaporated under reduced pressure to yield a clear oil (0.256 g, 1.10 mmol, 48% yield). TLC (AcOH/EtOH/DCM 0.1:0.5:9.4 v/v/v): R_{f}=0.38 ¹H NMR (300 MHz, CD₂Cl₂) δ 5.28 (d, J=8.1 Hz, 1H), 4.30 (d, J=7.0 Hz, 1H), 4.01-3.89 (m, J=3.7 Hz, 1H), 3.34 (s, 3H), 1.44 (s, 9H), 1.19 (d, J=6.3 Hz, 3H); ¹³C NMR (75 MHz, CD₂Cl₂) δ 174.77 (s), 156.46 (s), 80.37 (s), 76.63 (s), 58.07 (s), 57.11 (s), 28.39 (s), 15.44 (s); HRMS-ESI (m/z): [M+Na]⁺ calcd. for C₁₀H₁₉NO₅Na, 256.1161; found 256.1160.

### N-(((9H-Fluoren-9-yl)methoxy)carbonyl)-O-methyl-L-threonine

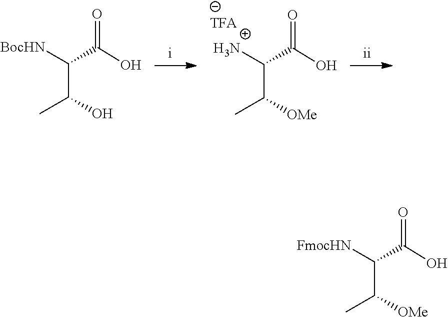

i) N-(tert-Butoxycarbonyl)-O-methyl-L-threonine (0.256 g, 1.10 mmol) was stirred in 45 mL 1:2 TFA/DCM for 2 hours. Upon completion of the reaction, the solvent was removed in vacuo and the crude oil was used in the next step without further purification. ii) 0-Methyl-L-threonine (1.11 mmol) was dissolved in 2 mL H₂O and N,N-diisopropylethylamine (DIEA, 0.192 mL, 1.1 mmol) was added to the stirring reaction mixture at room temperature. N-(9-Fluorenylmethoxycarbonyloxy)succinimide (Fmoc-OSu, 0.560 g, 1.66 mmol) dissolved in 2 mL MeCN was added to the stirring reaction mixture and DIEA was added until pH 9 was reached. The reaction was allowed to proceed at room temperature for 2 hours and it was left at 1° C. overnight. The reaction mixture was diluted with 50 mL sat. KH₂PO_{4(aq)} and the solution was acidified to pH 1 with ortho-phosphoric acid. The solution was then extracted with three 30 mL portions of EtOAc, the organic layers were pooled and washed with one 25 mL portion of sat. KH₂PO_{4(aq)} and one 25 mL portion of brine. The organic layer was then dried over magnesium sulfate, filtered into a round bottom flask and the solvent was evaporated under reduced pressure to yield a white solid. The crude product was purified by flash chromatography on silica (1.25″ diameter, 7″ height silica) using gradient elution from 0.1:0.2:9.7 (v/v/v) AcOH/EtOH/DCM to 0.1:0.3:9.6 (v/v/v) AcOH/EtOH/DCM. The pure fractions were pooled and evaporated under reduced pressure to yield a white solid (0.122 g, 0.343 mmol, 31% yield over two steps). TLC (AcOH/EtOH/DCM 0.1:0.3:9.6 v/v/v): R_{f}=0.15 ¹H NMR (400 MHz, CD₂Cl₂) δ 8.07 (d, J=11.2 Hz, 2H), 7.90 (d, J=7.6 Hz, 2H), 7.65 (t, J=11.0 Hz, 2H), 7.55 (t, J=11.0 Hz, 2H), 5.65 (s, 1H), 4.33-4.23 (m, 3H), 4.11 (t, J=10.1 Hz, 1H), 3.83 (d, J=6.9 Hz, 1H), 3.11 (s, 3H), 0.69 (d, J=9.1 Hz, 3H); ¹³C NMR (75 MHz, CD₂Cl₂) δ 175.34 (s), 157.13 (s), 144.30 (s), 141.65 (s), 128.07 (s), 127.45 (s), 125.51 (s), 120.31 (s), 76.66 (s), 67.54 (s), 58.76 (s), 57.13 (s), 47.55 (s), 15.53 (s); HRMS-ESI (m/z): [M+Na]⁺ calcd. for C₁₀H₁₉NO₅Na, 378.1317; found 378.1319.

### Synthesis of Fmoc-azaGly-Ile-OH

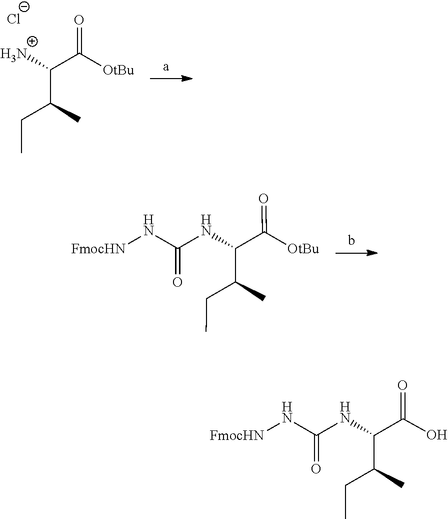

Reagents and conditions: (a) i) DIEA (1 eq), then Boc₂O (1.4 eq), and 4-dimethylaminopyridine (DMAP, 1 eq), dry DCM, 15 minutes, rt. ii) Fmoc-Hydrazine (1.4 eq) O.N. 67% yield over 2 steps (b) 1:2 TFA/DCM, rt, 1 h used without further purification.

### (9H-Fluoren-9-yl)methyl 2-(((2S,3S)-1-(tert-butoxy)-3-methyl-1-oxopentan-2-yl)carbamoyl)hydrazine-1-carboxylate

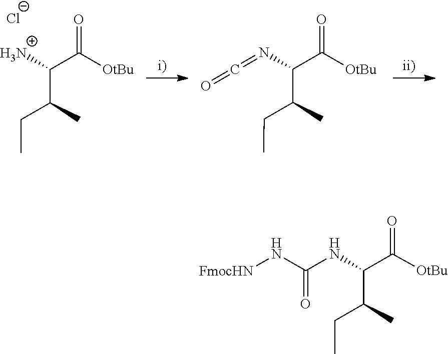

i) (2S,3S)-1-(tert-Butoxy)-3-methyl-1-oxopentan-2-aminium chloride (0.5 g, 2.235 mmol) was dissolved in 10 mL dry DCM and DIEA (0.39 mL, 2.235 mmol) was added dropwise to the stirring solution at room temperature. In a separate flask, Boc₂O (0.683 g, 3.13 mmol) and DMAP (0.273 g, 2.235 mmol) were dissolved in 10 mL dry DCM and stirred at room temperature. The solution containing the free-based amino acid was added dropwise to the stirring mixture of Boc₂O and DMAP and combined solutions were stirred at room temperature for 15 minutes. Upon completion of the reaction, the solution was transferred to a separatory funnel and washed with one 10 mL sat. KH₂PO_{4(aq)} and the organic layer was transferred back into the reaction flask. ii) Fmoc-Hydrazine (0.796 g, 3.13 mmol) was added to the reaction mixture and it was stirred at room temperature overnight. Upon completion of the reaction, the solution was washed with two 10 mL portions of sat. KH₂PO_{4(aq)}, 10 mL portions of sat. NaHCO_{3(aq)} and one 10 mL portion of brine. The organic layer was then dried over magnesium sulfate, filtered into a round bottom flask and the solvent was evaporated under reduced pressure to yield a white solid. The crude product was purified by flash chromatography on silica (1.25″ diameter, 9″ height silica) using isocratic elution with 1:1 (v/v) EtOAc/Hexanes. The pure fractions were pooled and evaporated under reduced pressure to yield a white solid (0.700 g, 1.50 mmol, 67% yield over two steps). ¹H NMR (300 MHz, CD₂Cl₂) δ 7.77 (d, J=7.5 Hz, 2H), 7.60 (dd, J=7.4, 2.1 Hz, 2H), 7.40 (t, J=7.4 Hz, 2H), 7.30 (t, J=7.4 Hz, 2H), 6.93 (s, 1H), 6.73 (s, 1H), 5.88 (d, J=8.2 Hz, 1H), 4.47-4.37 (m, 2H), 4.33 (dd, J=8.5, 4.6 Hz, 1H), 4.24 (t, J=7.1 Hz, 1H), 1.93-1.79 (m, 1H), 1.49-1.36 (m, 9H), 1.25-1.08 (m, 1H), 0.93-0.85 (m, 6H). ¹³C NMR (75 MHz, CD₂Cl₂) δ 172.04 (s), 158.19 (s), 157.21 (s), 144.00 (s), 141.65 (s), 128.17 (s), 127.54 (s), 125.53 (s), 120.35 (s), 82.37 (s), 68.41 (s), 58.05 (s), 47.33 (s), 38.66 (s), 28.17 (s), 25.59 (s), 15.59 (s), 11.92 (s); HRMS-ESI (m/z): [M+Na]⁺ calcd. for C₂₆H₃₃N₃O₅Na, 490.2318; found 490.2321.

### (2-(((9H-Fluoren-9-yl)methoxy)carbonyl)hydrazine-1-carbonyl)-L-isoleucine

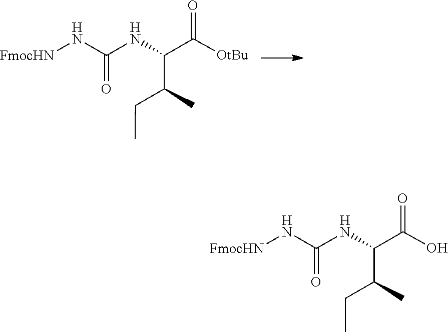

(9H-Fluoren-9-yl)methyl 2-(((2S,3S)-1-(tert-butoxy)-3-methyl-1-oxopentan-2-yl)carbamoyl)hydrazine-1-carboxylate (0.091 g, 0.195 mmol) was stirred in 1 mL 1:1 TFA/DCM for 2 hours. Upon completion of the reaction, the solvent was removed in vacuo and the crude oil was used in the next step without further purification.

### Synthesis of Fmoc-azaGly-Cys(STrt)-OH

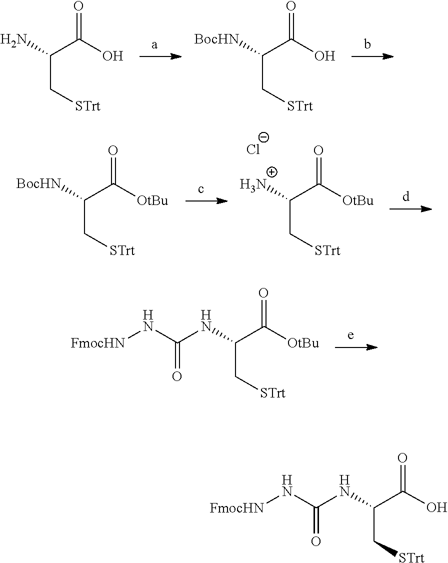

Reagents and conditions: (a) Boc₂O, Na₂CO₃, THF/H₂O 4° C. to rt, O.N. (b) N,N-diisopropyl-O-tertbutyl-isourea, dry DCM, rt, O.N. 62% over two steps (c) acetyl chloride (AcCl), MeOH, dry EtOAc 3 h, rt (d) i) DIEA (1 eq), then Boc₂O (1.4 eq), and DMAP (1 eq), dry DCM, 15 minutes, rt. ii) Fmoc-Hydrazine (1.4 eq) O.N. 43% yield over 2 steps (e) 1:2 TFA/DCM, rt, 1 h used without further purification.

### N-(tert-Butoxycarbonyl)-S-trityl-L-cysteine

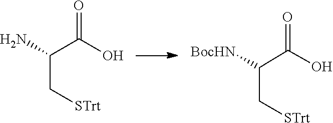

S-Trityl-L-cysteine (5.0 g, 13.76 mmol) was dissolved in H₂O (60 mL) and stirred at 4° C. Na₂CO₃ (3.1 g, 28.89 mol) was added slowly to the stirring mixture. Boc₂O (3.6 g, 16.61 mmol) was dissolved in THF (75 mL) and the organic solution was added to the aqueous dropwise. The mixture was stirred overnight at room temperature. Upon completion, the mixture was acidified to a pH of about 2 with sat. ortho phosphoric acid and then extracted with EtOAc (100 mL, 2×50 mL), washed with saturated KH₂PO₄ (100 mL), dried with MgSO₄, filtered and concentrated under reduced pressure. The crude product was purified by flask chromatography on silica gel with isocratic elution AcOH/EtOH/DCM (1:5:94, v/v). The pure fractions were pooled, and the solvent was removed under reduced pressure to yield a white solid. Yield: 4.9 g, 76%. ¹H NMR (300 MHz, CD₂Cl₂) δ 7.42 (dd, J=5.3, 3.4 Hz, 6H), 7.34-7.19 (m, 9H), 4.94 (d, J=6.6 Hz, 1H), 4.12-4.06 (m, 1H), 2.64 (d, J=5.4 Hz, 2H), 1.42 (s, 9H). ¹³C NMR (75 MHz, CD₂Cl₂) δ 174.79 (s), 156.43 (s), 145.11 (s), 130.27 (s), 128.85 (s), 127.71 (s), 81.34 (s), 67.83 (s), 34.10 (s), 28.78 (s), 27.93 (s). HRMS-ESI (m/z): [M+Na]⁺ calcd. for C₂₇H₂₉NO₄Na, 486.1715; found 486.1715.

### tert-Butyl N-(tert-butoxycarbonyl)-S-trityl-L-cysteinate

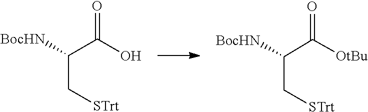

The N-Boc-S-trityl-L-cysteine (4.9 g, 10.57 mmol) was dissolved in dry DCM (50 mL) and N,N-diisopropyl-O-tert-butyl-isourea (8.0 g, 40.17 mmol) was added. The solution was stirred at reflux for 24 h. Upon completion of the reaction, the diisopropylurea by-product had precipitated and was removed by filtration. The reaction mixture was then concentrated under reduced pressure and purified by flash chromatography on silica using isocratic elution with Hexane/Diethyl Ether (2:1, v/v). The pure fractions were pooled, and the solvent was removed under reduced pressure to yield a white solid. Yield: 4.5 g, 82%. TLC (Hexanes/Et₂O=2:1 v/v): R_{f}=0.50 ¹H NMR (300 MHz, CD₂Cl₂) δ 7.45-7.38 (m, 6H), 7.35-7.20 (m, 9H), 5.08 (d, J=7.5 Hz, 1H), 4.19-4.08 (m, 1H), 2.50 (d, J=5.1 Hz, 2H), 1.45-1.40 (m, 18H). ¹³C NMR (75 MHz, CD₂Cl₂) δ 170.07 (s), 155.22 (s), 144.88 (s), 129.88 (s), 128.36 (s), 127.17 (s), 82.54 (s), 79.90 (s), 66.83 (s), 34.84 (s), 28.25 (s), 27.87 (s), 27.54 (s); HRMS-ESI (m/z): [M+Na]⁺ calcd. for C₃₁H₃₇NO₄Na, 542.2341; found 542.2347.

### (R)-1-(tert-Butoxy)-1-oxo-3-(tritylthio)propan-2-aminium Chloride

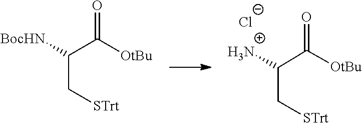

0.378 mL (5.30 mmol) Acetyl chloride was dissolved in 4 mL dry EtOAc and stirred at room temperature. Dry MeOH (0.214 mL, 5.30 mmol) was added dropwise, and the reaction mixture was stirred for a further 20 minutes at room temperature. Boc-Cys(Trt)-OtBu (0.562 g, 1.081 mmol) dissolved in 1.4 mL dry EtOAc was then added dropwise to the reaction mixture which was then stirred for 5 hours at room temperature. The reaction mixture was then concentrated in vacuo, triturated with Et₂O and evaporated twice. Purification was by flash chromatography on silica. Unreacted starting material eluted with 2:1 Hexane/Et₂O (0.224 g starting material recovered, 40%) then product eluted with 15:5:1 CHCl₃/EtOAc/MeOH. The pure fractions were pooled and evaporated under reduced pressure to yield a clear gum (0.184 g, 0.40 mmol, 38%). TLC (CHCl₃/EtOAc/MeOH=15:5:1 v/v/v): R_{f}=0.38 ¹H NMR (300 MHz, CD₂Cl₂) δ 7.75-6.90 (m, 15H), 3.44 (bs, 1H), 2.77 (bs, 2H), 1.37 (s, 9H). ¹³C NMR (75 MHz, CD₂Cl₂) δ 167.27 (s), 144.05 (s), 129.69 (s), 128.45 (s), 127.20 (s), 84.58 (s), 67.45 (s), 32.83 (s), 27.99 (s). HRMS-ESI (m/z): [M+Na]⁺ calcd. for C₂₆H₂₉NO₂Na, 442.1817; found 442.1822.

### (9H-fluoren-9-yl)methyl (R)-2-((1-(tert-butoxy)-1-oxo-3-(tritylthio)propan-2-yl)carbamoyl)hydrazine-1-carboxylate

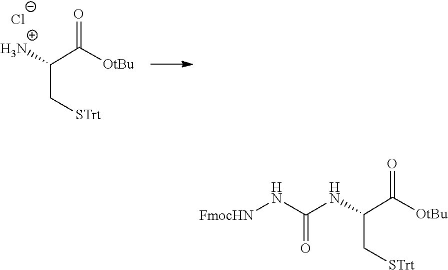

0.184 g (0.40 mmol) (R)-1-(tert-Butoxy)-1-oxo-3-(tritylthio)propan-2-aminium chloride was dissolved in 2.7 mL dry DCM. 0.071 μL (0.40 mmol) DIEA was then added dropwise to the reaction mixture, which was stirred at room temperature for 30 minutes. Simultaneously, Boc₂O (0.122 g, 0.57 mmol) and DMAP (0.049 g, 0.40 mmol) were dissolved in 2.7 mL dry DCM and stirred at room temperature for 30 minutes. After 30 minutes total reaction time for both reaction mixtures, the free based solution of (R)-1-(tert-butoxy)-1-oxo-3-(tritylthio)propan-2-aminium chloride in dry DCM was added dropwise to the stirring mixture of Boc₂O and DMAP in dry DCM. The resulting combined reaction mixture was then stirred at room temperature for a further 30 minutes. After 30 minutes, the reaction mixture was transferred to a separatory funnel and washed once with 3 mL sat. KH₂PO₄. The organic layer transferred was then transferred back into round bottom flask and then Fmoc-Hydrazine (0.143 g, 0.57 mmol) was added. The reaction mixture was then stirred at room temperature for 6 h. After 6 h, the reaction mixture was diluted to about 40 mL total volume with DCM and washed twice with 20 mL sat. KH₂PO_{4(aq)}, 20 mL sat. NaHCO_{3(aq)} and 20 mL brine. The organic layer was then concentrated under reduced pressure to yield an off white solid. The crude was purified by flash chromatography on silica using isocratic elution with 1:1 EtOAc/Hexanes. The pure fractions were pooled and evaporated under reduced pressure to yield a clear gum (0.121 g, 0.17 mmol, 43% yield). TLC (EtOAc/Hexanes=1:1 v/v): R_{f}=0.30 ¹H NMR (300 MHz, CD₂Cl₂) δ 7.78 (d, J=7.5 Hz, 2H), 7.59 (d, J=7.4 Hz, 2H), 7.47-7.12 (m, 19H), 6.64 (s, 1H), 6.35 (s, 1H), 5.78 (d, J=8.0 Hz, 1H), 4.48-4.40 (m, 2H), 4.34 (dd, J=13.1, 5.4 Hz, 1H), 4.24 (t, J=6.9 Hz, 1H), 2.63-2.43 (m, 2H), 1.39 (s, 9H). ¹³C NMR (75 MHz, CD₂Cl₂) δ 170.19 (s), 157.40 (s), 156.94 (s), 144.82 (s), 144.01 (s), 141.68 (s), 129.86 (s), 128.36 (s), 128.19 (s), 127.56 (s), 127.16 (s), 125.53 (s), 120.36 (s), 82.96 (s), 68.46 (s), 66.90 (s), 52.90 (s), 47.33 (s), 34.95 (s), 28.04 (s); HRMS-ESI (m/z): [M+Na]⁺ calcd. for C₄₂H₄₁N₃O₅SNa, 722.2665; found 722.2664.

### Fmoc-aza-Gly-S-trityl-L-cysteine

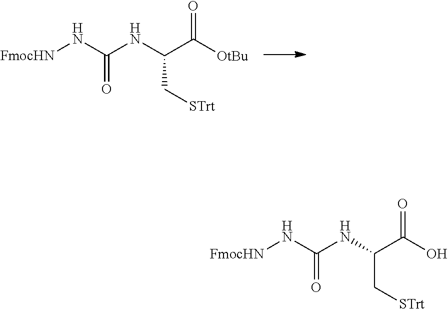

Fmoc-aza-Gly-S-trityl-L-cysteine-OtBu was dissolved in TFA (1-3 mL) and the solution was stirred for 15 min. The TFA was then removed under reduced pressure and the residue was placed under high vacuum for 2-3 days.

### Loading of 2-CTC Resin with Fmoc-Hyp(OtBu)-OH

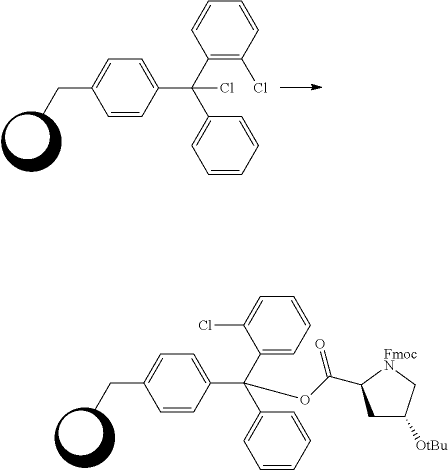

2-Chlorotrityl chloride (2-CTC) resin (1 g) was dried against P₂O₅ in a desiccator for about 4 h, and then transferred to a flame dried flask under argon. Fmoc-O-tert-butyl-L-hydroxyproline (Fmoc-Hyp(OtBu)-OH, 0.982 g, 2.4 mmol) was added to the flask under argon and the solids were dissolved/suspended in 7.5 mL dry DCM and stirred at room temperature. DIEA (1.05 mL, 6 mmol) was added dropwise to stirring reaction mixture and the reaction was allowed to proceed overnight. Upon completion of the reaction, the resin was transferred to a ZEBA Desalt spin column and washed with DCM. Fmoc loading test revealed a loading of about 0.8 mmol/g.

### General Procedure for Solid Phase Peptide Synthesis

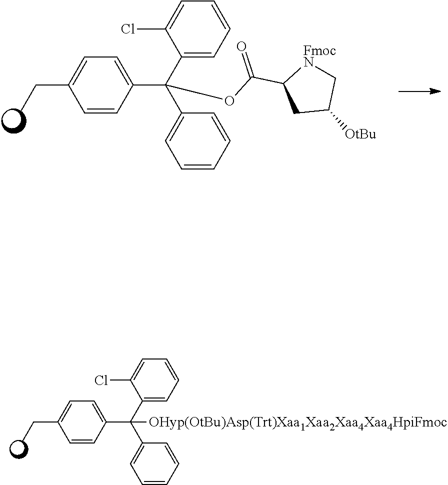

### i) Manual Solid Phase Peptide Synthesis

The dried resin contained in a ZEBA Desalt spin column was solvated in 5 mL DMF and shaken for at least 30 minutes. The solvent was drained and the resin was resuspended in 5 mL 2:8 piperidine/DMF (0.5M Oxyma) and shaken for 5 minutes. The solvent was drained and the resin was resuspended in 5 mL 2:8 piperidine/DMF (0.5M Oxyma) and shaken for a further 10 minutes. The solvent was drained, and the resin was washed with seven 5 mL portions of DMF, with shaking and draining. The resin was then Kaiser tested and if a positive result (purple or brown) was observed, the beads were then resuspended in a 5 mL solution of DMF which already contained 4 equivalents each of Fmoc-Xaa, Oxyma and (1-cyano-2-ethoxy-2-oxoethylidenaminooxy)dimethylamino-morpholino-carbenium hexafluorophosphate (COMU™) and about 11 equivalents DIEA. The resin was then shaken for 1-3 hours at room temperature. The solvent was drained, and the resin was washed with five 5 mL portions of DMF, with shaking and draining. The resin was then Kaiser tested and if a negative result (no change in coloration) was observed, the beads were then resuspended in a 5 mL solution of 1:2:2 Ac₂O/Collidine/EtOAc and shaken for 20 minutes at room temperature. If the result of the Kaiser test remained positive, another coupling with the same Fmoc amino acid was performed. Once the capping was complete, the solvent was drained and the resin was washed with five 5 mL portions of DMF, with shaking and draining. The resin was then washed with DCM and left to dry under reduced pressure if couplings were complete for the day or resuspended in 2:8 piperidine/DMF (0.5 M Oxyma) for the next Fmoc deprotection.

### ii) Automatic Solid Phase Peptide Synthesis

Using a Gyros PurePep Chorus GT outfitted with induction heating, preloaded CTC resin was solvated in 3 mL of DMF and mixed by bubbling nitrogen for 20 minutes and then drained. Resin was resuspended in 3 mL 2:8 piperidine/DMF and mixed by bubbling nitrogen for 1 minute at 50° C., the solution was drained, and the process was repeated with a fresh 3 mL portion of DMF. Resin was then washed three times with 3 mL portions of DMF (mixed by bubbling with nitrogen and then drained). Resin was then resuspended in 3 mL DMF containing Fmoc-Xaa (100 mM), 0-(1H-6-chlorobenzotriazole-1-yl)-1,1,3,3-tetramethyluronium hexafluorophosphate (HCTU, 100 mM), and N-methyl-morpholine (200 mM) with at least 7.5 equivalents of amino acid and coupling agent to initial resin loading. Resin suspended in coupling solution was mixed by bubbling with nitrogen for 5 minutes at 50° C. Exceptions were as follows: Fmoc-aza-Xaa-OH dipeptides were mixed by bubbling with nitrogen at 50° C. for 50 minutes. Fmoc-Hpi-OH or Boc-Fpi(5OTBS)-OH were mixed by bubbling with nitrogen at room temperature for 1 hour. Resin was then washed three times with 3 mL portions of DMF (mixed by bubbling with nitrogen and then drained). After the last coupling, resin was then washed four times with 3 mL portions of DCM (mixed by bubbling with nitrogen and then drained). The resin was then dried by flushing with nitrogen for 20 minutes. Fmoc-Hpi-OH was prepared per a previous report (Blanc et al., 2017) and Boc-Fpi(5OTBS)-OH was prepared per a previous report (Pryyma et al., 2020).

### General Procedure for Savige-Fontana Tryptathionylation

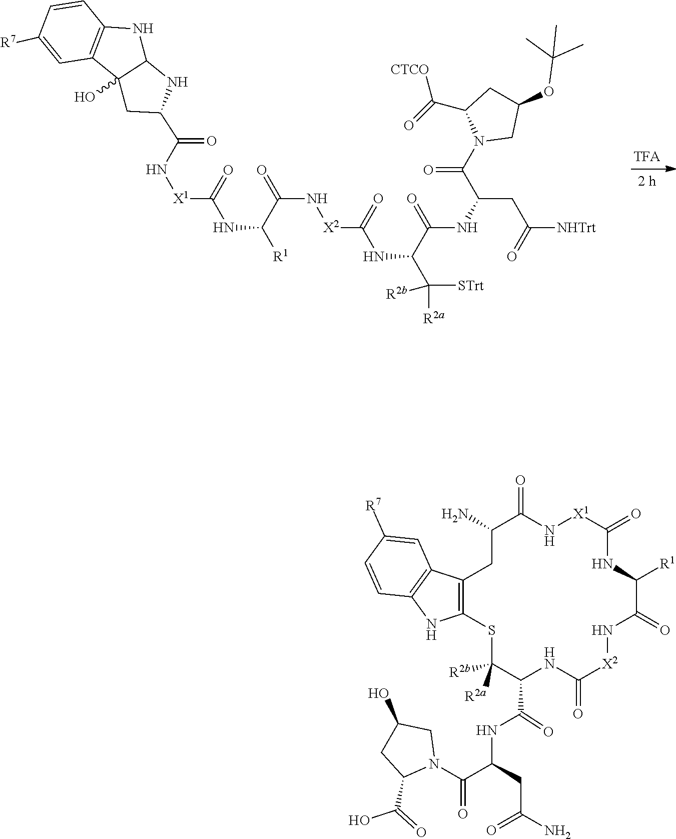

i) Cleavage Procedure from Manual Synthesis

To the spin column containing the free-base resin bound peptide was added 5 mL TFA and the beads were shaken at room temperature for 2 hours. The solution was then filtered into a round bottom flask containing 0.25 mL 1:1 v/v triisopropylsilane (TIS)/H₂O and the filtrate was evaporated under reduced pressure to yield a brown or pinkish solid. The last traces of TFA were removed by two successive rounds of co-evaporation with DCM. The crude peptide was then triturated with three 10 mL portions of diethyl ether (Et₂O) and allowed to air dry overnight to yield an off-white powder.

ii) Cleavage Procedure from Automatic Peptide Synthesis

Using a Gyros PurePep Chorus GT outfitted with induction heating, free amine or Boc protected Hpi bearing resin was suspended in TFA (3 mL) and mixed by gentle bubbling with nitrogen for 2 hours at room temperature. TFA solution was then collected into a 15 mL Falcon tube, 75 μL of TIS and 75 μL of H₂O were added and the solution was vortexed and allowed to react until the yellow color from trityl cation was no longer visible (around 30 minutes). The light orange/brown solution was diluted with Et₂O, and cooled to −20° C., resulting in a white precipitate. The suspension was spun down on a clinical centrifuge, supernatant was discarded, and the pellet was then resuspended in Et₂O, vortexed and then spun down on clinical centrifuge and the process of resuspension in Et₂O was repeated. The resulting white pellet was allowed to dry under ambient atmosphere.

### General Procedure for Crude Heptapeptide Purification

### i) SEP-PACK Purification

The crude material was purified on a reverse-phase SEP-PACK column (Waters, 3 cc) loaded with 0.1% formic acid H₂O (10 mL) and eluted with successive 10 mL portions of 5:95 MeCN/H₂O (0.1% formic acid), 10:90 MeCN/H₂O (0.1% formic acid), 15:85 MeCN/H₂O (0.1% formic acid), 20:80 MeCN/H₂O (0.1% formic acid), and 40:60 MeCN/H₂O (0.1% formic acid). Fractions were checked for purity and for presence of the desired product by electrospray ionization mass spectrometry (ESI MS) and/or HPLC. The fractions representing the purest and most abundant composition of product were pooled, frozen and lyophilized.

### ii) HPLC Purification

Crude peptide was dissolved in a minimal volume of 0.10% formic acid H₂O, with 0.1% formic acid added to aid dissolution if necessary. Once clear, the solution was purified on HPLC 1 using HPLC Method D with either column 1 or 2. Product peak was identified by ESI MS and frozen and lyophilized to yield a fluffy white powder.

TABLE 2
Heptapeptide Yields

| | Isolated Yield | | |
|---|---|---|---|
| | from Initial | Synthetic | Purification |
| Compound | Resin Loading | Method | Method |
| V(i) | 26% | Manual SPPS | HPLC/Column 1 |
| V(ii) | 36% | Manual SPPS | SEP-PACK |
| V(iii) | 23% | Automatic | HPLC/Column 1 |
| | | SPPS | |
| V(iv) | 48% | Automatic | Used as Crude |
| | | SPPS | |
| V(v) | 38% | Manual SPPS | SEP-PACK |
| V(vi) | 37% | Manual SPPS | SEP-PACK |
| V(vii) | 44% | Manual SPPS | HPLC/Column 1 |
| V(viii) | 20% | Manual SPPS | SEP-PACK |
| V(ix) | 17% | Automatic | Used as Crude |
| | | SPPS | |
| V(x) | 12% | Manual SPPS | HPLC/Column 1 |
| V(xi) | 48% | Manual SPPS | HPLC/Column 1 |
| V(xii) | 30% | Manual SPPS | HPLC/Column 1 |
| V(xiii) | 12% | Manual SPPS | HPLC/Column 1 |

### Compound V(i): (2R,3S)-β-Methyl-Cys⁸-Heptapeptide Monocycle

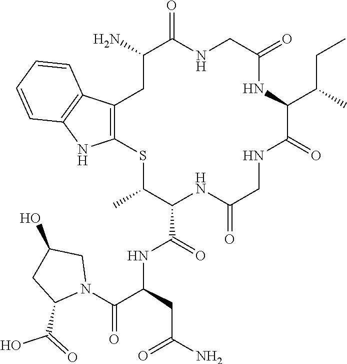 [SMILES: CC[C@H](C)[C@@H]1NC(=O)CNC(=O)[C@@H](N)Cc2c([nH]c3ccccc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=21.1 min; λₘₐₓ 290 nm; HRMS-ESI (m/z): [M+H]⁺ calcd. for C₃₄H₄₈N₉O₁₀S, 774.3245; found 774.3240. ¹H NMR (400 MHz, MeOD) δ 7.66 (d, J=8.0 Hz, 1H), 7.31 (d, J=8.2 Hz, 1H), 7.14 (t, J=7.5 Hz, 1H), 7.03 (t, J=7.5 Hz, 1H), 5.00 (m, 1H)*, 4.46 (m, 2H), 4.23 (d, J=16.8 Hz, 1H), 4.18-4.05 (m, 1H), 3.95 (t, J=7.9 Hz, 1H), 3.84 (dd, J=10.9, 4.8 Hz, 1H), 3.72 (t, J=7.3 Hz, 1H), 3.69-3.56 (m, 1H), 3.05 (dd, J=14.3, 8.0 Hz, 1H), 2.78 (dd, J=15.0, 4.7 Hz, 1H), 2.67-2.59 (m, 1H), 2.35-2.15 (m, 1H), 2.14-1.98 (m, 1H), 1.61 (m, 1H), 1.19 (d, J=7.0 Hz, 3H), 0.95 (d, J=6.0 Hz, 3H), 0.90 (t, J=7.3 Hz, 3H). *Assigned by COSY.

### Compound V(ii): (2R,3R)-β-Methyl-Cys⁸-Heptapeptide Monocycle

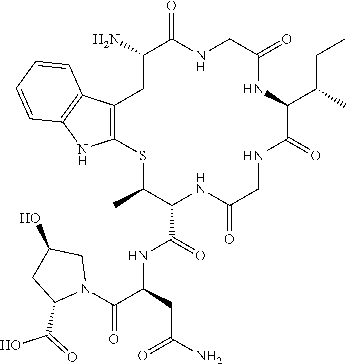 [SMILES: CC[C@H](C)[C@@H]1NC(=O)CNC(=O)[C@@H](N)Cc2c([nH]c3ccccc23)S[C@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=20.0 min; λₘₐₓ 290 nm; HRMS-ESI (m/z): [M+H]⁺ calcd. for C₃₄H₄₈N₉O₁₀S, 774.3245; found 774.3238. ¹H NMR (600 MHz, MeOD) δ 7.58 (d, J=7.9 Hz, 1H), 7.33 (d, J=8.2 Hz, 1H), 7.15 (t, J=7.6 Hz, 1H), 7.06 (t, J=6.9 Hz, 1H), 5.21 (d, J=9.4 Hz, 1H), 4.47 (s, 2H), 4.38-4.31 (m, 1H), 3.96-3.88 (m, 3H), 3.87-3.82 (m, 2H), 3.73-3.68 (m, 1H), 3.62-3.56 (m, 2H), 3.37 (dd, J=13.4, 3.7 Hz, 1H), 2.84 (dd, J=15.7, 2.1 Hz, 1H), 2.76-2.68 (m, 1H), 2.30-2.23 (m, 1H), 2.07 (ddd, J=13.2, 8.9, 4.6 Hz, 1H), 1.81-1.76 (m, 1H), 1.73-1.65 (m, 1H), 1.44 (d, J=7.1 Hz, 3H), 1.24 (dd, J=6.7, 2.7 Hz, 1H), 0.97-0.93 (m, 6H).

### Compound V(iii): Pen⁸-Heptapeptide Monocycle

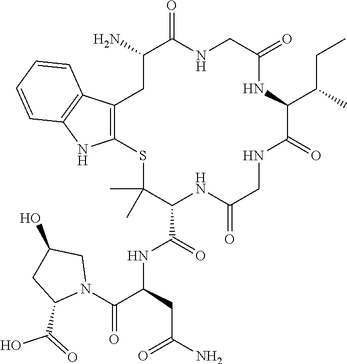 [SMILES: CC[C@H](C)[C@@H]1NC(=O)CNC(=O)[C@@H](N)Cc2c([nH]c3ccccc23)SC(C)(C)[C@@H](C(=O)N[C@@H](CC(N)=O)C(=O)N2C[C@H](O)C[C@H]2C(=O)O)NC(=O)CNC1=O]

HPLC 2 (Method D): t_{R}=22.1 min; λₘₐₓ 290 nm; HRMS-ESI (m/z) [M−H]⁻ calcd. for C₃₅H₅₀N₉O₁₀S, 788.3401; found 788.3401 ¹H NMR (600 MHz, MeOD) δ 7.67 (d, J=7.9 Hz, 1H), 7.39 (d, J=8.2 Hz, 1H), 7.21 (t, J=7.7 Hz, 1H), 7.10 (t, J=7.7 Hz, 1H), 5.01 (dd, J=7.8, 4.6 Hz, 1H), 4.69-4.60 (m, 1H), 4.52-4.47 (m, 1H), 4.43 (t, J=8.0 Hz, 1H), 4.17-4.14 (m, 1H), 4.12-4.08 (m, 1H), 3.95 (m, 2H), 3.87 (dd, J=10.8, 4.3 Hz, 1H), 3.81-3.76 (m, 1H), 3.63 (dd, J=14.0, 9.0 Hz, 1H), 3.47 (dd, J=14.0, 6.9 Hz, 1H), 2.84 (dd, J=15.5, 4.2 Hz, 1H), 2.68 (dd, J=15.5, 8.1 Hz, 2H), 2.29-2.22 (m, 1H), 2.09 (ddd, J=13.0, 7.9, 5.0 Hz, 1H), 1.89 (td, J=9.8, 3.1 Hz, 1H), 1.63-1.55 (m, 1H), 1.42 (s, 3H), 1.37-1.31 (m, 2H), 1.24 (s, 3H), 1.20-1.16 (m, 1H), 0.95 (d, J=6.7 Hz, 3H), 0.92 (t, J=7.5 Hz, 3H). ¹³C NMR Phase Edited HSQC (150 MHz, MeOD) δ 124.45 (1H), 120.79 (1H), 119.35 (1H), 112.59 (1H), 71.00 (1H), 62.29 (1H), 60.80 (1H), 60.17 (1H), 56.24 (1H), 56.15 (1H), 44.00 (2H), 38.83 (2H), 37.37 (2H), 37.24 (1H), 30.84 (1H), 28.56 (1H), 26.18 (3H), 25.81 (2H), 15.47 (3H), 11.15 (3H).

### Compound V(iv): aza-Gly⁷-Heptapeptide Monocycle

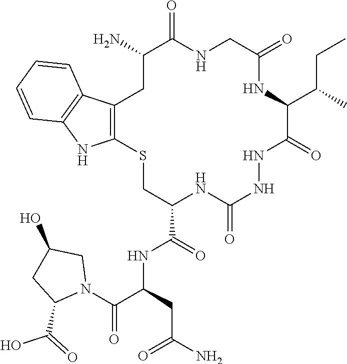 [SMILES: CC[C@H](C)[C@@H]1NC(=O)CNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)NNC1=O]

HPLC 2 (Method D): t_{R}=17.3 min; λₘₐₓ 290 nm; HRMS-ESI (m/z): [M+H]⁺ calcd. for C₃₂H₄₅N₁₀O₁₀S, 761.3041; found 761.3032. ¹H NMR (400 MHz, MeOD) δ 7.65-7.53 (m, 1H), 7.31 (d, J=8.3 Hz, 1H), 7.15 (t, J=7.6 Hz, 1H), 7.05 (m, 1H), 4.48 (m, 1H), 5.02 (m, 1H)*, 4.47-4.43 (m, 2H)*4.07 (m, 2H), 3.96-3.87 (m, 2H), 3.86-3.80 (m, 1H), 3.78-3.72 (m, 1H), 3.05-2.98 (m, 1H), 2.84-2.77 (m, 1H), 2.33-2.17 (m, 1H), 2.05 (m, 1H), 1.70-1.53 (m, 1H), 1.00-0.88 (m, 6H). *Assigned by COSY.

### Compound V(v): Thia-Ile⁶-Heptapeptide Monocycle

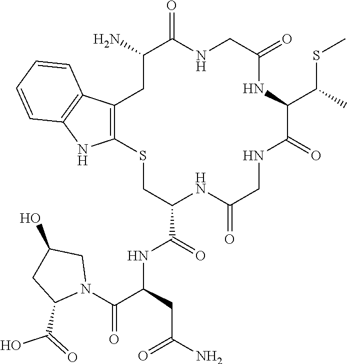 [SMILES: CS[C@H](C)[C@@H]1NC(=O)CNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=17.3 min; λₘₐₓ 290 nm; HRMS-ESI (m/z): [M+H]⁺ calcd. for C₃₂H₄₄N₉O₁₀S2, 778.2653; found 778.2650. ¹H NMR (400 MHz, MeOD) δ 7.62 (d, J=8.0 Hz, 1H), 7.34 (d, J=8.1 Hz, 1H), 7.17 (t, J=7.6 Hz, 1H), 7.06 (t, J=7.3 Hz, 1H), 4.98 (m, 1H)*, 4.48-4.27 (m, 3H), 4.26-4.09 (m, 1H), 4.07-3.99 (m, 1H), 3.93 (d, J=23.0 Hz, 1H), 3.88-3.75 (m, 1H), 3.74-3.59 (m, 1H), 3.53-3.42 (m, 2H), 3.22-3.01 (m, 2H), 2.78-2.69 (m, 1H), 2.62-2.54 (m, 1H), 2.39-2.16 (m, 1H), 2.10 (s, 3H), 2.08-1.89 (m, 1H), 1.32 (d, J=7.0 Hz, 3H). *Assigned by COSY.

### Compound V(vi): Oxo-Ile⁶-Heptapeptide Monocycle

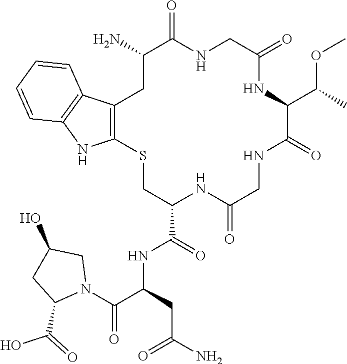 [SMILES: CO[C@H](C)[C@@H]1NC(=O)CNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}^{=14.3} min; λₘₐₓ 290 nm; HRMS-ESI (m/z): [M+H]⁺ calcd. for C₃₂H₄₄N₉O₁₁S, 762.2881; found 762.2879. ¹H NMR (400 MHz, MeOD) δ 7.67 (d, J=7.8 Hz, 1H), 7.36 (d, J=8.2 Hz, 1H), 7.19 (t, J=7.7 Hz, 1H), 7.09 (t, J=7.2 Hz, 1H), 4.47-4.33 (m, 3H), 4.23-4.07 (m, 1H), 4.03-3.95 (m, 1H), 3.96-3.90 (m, 1H), 3.56-3.43 (m, 2H), 3.34 (s, 3H), 3.14 (ddd, J=22.6, 14.0, 7.1 Hz, 1H), 2.72 (dd, J=15.0, 4.6 Hz, 1H), 2.55 (dd, J=14.5, 9.2 Hz, 1H), 2.32-2.18 (m, 1H), 2.09-2.00 (m, 1H), 1.19 (d, J=6.3 Hz, 3H).

### Compound V(vii): Diethylala⁶-Heptapeptide Monocycle

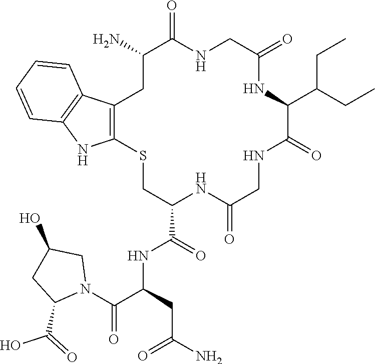 [SMILES: CCC(CC)[C@@H]1NC(=O)CNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=21.8 min; λₘₐₓ 290 nm; HRMS-ESI (m/z): [M+H]⁺ calcd. for C₃₄H₄₈N₉O₁₀S, 774.3245; found 774.3241. ¹H NMR (600 MHz, MeOD) δ 7.62 (d, J=7.7 Hz, 1H), 7.34 (d, J=8.2 Hz, 1H), 7.17 (t, J=7.5 Hz, 1H), 7.09-7.04 (m, 1H), 5.01 (dd, J=8.1, 3.4 Hz, 1H), 4.57-4.52 (m, 1H), 4.46 (s, 1H), 4.43-4.32 (m, 3H), 4.16 (t, J=7.4 Hz, 1H), 3.90 (q, J=16.1 Hz, 2H), 3.82 (dd, J=10.8, 4.1 Hz, 1H), 3.78-3.70 (m, 2H), 3.53 (dd, J=14.6, 7.4 Hz, 1H), 3.47-3.41 (m, 1H), 3.24-3.18 (m, 1H), 3.11 (dd, J=13.6, 7.5 Hz, 1H), 2.77-2.69 (m, 1H), 2.62-2.52 (m, 1H), 2.25-2.20 (m, 1H), 2.05 (ddd, J=22.3, 10.5, 7.2 Hz, 1H), 1.84-1.76 (m, 1H), 1.53 (ddd, J=13.7, 7.4, 3.9 Hz, 1H), 1.42-1.29 (m, 4H), 0.92 (t, J=7.4 Hz, 3H), 0.88 (t, J=7.4 Hz, 3H). NMR Phase Edited HSQC (150 MHz, MeOD) δ 123.79 (1H), δ 120.51 (1H), 119.15 (1H), 112.25 (1H), 70.93 (1H), 61.53 (1H), 57.75 (1H), 56.00 (2H), 55.54 (1H), 54.12 (1H), 50.21 (1), 44.08 (2H), 43.64 (2H), 42.38 (1H), 38.72 (2H), 38.68 (2H), 36.99 (2H), 22.67 (2H), 22.03 (2H), 11.07 (3H), 10.73 (3H).

### Compound V(viii): Cyclopentylgly⁶-Heptapeptide Monocycle

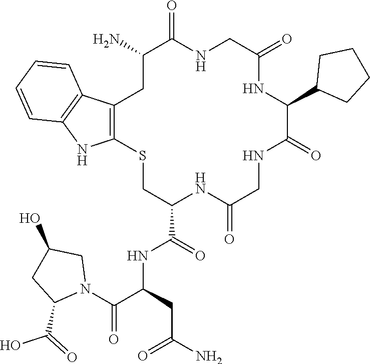 [SMILES: NC(=O)C[C@H](NC(=O)[C@@H]1CSc2[nH]c3ccccc3c2C[C@H](N)C(=O)NCC(=O)N[C@@H](C2CCCC2)C(=O)NCC(=O)N1)C(=O)N1C[C@H](O)C[C@H]1C(=O)O]

HPLC 2 (Method D): t_{R}=20.0 min; λₘₐₓ 290 nm; HRMS-ESI (m/z): [M+H]⁺ calcd. for C₃₄H₄₆N₉O₁₀S, 772.3088; found 772.3089. ¹H NMR (600 MHz, MeOD) δ 7.61 (d, J=7.9 Hz, 1H), 7.32 (d, J=8.1 Hz, 1H), 7.15 (t, J=7.6 Hz, 1H), 7.08-7.02 (m, 1H), 5.01 (m, 1H)*, 4.53-4.47 (m, 2H)*4.09 (dd, J=16.2, 6.9 Hz, 2H), 4.04-3.97 (m, 2H), 3.95-3.89 (m, 2H), 3.88-3.80 (m, 2H), 3.77-3.67 (m, 3H), 3.49 (dd, J=14.4, 8.1 Hz, 2H), 3.41 (dd, J=13.6, 4.4 Hz, 1H), 3.15 (dd, J=13.7, 7.1 Hz, 1H), 2.91-2.87 (m, 1H), 2.81-2.71 (m, 1H), 2.66-2.59 (m, 1H), 2.32-2.17 (m, 2=3H), 2.11-2.05 (m, 1H), 1.91-1.83 (m, 1H), 1.74-1.64 (m, 3H), 1.61-1.53 (m, 2H), 1.40-1.31 (m, 3H). *Assigned by COSY.

### Compound V(ix): aza-Gly⁵-Heptapeptide Monocycle

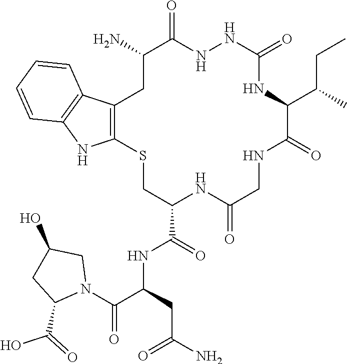 [SMILES: CC[C@H](C)[C@@H]1NC(=O)NNC(=O)[C@@H](N)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=18.2 min; λₘₐₓ 290 nm; HRMS-ESI (m/z): [M+H]⁺ calcd. for C₃₂H₄₅N₁₀O₁₀S, 761.3041; found 761.3040. Note: Due to very low sample amount, integrations and coupling constants are not reported for this molecule: ¹H NMR COSY (600 MHz, MeOD) δ 7.57, 7.40, 7.14, 7.03, 4.98, 4.47, 4.42, 4.18, 4.16, 3.98, 3.96, 3.81, 3.67, 3.54, 3.34, 3.17, 2.77, 2.21, 2.20, 2.03, 1.85, 1.58, 1.29, 0.89.

### Compound V(x): (2R,3S)-β-Methyl-Cys⁸-OH Trp⁴, Heptapeptide Monocycle

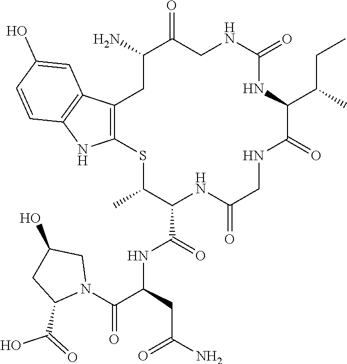 [SMILES: CC[C@H](C)[C@@H]1NC(=O)NCC(=O)[C@@H](N)Cc2c([nH]c3ccc(O)cc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=17.2 min; λₘₐₓ 288 nm; HRMS-ESI (m/z): [M+H]⁺ calcd. for C₃₄H₄₈N₉O₁₁S, 790.3194; found 790.3188. ¹H NMR (400 MHz, MeOD) δ 7.19 (d, J=8.7 Hz, 1H), 7.03 (s, 1H), 6.76 (dd, J=8.7, 2.4 Hz, 1H), 5.03-4.98 (m, 1H), 4.59 (s, 1H), 4.46 (dd, J=17.2, 7.8 Hz, 1H), 4.26 (t, J=7.7 Hz, 1H), 4.18 (d, J=17.0 Hz, 1H), 4.07 (d, J=8.0 Hz, 1H), 3.97-3.83 (m, 2H), 3.75 (d, J=14.6 Hz, 1H), 3.71-3.58 (m, 1H), 3.51 (dd, J=14.6, 8.8 Hz, 1H), 3.23-3.04 (m, 1H), 2.81 (dd, J=15.1, 4.7 Hz, 1H), 2.75-2.55 (m, 1H), 2.35-2.18 (m, 2H), 2.14-1.98 (m, 2H), 1.72-1.57 (m, 2H), 1.19 (d, J=6.9 Hz, 3H), 1.07-0.73 (m, 6H).

### Compound V(xi): 5-OH Trp⁴, Diethylala⁶-Heptapeptide Monocycle

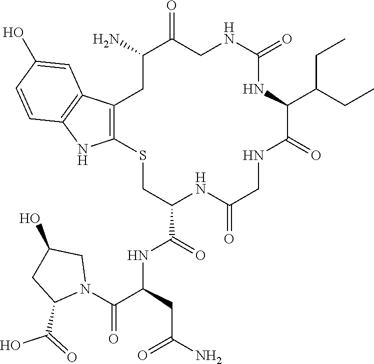 [SMILES: CCC(CC)[C@@H]1NC(=O)NCC(=O)[C@@H](N)Cc2c([nH]c3ccc(O)cc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=18.3 min; λₘₐₓ 288 nm; HRMS-ESI (m/z): [M+H]⁺ calcd. for C₃₄H₄₈N₉O₁₁S, 790.3194; found 790.3188. ¹H NMR (400 MHz, MeOD) δ 7.17 (d, J=8.7 Hz, 1H), 6.98 (d, J=2.3 Hz, 1H), 6.75 (dd, J=8.7, 2.3 Hz, 1H), 4.97 (dd, J=8.7, 4.3 Hz, 1H), 4.59 (s, 1H), 4.43 (m, 1H), 4.33 (d, J=7.4 Hz, 1H), 4.21-4.02 (m, 2H), 3.98-3.86 (m, 1H), 3.86-3.77 (m, 1H), 3.72 (d, J=16.2 Hz, 2H), 3.54-3.40 (m, 2H), 3.11 (m, 2H), 2.82-2.71 (m, 1H), 2.68-2.45 (m, 1H), 2.25 (m, 2H), 2.13-2.05 (m, 1H), 1.93-1.73 (m, 1H), 1.65-1.48 (m, 4H), 1.51-1.30 (m, 1H), 1.06-0.76 (m, 6H).

### Compound V(xii): Diethylala⁶, (2R,3S)-β-Methyl-Cys⁸-Heptapeptide Monocycle

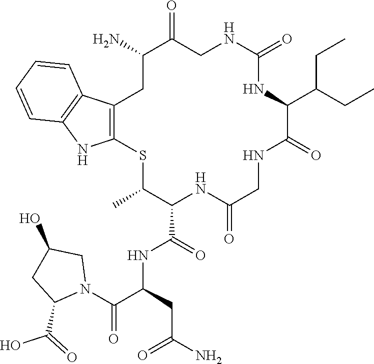 [SMILES: CCC(CC)[C@@H]1NC(=O)NCC(=O)[C@@H](N)Cc2c([nH]c3ccccc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=23.5 min; λₘₐₓ 290 nm; HRMS-ESI (m/z): [M+H]⁺ calcd. for C₃₅H₅₀N₉O₁₀S, 788.3401; found 788.3398. ¹H NMR (400 MHz, MeOD) δ 7.66 (d, J=8.0 Hz, 1H), 7.34 (d, J=8.2 Hz, 1H), 7.16 (t, J=7.5 Hz, 1H), 7.05 (t, J=7.5 Hz, 1H), 5.01 (m, 1H)*, 4.86 (m, 1H)*, 4.46 (m, 4.42-4.35 (m, 1H), 4.28-4.15 (m, 1H), 4.13-3.94 (m, 2H), 3.88-3.78 (m, 2H), 3.78-3.61 (m, 2H), 3.44 (dd, J=14.6, 6.8 Hz, 1H), 3.08 (dd, J=14.4, 8.6 Hz, 1H), 2.78 (dd, J=14.9, 4.7 Hz, 1H), 2.69-2.56 (m, 1H), 2.30-2.13 (m, 1H), 2.13-2.01 (m, 2H), 1.86-1.77 (m, 1H), 1.64-1.53 (m, 1H), 1.50-1.41 (m, 1H), 1.37-1.31 (m, 4H), 1.23 (d, J=7.1 Hz, 3H), 1.03-0.81 (m, 6H). *Assigned by COSY.

### Compound V(xiii): 5-OH Trp⁴, Diethylala⁶, (2R,3S)-β-Methyl-Cys⁸-Heptapeptide Monocycle

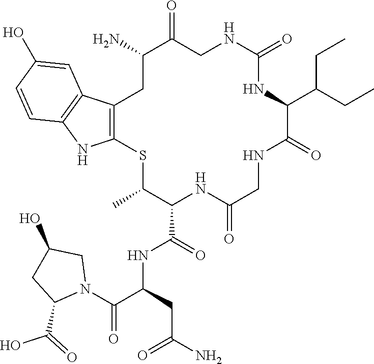 [SMILES: CCC(CC)[C@@H]1NC(=O)NCC(=O)[C@@H](N)Cc2c([nH]c3ccc(O)cc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=19.7 min; λₘₐₓ 288 nm; HRMS-ESI (m/z): [M+H]⁺ calcd. for C₃₅H₅₀N₉O₁₀S, 804.3350; found 804.3343. ¹H NMR (400 MHz, MeOD) δ 7.20 (d, J=8.8 Hz, 1H), 7.02 (d, J=2.4 Hz, 1H), 6.77 (dd, J=8.7, 2.3 Hz, 1H), 5.04-4.96 (m, 1H), 4.57 (m, 3H), 4.54-4.39 (m, 2H), 4.38-4.24 (m, 2H), 4.18 (d, J=16.9 Hz, 1H), 3.92 (s, 1H), 3.89-3.80 (m, 1H), 3.74 (d, J=16.9 Hz, 1H), 3.71-3.59 (m, 1H), 3.58-3.50 (m, 1H), 3.19 (dd, J=15.0, 7.7 Hz, 1H), 2.80 (dd, J=15.0, 4.9 Hz, 1H), 2.65 (dd, J=15.2, 7.7 Hz, 1H), 2.33-2.19 (m, 2H), 2.14-2.04 (m, 1H), 1.81-1.73 (m, 1H), 1.61-1.51 (m, 1H), 1.47-1.29 (m, 4H), 1.22 (d, J=6.9 Hz, 3H), 1.02-0.78 (m, 6H).

### General Procedure for Coupling of Fmoc-Dihydroxyisoleucine NHS Ester to Heptapeptide Monocycles

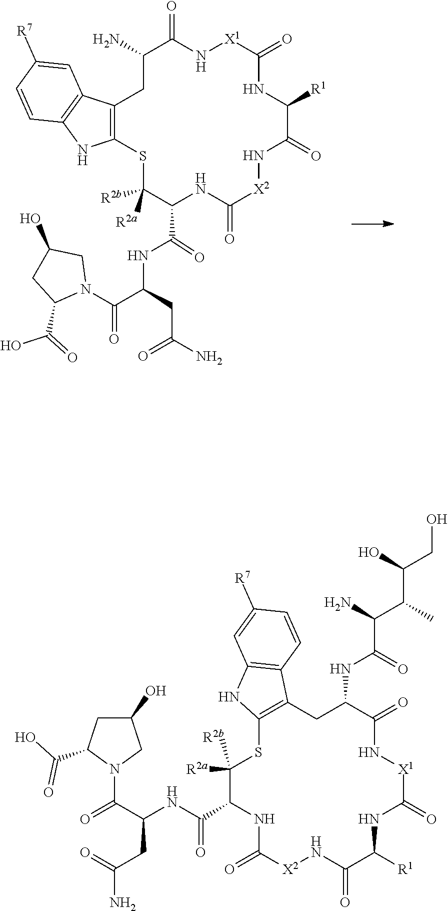

The lyophilized mono-cyclic heptapeptide, contained in a falcon tube (typically 2 μmoles), was dissolved in DMF so that the resulting solution had a concentration of 0.04M. Four equivalents of Fmoc-dihydroxyisoleucine-NHS ester (prepared as per a previous report; Matinkhoo et al., 2018) was added to the reaction mixture along with about 10 equivalents of DIEA. If the solution did not read a pH of about 9 when spotted on a pre-wetted piece of pH paper, more DIEA was added until this reading was reached. The reaction was allowed to proceed for 3 days at room temperature. Upon completion of the reaction, about 12 equivalents of diethylamine (Et₂NH) were added to the reaction mixture and it was allowed to proceed at room temperature for 2 hours. The Et₂NH was then removed by rotary evaporation and the remaining solution was diluted in 600 μL DMF. 45 μL 1M tetra-n-butylammonium fluoride TBAF (in THF) solution was added the reaction mixture and it was allowed to proceed at room temperature for 1 hour. The progress of the reaction was then evaluated by ESI-MS and if complete, the reaction mixture was diluted to 3 mL with 0.1% formic acid H₂O and 2.7 mL was purified by direct injection onto HPLC 1 with Method D with either column 1 or 2. Product peak was identified by ESI MS and the product was frozen and lyophilized to yield a fluffy white powder.

TABLE 3
Octapeptide Monocycle Yields

| Compound | Isolated Yield Over 3 Steps | Column |
|---|---|---|
| VI(i) | 28% | 2 |
| VI(ii) | 46% | 1 |
| VI(iii) | 40% | 1 |
| VI(iv) | 31% | 1 |
| VI(v) | 47% | 1 |
| VI(vi) | 27% | 1 |
| VI(vii) | 22% | 2 |
| VI(viii) | 32% | 1 |
| VI(ix) | 7% | 1 |
| VI(x) | 24% | 2 |
| VI(xi) | 29% | 2 |
| VI(xii) | 30% | 2 |
| VI(xiii) | 47% | 1 |

### Compound VI(i): (2R,3S)-β-Methyl-Cys⁸-Octapeptide Monocycle

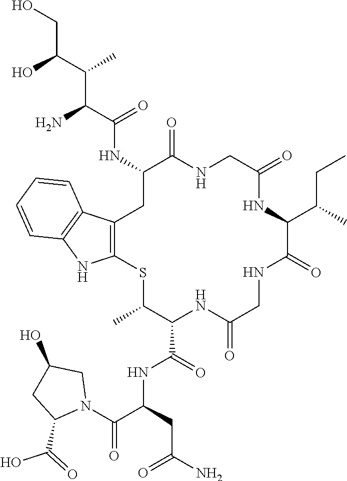 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=21.8 min; λₘₐₓ 290 nm; HRMS-ESI (m/z): [M+Na]⁺ calcd. for C₄₀H₅₈N₁₀O₁₃SNa, 941.3803; found 941.3799.

### Compound VI(ii): (2R,3R)-β-Methyl-Cys⁸-Octapeptide Monocycle

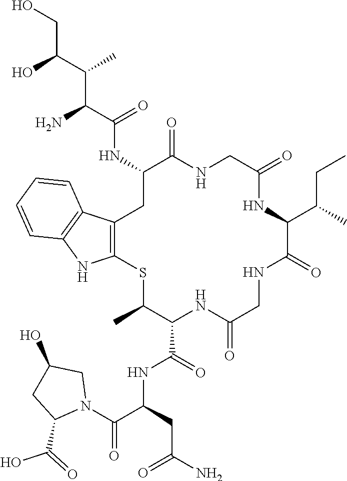 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)S[C@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=20.3 min; λₘₐₓ 290 nm; HRMS-ESI (m/z): [M+Na]⁺ calcd. for C₄₀H₅₈N₁₀O₁₃SNa, 941.3803; found 941.3798.

### Compound VI(iii): Pen⁸-Octapeptide Monocycle

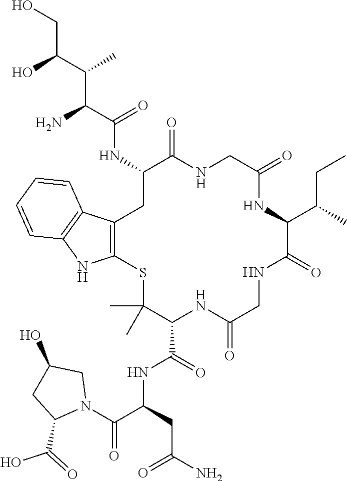 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC(C)(C)[C@@H](C(=O)N[C@@H](CC(N)=O)C(=O)N2C[C@H](O)C[C@H]2C(=O)O)NC(=O)CNC1=O]

HPLC 2 (Method D): t_{R}=22.3 min; λₘₐₓ 290 nm; HRMS-ESI (m/z) [M+H]⁺ calcd. for C₄₁H₆₁N₁₀O₁₃S, 933.4140; found 933.4135.

### Compound VI(iv): aza-Gly⁷-Octapeptide Monocycle

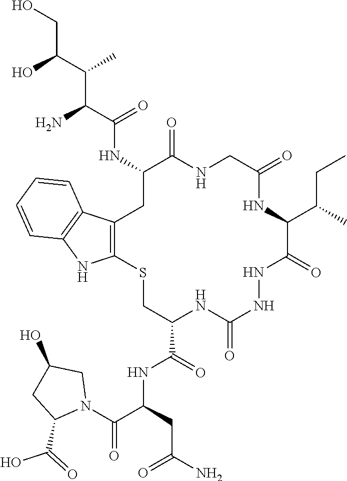 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)NNC1=O]

HPLC 2 (Method D): t_{R}=18.4 min; λₘₐₓ 290 nm; HRMS-ESI (m/z): [M+H]⁺ calcd. for C₃₈H₅₆N₁₁O₁₃S, 906.3780; found 906.3770.

### Compound VI(v): Thia-Ile⁶-Octapeptide Monocycle

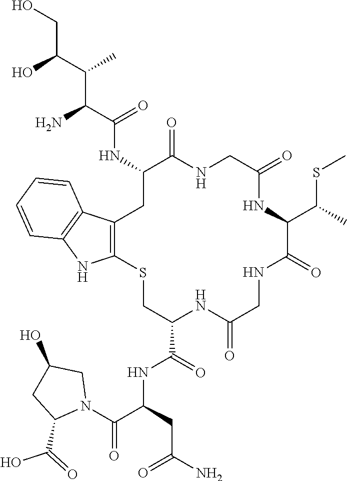 [SMILES: CS[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=18.7 min; λₘₐₓ 290 nm; HRMS-ESI (m/z): [M+Na]⁺ calcd. for C₃₈H₅₄N₁₀O₁₃S₂Na, 945.3211; found 945.3201.

### Compound VI(vi): Oxo-Ile⁶-Octapeptide Monocycle

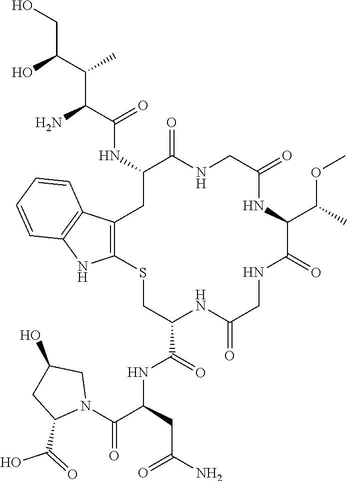 [SMILES: CO[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=15.9 min; λₘₐₓ 290 nm; HRMS-ESI (m/z): [M+H]⁺ calcd. for C₃₈H₅₅N₁₀O₁₄S, 907.3620; found 907.3612.

### Compound VI(vii): Diethylala⁶-Octapeptide Monocycle

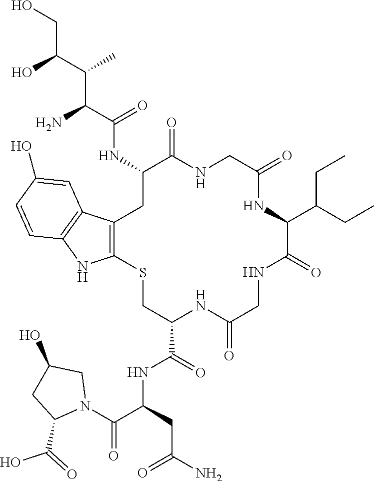 [SMILES: CCC(CC)[C@@H]1NC(=O)CNC(=O)[C@@H](NC(=O)[C@@H](N)[C@@H](C)[C@@H](O)CO)Cc2c([nH]c3ccc(O)cc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=22.8 min; λₘₐₓ 290 nm; HRMS-ESI (m/z): [M+Na]⁺ calcd. for C₄₀H₅₈N₁₀O₁₃SNa, 941.3803; found 941.3796.

### Compound VI(viii): Cyclopentylgly⁶-Octapeptide Monocycle

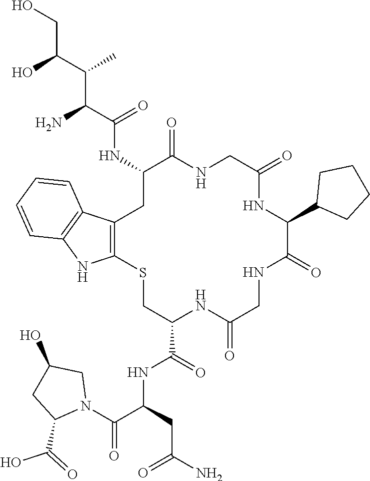 [SMILES: C[C@H]([C@H](N)C(=O)N[C@H]1Cc2c([nH]c3ccccc23)SC[C@@H](C(=O)N[C@@H](CC(N)=O)C(=O)N2C[C@H](O)C[C@H]2C(=O)O)NC(=O)CNC(=O)[C@H](C2CCCC2)NC(=O)CNC1=O)[C@@H](O)CO]

HPLC 2 (Method D): t_{R}=21.2 min; λₘₐₓ 290 nm; HRMS-ESI (m/z): [M+H]⁺ calcd. for C₄₀H₅₇N₁₀O₁₃S, 917.3827; found 917.3821.

### Compound VI(ix): aza-Gly⁵-Octapeptide Monocycle

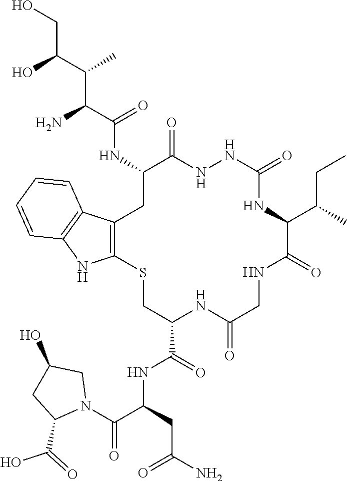 [SMILES: CC[C@H](C)[C@@H]1NC(=O)NNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=19.3 min; λₘₐₓ 290 nm; HRMS-ESI (m/z): [M−H]⁻ calcd. for C₃₈H₅₄N₁₁O₁₃S, 904.3623; found 904.3624.

### Compound VI(x): 5-OH Trp⁴, (2R,3S)-β-Methyl-Cys⁸-Octapeptide Monocycle

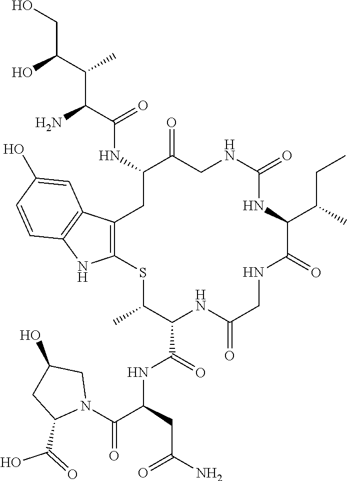 [SMILES: CC[C@H](C)[C@@H]1NC(=O)NCC(=O)[C@@H](NC(=O)[C@@H](N)[C@@H](C)[C@@H](O)CO)Cc2c([nH]c3ccc(O)cc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=18.2 min; λₘₐₓ 288 nm; HRMS-ESI (m/z): [M+H]⁺ calcd. for C₄₀H₅₉N₁₀O₁₄S, 935.3933; found 935.3931.

### Compound VI(xi): 5-OH Trp⁴, Diethylala⁶-Octapeptide Monocycle

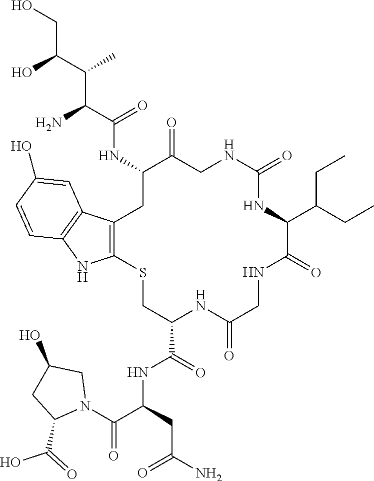 [SMILES: CCC(CC)[C@@H]1NC(=O)NCC(=O)[C@@H](NC(=O)[C@@H](N)[C@@H](C)[C@@H](O)CO)Cc2c([nH]c3ccc(O)cc23)SC[C@@H](C(=O)N[C@@H](CC(N)=O)C(=O)N2C[C@H](O)C[C@H]2C(=O)O)NC(=O)CNC1=O]

HPLC 2 (Method D): t_{R}=19.4 min; λₘₐₓ 288 nm; HRMS-ESI (m/z): [M+H]⁺ calcd. for C₄₀H₅₉N₁₀O₁₄S, 935.3933; found 935.3933.

### Compound VI(xii): Diethylala⁶, (2R,3S)-β-Methyl-Cys⁸-Octapeptide Monocycle

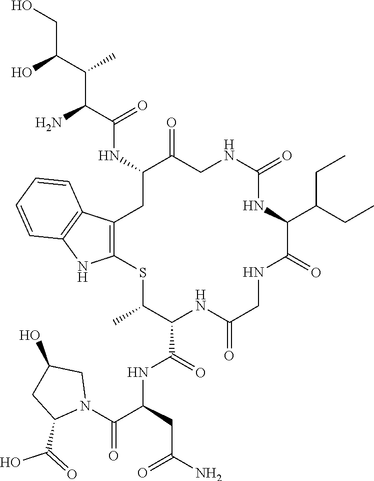 [SMILES: CCC(CC)[C@@H]1NC(=O)NCC(=O)[C@@H](NC(=O)[C@@H](N)[C@@H](C)[C@@H](O)CO)Cc2c([nH]c3ccccc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=24.1 min; λₘₐₓ 290 nm; HRMS-ESI (m/z): [M+H]⁺ calcd. for C₄₁H₆₁N₁₀O₁₃S, 933.4140; found 933.4133.

### Compound VI(xiii): 5-OH Trp⁴, Diethylala⁶, (2R,3S)-β-Methyl-Cys⁸-Octapeptide Monocycle

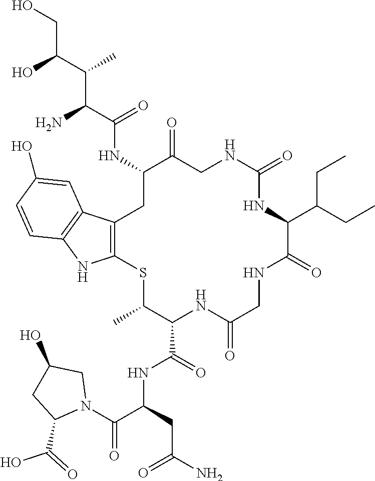 [SMILES: CCC(CC)[C@@H]1NC(=O)NCC(=O)[C@@H](NC(=O)[C@@H](N)[C@@H](C)[C@@H](O)CO)Cc2c([nH]c3ccc(O)cc23)S[C@@H](C)[C@@H](C(=O)N[C@@H](CC(N)=O)C(=O)N2C[C@H](O)C[C@H]2C(=O)O)NC(=O)CNC1=O]

HPLC 2 (Method D): t_{R}=20.5 min min; λₘₐₓ 288 nm; HRMS-ESI (m/z): [M+H]⁺ calcd. for C₄₁H₆₁N₁₀O₁₄S, 949.4089; found 949.4083.

### General Procedure for Macrolactamization

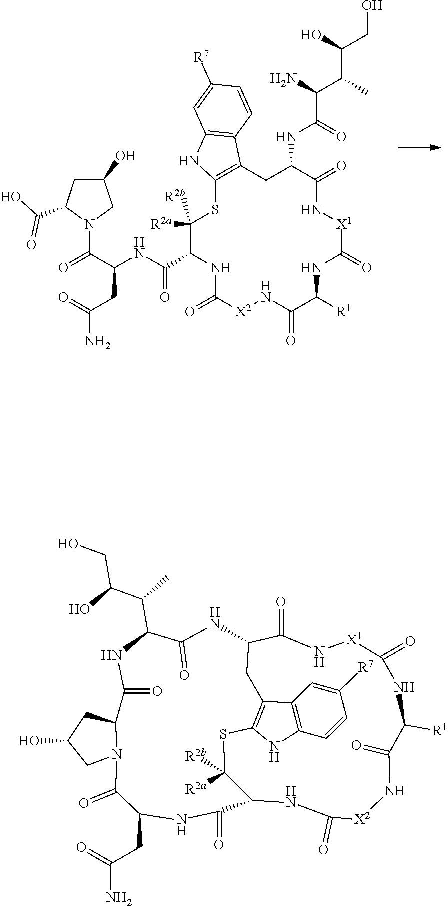

The lyophilized monocyclic octapeptide was dissolved in dry dimethylacetamide (DMA, solution concentration about 0.004M of octapeptide monocycle) and 9 equivalents of 1-[bis(dimethylamino)methylene]-1H-1,2,3-triazolo[4,5-b]pyridinium 3-oxide hexafluorophosphate (HATU) and about 10 equivalents of DIEA were added to the reaction mixture and it was vortexed and allowed to proceed at room temperature for 1-2 hours. The reaction mixture was then diluted with 0.10% formic acid H₂O and directly injected onto HPLC 1 for purification with Method E with either column 1 or 2. Product peak was identified by ESI MS and the product was frozen and lyophilized to yield a fluffy white powder.

TABLE 4
Octapeptide Bicycle Yields

| Compound | Isolated Yield Over 3 Steps | Column |
|---|---|---|
| I(i) | 30% | 2 |
| I(ii) | 25% | 1 |
| I(iii) | 34% | 1 |
| I(iv) | 41% | 1 |
| I(v) | 51% | 1 |
| I(vi) | 50% | 1 |
| I(vii) | 20% | 2 |
| I(viii) | 50% | 1 |
| I(ix) | 62% | 1 |
| I(x) | 41% | 2 |
| I(xi) | 36% | 2 |
| I(xii) | 42% | 2 |
| I(xiii) | 22% | 1 |

### Compound I(i): (2R,3S)-β-Methyl-Cys⁸-Dideoxyamanitin

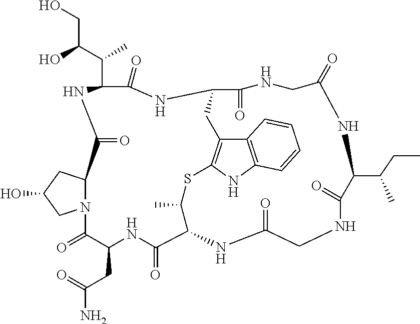 [SMILES: CC[C@H](C)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=17.4 min; λₘₐₓ 290 nm; HRMS-ESI (m/z): [M+Na]⁺ calcd. for C₄₀H₅₆N₁₀O₁₂SNa, 923.3698; found 923.3701. ¹H NMR (600 MHz, DMSO) δ 11.24-11.23 (m, 1H), 9.07-8.98 (m, 1H), 8.74 (d, J=4.3 Hz, 1H), 8.42 (s, 1H), 8.20 (s, 1H), 8.04 (d, J=10.5 Hz, 1H), 8.01-7.94 (m, 2H), 7.77 (d, J=8.2 Hz, 1H), 7.58 (d, J=7.9 Hz, 1H), 7.44 (s, 1H), 7.28 (d, J=8.2 Hz, 1H), 7.12 (t, J=7.6 Hz, 1H), 7.02 (t, J=7.6 Hz, 1H), 5.12-5.04 (m, 1H), 4.93-4.90 (m, 1H), 4.67 (d, J=3.4 Hz, 1H), 4.58-4.54 (m, 1H), 4.51 (dd, J=9.8, 5.4 Hz, 1H), 4.46 (m, 1H), 4.39 (s, 1H), 4.28-4.23 (m, 2H), 4.00 (dd, J=18.8, 8.1 Hz, 1H), 3.90 (dd, J=17.4, 7.5 Hz, 1H), 3.78 (s, 2H), 3.74-3.70 (m, 1H), 3.58-3.50 (m, 4H), 3.48 (m, 1H), 3.32 (m, 2H)*, 3.16 (m, 1H)*3.06-2.97 (m, 2H), 2.24-2.17 (m, 2H), 2.01 (t, J=7.3 Hz, 2H)**, 1.92-1.81 (m, 1H)**, 1.58-1.55 (m, 2H)**, 1.47-1.37 (m, 1H)**, 0.92 (d, J=6.3 3H), 0.87 (d, J=7.0 Hz, 3H), 0.82 (t, J=7.3 Hz, 3H), 0.78 (d, J=6.7 Hz, 3H). ¹³C NMR HSQC Phase Edited (150 MHz, DMSO) δ 121.82 (1H), 119.97 (1H), 118.32 (1H), 110.96 (1H), 72.23 (1H), 68.43 (1H), 62.83 (2H), 61.96 (1H), 59.11 (1H), 57.96 (1H), 55.52 (2H), 54.70 (1H), 52.85 (1H), 50.58 (1H), 48.41 (1H), 42.07 (2H), 41.42 (2H), 38.09 (1H), 37.44 (2H), 34.04 (1H), 34.00 (2H), 33.50 (2H), 24.76 (2H), 17.75 (3H), 14.95 (3H), 13.45 (3H), 10.18 (3H). *Assigned by HSQC. **Integrations determined by HSQC.

### Compound I(ii): (2R,3R)-β-Methyl-Cys⁸-Dideoxyamanitin

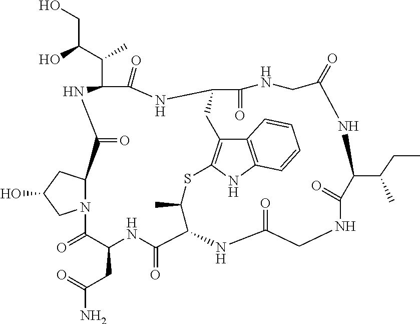 [SMILES: CC[C@H](C)[C@@H]1NC(=O)CNC(=O)[C@H]2Cc3c([nH]c4ccccc34)S[C@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method C): t_{R}=16.8 min; λₘₐₓ 290 nm; HRMS-ESI (m/z): [M+Na]⁺ calcd. for C₄₀H₅₆N₁₀O₁₂SNa, 923.3698; found 923.3701. NMR chemical shifts included numerous instances of doubling of resonances, especially around Tryptophan and Cysteine associated peaks, while not wishing to be limited by theory, suggesting multiple conformational populations.

### Compound I(iii): Pen⁸-Dideoxyamanitin

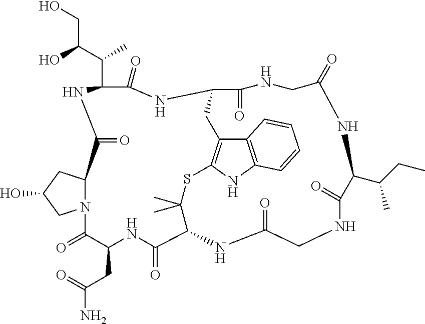 [SMILES: CC[C@H](C)[C@@H]1NC(=O)CNC(=O)[C@H]2Cc3c([nH]c4ccccc34)SC(C)(C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=18.5 min; λₘₐₓ 290 nm HRMS-ESI (m/z) [M−H]⁻ calcd. for C₄₁H₅₇N₁₀O₁₂S, 913.3878; found 913.3887. ¹H NMR (600 MHz, DMSO) δ 11.02 (s, 1H), 9.05-8.97 (m, 1H), 8.62 (d, J=4.3 Hz, 1H), 8.57 (d, J=5.2 Hz, 1H), 8.29 (d, J=4.4 Hz, 1H), 8.03-7.93 (m, 2H), 7.69 (d, J=9.0 Hz, 1H), 7.44 (d, J=8.0 Hz, 1H), 7.39 (d, J=5.5 Hz, 1H), 7.32 (d, J=8.2 Hz, 1H), 7.15 (t, J=7.9 Hz, 1H), 7.04 (t, J=7.6 Hz, 1H), 4.82 (d, J=10.6 Hz, 1H), 4.78 (q, J=4.2 Hz, 1H), 4.54-4.49 (m, 1H), 4.47 (dd, J=9.4, 4.2 Hz, 1H), 4.40 (s, 1H), 4.28 (dd, J=11.4, 6.9 Hz, 1H), 4.16 (dd, J=17.5, 7.7 Hz, 1H), 3.85-3.79 (m, J=18.1, 5.7 Hz, 2H), 3.78-3.72 (m, 2H), 3.52 (m, 2H)*, 3.36 (m, 2H)*3.11-3.06 (m, 1H), 3.02 (dd, J=13.5, 5.8 Hz, 1H), 2.87 (dd, J=15.2, 4.2 Hz, 1H), 2.26-2.19 (m, 2H), 1.90 (td, J=12.5, 3.2 Hz, 1H), 1.67-1.62 (m, 1H), 1.60-1.55 (m, 1H), 1.48 (s, 3H), 1.11 (s, 3H), 0.90 (d, J=7.0 Hz, 3H), 0.83 (t, J=7.4 Hz, 3H), 0.78 (d, J=6.7 Hz, 3H). Phase Edited (150 MHz, DMSO) δ 122.22 (1H), 118.66 (1H), 118.16 (1H), 110.95 (1H), 72.16 (1H), 68.22 (1H), 63.90 (1H), 63.56 (2H), 61.58 (1H), 58.85 (1H), 55.96 (2H), 54.53 (1H), 54.47 (1H), 50.75 (1H), 41.81 (2H), 41.79 (2H), 37.64 (2H), 34.94 (2H), 33.80 (1H), 32.32 (3H), 31.07 (3H), 29.21 (2H), 14.94 (3H), 13.56 (3H), 9.70 (3H). *Assigned by HSQC.

### Compound I(iv): aza-Gly⁷-Dideoxyamanitin

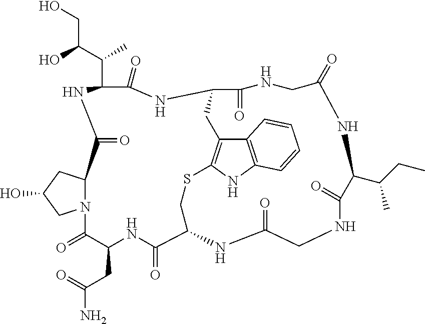 [SMILES: CC[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}^{=15.9} min; λₘₐₓ 290 nm; HRMS-ESI (m/z): C₃₈H₅₄N₁₁O₁₂SNa (M+H) calc. 888.3674 obs. 888.3672. ¹H NMR (600 MHz, DMSO) δ 11.26 (s, 1H), 10.02 (s, 1H), 8.51 (d, J=4.1 Hz, 1H), 8.43 (s, 3H), 8.33 (d, J=3.3 Hz, 1H), 8.28 (s, 1H), 8.15 (s, 1H), 8.11 (d, J=9.0 Hz, 1H), 7.96 (d, J=9.5 Hz, 1H), 7.87 (d, J=8.0 Hz, 1H), 7.57 (d, J=8.0 Hz, 1H), 7.44 (s, 1H), 7.24 (d, J=8.2 Hz, 1H), 7.10 (t, J=7.8 Hz, 1H), 7.00 (t, J=7.6 Hz, 1H), 6.95 (d, J=10.2 Hz, 1H), 4.97-4.91 (m, 1H), 4.71 (dd, J=7.3, 3.6 Hz, 1H), 4.44 (dd, J=9.4, 5.8 Hz, 1H), 4.42-4.37 (m, 2H), 4.29 (dd, J=11.5, 7.1 Hz, 1H), 4.22 (dd, J=18.6, 8.6 Hz, 1H), 3.78 (dd, J=28.1, 9.9 Hz, 2H), 3.63 (dd, J=8.6, 4.3 Hz, 1H), 3.53-3.48 (m, 1H), 3.37 (m, 1H)*, 3.33 (m, 1H)*, 3.02 (m, 2H), 2.98 (dd, J=13.0, 6.4 Hz, 1H), 2.77-2.74 (m, 1H), 2.19 (m, 2H), 1.87 (td, J=12.6, 3.4 Hz, 1H), 1.59-1.51 (m, 2H), 0.88 (d, J=7.1 Hz, 3H), 0.83-0.77 (m, 6H); ¹³C NMR HSQC Phase Edited (150 MHz, DMSO) δ 122.02 (1H), 120.14 (1H), 118.37 (1H), 110.94 (1H), 72.20 (1H), 68.44 (1H), 63.31 (2H), 61.62 (1H), 57.15 (1H), 55.62 (2H), 54.90 (1H), 53.21 (1H), 53.20 (1H), 50.30 (1H), 41.08 (2H), 37.85 (1H), 37.56 (2H), 34.24 (1H), 34.05 (2H), 29.99 (2H), 21.46 (1H), 14.90 (3H), 13.45 (3H), 10.24 (3H). *Assigned by HSQC.

### Compound I(v): Thia-Ile⁶-Dideoxyamanitin

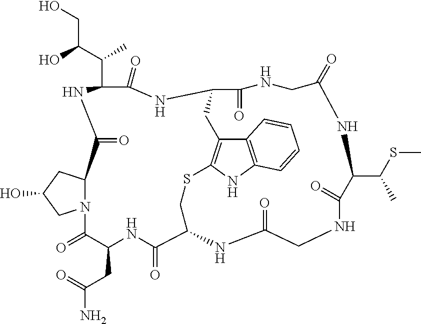 [SMILES: CS[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method C): t_{R}=15.7 min; λₘₐₓ 290 nm; HRMS-ESI (m/z): [M+Na]⁺ calcd. for C₃₈H₅₂N₁₀O₁₂S₂Na, 927.3105; found 927.3108. ¹H NMR (600 MHz, DMSO) δ 11.23 (s, 1H), 9.00-8.97 (m, 1H), 8.61 (d, J=3.9 Hz, 1H), 8.41 (s, 1H), 8.12 (s, 1H), 8.08 (d, J=7.8 Hz, 1H), 7.98 (d, J=9.8 Hz, 1H), 7.97-7.93 (m, 1H), 7.91 (d, J=8.2 Hz, 1H), 7.56 (d, J=8.0 Hz, 1H), 7.45 (s, 1H), 7.25 (d, J=8.1 Hz, 1H), 7.11 (t, J=7.5 Hz, 1H), 7.01 (t, J=7.6 Hz, 1H), 4.96-4.88 (m, 1H), 4.70 (d, J=4.1 Hz, 1H), 4.60-4.54 (m, 1H), 4.45-4.36 (m, 2H), 4.28 (dd, J=11.4, 7.0 Hz, 1H), 4.28 (dd, J=11.4, 7.0 Hz, 1H), 4.20 (dd, J=18.9, 8.5 Hz, 1H), 3.92 (m, 1H), 3.75 (m, 2H)*, 3.50 (m, 1H)*, 3.43-3.30 (m, 4H)*, 3.30 (m, 2H)*, 3.09 (t, J=11.0 Hz, 1H), 3.04-2.96 (m, 1H), 2.90 (d, J=10.9 Hz, 1H), 2.29-2.13 (m, 2H), 2.00 (s, 3H), 1.88 (t, J=10.5 Hz, 1H), 1.15 (d, J=6.7 Hz, 3H), 0.88 (d, J=6.7 Hz, 3H). ¹³C NMR Phase Edited HSQC (150 MHz, DMSO) δ 122.37 (1H), 120.21 (1H), 118.77 (1H), 111.03 (1H), 72.51 (1H), 68.58 (1H), 63.40 (2H), 61.92 (1H), 57.79 (1H), 55.84 (2H), 55.10 (1H), 53.29 (1H), 52.32 (1H), 50.51 (1H), 42.26 (2H), 41.35 (2H), 39.65 (1H), 38.06 (2H), 37.41 (1H), 37.15 (2H), 33.88 (2H), 29.80 (2H), 16.97 (3H), 13.65 (3H), 11.41 (3H). ¹³C NMR HMBC (150 MHz, DMSO) δ 136.69 (0H), 127.11 (0H), 124.77 (0H), 116.16 (0H). *Assigned by HSQC.

### Compound I(vi): Oxo-Ile⁶-Dideoxyamanitin

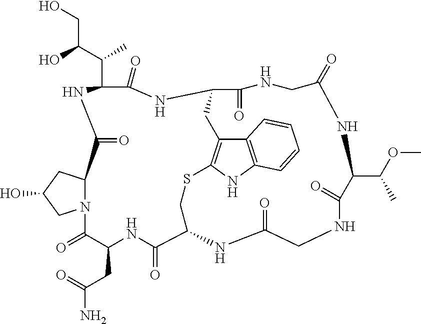 [SMILES: CO[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method C): t_{R}=14.7 min; λₘₐₓ 290 nm; HRMS-ESI (m/z): [M+Na]⁺ calcd. for C₃₈H₅₂N₁₀O₁₃SNa, 911.3334; found 911.3332. ¹H NMR (600 MHz, DMSO) δ 11.25 (s, 1H), 9.02-8.95 (m, 1H), 8.54 (d, J=3.1 Hz, 1H), 8.46-8.41 (m, 5H), 8.18 (s, 1H), 8.13-8.03 (m, 1H), 7.96 (d, J=9.4 Hz, 1H), 7.92 (s, 1H), 7.58 (d, J=8.2 Hz, 1H), 7.46 (s, 1H), 7.25 (d, J=8.2 Hz, 1H), 7.11 (t, J=7.6 Hz, 1H), 7.00 (t, J=7.5 Hz, 1H), 5.00-4.91 (m, 1H), 4.70 (d, J=3.8 Hz, 1H), 4.59-4.52 (m, 1H), 4.42 (dd, J=9.4, 6.1 Hz, 1H), 4.40-4.36 (m, 1H), 4.32-4.22 (m, 2H), 3.89-3.84 (m, 2H), 3.77 (dd, J=29.7, 9.6 Hz, 2H), 3.50 (dd, J=10.5, 6.3 Hz, 1H), 3.36 (m, 1H)*, 3.31 (m, 2H)*, 3.22 (s, 3H), 3.12-3.00 (m, 2H), 2.93 (dd, J=15.9, 4.7 Hz, 1H), 2.78 (dd, J=10.9, 3.3 Hz, 1H), 2.24-2.14 (m, 2H), 1.95-1.83 (m, 1H), 1.01 (d, J=6.2 Hz, 3H), 0.88 (d, J=7.1 Hz, 3H); ¹³C NMR HSQC Phase Edited (150 MHz, DMSO) δ 121.97 (1H), 120.30 (1H), 118.37 (1H), 110.94 (1H), 74.35 (1H), 72.03 (1H), 68.45 (1H), 63.21 (2H), 61.69 (1H), 59.24 (1H), 55.51 (2H), 55.29 (3H), 54.83 (1H), 53.09 (1H), 52.29 (1H), 50.43 (1H), 42.32 (2H), 41.03 (2H), 38.05 (2H), 37.76 (1H), 37.15 (2H), 33.78 (2H), 29.61 (2H), 14.70 (3H), 13.31 (3H). *Assigned by HSQC.

### Compound I(vii): Diethylala⁶-Dideoxyamanitin

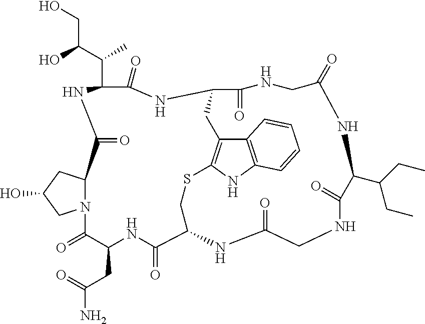 [SMILES: CCC(CC)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=18.9 min; λₘₐₓ 290 nm; HRMS-ESI (m/z) (M+H) calc 901.3878 obs. 901.3875. ¹H NMR (600 MHz, DMSO) δ 11.25 (s, 1H), 8.95-8.88 (m, 1H), 8.46 (d, J=4.4 Hz, 1H), 8.44 (d, J=3.4 Hz, 1H), 8.14 (s, 1H), 8.09 (d, J=8.4 Hz, 1H), 7.99-7.93 (m, 2H), 7.89 (d, J=8.3 Hz, 1H), 7.56 (d, J=8.0 Hz, 1H), 7.44 (s, 1H), 7.25 (d, J=8.2 Hz, 1H), 7.11 (t, J=7.7 Hz, 1H), 7.01 (t, J=7.5 Hz, 1H), 4.96-4.90 (m, 1H), 4.71 (dd, J=7.4, 3.8 Hz, 1H), 4.62-4.55 (m, 1H), 4.44 (dd, J=9.3, 5.6 Hz, 1H), 4.40 (s, 1H), 4.27 (dd, J=11.5, 7.0 Hz, 1H), 4.19-4.11 (m, 1H), 3.91 (dd, J=17.3, 7.7 Hz, 1H), 3.85-3.78 (m, 2H), 3.74 (d, J=11.0 Hz, 1H), 3.50 (m, 1H)*, 3.43-3.39 (m, 2H)*, 3.33 (m, 2H)*, 3.30 (m, 1H)*, 3.09 (t, J=11.3 Hz, 1H), 3.01 (dd, J=14.6, 6.2 Hz, 1H), 2.92 (dd, J=15.8, 4.8 Hz, 1H), 2.80 (dd, J=11.0, 3.1 Hz, 1H), 2.23-2.16 (m, 2H), 1.87 (td, J=12.5, 3.1 Hz, 1H), 1.38-1.32 (m, 2H), 1.29-1.24 (m, 3H), 0.88 (d, J=7.0 Hz, 3H), 0.80-0.74 (m, J=7.1 Hz, 6H). ¹³C NMR HSQC Phase Edited (150 MHz, DMSO) δ 121.90 (1H), 119.89 (1H), 118.41 (1H), 110.83 (1H), 72.18 (1H), 68.41 (1H), 63.33 (2H), 61.59 (1H), 56.43 (1H), 55.68 (2H), 54.83 (1H), 53.18 (1H), 52.04 (1H), 50.29 (1H), 42.18 (2H), 41.32 (2H), 39.87 (1H), 38.14 (2H), 37.79 (1H), 37.26 (2H), 33.92 (2H), 29.60 (2H), 22.02 (2H), 20.53 (2H), 13.53 (3H), 10.63 (3H), 9.34 (3H). *Assigned by HSQC.

### Compound I(viii): Cyclopentylgly⁶-Dideoxyamanitin

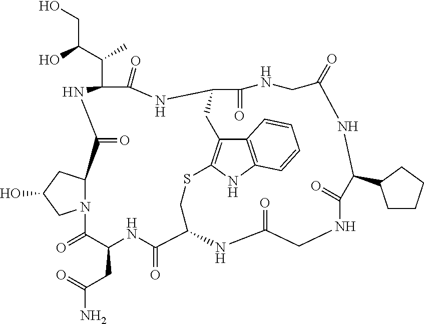 [SMILES: C[C@H]([C@@H]1NC(=O)[C@@H]2C[C@@H](O)CN2C(=O)[C@H](CC(N)=O)NC(=O)[C@@H]2CSc3[nH]c4ccccc4c3C[C@@H](NC1=O)C(=O)NCC(=O)N[C@@H](C1CCCC1)C(=O)NCC(=O)N2)[C@@H](O)CO]

HPLC 2 (Method E): t_{R}=18.3 min; λₘₐₓ 290 nm HRMS-ESI (m/z) [M−H]⁻ calcd. for C₄₀H₅₃N₁₀O₁₁S, 897.3565; found 897.3574 ¹H NMR (600 MHz, DMSO) δ 11.25 (s, 1H), 8.92-8.87 (m, 1H), 8.53 (d, J=3.9 Hz, 1H), 8.50-8.42 (m, 4H), 8.16 (s, 1H), 8.09 (d, J=9.0 Hz, 1H), 8.01 (s, 1H), 7.96 (d, J=9.3 Hz, 1H), 7.91 (d, J=8.1 Hz, 1H), 7.57 (d, J=8.0 Hz, 1H), 7.45 (s, 1H), 7.25 (d, J=8.2 Hz, 1H), 7.11 (t, J=7.5 Hz, 1H), 7.01 (t, J=7.6 Hz, 1H), 4.93 (dt, J=22.3, 8.2 Hz, 1H), 4.70 (dd, J=7.7, 4.0 Hz, 1H), 4.60-4.54 (m, 1H), 4.43 (dd, J=9.4, 5.9 Hz, 1H), 4.41-4.35 (m, 1H), 4.27 (dd, J=11.5, 7.0 Hz, 1H), 4.17 (dd, J=18.5, 8.4 Hz, 1H), 3.91 (dd, J=17.3, 7.6 Hz, 1H), 3.77 (dd, J=33.9, 9.8 Hz, 2H), 3.65 (dd, J=10.0, 4.1 Hz, 1H), 3.50 (dd, J=10.3, 6.3 Hz, 1H), 3.08 (t, J=11.5 Hz, 1H), 3.04-3.00 (m, 1H), 2.79 (dd, J=10.7, 3.4 Hz, 1H), 2.22-2.15 (m, 2H), 1.93 (dd, J=17.1, 7.7 Hz, 1H), 1.90-1.81 (m, 2H), 1.57-1.54 (m, 2H), 1.49-1.41 (m, 2H), 1.31-1.15 (m, 4H), 0.88 (d, J=7.0 Hz, 3H). ¹³C NMR HSQC Phase Edited (150 MHz, DMSO) δ 121.89 (1H), 119.72 (1H), 118.30 (1H), 110.82 (1H), 72.10 (1H), 68.38 (1H), 63.25 (2H), 61.69 (1H), 58.88 (1H), 55.49 (2H), 54.81 (1H), 53.03 (1H), 52.11 (1H), 50.38 (1H), 41.88 (2H), 39.80 (1H), 37.84 (2H), 37.3 (1H), 37.31 (2H), 33.78 (2H), 29.52 (2H), 28.46 (2H), 24.09 (2H), 13.43 (3H).

### Compound I(ix): aza-Gly⁵-Dideoxyamanitin

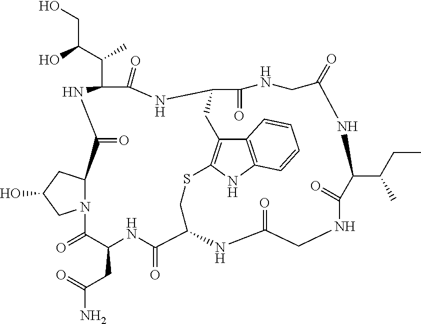 [SMILES: CC[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method C): t_{R}=17.4 min; λₘₐₓ 290 nm; HRMS-ESI (m/z): [M+Na]⁺ calcd. for C₃₈H₅₃N₁₁O₁₂SNa, 910.3494; found 910.3492. ¹H NMR (600 MHz, DMSO) δ 11.27 (s, 1H), 10.00 (s, 1H), 8.82-8.75 (m, 1H), 8.58 (s, 1H), 8.40 (d, J=10.2 Hz, 1H), 8.30 (s, 1H), 8.02 (d, J=7.2 Hz, 1H), 7.95 (d, J=9.8 Hz, 1H), 7.59 (d, J=8.1 Hz, 2H), 7.56 (s, 1H), 7.25 (d, J=8.1 Hz, 2H), 6.86 (s, 1H), 5.01-4.94 (m, 1H), 4.66 (d, J=2.9 Hz, 1H), 4.54-4.49 (m, 1H), 4.44 (dd, J=9.5, 7.5 Hz, 1H), 4.39-4.36 (m, 1H), 4.27 (dd, J=11.9, 6.7 Hz, 1H), 3.87-3.74 (m, 3H), 3.55 (m, 3H), 3.31 (m, 1H)*, 3.09 (dd, J=15.0, 6.5 Hz, 1H), 3.05-2.99 (m, 1H), 2.95 (dd, J=16.1, 3.7 Hz, 1H), 2.72 (dd, J=10.8, 3.7 Hz, 1H), 2.20 (dd, J=12.6, 6.6 Hz, 1H), 2.14 (dd, J=13.4, 6.9 Hz, 1H), 1.52-1.47 (m, 1H), 1.23 (m, 2H)* 0.87 (d, J=7.0 Hz, 3H), 0.80 (t, J=7.2 Hz, 3H), 0.76 (d, J=6.6 Hz, 3H). ¹³C NMR HSQC Phase Edited (150 MHz, DMSO) δ 122.02 (1H), 120.21 (1H), 118.38 (1H), 110.92 (1H), 71.95 (1H), 68.36 (1H), 62.98 (2H), 61.84 (1H), 58.37 (1H), 55.66 (2H), 55.00 (1H), 52.66 (1H), 52.06 (1H), 50.64 (1H), 42.30 (2H), 38.54 (2H), 37.97 (1H), 37.22 (1H), 34.74 (1H), 33.38 (2H), 29.40 (2H), 28.61 (2H), 14.50 (3H), 13.17 (3H), 10.09 (3H). *Assigned by HSQC.

### Compound I(x): 5-OH Trp⁴, (2R,3S)-β-Methyl-Cys⁸-Deoxyamanitin

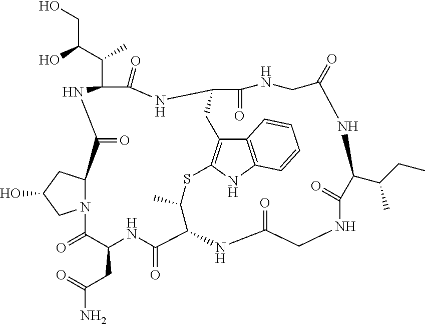 [SMILES: CC[C@H](C)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=14.6 min; λₘₐₓ 288 nm; HRMS-ESI (m/z): C₄₀H₅₇N₁₀O₁₃S (M+H) calc 917.3827 obs. 917.3819. ¹H NMR (600 MHz, DMSO) δ 10.91 (s, 1H), 9.06-8.99 (m, 1H), 8.86 (s, 1H), 8.75 (d, J=4.1 Hz, 1H), 8.41 (s, 1H), 8.05-7.93 (m, 3H), 7.74 (d, J=8.2 Hz, 1H), 7.43 (s, 1H), 7.07 (d, J=8.7 Hz, 1H), 6.95 (s, 1H), 6.64 (dd, J=8.7, 2.2 Hz, 1H), 5.03 (dd, J=16.3, 9.1 Hz, 1H), 4.91 (d, J=2.4 Hz, 1H), 4.66 (d, J=2.8 Hz, 1H), 4.51 (m, 2H), 4.39 (s, 1H), 4.29-4.21 (m, 2H), 4.01 (dd, J=18.8, 7.9 Hz, 1H), 3.90 (dd, J=17.3, 7.3 Hz, 1H), 3.77 (s, 1H), 3.73 (dd, J=9.0, 4.5 Hz, 2H), 3.55 (d, J=18.3 Hz, 2H), 3.50-3.41 (m, 3H)*, 3.32 (m, 2H)*, 3.15 (m, 1H)*, 3.07 (t, J=13.9 Hz, 1H), 2.99 (dd, J=16.1, 4.0 Hz, 1H), 2.96-2.91 (m, 2H), 2.23-2.17 (m, 2H), 1.87 (td, J=12.5, 3.2 Hz, 1H), 1.63-1.54 (m, 4H), 0.90 (d, J=6.3 Hz, 3H), 0.86 (d, J=7.0 Hz, 3H), 0.82 (t, J=7.3 Hz, 3H), 0.78 (d, J=6.7 Hz, 3H). HSQC Phase Edited (150 MHz, DMSO) δ 112.82 (1H), 111.46 (1H), 103.64 (1H), 72.23 (1H), 68.26 (1H), 62.92 (2H), 61.94 (1H), 59.15 (1H), 58.06 (1H), 55.61 (2H), 54.71 (1H), 50.52 (1H), 46.70 (1H), 42.31 (2H), 41.50 (2H), 38.11 (1H), 37.38 (2H), 33.95 (1H), 33.64 (2H), 30.83 (2H), 22.11 (2H), 17.75 (3H), 14.78 (3H), 13.46 (3H), 10.22 (3H). *Assigned by HSQC.

### Compound I(xi): 5-OH Trp⁴, Diethylala⁶-Deoxyamanitin

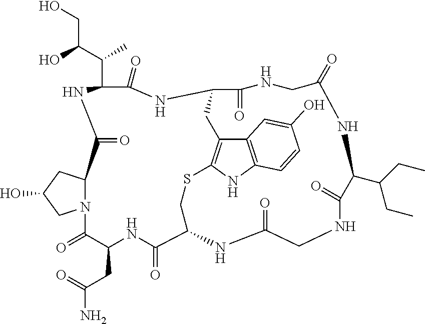 [SMILES: CCC(CC)[C@@H]1NC(=O)CNC(=O)[C@H]2Cc3c([nH]c4ccc(O)cc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=16.6 min; λₘₐₓ 288 nm; HRMS-ESI (m/z) C₄₀H₅₇N₁₀O₁₃S (M+H) calc 917.3827 obs. 917.3820. ¹H NMR (600 MHz, DMSO) δ 10.94-10.92 (m, 1H), 8.93 (s, 1H), 8.84 (s, 1H), 8.47 (d, J=4.8 Hz, 1H), 8.43 (d, J=3.3 Hz, 1H), 8.14 (s, 1H), 8.09 (d, J=8.2 Hz, 1H), 7.97 (d, J=9.2 Hz, 1H), 7.94 (d, J=9.9 Hz, 1H), 7.87 (d, J=8.3 Hz, 1H), 7.43 (s, 1H), 7.04 (d, J=8.7 Hz, 1H), 6.92 (d, J=2.0 Hz, 1H), 6.62 (dd, J=8.7, 2.2 Hz, 1H), 4.93-4.87 (m, 1H), 4.73-4.69 (m, 1H), 4.59-4.54 (m, 1H), 4.43 (dd, J=9.3, 5.5 Hz, 1H), 4.39 (s, 1H), 4.27 (dd, J=11.4, 7.0 Hz, 1H), 4.16 (dd, J=18.6, 8.1 Hz, 1H), 3.91 (dd, J=17.3, 7.6 Hz, 1H), 3.84 (dd, J=8.0, 4.9 Hz, 2H), 3.50-3.41 (m, 3H)*, 3.32 (m, 2H)*, 3.28 (m, 1H)*3.08 (t, J=11.2 Hz, 1H), 2.94-2.89 (m, 2H), 2.77 (dd, J=10.7, 2.7 Hz, 1H), 2.20 (m, 2H), 1.87 (td, J=12.2, 3.0 Hz, 1H), 1.59-1.53 (m, 4H), 1.48-1.43 (m, 2H), 0.88 (d, J=7.0 Hz, 3H), 0.80-0.75 (m, 6H). ¹³C NMR HSQC Phase Edited (150 MHz, DMSO) δ 112.76 (1H), 111.33 (1H), 103.59 (1H), 72.32 (1H), 68.40 (1H), 62.91 (2H), 61.70 (1H), 56.42 (1H), 55.51 (2H), 54.85 (1H), 53.07 (1H), 52.07 (1H), 50.30 (1H), 42.20 (2H), 41.20 (2H), 39.78 (1H), 38.00 (2H), 37.58 (1H), 36.99 (2H), 34.01 (2H), 29.80 (2H), 22.85 (2H), 20.42 (2H), 13.24 (3H), 10.44 (3H), 9.15 (3H). *Assigned by HSQC.

### Compound I(xii): Diethylala⁶, (2R,3S)-β-Methyl-Cys⁸-Dideoxyamanitin

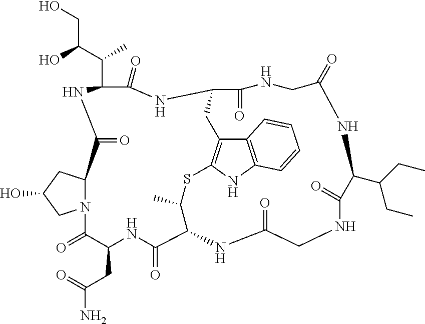 [SMILES: CCC(CC)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}=17.3 min; λₘₐₓ 290 nm; HRMS-ESI (m/z): C₄₁H₅₈N₁₀O₁₂SNa (M+Na) calc 937.3854 obs. 937.3848. ¹H NMR (600 MHz, DMSO) δ 11.23 (s, 1H), 9.09 (t, J=6.0 Hz, 1H), 8.68 (d, J=4.4 Hz, 1H), 8.41 (s, 1H), 8.19 (s, 1H), 8.06-7.93 (m, 3H), 7.76 (d, J=8.2 Hz, 1H), 7.58 (d, J=8.0 Hz, 1H), 7.43 (s, 1H), 7.28 (d, J=8.1 Hz, 1H), 7.12 (t, J=7.6 Hz, 1H), 7.02 (t, J=7.6 Hz, 1H), 5.13-5.03 (m, 1H), 4.89 (d, J=5.0 Hz, 1H), 4.67 (d, J=2.7 Hz, 1H), 4.56-4.48 (m, 2H), 4.39 (s, 1H), 4.30-4.23 (m, 2H), 3.99 (dd, J=18.8, 8.0 Hz, 1H), 3.90 (dd, J=17.4, 7.5 Hz, 1H), 3.85 (dd, J=8.9, 4.6 Hz, 1H), 3.78 (s, 2H), 3.55 (d, J=18.6 Hz, 1H), 3.48-3.42 (m, 2H)*, 3.33 (m, 2H)*, 3.16-3.09 (m, 1H), 3.05-2.95 (m, 2H), 2.24-2.17 (m, 2H), 1.91-1.85 (m, 1H), 1.51-1.45 (m, 2H), 1.39-1.32 (m, 1H), 1.29-1.20 (m, 2H), 1.20-1.12 (m, 1H), 0.91 (d, J=6.2 Hz, 3H), 0.87 (d, J=7.0 Hz, 3H), 0.79-0.75 (m, J=7.3, 3.9 Hz, 6H). ¹³C NMR HSQC Phase Edited (150 MHz, DMSO) δ 121.88 (1H), 119.86 (1H), 118.39 (1H), 111.01 (1H), 71.98 (1H), 68.39 (1H), 63.03 (2H), 61.93 (1H), 58.03 (1H), 56.78 (1H), 55.51 (2H), 54.60 (1H), 52.79 (1H), 50.55 (1H), 47.11 (1H), 42.22 (2H), 41.36 (2H), 39.40 (1H), 37.94 (1H), 37.29 (2H), 30.65 (2H), 20.12 (2H), 19.94 (2H), 17.81 (3H), 13.45 (3H), 10.24 (1H), 9.12 (3H). *Assigned by HSQC.

### Compound I(xiii): 5-OH Trp⁴, Diethylala⁶, (2R,3S)-β-Methyl-Cys⁸-Deoxyamanitin

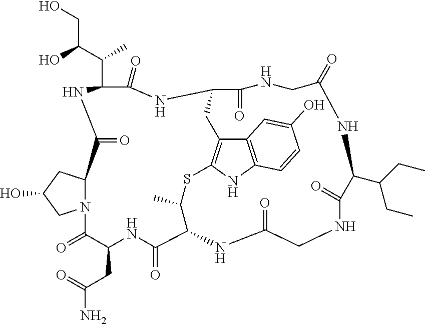 [SMILES: CCC(CC)[C@@H]1NC(=O)CNC(=O)[C@H]2Cc3c([nH]c4ccc(O)cc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

HPLC 2 (Method E): t_{R}^{=16.9} min; ax 288 nm; HRMS-ESI (m/z): C₄₁H₅₉N₁₀O₁₃S (M+H) calc 931.3984 obs. 931.3967. ¹H NMR (600 MHz, DMSO) δ 10.90 (s, 1H), 9.07 (s, 1H), 8.82 (s, 1H), 8.68 (d, J=4.0 Hz, 1H), 8.48 (s, 2H), 8.40 (s, 1H), 8.19 (s, 1H), 8.03-7.95 (m, 3H), 7.73 (d, J=8.2 Hz, 1H), 7.42 (s, 1H), 7.07 (d, J=8.7 Hz, 1H), 6.99-6.85 (m, 3H), 6.63 (dd, J=8.7, 2.1 Hz, 1H), 5.33 (s, 1H), 5.04 (s, 1H), 4.86 (d, J=4.4 Hz, 1H), 4.66 (s, 1H), 4.52 (dd, J=8.6, 6.0 Hz, 1H), 4.39 (s, 1H), 4.25 (m, 2H), 3.99 (d, J=7.6 Hz, 1H), 3.90 (dd, J=17.3, 7.1 Hz, 1H), 3.86-3.83 (m, 1H), 3.77 (dd, J=18.9, 10.7 Hz, 2H), 3.56 (d, J=18.7 Hz, 2H), 3.07 (t, J=14.1 Hz, 1H), 2.99 (d, J=15.0 Hz, 1H), 2.93 (dd, J=14.5, 5.9 Hz, 1H), 2.24-2.17 (m, 2H), 1.87 (td, J=12.0, 2.3 Hz, 1H), 1.52-1.45 (m, 2H), 1.41-1.31 (m, 1H), 1.31-1.20 (m, 3H), 0.89 (d, J=6.0 Hz, 3H), 0.86 (d, J=7.0 Hz, 3H), 0.78-0.76 (m, 6H). ¹³C NMR HSQC Phase Edited (150 MHz, DMSO) δ 112.49 (1H), 111.04 (1H), 103.36 (1H), 71.79 (1H), 68.17 (1H), 63.09 (2H), 61.72 (1H), 57.72 (1H), 56.63 (1H), 55.36 (2H), 54.55 (1H), 52.46 (1H), 50.32 (1H), 46.69 (1H), 42.02 (2H), 41.36 (1H), 41.32 (1H), 39.36 (1H), 37.80 (1H), 37.13 (2H), 30.49 (1H), 28.54 (2H), 20.15 (2H), 19.80 (2H), 17.44 (3H), 13.34 (3H), 9.91 (1H), 8.61 (3H).

### III. Discussion

The synthesis and biochemical evaluation of amatoxin analogs, several of which are as toxic as α-amanitin is reported herein, including the first super-toxic α-amanitin analog on record. The tryptathionine staple is important as it rigidifies both β-turns while orienting the indole towards the binding site. Notably, while the synthetically challenging indole-6-hydroxyl is dispensable in terms of cytotoxicity, its presence affords greater inhibitory activity against Pol II in vitro owing to a stronger π-cation interaction as discussed in a report on the less cytotoxic 5′-hydroxy-6′-deoxy-α-amanitin. Hence, toxins were synthesized with or without a 5′-hydroxyl group on the tryptathionine staple. The thio-ether, (R)-sulfoxide and sulfone of both the natural product and the 5′-hydroxy-6′-deoxy-α-amanitin are equi-cytotoxic whilst the (S)-sulfoxide is about 10% as toxic (Wieland et al, 1983). Hence, for this work thioethers were used. The “eastern”-turn is of known importance to toxicity (Baumann et al., 1993). Previously, an alanine-scan of the eastern ring (Gly⁵-Ile⁶-Gly⁷) resulted in substantial loss of Pol II inhibition, most significantly at Ile⁶ with >20,000 fold loss of activity (Zanotti et al., 1987). We focused on subtle side-chain and backbone modifications that would conserve the non-covalent interactions observed in x-ray/cryo-EM structures. Due to the sensitivity of the toxin to modification at Ile⁶ we altered the shape and polarity with branching alkyl groups or with oxy- and thioethers. In addition, to probe the β-turn, we introduced aza-glycines at positions 5 and 7. To constrain the staple pitch angle, we introduced β-methyl groups on the cysteine. Finally, we combined certain of these modifications.

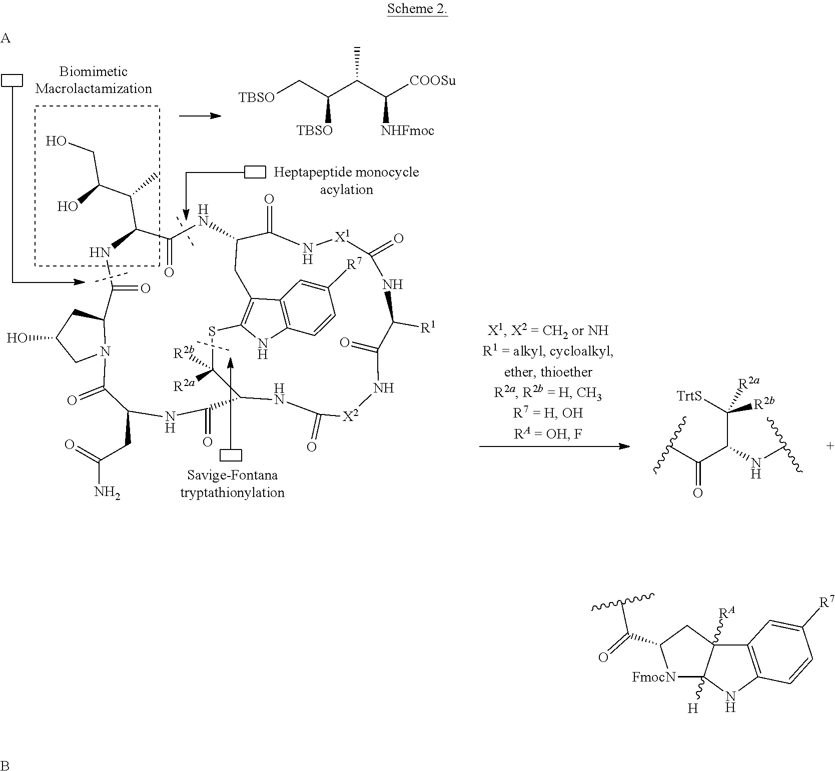

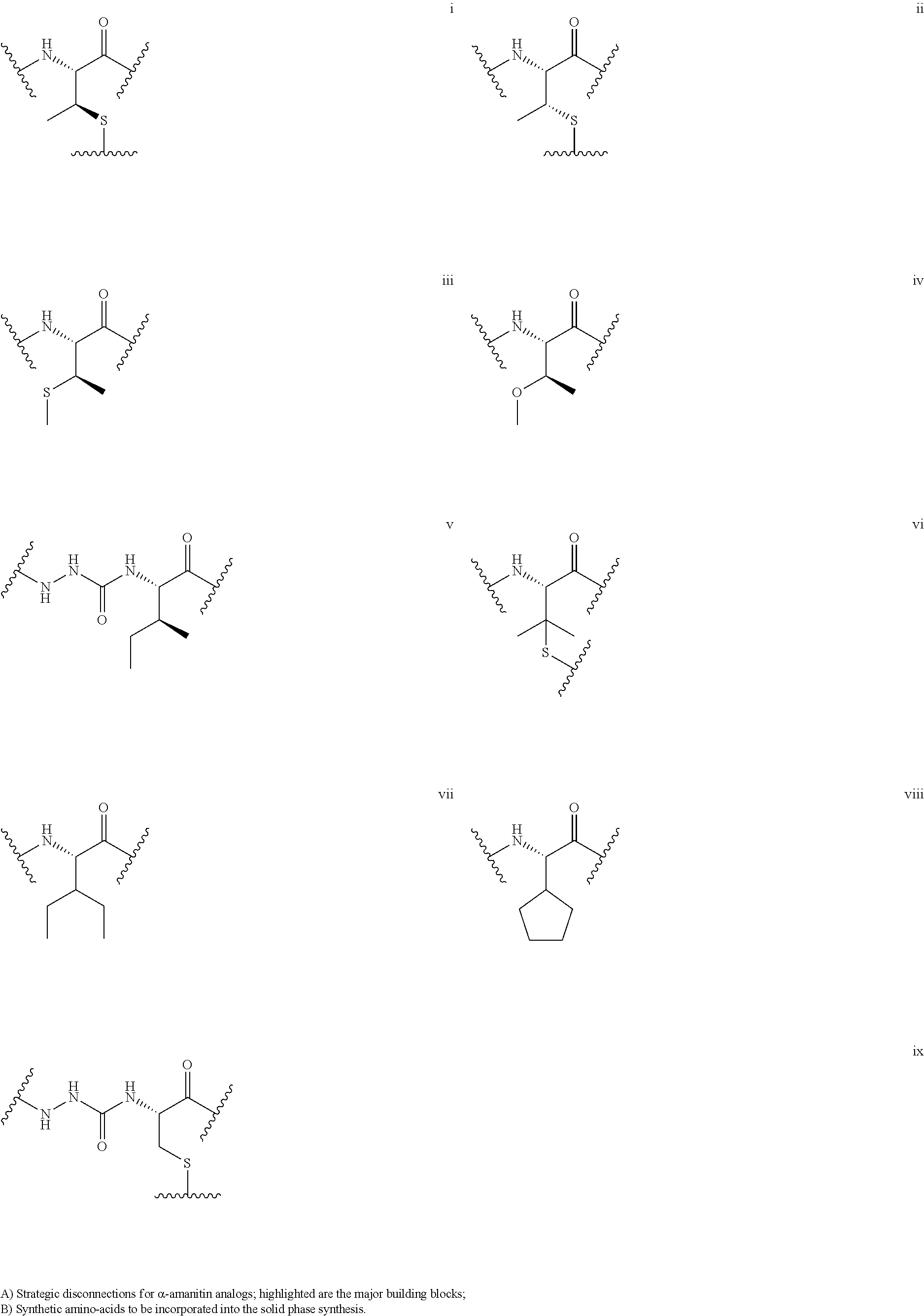

The present studies required synthesis of novel monomers (Scheme 3); thia-isoleucine and the threo- and erythro-diastereomers of β-methylcysteine were readily accessed via allo-threonine (Scheme 2A,B). Fmoc-oxo-Ile-OH was readily achieved by methylation of N^{α}-Boc-L-Thr-OH (Scheme 2C). Finally, the two aza-glycines were obtained from semicarbazides generated by reaction of Fmoc-NHNH₂ with isocyanate precursors (Scheme 2D).

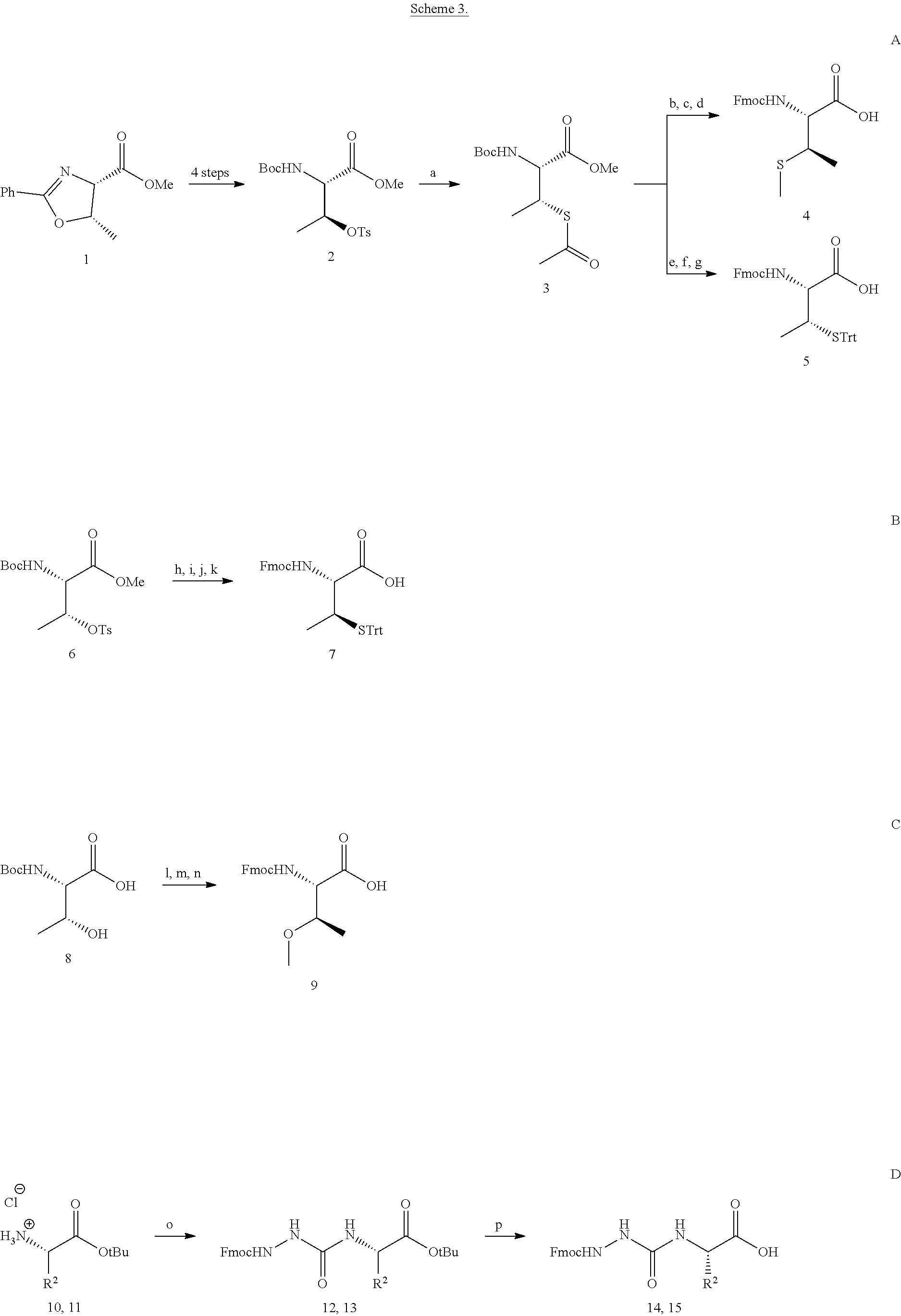

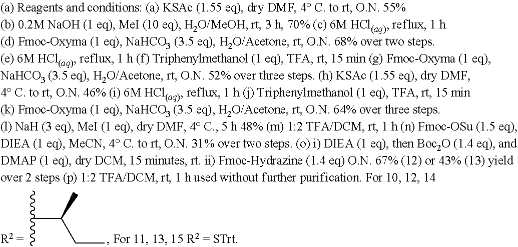 [SMILES: *[C@@H](C)CC]

Nine resin-bound heptapeptides were readily assembled via a linear solid phase peptide synthesis (SPPS) involving hydroxypyrroloindoline (Hpi) (Scheme 4) in line with procedures previously reported in the literature (May et al., 2005; May et al., 2007; May et al., 2008).

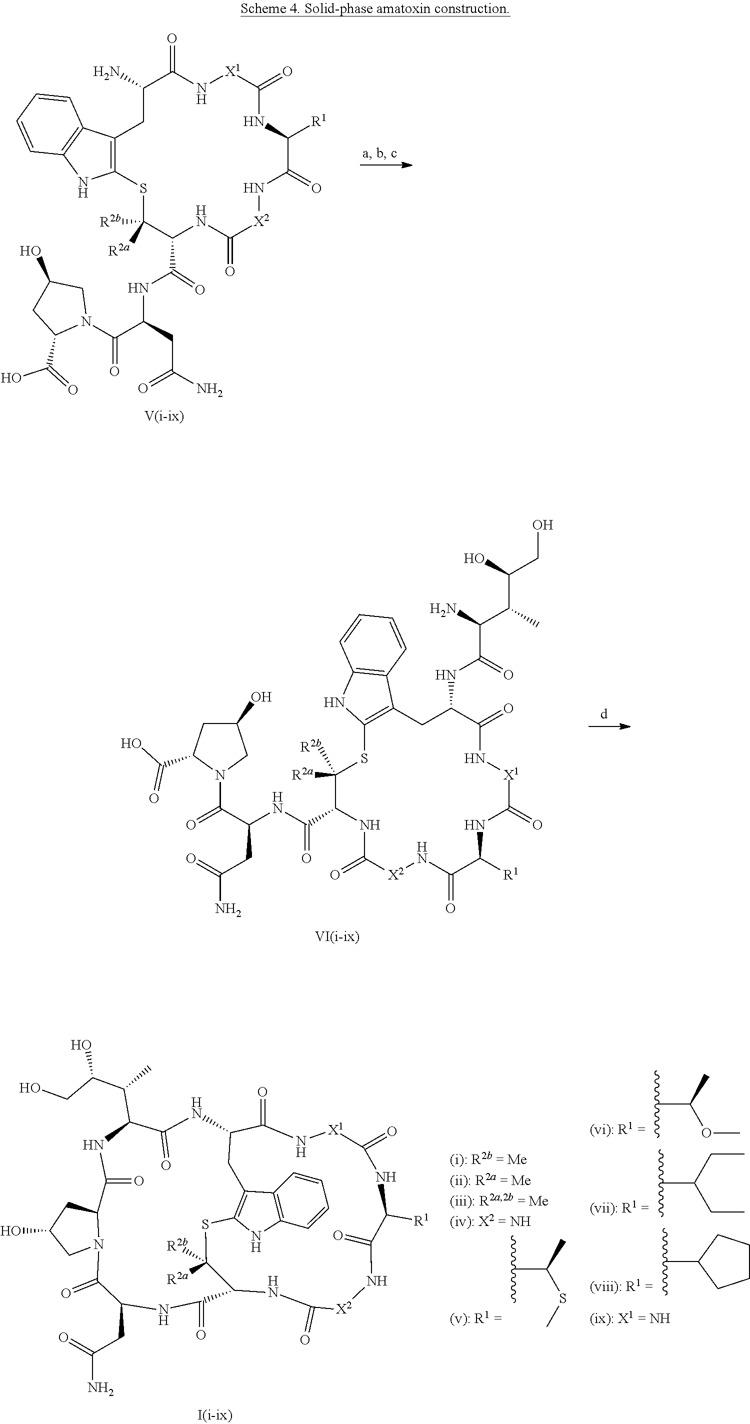

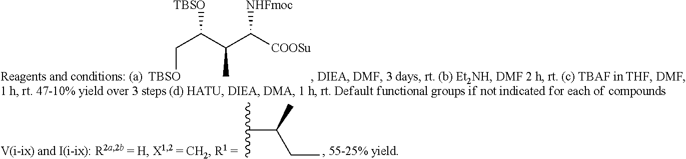

Our choice of Hpi over the well-known I₂-mediated cyclization (Yao et al., 2021; Schuresko et al., 2007) was predicated on convenient preparation of Hpi on scale using an admixture of Oxone in acetone (Blanc et al., 2017) and an appreciation of the incompatibility of I₂ with thia-Ile here and the eventual use of other oxidatively sensitive amino acids. Furthermore, the use of Hpi preserved the biomimetic macrolactamization between DHIle³ and Hyp (Wieland & Faulstich, 1991; Vetter, 1998; Luo et al., 2014) an important consideration for the eventual use of analogs which add structural constraints that may otherwise complicate cyclization.

Yields were high in all cases except for aza-gly⁵ heptapeptide (V(ix)), which proved more challenging to acylate and elongate under the conditions used. TFA treatment cleaved all heptapeptides from the resin while concomitantly effecting tryptathionylation and global deprotection. The resulting monocycles were purified, acylated with Fmoc-DHIle-NHS ester, and deprotected by Et₂NH followed by TBAF-dioxane. Following purification, the octapeptide monocycles were macrolactamized to yield the final toxins I(i-ix) (Scheme 3).

The activities of I(i-ix) were assessed on CHO cells using an MTT assay and in-vitro using a runoff transcription (RT) assay (Table 5). All but two amatoxins were greater than 10% as toxic as α-amanitin. Since toxins containing erythro-β-methylcysteine (I(v)) and diethylalanine (I(vii)) were each nearly as cytotoxic as α-amanitin, both analogs seemed attractive for combining with other substitutions in a focused array. To interrogate the effect of increasing indole electron density while providing an exemplary handle for eventual bioconjugation, a 5′-hydroxy-tryptathionine was also included as amanitins have been conjugated to the hydroxyindole with immolating linkers (Bodero et al., 2018; Park et al., 2019a; Gallo et al., 2021). The synthesis of these second-generation amatoxins that combine motifs the results of the initial experiments indicated were likely to improve cytotoxicity proceeded analogously per Scheme 3 and these were evaluated by MTT and RT assays, the results of which are also summarized in Table 5 (I(x-xiii)).

The combination of erythro-β-methylcysteine and diethylalanine gave a toxin that was nearly twice as cytotoxic as α-amanitin. Interestingly, the 5′-hydroxytryptathionine analog (Table 5) showed significantly reduced cytotoxicity compared with dideoxy-α-amanitin suggesting that the increased electron density in the indole does not necessarily enhance cytotoxicity.

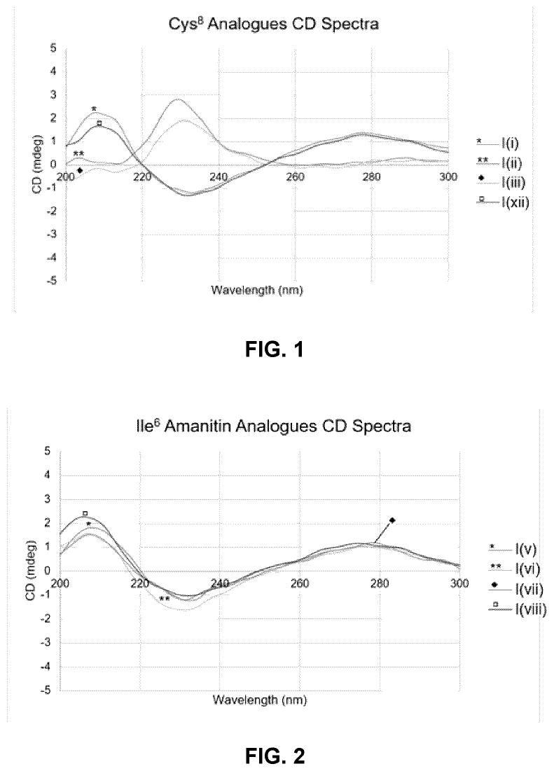

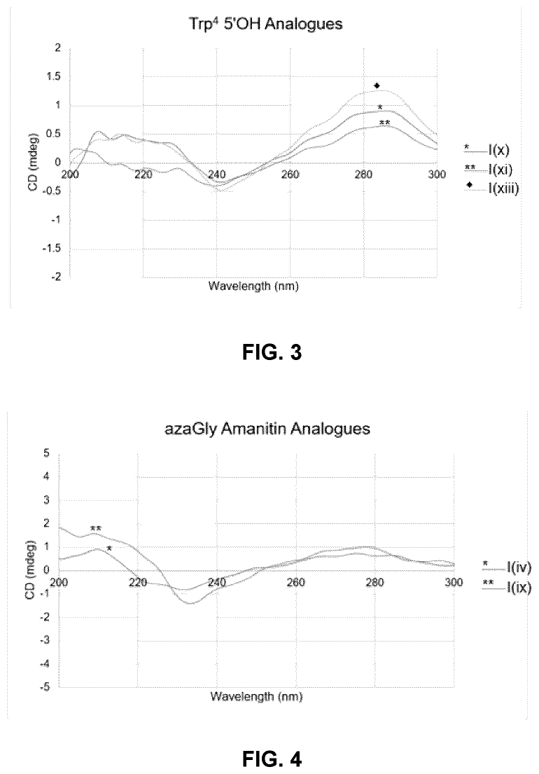

To address the structural aspect of these toxins, circular dichroism (CD) spectra (ESI) were acquired. Nearly all of the toxins showed similar CD spectra suggesting considerable structural similarities. CD spectra of erythro-β-Methyl Cys⁸ containing analogues (FIG. 1), I(i) and I(xii), show spectral characteristics highly consistent with previous reports of CD spectra of unsubstituted tryptathionine thioether amanitin analogues (Matinkhoo et al., 2021; Zanotti et al., 1981). Key features include a single trough at 230 nm, and peaks around 275 nm and 210 nm. Conversely, the threo-β-Methyl Cys⁸ analogue, I(ii), and the Pen⁸ analogue, I(iii), show an inverted cotton effect (FIG. 1). The spectra appear to be mirror images of I(i) and I(xii) between 275 nm and 210 nm, with a peak at 230 nm. While not wishing to be limited by theory, these aspects suggest a significantly different turn structure compared with unsubstituted cysteine or erythro-β-Methyl Cys⁸ bearing toxins. CD spectra of all Ile⁶ amanitin analogues (FIG. 2) show the same features as compounds I(i) and I(iii). They overlap with one another and, while not wishing to be limited by theory, suggest global structural similarity to their base compound, dideoxyamanitin. CD spectra of compounds with 5′ OH tryptathionine staples (FIG. 3), I(x), I(xi) and I(xiii), all show the characteristic features of 5′ OH tryptathionine thioether amanitin analogues (Pryyma et al., 2020). While the overall shape is similar to the unsubstituted tryptathionine series, the trough is shifted to 240 nm, while the peaks are at about 290 nm and about 205 nm. The CD spectra of both aza-Gly amanitins (FIG. 4), I(iv) and I(ix), bear general similarity to literature reports of unsubstituted tryptathionine thioether amanitin analogues and the erythro-β-Methyl cysteine⁸ and Ile⁶ analogues described herein. The peaks and troughs occur at similar at wavelengths, but the slopes of the rising and falling features differ.

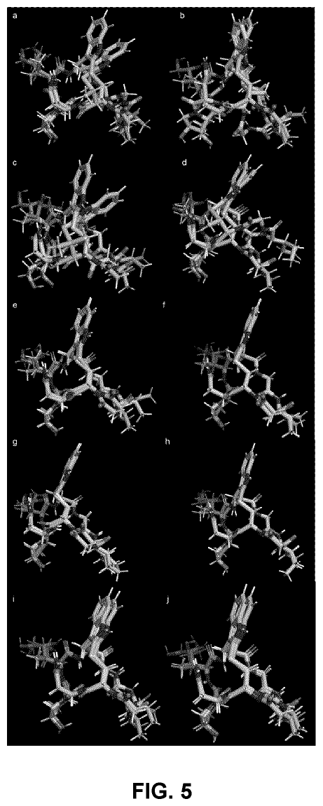

Yet, as seen with sulfoxides and sulfones, CD spectra are not predictive of cytotoxicity (Pryyma et al., 2020). To contextualize these results, we constructed energy-minimized models of the new analogs and of dideoxy-α-amanitin using Avogadro and overlaid them in PyMOL (FIG. 5); the greatest variance was found between the β-methylcysteine analogs (FIG. 5, structures a-d), wherein the erythro analog (FIG. 5, structure a) appears similar to α-amanitin while the threo analog (FIG. 5, structure b) is highly deviant. In validation of this modelling is the cytotoxicity and to a lesser extent the Kᵢ value for transcription inhibition. For the remaining compounds, modelling showed similarity to dideoxy-α-amanitin (FIG. 5, structures e-j) yet did not explain greater differences in IC₅₀ or Kᵢ values. In the case of I(i), the introduction of a β-methyl only slightly altered the pitch angle of the tryptathionine staple, while the overall peptide structure was preserved, as corroborated by molecular modelling (FIG. 5). Conversely, its epimer I(ii) significantly perturbs the staple angle relative to the macrolactam while grossly distorting the global peptide structure and critical hydroxyproline residue. Consistent with intermediate structural perturbation, the penicillamine analog I(iii) was intermediately toxic.

¹H, COSY, HSQC, HMBC, and NOESY were obtained for I(iv), I(v) and I(xii), and the remaining amatoxin analogs were assigned based on ¹H, COSY and phase edited HSQC. The NMR data yielded some interesting observations, for example the high degree of similarity between all Ile⁶ analogues (I(v-viii) and I(xi)). While not wishing to be limited by theory, this similarity for all key chemical shifts suggests a high degree of structural similarity between the compounds in question, an observation further corroborated by molecular modelling and circular dichroism spectra. Given the modifications are only to the amino acid side chain, while not wishing to be limited by theory, the high degree of variance in toxicity and polymerase inhibition between the amatoxin analogs suggests a highly specific interaction between this position on the toxin and the corresponding groove on Pol II is important for inhibition and toxicity, as the differences cannot be explained by gross structural changes induced by amino acid substitution. Conversely, substitution of Cys⁸ with analogues bearing methyl groups on the beta carbon does induce some changes in chemical shifts. The erythro-β-Methyl Cys⁸ bearing I(i), I(x), I(xii) and I(xiii), all induce very similar changes to select chemical shifts, namely the Trp⁴ CH-alpha moving from about 4.9 ppm to about 5.05 ppm and the CH-alpha of DhIle³ moving from about 4.44 ppm to about 4.26 ppm. The remaining chemical shifts are very similar to the Ile⁶ analogue series. I(ii) and I(iii) on the other hand, cause significant perturbation to chemical shifts of numerous residues, including Asn¹, Trp⁴, both glycines and Cys⁸, while not wishing to be limited by theory, these observations support the large changes to global structure for these compounds suggested by circular dichroism and molecular modelling. The aza-Gly analogues show local perturbations in chemical shift for residues adjacent to the aza-glycine, Cys⁸ for aza-Gly⁷, Ile⁶ and Gly⁷ for aza-Gly⁵. The remaining chemical shifts of other residues remain very similar and while not wishing to be limited by theory, support the minimal changes to gross structure suggested by molecular modelling and circular dichroism.

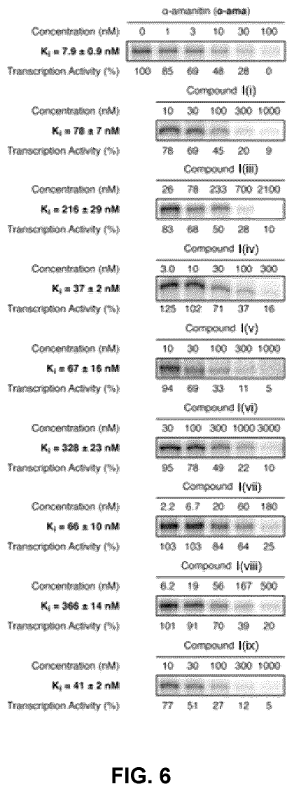

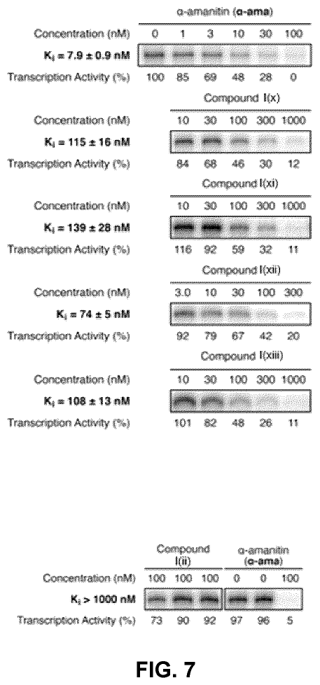

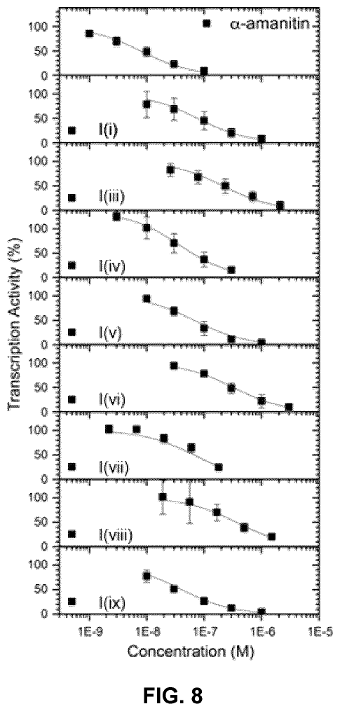

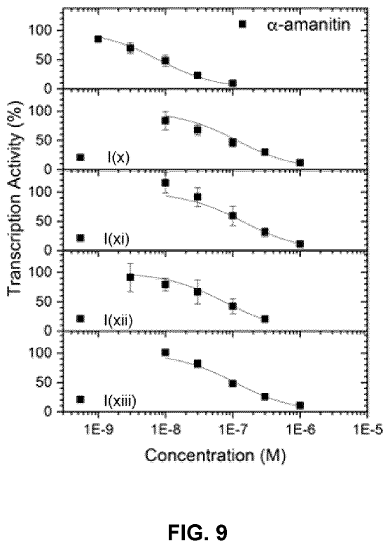

Despite the fact that both CD spectra and molecular modelling suggest nearly total structural overlap between several analogs, cytotoxicity and Pol II inhibition assays showed significant differences. This was particularly evident at position 6. For example, the thia-Ile amatoxin I(v) showed only slightly reduced cytotoxicity whilst the oxy-Ile I(vi) displayed no observable cytotoxicity (Table 5). While not wishing to be limited by theory, this trend may be rationalized based on relative hydrophobicity as well as reduced van der Waals radius of oxy-analog along with greater propensity for H-bonding. The requirement for conformational flexibility at the β-position of Ile was evidenced by the equipotency seen with diethylalanine I(vii) while the cyclopentyl glycine I(viii) showed considerably diminished toxicity. Modelling did not predict this difference since both compounds adopted the same conformation as α-amanitin (FIG. 5, structures e and f). Both aza-gly analogs proved potent; I(iv) is the most toxic synthetic amatoxin on record. As modelling suggests great structural similarity to α-amanitin (FIG. 5, structure j) its activity was generally not surprising, but the extent of toxicity was unexpected and may, while not wishing to be limited by theory, result from a subtle interplay of factors influencing conformation. Despite literature precedence suggesting that aza amino acids significantly destabilize β-hairpin structures (McMechen et al., 2019) aza-gly⁵ I(ix) showed only a two-fold reduction in cytotoxicity. Table 6 provides a summary of transcription activities of HeLa nuclear extract in varying amanitin analog concentrations (n=3). FIGS. 6-9 show characteristic inhibition of transcription by α-amanitin and amatoxin analogs I(i-xiii) under varied concentrations (n=3). While all Kᵢ values for polymerase inhibition were higher than α-amanitin, they were similar to dideoxyamanitin, which is known to be less potent due to the absence of the indole-OH yet equally cytotoxic. Taken together, these findings suggest that the polymerase binding pocket shows considerable specificity for a non-polar and branched side chain at this position, which we have perturbed in subtle ways using otherwise structurally similar toxins. However, cytotoxicity may depend on other factors including but not limited to cellular uptake, nuclear internalization, metabolism and efflux, and/or genetic susceptibility to apoptotic arrest (Li et al., 2018; Li et al., 2021). In so far as certain modifications e.g. the β-methyl-cysteine increased the lipophilicity as gauged by HPLC retention times, such may ultimately improve cellular uptake, making this analog desirable as an ADC payload.

We built a select number of amatoxins to generate a structure-activity relationship of the eastern ring of α-amanitin. This work highlights the use of synthesis for precise structural perturbations that led to unexpected results and a deeper understanding of the limited plasticity of α-amanitin while accessing analogs that are even more toxic than the title compound.

Combining these features resulted in second generation toxins that included one example that was 3-fold more toxic than α-amanitin. While all analogs were highly cytotoxic, they were less inhibitory than α-amanitin in-vitro, suggesting that the modifications could also affect bioavailability and/or cell uptake. These analogs may be used, for example, in the design of novel targeted bioconjugates and anti-cancer therapeutics.

### Example 2: Biochemical Evaluation Amatoxins in the Context of 3 Different Cell Lines

Here we report the biochemical evaluation of 13 strategically designed amatoxins, several of which are as toxic as α-amanitin in the context of 3 different cell lines. We assayed synthetic amatoxins on Chinese hamster ovary (CHO) cells owing to their noted sensitivity to α-amanitin. Other reports have employed HEK293 cells (Yao et al., 2021). In expanding the pharmacopeia of amatoxins, herein we explore cytotoxicity of the most potent synthetic amatoxins on several cell lines and we show differential cytotoxicity that depend on the cell-lines being evaluated, with one amatoxin being up to 5-fold more toxic than α-amanitin when evaluated on HEK293 cells.

### I. Results

The activities of I(i-xiii) were assessed on CHO cells using a standard 3-(4,5-dimethylthiazol-2-yl)-2,5-diphenyltetrazolium bromide (MTT) assay as described in Example 1. The calculated cytotoxicity IC₅₀ value for α-amanitin varied from day to day; hence, all assays were performed in triplicate, and in each set, α-amanitin was used as a positive control. In light of this variability, IC₅₀ values are reported relative to α-amanitin in the tables. Notably, all but two amatoxins were at least 10% as toxic as α-amanitin when evaluated on CHO and HEK293 cells (Table 7 and Table 8). The two compounds, i.e., I(iv) and I(xii), which showed the highest cytotoxicity against CHO cells as well as compound I(v) that showed high Pol II inhibition, but lower cytotoxicity, were then selected for further biological evaluation in a panel of three additional cell lines (Table 7 and Table 8). HEK293 cells were chosen due to their frequent use in the literature for evaluating the cytotoxicity of α-amanitin analogues. HeLa cells were chosen as these represent another commonly used model cancer cell line. Finally, HepG2 cells were chosen because they represent well-known models for probing human hepatocyte toxicity and metabolism (Gerets et al., 2012). The increased cytotoxicity of compounds I(iv) and I(xii) observed with CHO cells was also manifest in HEK293 and HeLa cells. Against CHO cells, compound I(xii) was found to be 2 times as toxic as α-amanitin and against HEK293 cells to be 5 times as toxic as α-amanitin. Against HepG2 (liver-derived cells), I(iv), I(v), and I(xii) were found to be significantly less toxic than α-amanitin. As HEK293 cells proved most sensitive, the remaining cytotoxic α-amatoxins were evaluated in this cell line. All of the tested compounds are highly cytotoxic against HEK293 cells, with most compounds exceeding their respective observed toxicities against CHO cells (Table 7 and Table 8). Additionally, whereas indole hydroxylation reduced cytotoxicity when measured against CHO cells, this effect was attenuated in HEK293 cells for compounds I(x), I(xi), and I(xiii), which were comparable to I(i), I(vii), and I(iii), respectively. This finding supports the potential use of such 5′-OH tryptathionine-containing analogues for bioconjugation through the indole hydroxyl.

### II. Discussion

Differential cytotoxicity was observed depending on the cell line tested. Many analogues were highly cytotoxic, the most potent of which, I(xii), is nearly twice as toxic as α-amanitin against CHO cells and nearly 5-fold more cytotoxic than α-amanitin against HEK293 cells. The most cytotoxic amatoxins I(iv), I(v), and I(xii) were less toxic than α-amanitin against liver-derived HepG2 cells. However, inasmuch as HepG2 cells mimic hepatocytes, this relative lack of cytotoxicity compared to that of α-amanitin, while not wishing to be limited by theory, suggests that different amatoxins may be designed for enhanced selectivity against specific cancer cell lines while sparing hepatocytes. In addition, we anticipate that the differential cytotoxicity observed (HEK293 vs HepG2) points to the potential for further enhancing the specificity of amatoxins to kill specific subsets of cancer cells.

### Example 3: Amanitin Eastern Fragment Analog with N-substituted azaGly⁷

Compound I(xiv) with an isopropyl group on azaGly⁷ was prepared according to Schemes 4-10 which included synthetic procedures described hereinabove in Example 1.

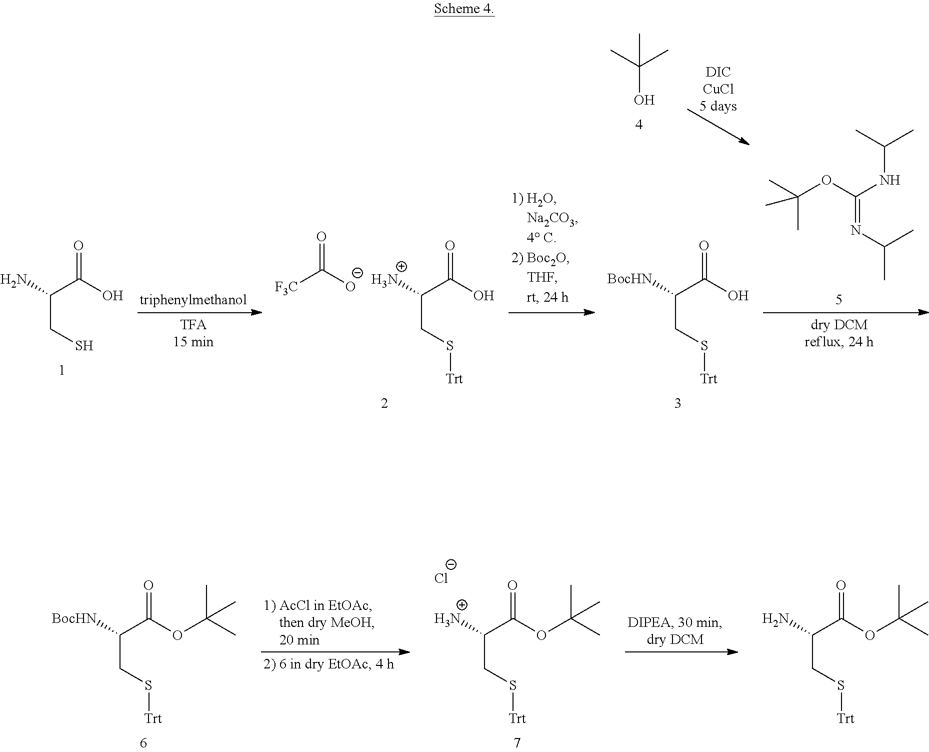

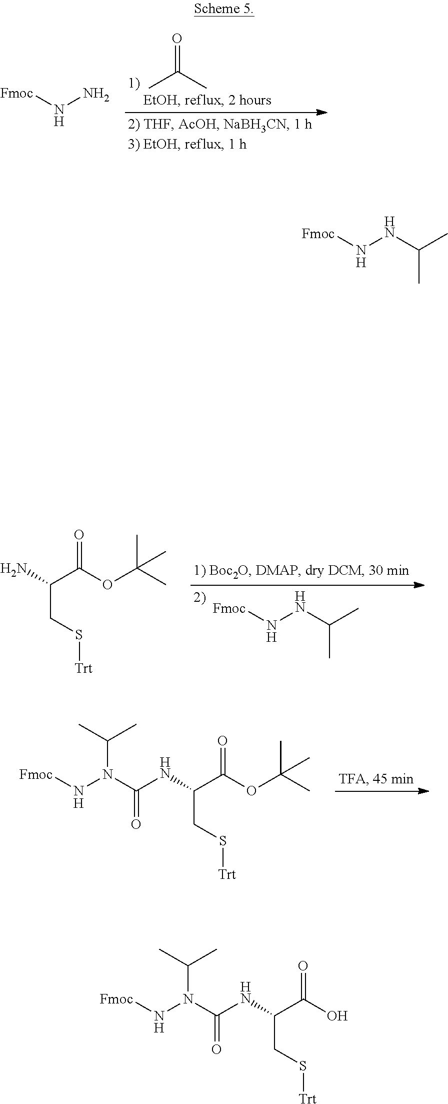

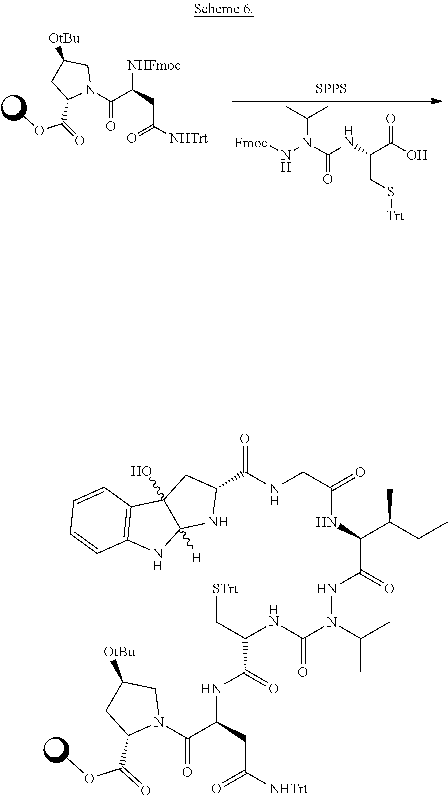

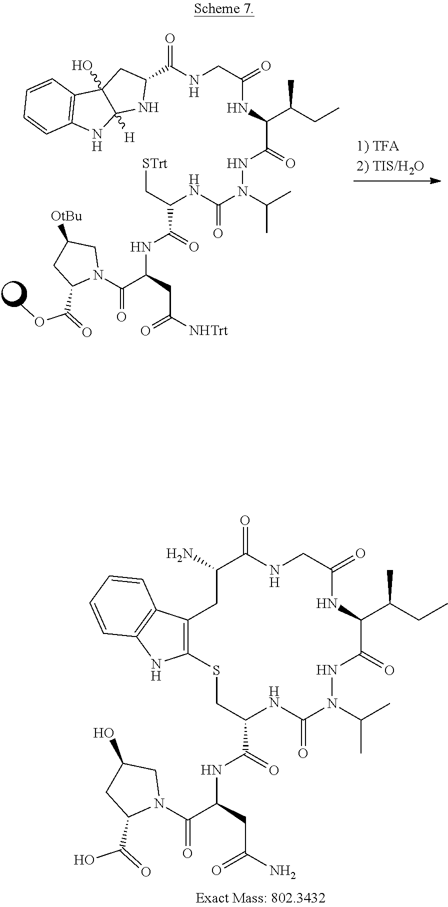

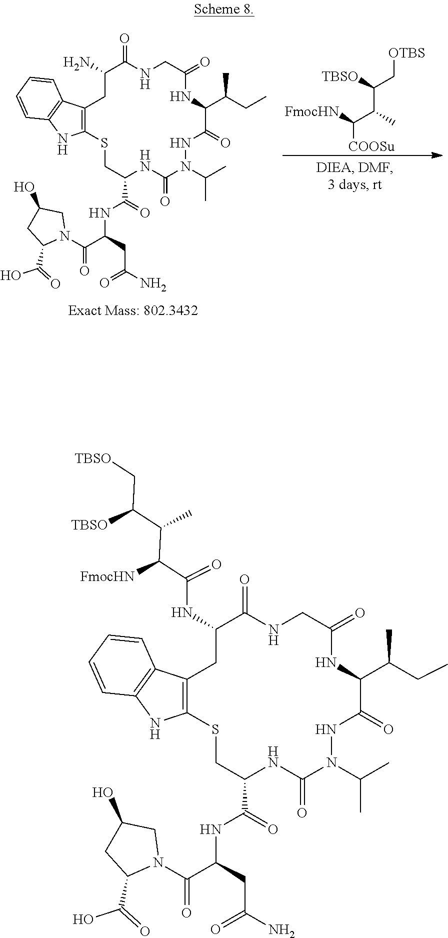

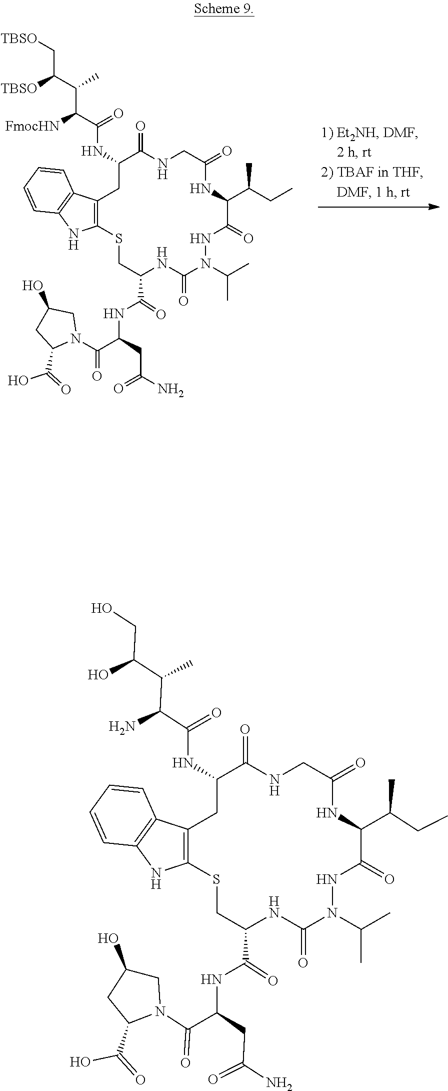

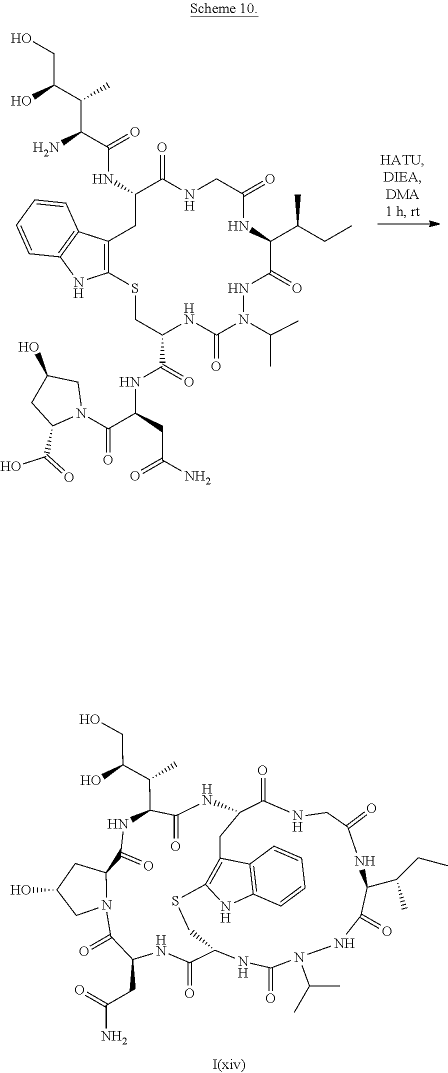 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](O)C[C@H]2C(=O)O)NC(=O)N(C(C)C)NC1=O ; CC[C@H](C)[C@@H]1NC(=O)CNC(=O)[C@@H]2Cc3c([nH]c4ccccc34)SC[C@H](NC(=O)N(C(C)C)NC1=O)C(=O)N[C@@H](CC(N)=O)C(=O)N1C[C@H](O)C[C@H]1C(=O)N[C@@H]([C@@H](C)[C@@H](O)CO)C(=O)N2]

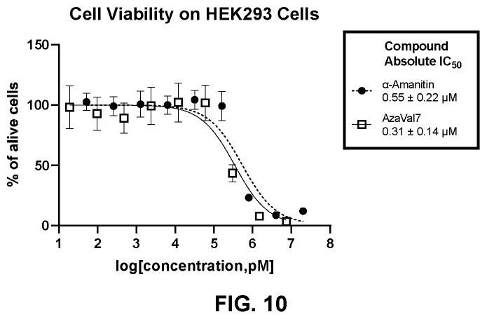

Compound I(xiv) was purified by HPLC. ESI-MS was performed on all of the precursors. Coupling was peak-to-peak and the corresponding peak was taken. Molecular modelling/docking suggested that Compound I(xiv) should be potent; e.g., have a useful IC₅₀ value. HEK293 cells were evaluated for %-living/dead as and this value was plotted vs. the amount of Compound I(xiv) dosed as given and plotted in a standard dose-response curve %-living on the y-axis and log(pmol) on the x-axis in line with procedures described in greater detail hereinabove (FIG. 10).

### Example 4: Bioconjugation of Amanitins

### I. Materials and Methods

Bioconjugation of Amanitin to Trastuzumab: General Procedure: 7.57 μL of dry pyridine was added to an Eppendorf tube containing 100 nmol of lyophilized Amanitin. 2.74 mg of glutaric anhydride (24 umol, 240 eq.) was dissolved in 30.3 μL of dry pyridine. This solution was immediately added to Amanitin in pyridine. The reaction mixture was then mixed thoroughly by pipetting and was allowed to react for 24 hours at room temperature. Then, Amanitin-glutarate was triturated with 800 μL Et₂O and pelleted by microcentrifugation. The pellet was washed two more times with 800 μL Et₂O to produce a dry brown pellet. A solution of N,N′-diisopropylcarbodiimide (DIC) in dry DMF was prepared to a concentration 0.125 mg/mL and a solution of NHS in dry DMF was prepared to a concentration of 0.265 mg/mL. The Amanitin-glutarate pellet from the previous step was dissolved in 1.5 μL of dry DMF and 30.3 μL of DIC in DMF (30 umol, 300 eq.) was added to this solution after which 0.3 μL of NHS in DMF (700 nmol, 7 eq.) was added to the reaction mixture. The reaction mixture was mixed thoroughly by pipetting and was allowed to react for 24 hours at room temperature. The Amanitin-glutarate-NHS was triturated with 800 μL Et₂O and the resulting pellet was washed twice with 800 μL Et₂O. Then, the pellet was dissolved in 3 μL DMF and 500 μL of 3 mg/mL Trastuzumab (Roche, 10 nmol) in PBS was added and the reaction mixture was mixed thoroughly by pipetting. The reaction was carried out at room temperature for 24 hrs with slow rotation. The Amanitin-Trastuzumab bioconjugate was purified using an Amicon μLtra-0.5 100 kDa MolecμLar Weight Cut-Off centrifugal filter unit and the concentrate was washed 3 times with PBS; the final concentrate was collected and diluted to 500 μL with PBS.

Amanitin-Trastuzumab Bioconjugate Quantification: A stock solution of bovine serum albumin (BSA) in PBS was prepared to 1 mg/mL concentration. 5 calibration solutions were prepared using 0, 6.4, 12.8, 19.2, 25.6, and 32 μL of stock BSA and making up to 32 μL with PBS. The final concentration of each calibration solution was 0, 0.2, 0.4, 0.6, 0.8, and 1 mg/mL, respectively. 968 μL of ThermoFischer Ready-to-Use Bradford Reagent was added to the calibration solutions and the resulting solutions were incubated at room temperature for 10 minutes. Absorbance was recorded at 595 nm to obtain a calibration curve. At the same time, 5 μL of Amanitin-Trastuzumab bioconjugate was dissolved in 27 μL PBS and 968 μL of ThermoFischer Ready-to-Use Bradford Reagent was added and the resulting solution was incubated at room temperature for 10 minutes. Absorbance was recorded at 595 nm, and concentration is interpolated from calibration curve with BSA.

Amanitin-Trastuzumab Bioconjugate DAR Analysis: Intact protein molecular weight was determined using Matrix-Assisted Laser Desorption/Ionization (MALDI). The saturated sinapic acid (SA) matrix was prepared in 30% MeCN/H₂O with 0.3% TFA. 1 μL of Amanitin-Trastuzumab bioconjugate was mixed thoroughly with 10 μL of saturated SA matrix solution and 5 μL of the resulting mixture was pipetted onto a target plate. MALDI analysis was carried out with a Bruker AUTOFLEX MALDI-TOF. Drug-to-antibody ratio (DAR) was then calculated as follows:

$${DAR}_{Average} = \frac{{MW}_{Conjugate} - {MW}_{Trastuzumab}}{{MW}_{Payload}}$$

Cell Culture and Cytotoxicity Assays: Cells were cultured in high-sucrose DMEM or McCoy 5A (Modified) medium, purchased from Gibco. Fetal bovine serum (FBS), 0.25% trypsin (with 1.3 mM EDTA), 0.85% Trypan blue, and the antibiotic mixture (100 units/mL penicillin and 100 μg/mL streptomycin) were also purchased from Gibco. MTT was purchased from Invitrogen. All cell culture mediums contained 10% FBS and 100 units/mL penicillin and 100 μg/mL streptomycin. All cell culture plastics were purchased from Corning or Falcon. Cells were cultured in a humified incubator at 37° with 5% CO₂. All experiments were carried out in a biological safety cabinet and performed in triplicate. For cell revival from liquid nitrogen storage, a 1 mL vial of cells in culture medium with 10% DMSO (purified through a 0.2 mm filter) was thawed at room temperature for two minutes, then warmed in a 370 incubator for five minutes. The storage medium was transferred to a T25 culture flask containing 2 mL of fresh culture medium and incubated at 37° at 5% CO₂. After 24 hours, the medium was aspirated and replaced with warmed, fresh medium. When cells reached a level of 80-95% confluence, they were subcultured by removing the medium and treating the cells with 0.25% trypsin containing 1.3 mM EDTA. The cells were incubated for 10 minutes to allow for sufficient detachment, quenched with 1 mL medium, and transferred into a 15 mL falcon tube. The cell suspension was centrifuged for 5 minutes at 8000 rpm and the supernatant discarded. The resulting pellet was resuspended in cell medium, diluted, and transferred to new culture flasks for incubation. To assess cell viability, the suspended cells were treated with Trypan blue and counted using a hemacytometer. The cells were then diluted to suitable concentrations and pipetted into the interior 60 wells of 96-well plates at a volume of 50 μL. The 96-well plates were incubated for 24 hours to allow for adherence. The desired additives were dissolved in PBS, serial diluted in cell medium (for a PBS maximum final concentration of 1%) and added to the wells in 50 μL aliquots. The plates were then incubated for a further 72 hours before 50 μL of 2.5 mg/mL MTT in PBS was added to each well. The cells were incubated for 3 hours, after which the medium was carefully aspirated from all wells and the resulting purple formazan crystals were dissolved in DMSO. The plates were shaken for two minutes and the absorbance of all wells was measured with a Beckman-Coulter DTX 880 multimode detector at a wavelength of 595 nm. The absorbance data was processed in Microsoft™ Excel and GraphPad Prism 9.0, with error bars calculated as the standard error of the mean. SK-BR-3 cells were diluted to a concentration of 100,000 cells/mL and 5,000 cells were seeded in each interior well; MCF7 cells were diluted to 200,000 cells/mL and 10,000 cells were seeded per well. Stocks of alpha-amanitin, Herceptin™, and antibody-drug conjugates (ADCs) were prepared at various concentrations; for alpha-amanitin: 20, 4, 0.8, 0.16, 0.032, 0.0064, 0.00128, 0.000256, 0.0000512 μM; for Herceptin and ADCs: 1000, 100, 10, 1, 0.1, 0.01, 0.001, 0.0001, 0.00001, 0.000001, 0.0000001, 0.00000001 nM. From the above stock solutions, 50 μL was removed and applied to a cell culture of volume 50 μL.

### II. Results and Discussion

Certain synthetic amatoxins herein that showed excellent cytotoxicity against HEK cells were conjugated to trastuzumab (Herceptin™) via a linker in a two-step method in line with that reported by Moldenhauer et al. (JNCI 2012 104(8):622-34) to obtain exemplary conjugates (Scheme 11). Conjugates were purified by HPLC and evaluated by MALDI-TOF to calculate the drug-to-antibody ratio (DAR). The same method was applied to alpha-amanitin as a control.

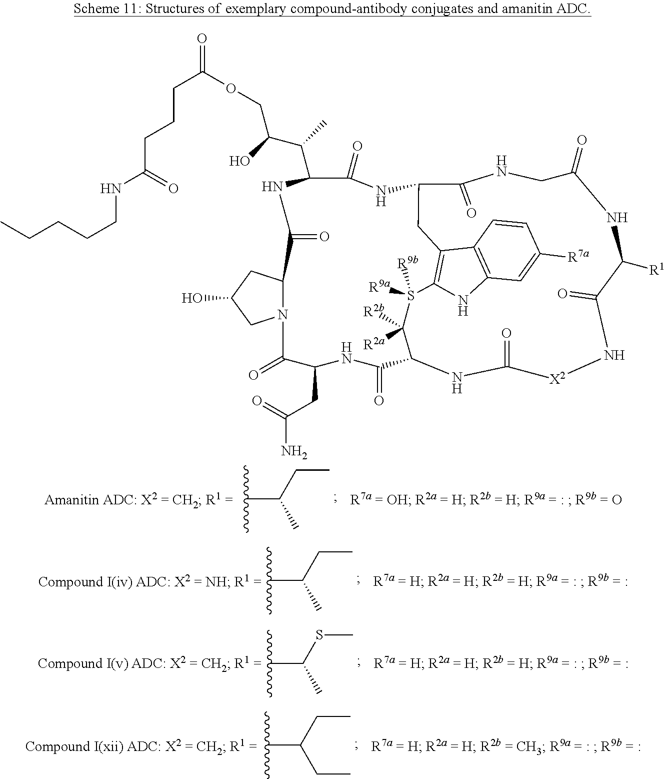

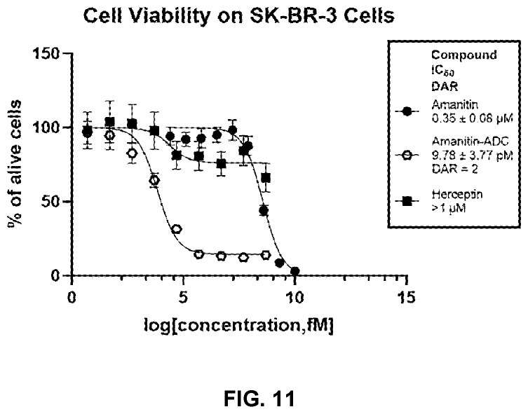

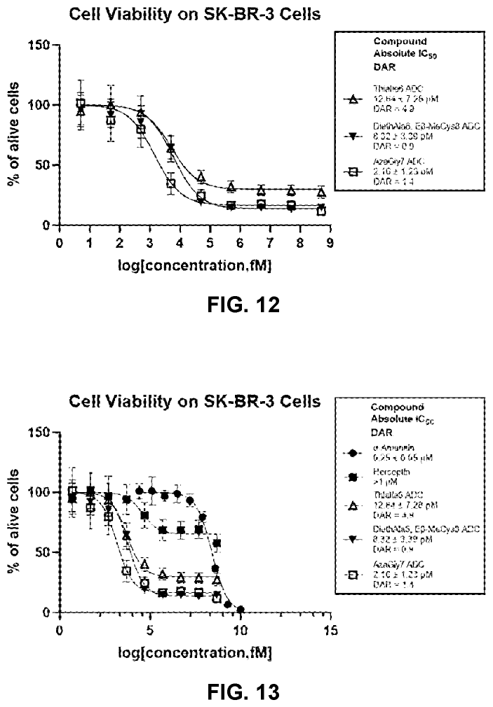

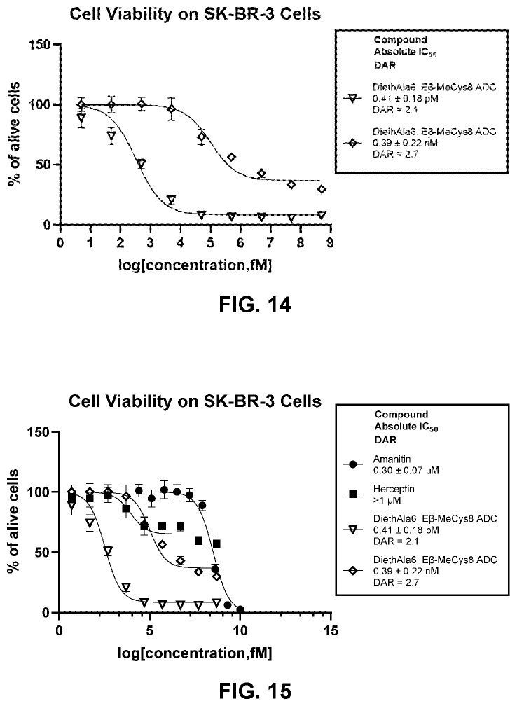

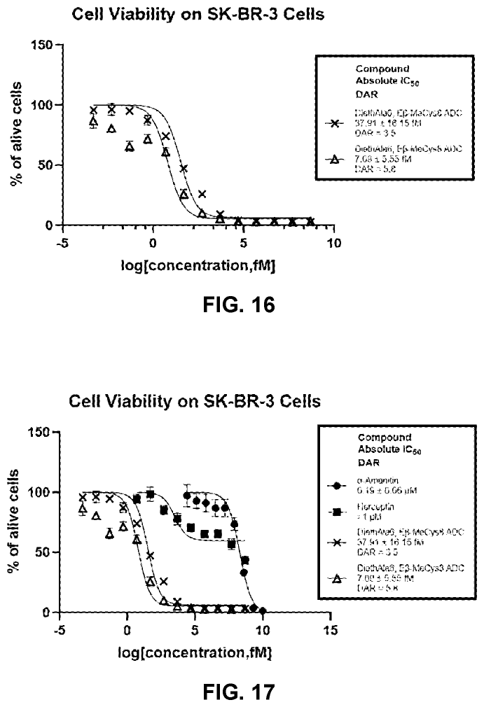

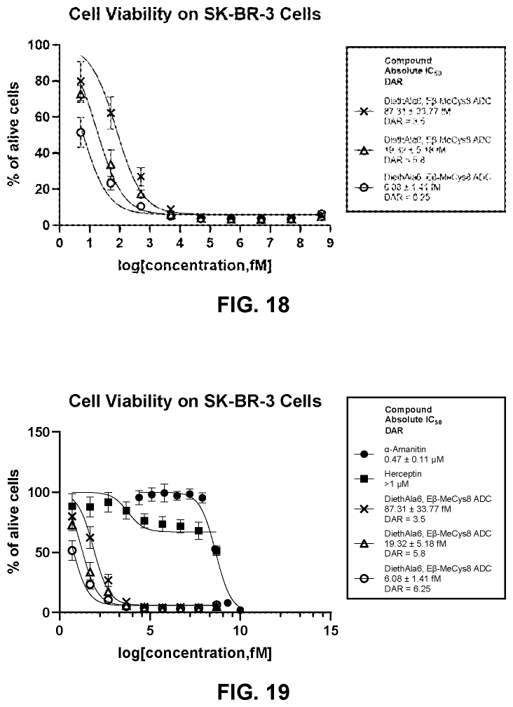

ADCs, at DAR values calculated accordingly, were subjected to serial dilution and added to SK-BR-3 cells. After a period of 72-96 hr, the cells were evaluated for %-living/dead and this value was plotted vs. the amount of ADC dosed as given in fmol and plotted in a standard dose-response curve %-living on the y-axis and log(fmol) on the x-axis (FIGS. 11-19). While not wishing to be limited by theory, with higher DAR antibodies may aggregate and act as dimers which gives higher apparent avidity. At the highest DAR some mAb precipitate was observed which, while not wishing to be limited by theory is suggestive of aggregation. Chromatography can be readily used by the skilled person to assess whether there is aggregation into dimers.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION

Andera, L.; Wasylyk, B., Mol. Med. 1997, 3 (12), 852-863.
Anima, Y.; Nitta, M.; Kuninaka, S.; Zhang, D. W.; Fujiwara, T.; Taya, Y.; Nakao, M.; Saya, H., J Biol. Chem. 2005, 280 (19), 19166-19176.
Baumann, K.; Munter, K.; Faulstich, H., Biochemistry 1993, 32 (15), 4043-4050.
Blanc, A.; Xia, F.; Todorovic, M.; Perrin, D. M., Amino Acids 2017, 49 (2), 407-414.
Bodero, L.; Rivas, P. L.; Korsak, B.; Hechler, T.; Pahl, A.; Muller, C.; Arosio, D.; Pignataro, L.; Gennari, C.; Piarulli, U., Beilstein J Org. Chem. 2018, 14, 407-415.
Brueckner, F.; Cramer, P., Nat. Struct. Mol. Biol. 2008, 15 (8), 811-818.
Bushnell, D. A.; Cramer, P.; Kornberg, R. D., Proc. Natl. Acad. Sci. USA 2002, 99 (3), 1218-1222.
Casse, C.; Giannoni, F.; Nguyen, V. T.; Dubois, M. F.; Bensaude, O., J. Biol. Chem. 1999, 274 (23), 16097-16106.
Cheung, A. C. M.; Cramer, P., Cell 2012, 149 (7), 1431-1437.
Cochetme, M.; Chambon, P., Biochim. Biophys. Acta 1974, 353 (2), 160-184.
Davis, M. T. B.; Preston, J. F., Science 1981, 213 (4514), 1385-1388.
Figueroa-Vazquez, V.; Ko, J.; Breunig, C.; Baumann, A.; Giesen, N.; Palfi, A.; Muller, C.; Lutz, C.; Hechler, T.; Kulke, M.; Muller-Tidow, C.; Kramer, A.; Goldschmidt, H.; Pahl, A.; Raab, M. S., Mol. Cancer Ther. 2021, 20 (2), 367-378.
Gallo, F.; Korsak, B.; Muller, C.; Hechler, T.; Yanakieva, D.; Avrutina, O.; Kolmar, H.; Pahl, A., J. Med. Chem. 2021, 64 (7), 4117-4129.
Gerets, H. H. J.; Tilmant, K.; Germ, B.; Chanteux, H.; Depelchin, B. O.; Dhalluin, S.; Atienzar, F. A. Characterization of primary human hepatocytes, HepG2 cells, and HepaRG cells at the mRNA level and CYP activity in response to inducers and their predictivity for the detection of human hepatotoxins. Cell Biol. Toxicol. 2012, 28, 69-87.
Hambira, C. M.; Matinkhoo, K.; Pryyma, A.; Patrick, B. O.; Perrin, D. M., J Org. Chem. 2021, 86 (7), 5362-5370.
Kedinger, C.; Gniazdow, M.; Mandel, J. L.; Gissinge, F.; Chambon, P., Biochem. Biophys. Res. Commun. 1970, 38 (1), 165-71.
Lee, K. B.; Wang, D.; Lippard, S. J.; Sharp, P. A., Proc. Natl. Acad. Sci. USA 2002, 99 (7), 4239-4244.
Li, Y. J.; Liu, Y. H.; Xu, H. C.; Jiang, G. L.; Van der Jeught, K.; Fang, Y. Z.; Zhou, Z. L.; Zhang, L.; Frieden, M.; Wang, L. F.; Luo, Z. H.; Radovich, M.; Schneider, B. P.; Deng, Y. B.; Liu, Y. L.; Huang, K.; He, B.; Wang, J.; He, X. M.; Zhang, X. N.; Ji, G.; Lu, X. B., Nat. Commun. 2018, 9, 15.
Li, Y. J.; Sun, Y. F.; Kulke, M.; Hechler, T.; Van der Jeught, K.; Dong, T. H.; He, B.; Miller, K. D.; Radovich, M.; Schneider, B. P.; Pahl, A.; Zhang, X. N.; Lu, X. B., Sci. Transl. Med. 2021, 13 (580), 17.
Liu, X. Y.; Farnung, L.; Wigge, C.; Cramer, P., J. Biol. Chem. 2018, 293 (19), 7189-7194.
Luo, H.; Hong, S. Y.; Sgambelluri, R. M.; Angelos, E.; Li, X.; Walton, J. D., Chem. Biol. 2014, 21 (12), 1610-1617.
Luo, H.; DuBois, B.; Sgambelluri, R. M.; Angelos, E. R.; Li, X.; Holmes, D.; Walton, J. D., Toxicon 2015, 103, 60-64.
Lutz, C.; Simon, W.; Werner-Simon, S.; Pahl, A.; Muller, C., Angew. Chem.-Int. Edit. 2020, 59 (28), 11390-11393.
Matinkhoo, K.; Pryyma, A.; Todorovic, M.; Patrick, B. O.; Perrin, D. M., J Am. Chem. Soc. 2018,140 (21), 6513-6517.
Matinkhoo, K.; Wong, A. A.; Hambira, C. M.; Kato, B.; Wei, C.; Hechler, T.; Muller, C.; Braun, A.; Gallo, F.; Pahl, A.; Perrin, D., Chemistry—A European Journal 2021, 27, 10282-10292.
Matinkhoo, K.; Pryyma, A.; Wong, A. A. W. L.; Perrin, D. M. Chem. Commun. 2021b, 57 (75), 9558-9561.
May, J. P.; Fournier, P.; Pellicelli, J.; Patrick, B. O.; Perrin, D. M., J Org. Chem. 2005, 70 (21), 8424-8430.
May, J. P.; Perrin, D. M., Biopolymers 2007, 88 (5), 714-724.
May, J P.; Fournier, P.; Patrick, B. O.; Perrin, D. M., Chem.-Eur. J 2008, 14 (11), 3410-3417.
McMechen, M. A.; Willis, E. L.; Gourville, P. C.; Proulx, C., Molecules 2019, 24 (10), 12.
Moldenhauer, G.; Salnikov, A. V.; Luttgau, S.; Herr, I.; Anderl, J.; Faulstich, H., J Natl. Cancer Inst. 2012, 104 (8), 622-634.
Pahl, A.; Lutz, C.; Hechler, T., Drug Discov Today Technol 2018, 30, 85-89.
Park, S.; Kim, S. Y.; Cho, J.; Jung, D.; Seo, D.; Lee, J.; Lee, S.; Yun, S.; Lee, H.; Park, O.; Seo, B.; Kim, S.; Seol, M.; Woo, S. H.; Park, T. K., Bioconjug. Chem. 2019a, 30 (7), 1969-1978.
Park, S.; Kim, S. Y.; Cho, J.; Jung, D.; Seo, D.; Lee, J.; Lee, S.; Yun, S.; Lee, H.; Park, O.; Seo, B.; Woo, S. H.; Park, T. K., Bioconjug. Chem. 2019b, 30 (7), 1957-1968.
Pryyma, A.; Matinkhoo, K.; Wong, A.; Perrin, D. M., Chem. Sci. 2020, 11 (43), 11927-11935.
Schneider, C. A.; Rasband, W. S.; Eliceiri, K. W. Nat. Methods 2012, 9 (7), 671-675.
Schuresko, L. A.; Lokey, R. S., Angew. Chem.-Int. Edit. 2007, 46 (19), 3547-3549.
Siegert, M. A. J.; Knittel, C. H.; Sussmuth, R. D., Angew. Chem.-Int. Edit. 2020, 59 (14), 5500-5504.
Vetter, J., Toxicon 1998, 36 (1), 13-24.
Wang, D.; Bushnell, D. A.; Huang, X. H.; Westover, K. D.; Levitt, M.; Kornberg, R. D., Science 2009, 324 (5931), 1203-1206.
Wieland, T.; Faulstich, H., Experientia 1991, 47 (11-12), 1186-1193.
Wieland, T.; Gotzendorfer, C.; Dabrowski, J.; Lipscomb, W. N.; Shoham, G., Biochemistry 1983, 22 (5), 1264-1271.
Yao, G. Y.; Knittel, C. H.; Kosol, S.; Wenz, M. T.; Keller, B. G.; Gruss, H.; Braun, A. C.; Lutz, C.; Hechler, T.; Pahl, A.; Sussmuth, R. D., J. Am. Chem. Soc. 2021, 143 (35), 14322-14331.
Zanotti, G.; Mohringer, C.; Wieland, T., Int. J Pept. Protein Res. 1987, 30 (4), 450-459.
Zanotti, G.; Birr, C.; Wieland, T. Int. J. Pept. Protein Res. 1981, 18, 162-168.

TABLE 1
Exemplary Amatoxins.

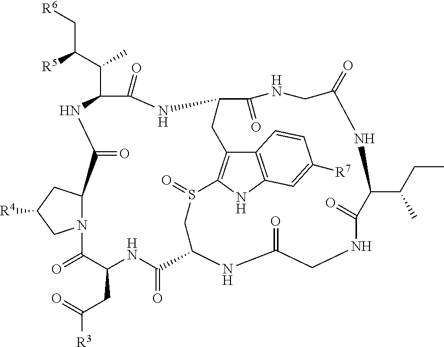

| | R³ | R⁴ | R⁵ | R⁶ | R⁷ | Kᵢ (nM)* | LD₅₀ (mg/kg)** |
|---|---|---|---|---|---|---|---|
| α-amanitin | NH₂ | OH | OH | OH | OH | 2.3 | 0.3 |
| β-amanitin | OH | OH | OH | OH | OH | 2.5 | 0.5 |
| γ-amanitin | NH₂ | OH | OH | H | OH | 5 | 0.2 |
| ε-amanitin | OH | OH | OH | H | OH | — | 0.3-0.6 |
| amanin | OH | OH | OH | OH | H | 5 | 0.3 |
| amaninamide | NH₂ | OH | OH | OH | H | 5 | 20 |
| amanullin | NH₂ | OH | H | H | OH | 10 | >20 |
| amanullinic acid | OH | OH | H | H | OH | — | >20 |
| proamanullin | OH | H | H | H | OH | — | >20 |

*Determined against calf thymus RNAP II.
**As observed in white mice.

TABLE 5

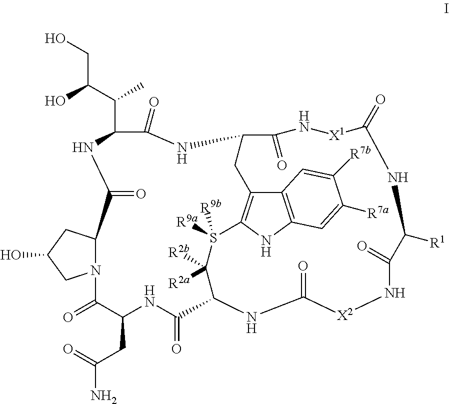

| | | | | | | | | | | | IC₅₀ |
|---|---|---|---|---|---|---|---|---|---|---|---|
| | | | | | | | | | | IC₅₀ | (Pol II) |
| Compound | X¹ | X² | R¹ | R^{2a} | R^{2b} | R7^{a} | R7^{b} | R5^{a} | R^{5b} | (CHO) | normalized |
| S. amanatin | CH | CH | 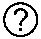 | H | | OH | H | — | O | [symbol] | 1 |
| S-Deoxy S-Deoxy Amarmamide | CH | CH | 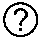 | H | H | H | H | — | — | 1.3* | 10* |
| I(i) | CH | CH | 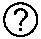 | H | CH | H | H | — | — | [symbol] | 10 |
| 2R,3S-β-Methyl-Cy [symbol] - | | | | | | | | | | | |
| Dideoxyamanitin | | | | | | | | | | | |
| I(ii) | CH | CH | 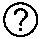 | CH | H | H | H | — | — | >22 | >128 |
| 2R,3R-β-Methyl-Cy [symbol] - | | | | | | | | | | | |
| Dideoxyamanitin | | | | | | | | | | | |
| I(ii) | CH | CH | 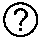 | CH | CH | H | H | — | — | 14.1 | [symbol] |
| Pen-Dideoxyamanitin | | | | | | | | | | | |
| I(iv) | CH | NH | 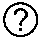 | H | H | H | H | — | — | [symbol] | 4.7 |
| aza-Gly-Dideoxyamanitin | | | | | | | | | | | |
| I(v) | CH | CH | 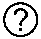 | H | H | H | H | — | — | 6.5 | 8.6 |
| Thia-Ile-Dideoxyamaritin | | | | | | | | | | | |
| I(vi) | CH | CH | 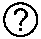 | H | H | H | H | — | — | >22 | 42 |
| Oxo-Ile-Dideoxyamanitin | | | | | | | | | | | |
| I(vii) | CH | CH | 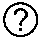 | H | H | H | H | — | — | 0.80 | 8.5 |
| Diethyl-Ala- | | | | | | | | | | | |
| Dideoxyamanitin | | | | | | | | | | | |
| I(viii) | CH | CH | 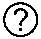 | H | H | H | H | — | — | 5.4 | 4 [symbol] |
| Cyclopentylgly [symbol] | | | | | | | | | | | |
| Dideoxyamanitin | | | | | | | | | | | |
| I(ix) | NH | CH | 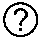 | H | H | H | H | — | — | 2.1 | 5.3 |
| aza-Gly-Dideoxyamanine | | | | | | | | | | | |
| I(x) | CH | CH | 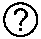 | H | CH | H | OH | — | — | 6.9 | [symbol] |
| 5-OH Trp', 2R,3S β-Methyl- | | | | | | | | | | | |
| Cys [symbol] Deoxyaminitin | | | | | | | | | | | |
| I(xi) | CH | CH | 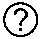 | H | H | H | OH | — | — | 2.4 | 17.8 |
| 5-OH Trp'-Diethylala' - | | | | | | | | | | | |
| Deoxyamanitin | | | | | | | | | | | |
| I(xii) | CH | CH | 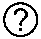 | H | CH | H | H | — | — | 0.60 | 9.5 |
| Diethylala [symbol] 2R,3S β | | | | | | | | | | | |
| Methyl-Cys- | | | | | | | | | | | |
| Dideoxyamantin | | | | | | | | | | | |
| I(xiii) | CH | CH | 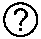 | H | CH | H | OH | — | — | 2.4 | 13.8 |
| 5-OH Trp [symbol] , Diethylala | | | | | | | | | | | |
| 2R,3S β-Methyl-Cys [symbol] | | | | | | | | | | | |
| Deoxyamanitin | | | | | | | | | | | |

*Wieland et al.. 1983. [symbol]
indicates data missing or illegible when filed

TABLE 6
Transcription activities of HeLa nuclear extract in varying amanitin analog concentrations (n = 3).

| Conc. | Transcription Activity [symbol] | | | | | | | | | | | | | |
|---|---|---|---|---|---|---|---|---|---|---|---|---|---|---|
| (Nm) | [symbol] | I(i) | I(ii) | I(ii) | I(iv) | I(v) | I(vi) | I(vii) | I(viii) | I(ix) | I(x) | I(xi) | I(xii) | I(xiii) |
| 1 | 85.0 = 2.4 | — | — | — | — | — | — | — | — | — | — | — | — | — |
| 2.2 | — | — | — | — | — | — | — | 103 = 8.3 | — | — | — | — | — | — |
| 3 | 69.5 = 9.4 | — | — | — | 125 = 8.6 | — | — | — | — | — | — | — | 91.5 = 13.9 | — |
| 6.7 | — | — | — | — | — | — | — | 103 = 6.0 | — | — | — | — | — | — |
| 10 | 48.4 = 9.8 | [symbol] | — | — | 102 = 23 | 94.3 = 3.3 | — | — | — | 77.3 = 12.4 | 83.9 = 15.7 | 116 = 17 | [symbol] 2 = 10.8 | 101 = 43 |
| 19 | — | — | — | — | — | — | — | — | 101 = 34.5 | — | — | — | — | — |
| 20 | — | — | — | — | — | — | — | 83.6 = 9.2 | — | — | — | — | — | — |
| 26 | — | — | — | 82.7 = 13.0 | — | — | — | — | — | — | — | — | — | — |
| 30 | 22.9 = 2.6 | 68.9 = 22.6 | — | — | [symbol] 5 = 19.9 | 68.7 = 9.2 | 94.5 [symbol] | — | — | [symbol] 1.3 = 6.5 | 6 [symbol] 9 = 8.6 | 91.5 = 16.1 | 66 [symbol] = 20 [symbol] | 82.0 = 6.6 |
| 56 | — | — | — | — | — | — | — | — | 91.4 = 43.5 | — | — | — | — | — |
| 60 | — | — | — | — | — | — | — | 64 [symbol] 4 = 9.2 | — | — | — | — | — | — |
| [symbol] | — | — | — | 68.0 = 13.5 | — | — | — | — | — | — | — | — | — | — |
| 100 | 9.5 = 4.1 | 45.1 = 18.7 | 84.9 = 10.7 | — | 36.6 = 1 [symbol] | 33 [symbol] = 14.3 | 78.1 = 6.6 | — | — | 26.8 = 7.6 | 46.2 = 6.8 | 59.3 = 16.7 | 42.3 = 13.0 | 48.2 = [symbol] |
| 167 | — | — | — | — | — | — | — | — | [symbol] 0 = 16 [symbol] | — | — | — | — | — |
| [symbol] | — | — | — | — | — | — | — | 24.5 = 2.5 | — | — | — | — | — | — |
| 233 | — | — | — | 49.6 = 15.2 | — | — | — | — | — | — | — | — | — | — |
| 300 | — | 20.2 = 8.9 | — | — | [symbol] | 11.3 = 4.4 | 48.6 = 10.7 | — | — | 12.2 = 1.1 | 30.0 = 6.2 | 31.5 = 7.5 | 20.4 = 2.8 | 25.7 = 2.3 |
| 500 | — | — | — | — | — | — | — | — | 39.1 = 9.2 | — | — | — | — | — |
| 700 | — | — | — | 28.4 = 10.6 | — | — | — | — | — | — | — | — | — | — |
| 1000 | — | 8.6 = 6.1 | — | — | — | [symbol] | 21.8 = 13.6 | — | — | 4.5 = 0.8 | 12.0 = 1.6 | 11.0 = [symbol] .6 | — | 10.7 = 4.3 |
| 1500 | — | — | — | — | — | — | — | — | [symbol] = 3.2 | — | — | — | — | — |
| 2100 | — | — | — | 9.6 = 9.5 | — | — | — | — | — | — | — | — | — | — |
| 3000 | — | — | — | — | — | — | 10.1 = 6.9 | — | — | — | — | — | — | — |

Transcription runoff assay (HeLaScribe [symbol] Nuclear Extract)

| | | | | | | | | | | | | | | |
|---|---|---|---|---|---|---|---|---|---|---|---|---|---|---|
| K (nM) | [symbol] = 09 | [symbol] | >1000 | 216 = 29 | [symbol] | 6 [symbol] = 16 | [symbol] 8 = 23 | 66 = 10 | 366 = 14 | 41 = 2 | 11 [symbol] = 16 | 139 = 28 | [symbol] | 108 = 13 |
| R | 0.991 | 0.98 [symbol] | — | 0.9 [symbol] | 0.999 | 0.981 | 0.996 | 0.968 | 0.990 | 0.986 | 0.949 | 0.96 [symbol] | 0.980 | 0.9 [symbol] |

CHO cell viability assay (MTT)

| | | | | | | | | | | | | | | |
|---|---|---|---|---|---|---|---|---|---|---|---|---|---|---|
| IC (nM) | 500 = 200 | 610 = 1 [symbol] 0 | >15000 | 2700 = [symbol] 90 | 1 [symbol] 0 = 30 | 1140 = [symbol] 0 | >15000 | [symbol] 80=110 | 1 [symbol] 00 = [symbol] | 1900 = [symbol] 20 | 3300 = 1300 | 1200 = 330 | 290 =70 | 1200 = 370 |
| R | — | 0.9 [symbol] | -2.114 | 0. [symbol] | 0.964 | 0.91 [symbol] | [symbol] | 0.911 | 0.890 | 0. [symbol] | 0. [symbol] | 0.9 [symbol] | 0.942 | 0.90 [symbol] |

[symbol]

4 experimental replicates completed, with IC [symbol] values 290, 460, 790, 480 nM. [symbol]
DNC: (regression) did not converge. [symbol]
indicates data missing or illegible when filed

TABLE 7

| | IC₅₀ | IC₅₀ | IC₅₀ | IC₅₀ |
|---|---|---|---|---|
| Compound | (CHO) | (HEK293) | (HeLa) | (HepG2) |
| α-amanitin | 1^{a} | 1^{b} | 1^{c} | 1^{d} |
| I(i) | 1.3 | 0.51 | — | — |
| 2R,3S-β-Methyl-Cys⁸-Dideoxyamanitin | | | | |
| I(ii) | >20 | — | — | — |
| 2R,3R-β-Methyl-Cys⁸-Dideoxyamanitin | | | | |
| I(iii) | 9.3 | — | — | — |
| Pen⁸-Dideoxyamanitin | | | | |
| I(iv) | 0.38 | 0.46 | 0.83 | 2.7 |
| aza-Gly⁷-Dideoxyamanitin | | | | |
| I(v) | 5.6 | 1.5 | 4.0 | >10 |
| Thia-Ile⁶-Dideoxyamanitin | | | | |
| I(vi) | >20 | — | — | — |
| Oxo-Ile⁶-Dideoxyamanitin | | | | |
| I(vii) | 0.78 | 0.37 | — | — |
| Diethyl-Ala⁶-Dideoxyamanitin | | | | |
| I(viii) | 6.9 | — | — | — |
| Cyclopentylgly⁶-Dideoxyamanitin | | | | |
| I(ix) | 2.4 | 0.45 | — | — |
| aza-Gly⁵-Dideoxyamanitin | | | | |
| I(x) | 6.9 | 0.88 | — | — |
| 5-OH Trp⁴, 2R,3S β-Methyl-Cys⁸-Deoxyamanitin | | | | |
| I(xi) | 2.5 | 0.32 | — | — |
| 5-OH Trp⁴, Diethylala⁶-Deoxyamanitin | | | | |
| I(xii) | 0.60 | 0.20 | 0.60 | 2.7 |
| Diethylala⁶, 2R,3S β-Methyl-Cys⁸- | | | | |
| Dideoxyamanitin | | | | |
| I(xiii) | 2.4 | 0.51 | — | — |
| 5-OH Trp⁴, Diethylala⁶, 2R,3S β-Methyl-Cys⁸- | | | | |
| Deoxyamanitin | | | | |

* Wieland et al., 1983.
^{a}The average IC₅₀ value for α-amanitin on CHO cells repeated in triplicate 4 times; see Table 8.
^{b}The average IC₅₀ value for α-amanitin on HEK293 cells repeated in triplicate 3 times; see Table 8.
^{c}The average IC₅₀ value for α-amanitin on HeLa cells performed in triplicate once; see Table 8.
^{d}The average IC₅₀ value for amanitin on HepG2 cells performed in triplicate once; see Table 8. Relative IC₅₀ values are given for amatoxins.

TABLE 8

| | IC₅₀ | IC₅₀ | IC₅₀ | IC₅₀ |
|---|---|---|---|---|
| Compound | (CHO, nM) | (HEK293, nM) | (HeLa, nM) | (HepG2, nM) |
| α-amanitin | 790 ± 120^{a} | 560 ± 100^{e} | 4000 ± 410 | 2100 ± 260 |
| | 290 ± 35^{b} | 470 ± 90^{f} | | |
| | 480 ± 130^{c} | 260 ± 40^{g} | | |
| | 450 ± 100^{d} | | | |
| I(i) | 610 ± 170^{c} | 240 ± 50^{f} | — | — |
| 2R,3S-β-Methyl-Cys⁸- | | | | |
| Dideoxyamanitin | | | | |
| I(ii) | >15000^{a} | — | — | — |
| 2R,3R-β-Methyl-Cys⁸- | | | | |
| Dideoxyamanitin | | | | |
| I(iii) | 2700 ± 590^{b} | — | — | — |
| Pen⁸-Dideoxyamanitin | | | | |
| I(iv) | 170 ± 30^{d} | 260 ± 70^{e} | 3300 ± 390 | 5600 ± 780 |
| aza-Gly⁷-Dideoxyamanitin | | | | |
| I(v) | 4400 ± 730^{a} | 830 ± 150^{e} | 16000 ± 2300 | >20000 |
| Thia-Ile⁶-Dideoxyamanitin | | | | |
| I(vi) | >15000^{a} | — | — | — |
| Oxo-Ile⁶-Dideoxyamanitin | | | | |
| I(vii) | 380 ± 110^{c} | 170 ± 30^{f} | — | — |
| Diethyl-Ala⁶-Dideoxyamanitin | | | | |
| I(viii) | 1700 ± 270^{b} | — | — | — |
| Cyclopentylgly⁶- | | | | |
| Dideoxyamanitin | | | | |
| I(ix) | 1900 ± 520^{a} | 210 ± 50^{f} | — | — |
| aza-Gly⁵-Dideoxyamanitin | | | | |
| I(x) | 3300 ± 1300^{c} | 230 ± 30^{g} | — | — |
| 5-OH Trp⁴, 2R,3S β-Methyl- | | | | |
| Cys⁸-Deoxyamanitin | | | | |
| I(xi) | 1200 ± 330^{c} | 80 ± 10^{g} | — | — |
| 5-OH Trp⁴, Diethylala⁶- | | | | |
| Deoxyamanitin | | | | |
| I(xii) | 290 ± 70^{c} | 110 ± 30^{e} | 2400 ± 300 | 5700 ± 1100 |
| Diethylala⁶, 2R,3S β-Methyl- | | | | |
| Cys⁸-Dideoxyamanitin | | | | |
| I(xiii) | 1170 ± 370^{c} | 240 ± 50^{f} | — | — |
| 5-OH Trp⁴, Diethylala⁶, 2R,3S β- | | | | |
| Methyl-Cys⁸-Deoxyamanitin | | | | |

^{a, b, c, d, e, f, g}are different individual assay runs.

What is claimed is:

1. An amatoxin analog comprising at least one of the following eastern ring residues:
(a) a Gly⁵ residue of the formula: 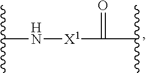
wherein X¹ is (CH₂)ₘ, CHCH₃, CHCH₂CH₃, C(CH₃)₂ or NR′;
(b) an Ile⁶ residue of the formula: 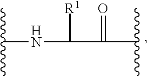
wherein R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S;
each Z is independently (CH₂)ₘO(CH₂)ₚ, (CH₂)ₙS(CH₂)ₚ, (CH₂)ₙSe(CH₂)ₚ or (CH₂)_{q};
each n is independently an integer from 0 to 4;
each p is independently an integer from 0 to 4;
n+p in each Z, is independently ≤4;
each q is independently an integer from 0 to 4; and
wherein one or more single bonds between two available carbon atoms in R¹ is/are optionally replaced by a double bond;
(c) a Gly⁷ residue of the formula: 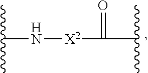
wherein X² is (CH₂)ₘ, CHCH₃, CHCH₂CH₃, C(CH₃)₂ or NR′; and
(d) a Cys⁸ residue of the formula: 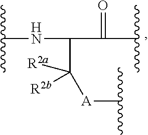
wherein
A is S, S(O), SO₂, Se, Se(O) or SeO₂; and
R^{2a} and R^{2b} are each independently H or C₁₋₁₀alkyl; or R^{2a} and R^{2b} together with the carbon atom to which they are attached, together form C₃₋₁₀cycloalkyl;
each m is independently an integer from 1 to 5; and
each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl,
or a pharmaceutically acceptable salt thereof,
provided that the amatoxin analog or the pharmaceutically acceptable salt thereof has one or more of the following:
(i) R¹ other than 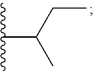 [SMILES: *C(C)CC]
(ii) X¹ other than CH₂;
(iii) X² other than CH₂;
(iv) R^{2a} other than H; and
(v) R^{2b} other than H, and
wherein in the amatoxin analog or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

2. The amatoxin analog of claim 1, that is a compound of Formula I: 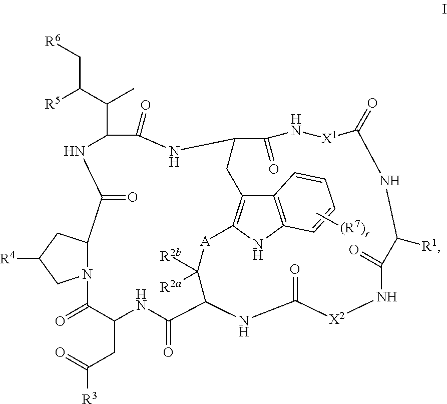
wherein
A is S, S(O), SO₂, Se, Se(O) or SeO₂;
X¹ and X² are each independently (CH₂)ₘ, CHCH₃, CHCH₂CH₃, C(CH₃)₂ or NR′;
R¹ is CH(Z—CH₃)₂, wherein one or more available hydrogens is/are optionally substituted with SH; or R¹ is C₃₋₁₀cycloalkyl, wherein one or more available hydrogens on the C₃₋₁₀cycloalkyl is/are optionally substituted with C₁₋₁₀alkyl and/or one or more available carbon atoms in the C₃₋₁₀cycloalkyl is/are optionally replaced by O or S; and wherein one or more single bonds between two available carbon atoms in R¹ is/are optionally replaced by a double bond;
R^{2a} and R^{2b} are each independently H or C₁₋₁₀alkyl; or R^{2a} and R^{2b} together with the carbon atom to which they are attached, together form C₃₋₁₀cycloalkyl;
R³ is OR′, NH(OR′), NH—NHR′ or N(R′)₂;
R⁴ is H or OH;
R⁵ and R⁶ are each independently H, OR′, NHR′, SR′ or halo;
each R⁷ is independently OR′, N(R′)₂, NO₂, SR′, CN, COOR′, N₃, NR′NR′, ON(R′)₂, NR′—OR′, SeR′, SO₃R′, P(O)(OR′)₂, C(O)N(R′)₂, CHO, B(OR′)₂, CH₃, CH₂OH, or halo;
each Z is independently (CH₂)ₙO(CH₂)ₚ, (CH₂)ₙS(CH₂)ₚ, (CH₂)ₙSe(CH₂)ₚ or (CH₂)_{q};
each m is independently an integer from 1 to 5;
each n is independently an integer from 0 to 4;
each p is independently an integer from 0 to 4;
n+p in each Z, is independently ≤4;
each q is independently an integer from 0 to 4;
r is an integer from 0 to 4; and
each R′ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, wherein one or more available carbon atoms in the C₁₋₂₀alkyl, C₂₋₂₀alkenyl and C₂₋₂₀alkynyl is optionally replaced by a heteroatom, and wherein the C₁₋₂₀alkyl, C₂₋₂₀alkenyl, C₂₋₂₀alkynyl, C₃₋₂₀cycloalkyl, C₆₋₃₀aryl, heterocycloalkyl and heteroaryl is optionally further substituted with one or more groups selected from OR″, SR″, halo, azide and nitrile, wherein R″ is independently H, C₁₋₂₀alkyl, C₂₋₂₀alkenyl, C₂₋₂₀alkynyl, C₃₋₂₀cycloalkyl, C₆₋₃₀aryl, heterocycloalkyl or heteroaryl, or a pharmaceutically acceptable salt thereof,
provided that the compound of Formula I or the pharmaceutically acceptable salt thereof has one or more of the following:
(i) R¹ other than 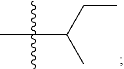 [SMILES: *C(C)CC]
(ii) X¹ other than CH₂;
(iii) X² other than CH₂;
(iv) R^{2a} other than H; and
(v) R^{2b} other than H, and
wherein in the compound of Formula I or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

3. The amatoxin analog of claim 2, having the following stereochemistry:

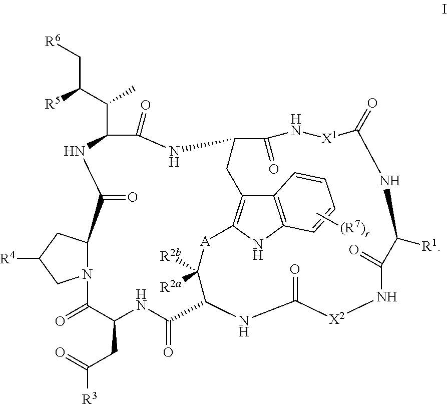

4. The amatoxin analog of claim 2 or 3, wherein R³ is NH₂.

5. The amatoxin analog of any one of claims 2 to 4, wherein R⁴, R⁵ and R⁶ are each OH.

6. The amatoxin analog of any one of claims 2 to 5, wherein r is 0.

7. The amatoxin analog of any one of claims 2 to 5, wherein r is 1.

8. The amatoxin analog of claim 7, wherein R⁷ is 4-OH, 5-OH, 6-OH or 7-OH.

9. The amatoxin analog of claim 8, wherein R⁷ is 5-OH.

10. The amatoxin analog of any one of claims 1 to 9, wherein R¹ is:
(a) CH(Z—CH₃)₂ other than 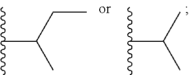
or
(b) unsubstituted C₃₋₁₀cycloalkyl.

11. The amatoxin analog of claim 10, wherein R¹ is 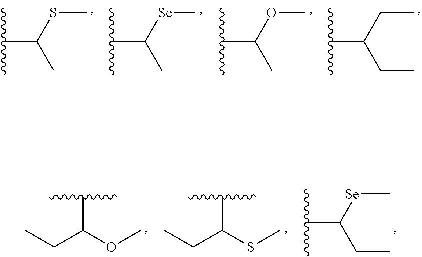
cyclopentyl, 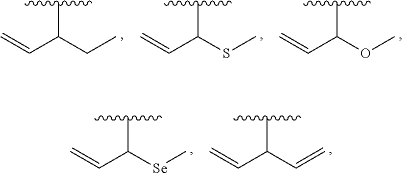
tetrahydrofuranyl, or tetrahydrothiophenyl.

12. The amatoxin analog of claim 10, wherein R¹ is

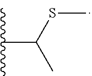 [SMILES: *C(C)SC]

13. The amatoxin analog of claim 10, wherein R¹ is

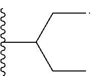 [SMILES: *C(CC)CC]

14. The amatoxin analog of any one of claims 1 to 13, wherein X¹ is NH.

15. The amatoxin analog of any one of claims 1 to 14, wherein X² is NH.

16. The amatoxin analog of any one of claims 1 to 14, wherein X² is N—CH(CH₃)₂.

17. The amatoxin analog of any one of claims 1 to 16, wherein at least one of R^{2a} and R^{2b} is C₁₋₁₀alkyl.

18. The amatoxin analog of claim 17, wherein R^{2a} is CH₃ and R^{2b} is CH₃.

19. The amatoxin analog of claim 17, wherein R^{2a} is H and R^{2b} is CH₃.

20. The amatoxin analog of any one of claims 1 to 19, wherein A is S or (R)—S(O).

21. The amatoxin analog of claim 20, wherein A is S.

22. The amatoxin analog of claim 20, wherein A is (R)—S(O).

23. The amatoxin analog of claim 1, wherein the amatoxin analog is:

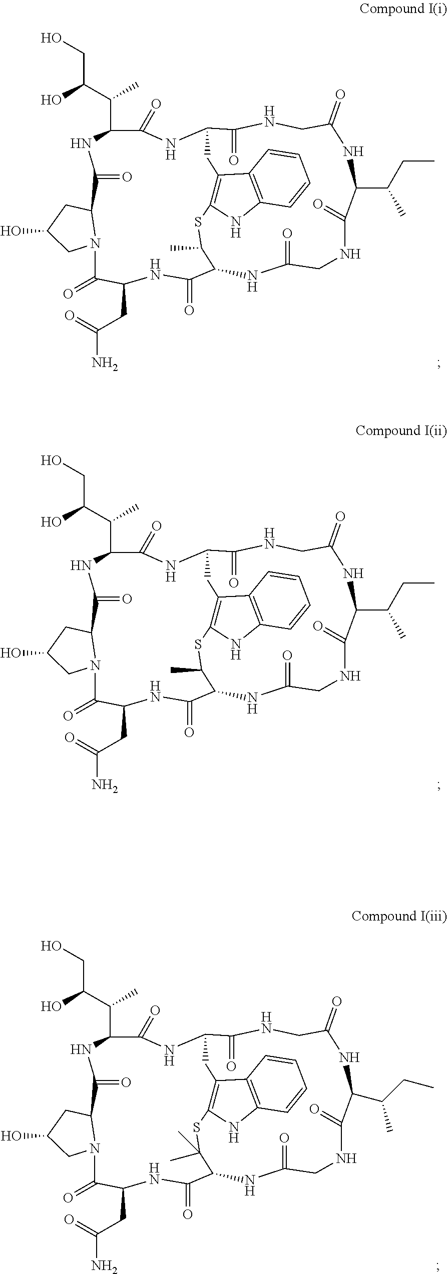

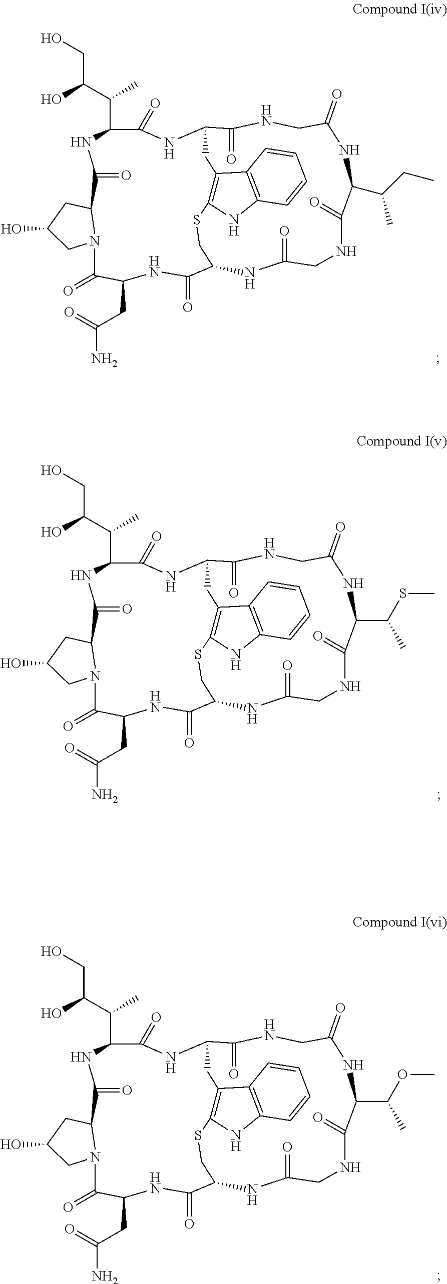 [SMILES: CC[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; CS[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; CO[C@H](C)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

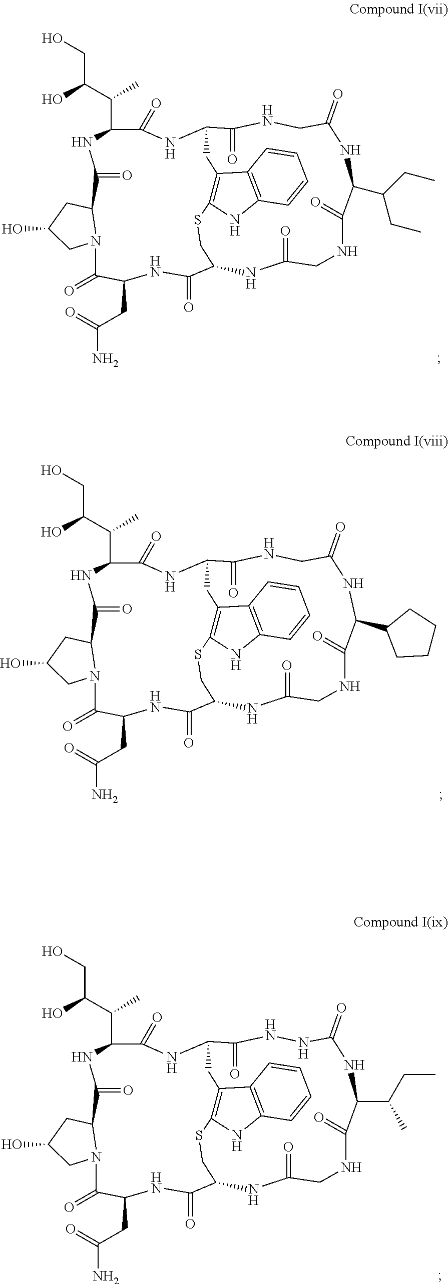 [SMILES: CCC(CC)[C@@H]1NC(=O)CNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2 ; C[C@H]([C@@H]1NC(=O)[C@@H]2C[C@@H](O)CN2C(=O)[C@H](CC(N)=O)NC(=O)[C@@H]2CSc3[nH]c4ccccc4c3C[C@@H](NC1=O)C(=O)NCC(=O)N[C@@H](C1CCCC1)C(=O)NCC(=O)N2)[C@@H](O)CO ; CC[C@H](C)[C@@H]1NC(=O)NNC(=O)[C@H]2Cc3c([nH]c4ccccc34)SC[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

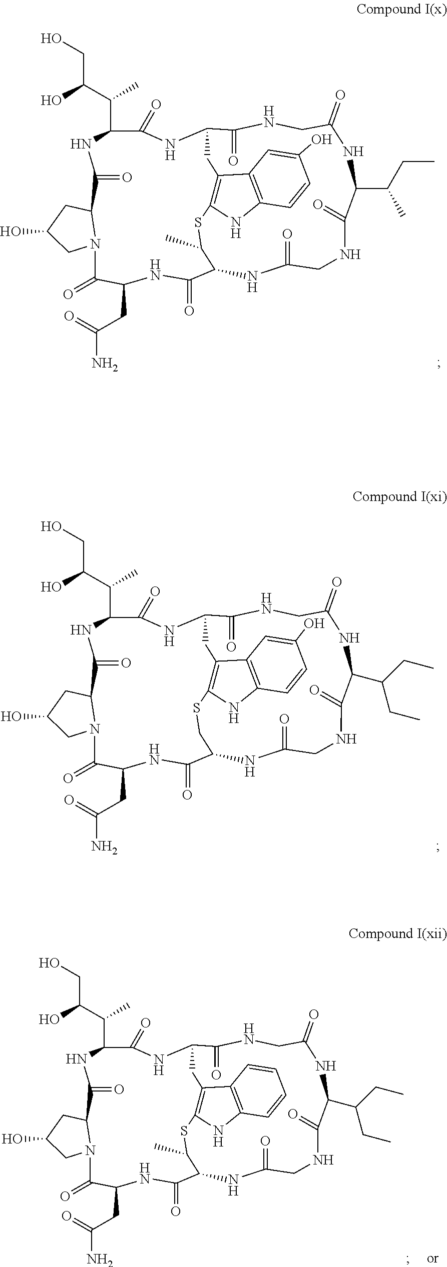

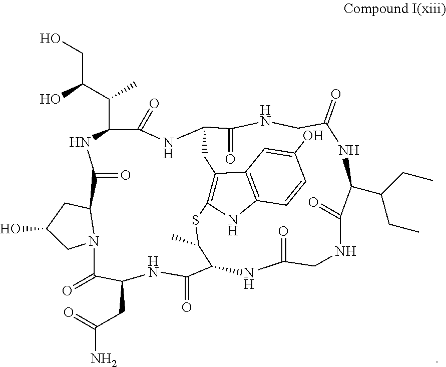 [SMILES: CCC(CC)[C@@H]1NC(=O)CNC(=O)[C@H]2Cc3c([nH]c4ccc(O)cc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

24. The amatoxin analog of claim 1, wherein the amatoxin analog is:

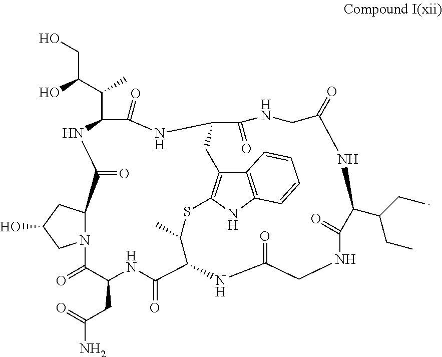 [SMILES: CCC(CC)[C@@H]1NC(=O)CNC(=O)[C@H]2Cc3c([nH]c4ccccc34)S[C@@H](C)[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

25. The amatoxin analog of claim 1, wherein the amatoxin analog is:

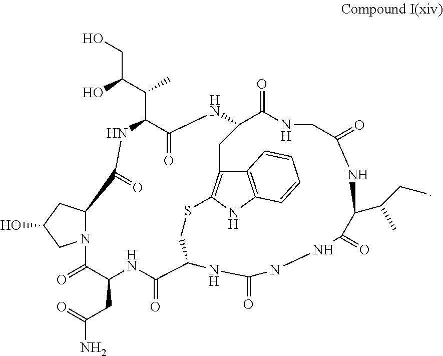 [SMILES: CC[C@H](C)[C@@H]1NC(=O)CNC(=O)[C@@H]2Cc3c([nH]c4ccccc34)SC[C@H](NC(=O)NNC1=O)C(=O)N[C@@H](CC(N)=O)C(=O)N1C[C@H](O)C[C@H]1C(=O)N[C@@H]([C@@H](C)[C@@H](O)CO)C(=O)N2]

26. The amatoxin analog of claim 1, that is a compound of Formula II: 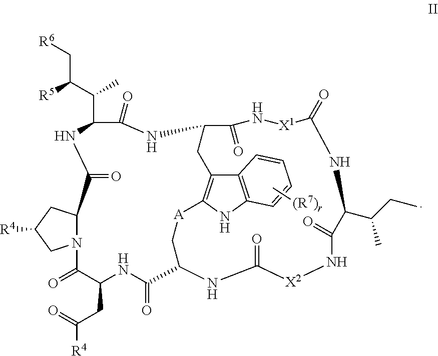
wherein
X¹ and X² are each independently (CH₂)ₘ, CHCH₃, CHCH₂CH₃, C(CH₃)₂ or NR′, wherein m is an integer from 1 to 5 and provided that at least one of X¹ and X² is NR′; and
A, R′, R³, R⁴, R⁵, R⁶, R⁷ and r are as defined in any one of claims 2, 4 to 9 and 20 to 22,
or a pharmaceutically acceptable salt thereof,
wherein in the compound of Formula II or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

27. The amatoxin analog of claim 26, wherein X¹ is NH.

28. The amatoxin analog of claim 26, wherein X² is NH.

29. The amatoxin analog of claim 26, wherein X² is N—CH(CH₃)₂.

30. The amatoxin analog of claim 1, that is a compound of Formula III: 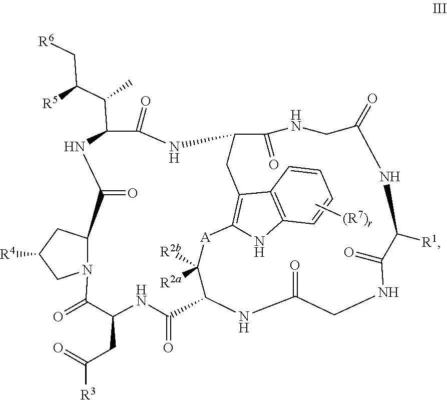
wherein
A, R¹, R³, R⁴, R⁵, R⁶, R⁷ and r are as defined in any one of claims 2, 4 to 13 and 20 to 22; and
R^{2a} and R^{2b} are each independently H or C₁₋₁₀alkyl, provided that at least one of R^{2a} and R^{2b} is C₁₋₁₀alkyl; or R^{2a} and R^{2b} together with the carbon atom to which they are attached, together form C₃₋₁₀cycloalkyl,
or a pharmaceutically acceptable salt thereof,
wherein in the compound of Formula III or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

31. The amatoxin analog of claim 30, wherein R¹ is

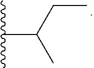 [SMILES: *C(C)CC]

32. The amatoxin analog of claim 30, wherein R¹ is

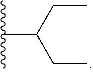 [SMILES: *C(CC)CC]

33. The amatoxin analog of any one of claims 30 to 32, wherein R^{2a} and R^{2b} are as defined in any one of claims 17 to 19.

34. The amatoxin analog of claim 1, that is a compound of Formula IV: 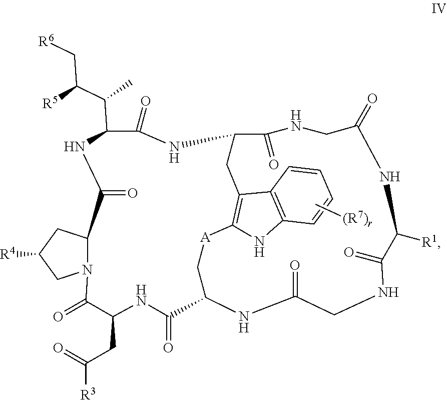
wherein
R¹ is CH(Z—CH₃)₂, wherein one or more available hydrogens is/are optionally substituted with SH; or R¹ is C₃₋₁₀cycloalkyl, wherein one or more available hydrogens on the cycloalkyl are optionally substituted with C₁₋₁₀alkyl and/or one or more available carbons in the cycloalkyl is optionally replaced by O or S; and wherein one or more single bonds between two available carbon atoms in R¹ is/are optionally replaced by a double bond;
each Z is independently (CH₂)ₙO(CH₂)ₚ, (CH₂)ₙS(CH₂)ₚ, (CH₂)ₙSe(CH₂)ₚ or (CH₂)_{q};
each n is independently an integer from 0 to 4;
each p is independently an integer from 0 to 4;
n+p in each Z, is independently ≤4;
each q is independently an integer from 0 to 4,
provided that R¹ is other than 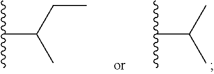
and
A, R³, R⁴, R⁵, R⁶, R⁷ and r are as defined in any one of claims 2, 4 to 9 and 20 to 22, or a pharmaceutically acceptable salt thereof,
wherein in the compound of Formula IV or the pharmaceutically acceptable salt thereof, one or more available hydrogens is/are optionally replaced with deuterium and/or one or more available hydrogens is/are optionally replaced with fluorine and/or one or more available atoms is/are optionally replaced with its radioactive isotope.

35. A compound-linker construct comprising the amatoxin analog of any one of claims 1 to 34 coupled to a linker, wherein the linker comprises a reactive group R⁸ for conjugating the compound-linker construct to a target-binding moiety.

36. The compound-linker construct of claim 35, wherein the linker is coupled to the amatoxin analog or the salt thereof at a site indicated by R³, R⁴, R⁵, R⁶ and/or R⁷ in an amatoxin analog as defined in any one of claims 2 to 34.

37. The compound-linker construct of claim 35 or 36, wherein the linker is coupled to the amatoxin analog or the salt thereof through a moiety obtained from the reaction of R³, R⁴, R⁵, R⁶ and/or R⁷ as defined in any one of claims 2 to 34 with a complementary functional group on the linker.

38. The compound-linker construct of claim 35,
wherein at a site indicated by R³, R⁴, R⁵, R⁶ and/or R⁷ in the amatoxin analog as defined in any one of claims 2 to 34, the compound-linker construct comprises a group of the formula —O-L, wherein L represents the linker;
wherein at a site indicated by R³, R⁴, R⁵, R⁶ and/or R⁷ in the amatoxin analog as defined in any one of claims 2 to 34, the compound-linker construct comprises a group of the formula —O—C(O)-L, wherein L represents the linker; or
wherein at a site indicated by R³, R⁵, R⁶ and/or R⁷ in the amatoxin analog as defined in any one of claims 2 to 34, the compound-linker construct comprises a group of the formula-NR′-L, wherein R′ is as defined in claim 2 and L represents the linker.

39. The compound-linker construct of any one of claims 35 to 38, wherein the linker is a stable linker.

40. The compound-linker construct of any one of claims 35 to 38, wherein the linker is a cleavable linker.

41. The compound-linker construct of claim 40, wherein the linker further comprises a self-immolating moiety.

42. The compound-linker construct of any one of claims 35 to 38, wherein the linker is cleavable by at least one agent selected from the group consisting of cysteine protease, metalloproteinase, serine protease, threonine protease, aspartic protease, glycosidase, phosphodiesterase, and reductase.

43. The compound-linker construct of any one of claims 35 to 38, wherein the linker comprises a motif selected from the group consisting of: Val-Ala, Val-Cit, Val-Lys, Val-Arg, Phe-Lys-Gly-Pro-Leu-Gly, Ala-Ala-Pro-Val, β-glucuronide and β-galactoside.

44. The compound-linker construct of any one of claims 35 to 38, wherein:
(a) at a site indicated by R³, R⁴, R⁵, R⁶ or R⁷ in the amatoxin analog as defined in any one of claims 2 to 34, the compound-linker construct comprises a group of the formula: 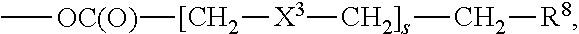
wherein X³ is O, CH₂ or S, s is an integer from 1 to 8 and R⁸ is as defined in claim 35;
(b) at a site indicated by R³, R⁵, R⁶ or R⁷ in the amatoxin analog as defined in any one of claims 2 to 34, the compound-linker construct comprises a group of the formula: 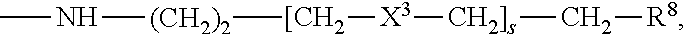
wherein X³ is O, CH₂ or S, s is an integer from 1 to 8 and R⁸ is as defined in claim 35;
(c) at a site indicated by R³, R⁵, R⁶ or R⁷ in the amatoxin analog as defined in any one of claims 2 to 34, the compound-linker construct comprises a group of the formula: 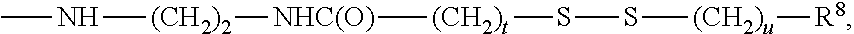
wherein t and u are independently an integer from 1 to 6 and R⁸ is as defined in claim 35;
(d) at a site indicated by R³, R⁵, R⁶ or R⁷ in the amatoxin analog as defined in any one of claims 2 to 34, the compound-linker construct comprises a group of the formula: 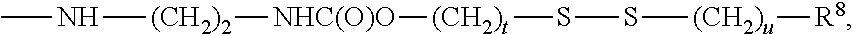
wherein t and u are independently an integer from 1 to 6 and R⁸ is as defined in claim 35;
(e) at a site indicated by R³, R⁵, R⁶ or R⁷ in the amatoxin analog as defined in any one of claims 2 to 34, the compound-linker construct comprises a group of the formula: 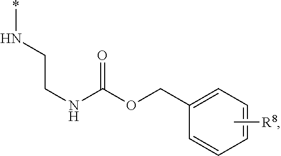
wherein * represents the site of attachment to the remainder of the compound-linker construct and R⁸ is as defined in claim 35;
(f) at a site indicated by R³, R⁵, R⁶ or R⁷ in the amatoxin analog as defined in any one of claims 2 to 34, the compound-linker construct comprises a group of the formula: 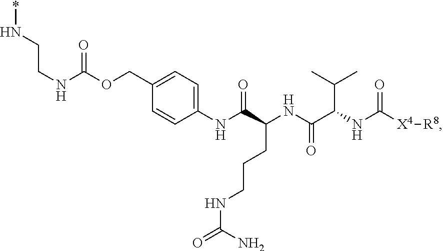
wherein * represents the site of attachment to the remainder of the compound-linker construct, X⁴ is C₁₋₁₀alkylene and R⁸ is as defined in claim 35; or
(g) at a site indicated by R³, R⁴, R⁵, R⁶ or R⁷ in the amatoxin analog as defined in any one of claims 2 to 34, the compound-linker construct comprises a group of the formula: 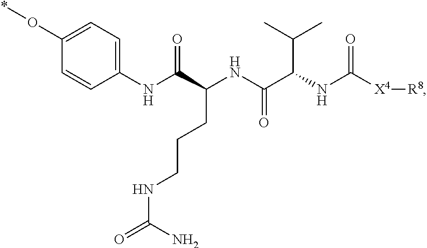
wherein * represents the site of attachment to the remainder of the compound-linker construct, X⁴ is C₁₋₁₀alkylene and R⁸ is as defined in claim 35.

45. The compound-linker construct of any one of claims 35 to 38, wherein: at a site indicated by R³, R⁴, R⁵, R⁶ or R⁷ in the amatoxin analog as defined in any one of claims 2 to 34, the compound-linker construct comprises a group of the formula: 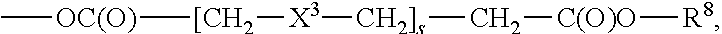
wherein X³ is a bond, O, CH₂ or S, s is an integer from 1 to 8 and R⁸ is as defined in claim 35.

46. The compound-linker construct of any one of claims 35 to 45, wherein R⁸ is selected from: 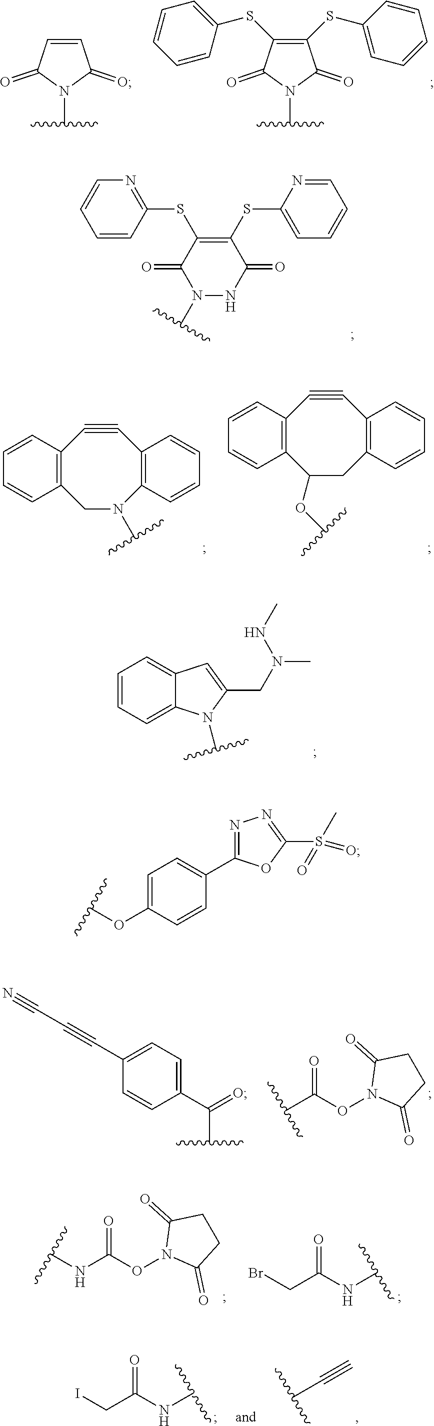
wherein [symbol] represents the site of attachment of R⁸ to the remainder of the linker; or R⁸ comprises an azide, thiol, tetrazine, trans-cyclooctene or acrylamide functional group.

47. The compound-linker construct of claim 46, wherein R⁸ is

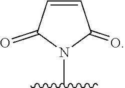 [SMILES: *N1C(=O)C=CC1=O]

48. The compound-linker construct of claim 35 or 45, wherein at a site indicated by R⁶ in the amatoxin analog as defined in any one of claims 2 to 34, the compound-linker construct comprises a group of the formula: 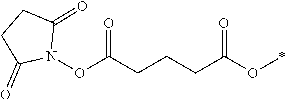 [SMILES: *OC(=O)CCCC(=O)ON1C(=O)CCC1=O]
wherein * represents the site of attachment to the remainder of the compound-linker construct.

49. A conjugate comprising a target-binding moiety conjugated to an amatoxin analog of any one of claims 1 to 34 or a compound-linker construct of any one of claims 35 to 48.

50. The conjugate of claim 49, wherein the conjugate comprises the compound-linker construct, the target-binding moiety comprises an engineered acceptor residue, R⁸ is a reactive group to a moiety comprised in the engineered acceptor residue, and the compound-linker construct is conjugated to the target-binding moiety via a moiety resulting from the reaction of the moiety comprised in the engineered acceptor residue with R⁸.

51. The conjugate of claim 50, wherein the engineered acceptor residue is an engineered cystine residue and R⁸ is a thiol-reactive group.

52. The conjugate of claim 49, wherein the target-binding moiety comprises a biorthogonal functional group, R⁸ is a complementary biorthogonal functional group, and the compound-linker construct is conjugated to the target-binding moiety via a moiety resulting from the reaction of the biorthogonal functional group on the target-binding moiety with R⁸.

53. The conjugate of claim 49, wherein the target-binding moiety comprises a primary amino group, R⁸ is a reactive group to the primary amino group, and the compound-linker construct is conjugated to the target-binding moiety via a moiety resulting from the reaction of the primary amino group with R⁸.

54. The conjugate of claim 53, wherein the primary amino group is a lysine side chain.

55. The conjugate of any one of claims 49 to 54, wherein the target-binding moiety is a peptide, an oligonucleotide, a lipid, a lipid carrier, a nanoparticle or combinations thereof.

56. The conjugate of any one of claims 49 to 55, wherein the target-binding moiety is an antibody, an antigen-binding fragment thereof, an antibody-like protein, an aptamer, a vitamin, an oligonucleotide, an affibody, a minibody, a lipid, a lipid carrier, a nanoparticle or combinations thereof.

57. The conjugate of claim 56, wherein the target-binding moiety is an antibody.

58. A pharmaceutical composition comprising the amatoxin analog of any one of claims 1 to 34, or the conjugate of any one of claims 49 to 57 and a pharmaceutically acceptable carrier.

59. A use of an effective amount of an amatoxin analog of any one of claims 1 to 34, or a conjugate of any one of claims 49 to 57 for treatment of a disease associated with cells presenting a target in a subject in need thereof, wherein the target-binding moiety is specific for the target.

60. A use of an effective amount of an amatoxin analog of any one of claims 1 to 34, or a conjugate of any one of claims 49 to 57 for preparation of a medicament for treatment of a disease associated with cells presenting a target in a subject in need thereof, wherein the target-binding moiety is specific for the target.

61. The use of claim 59 or 60, wherein the disease associated with cells presenting a target is cancer, an autoimmune disease or a viral infection.

62. A use of an effective amount of an amatoxin analog of any one of claims 1 to 34, or a conjugate of any one of claims 49 to 57 for treatment of cancer in a subject in need thereof.

63. A use of an effective amount of an amatoxin analog of any one of claims 1 to 34, or a conjugate of any one of claims 49 to 57 for preparation of a medicament for treatment of cancer in a subject in need thereof.

64. The use of claim 62 or 63, wherein the cancer is breast cancer.

65. An amatoxin analog of any one of claims 1 to 34, or a conjugate of any one of claims 49 to 57 for use to treat a disease associated with cells presenting a target in a subject, wherein the target-binding moiety is specific for the target.

66. The amatoxin analog for use of claim 65, wherein the disease associated with cells presenting a target is cancer, an autoimmune disease or a viral infection.

67. An amatoxin analog of any one of claims 1 to 34, or a conjugate of any one of claims 49 to 57 for use to treat cancer in a subject.

68. The amatoxin analog or conjugate for use of claim 67, wherein the cancer is breast cancer.

69. A method for controlling pests, the method comprising contacting a pest with an amatoxin analog of any one of claims 1 to 34 or a conjugate of any one of claims 49 to 57.
